(19) United States
(12) Patent Application Publication
(10) Pub. No.: US 2022/0290114 A1
(43) Pub. Date: Sep. 15, 2022

(54) ANTIMICROBIAL ENDOLYSIN POLYPEPTIDES, COMPOSITIONS AND FORMULATIONS

(71) Applicant: AXITAN LIMITED, London (GB)

(72) Inventors: Weiluo LEE, London (GB); Kane MILLER, London (GB); Francisco J. NAVARRO, London (GB)

(21) Appl. No.: 17/427,453

(22) PCT Filed: Jan. 31, 2020
(86) PCT No.: PCT/GB2020/050237

§ 371 Date: Jul. 30, 2021

(30) Foreign Application Priority Data
Jan. 31, 2019 (GB) 1901364.8

(51) Int. Cl. C12N 9/36; A23K 10/12; A23K 20/189; A23K 50/30; A23K 50/50; A23K 50/75

(52) U.S. Cl. CPC C12N 9/2462; A23K 10/12; A23K 20/189; A23K 50/30; A23K 50/50; A23K 50/75; C12N 2330/50

(57) ABSTRACT

The present invention relates to novel endolysin polypeptides or fragments thereof which possess antimicrobial activity, preferably antibacterial activity against Clostridium perfringens. The invention also relates to a nucleic acid molecule encoding the endolysin polypeptide or fragment, a recombinant polynucleotide expression vector comprising such a nucleic acid molecule, as well as a host cell comprising such a nucleic acid molecule or comprising such a recombinant polynucleotide expression vector. The invention also relates to compositions and foodstuffs comprising the endolysin polypeptides or fragments and uses thereof in the treatment of diseases or disorders in animals.

### FIELD OF THE INVENTION

The present invention relates to novel endolysin polypeptides or fragments thereof which possess antimicrobial activity, preferably antibacterial activity against Clostridium perfringens. The invention also relates to a nucleic acid molecule encoding the endolysin polypeptide or fragment, a recombinant polynucleotide expression vector comprising such a nucleic acid molecule, as well as a host cell comprising such a nucleic acid molecule or comprising such a recombinant polynucleotide expression vector. The invention also relates to compositions and foodstuffs comprising the endolysin polypeptides or fragments and uses thereof in the treatment of diseases or disorders in animals.

### BACKGROUND TO THE INVENTION

Concerns over antibiotic resistance have resulted in public and regulatory pressure to ensure their judicious use, in both human and veterinary medicine. In food-animal production, the prevention of animal diseases caused in particular by Clostridium perfringens has become a far greater problem. In order to achieve the same high level of food-animal production, it is imperative that alternative antimicrobials are developed. Reagents developed specifically for the relevant microbial species of concern would function as effective tools for controlling economically important diseases, and therefore are ideal candidates for therapeutic treatments.

Several novel bacteriophage-derived endolysins with lytic activity against C. perfringens Type A have been identified and characterised. Synergistic effects between certain endolysins that increase both the antimicrobial activity on a single strain and across a broad spectrum of C. perfringens Type A strains have also been elucidated.

Clostridium perfringens is a Gram-positive, rod-shaped, anaerobic, spore-forming bacterium of the genus Clostridium. It is typically found in decaying vegetation, marine sediment, the intestinal tract of humans and other vertebrates, insects, and soil. Although ubiquitous and often benign, C. perfringens is also the cause of many severe infections of animals and humans. Indeed, C. perfringens is known to be the cause of food poisoning, gas gangrene (clostridial myonecrosis), necrotic enteritis, and non-foodborne gastrointestinal infections. C. perfringens strains are classified into five toxin types based on the type of toxin expression: A, B, C, D and E.

C. perfringens type A is a particular concern for the poultry industry. The presence of C. perfringens type A in the intestinal tract of poultry, in particular broiler chickens, has been linked with various conditions, such as gut lesions and necrotic enteritis, and can result in a significant reduction in the growth of poultry. Antibiotics such as bacitracin, virginiamycin, and penicillin in addition to ionophores like monensin and salinomycin, amongst others, have been used to mitigate clinical and sub-clinical necrotic enteritis in poultry. However, the increase in resistance in recent years has significantly reduced the reliability of these antibiotics. Of particular concern is the increase in prevalence and range of multi-drug resistant strains. In addition to the increasing occurrence of resistance against conventional antibiotics, most antibiotics have a broad-spectrum activity against a range of bacterial groups. Therefore, antibiotic treatments not only affect the pathogen bacteria but also detrimentally affect the beneficial and protective bacterial flora in the chicken gut. As such, it would be highly desirable to develop narrow-spectrum antimicrobials that specifically kill C. perfringens, particularly C. perfringens type A, with little or no collateral effects on the beneficial bacterial flora.

Bacteriophage-encoded endolysins can be employed as antimicrobial agents that are specific to their host species of bacteria. Bacteriophages are viruses that infect bacteria. The expansion of understanding on bacteriophage biology, including the increase in antibiotic resistant strains of bacteria, has led to increased interest in these specific bacteriolytic enzymes. Endolysins are used by bacteriophage to degrade the bacterial cell wall peptidoglycans from within the bacteria in order to facilitate the release of bacteriophage progeny. Studies have shown that the endolysins can also have antimicrobial effects when applied externally to Gram-positive bacteria. Endolysins that are specific for Gram-positive bacteria usually have a modular organisation. Many of these endolysins have a two-domain structure with an N-terminal catalytic domain and a C-terminal cell wall binding domain connected by a linker. Endolysins with different domain architectures have also been discovered.

The N-terminal catalytic domain of the endolysin is responsible for the enzymatic cleavage of the peptidoglycan layer of the cell wall. The peptidoglycan layer consists of linear carbohydrate backbones with alternating (β1-4)-linked N-acetylglucosamine and N-acetylmuramic acid monomers. The carbohydrate backbones are cross-linked by species-specific peptide side chains. Depending on the type of bond that they cleave, endolysins can be N-acetyl-β-D-glucosaminidases or N-acetyl-β-D-muramidase. Alternatively, some endolysins have endopeptidase activity, cleaving a specific bond of the peptide side chain. Finally, a fourth type of endolysins are N-acetylmuramoyl-L-alanine amidases, which hydrolyse the amide bond between the sugar strand and the peptide chain.

The C-terminal cell wall binding domain is largely responsible for the specificity of the endolysin against its target species or genera. The domain binds noncovalently to the cell envelope, which can be part of the peptidoglycan or other cell wall associated molecules. The binding to the cell wall is tight and irreversible, which also minimizes the endolysins from attacking the surrounding cells of the lysed bacterium. In addition, the catalytic domains also have an influence on the specificity of an endolysin. The peptidoglycan structures differ between Gram-types and also between different bacterial species. Therefore, the absence or presence of a specific target bond will contribute to the specificity of endolysins.

Due to their specificity and less likely emergence of resistant strains, endolysins can be advantageous antimicrobials against C. perfringens, particularly C. perfringens type A, and they may become an effective way to control the occurrence of diseases in animals, such as necrotic enteritis in poultry. To this aim, the inventors have identified and characterised several novel bacteriophage-derived endolysins with high specificity and bacteriolytic activity against a broad spectrum of C. perfringens strains, such as Type A strains. Synergistic effects between the identified endolysins were also found, suggesting that the use of a cocktail of endolysins can potentiate their antimicrobial efficacy.

### SUMMARY OF THE INVENTION

The Inventors have identified endolysin polypeptides, which possess surprisingly advantageous functional antimicrobial activities, including as described and defined herein according to SEQ ID numbers. Accordingly, the invention encompasses any of such specific endolysin polypeptides, as well as any polypeptide comprising a fragment or variant of such an endolysin polypeptide, or any polypeptide which comprises any one or more fragments of any such endolysin polypeptide(s) as well as any polypeptide which comprises any one or more sequence variant(s) thereof, provided that any such endolysin polypeptide retains any of the antimicrobial activities as described and defined herein.

In addition to the specific endolysin polypeptides disclosed herein and defined herein according to SEQ ID numbers, the invention also provides any endolysin polypeptide and any polypeptide comprising a fragment or variant of such an endolysin polypeptide, or any polypeptide which comprises any one or more fragments of any such endolysin polypeptide as well as any one or more sequence variant(s) thereof, provided that any such polypeptide possesses any one or more of the surprising antimicrobial activities as described and defined herein. Any such endolysin polypeptide may therefore be regarded as functionally equivalent to any specific polypeptide disclosed herein and defined herein according to SEQ ID number.

The inventors have surprisingly determined that multiple endolysin polypeptides of the invention used together, such as two or more endolysin polypeptides of the invention used together, may possess synergistic effects.

The inventors have surprisingly determined that cell extract, including algal cell extract, can enhance the functional properties of endolysin polypeptides of the invention. Accordingly, endolysin polypeptides of the invention expressed in cells, such as algal cells, may possess enhanced functional properties, and endolysin polypeptides of the invention mixed with cell extract, such as algal cell extract, may possess enhanced functional properties.

An endolysin polypeptide of the invention is an isolated endolysin polypeptide which has antimicrobial activity, and wherein the isolated polypeptide comprises or consists of an amino acid sequence:

which is an N-terminal Clostridium perfringens cell wall peptidoglycan catalytic domain polypeptide,

or which is a fragment of said N-terminal catalytic domain polypeptide; or

which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11,

or which is a fragment of said N-terminal catalytic domain polypeptide, and which is at least 80% identical to the amino acid sequence of the sequence set forth in SEQ ID NOs: 1, 2, 3, 4, 5, 6, 7, 8 or 11 which corresponds to the amino acid sequence of the fragment.

The isolated endolysin polypeptide may comprise or consist of an amino acid sequence which is an N-terminal Clostridium perfringens cell wall peptidoglycan catalytic domain and which is at least 80%, 85%, 90%, 91%, 92%, 93%, 94%, 95%, 96%, 97%, 98%, 99% or 100% identical to the amino acid sequence of the C. perfringens bacteriophage endolysin polypeptide set forth in SEQ ID NOs: 1, 2, 3, 4, 5, 6, 7, 8 or 11.

In the case of a fragment of said amino acid sequence, the endolysin polypeptide may comprise or consist of an amino acid sequence which is at least 80%, 85%, 90%, 91%, 92%, 93%, 94%, 95%, 96%, 97%, 98%, 99% or 100% identical to the amino acid sequence of the sequence set forth in SEQ ID NOs: 1, 2, 3, 4, 5, 6, 7, 8 or 11 which corresponds to the amino acid sequence of the fragment.

The N-terminal Clostridium perfringens cell wall peptidoglycan catalytic domains of the endolysin polypeptides defined according to the amino acid sequences set forth in SEQ ID NOs: 1, 2, 3, 4, 5, 6, 7 8 or 11 are represented respectively by the amino acid sequence set forth in SEQ ID NOs: 12, 13, 14, 15, 16, 17, 19 and 20 XX. In addition, the N-terminal Clostridium perfringens cell wall peptidoglycan catalytic domain of the endolysin polypeptide defined according to the amino acid sequences set forth in SEQ ID NO 6 may be represented by the amino acid sequence set forth in SEQ ID NO: 18.

Any of the endolysin polypeptides defined above may have any antimicrobial activity as defined herein.

An endolysin polypeptide of the invention may be an isolated endolysin polypeptide which has antimicrobial activity as defined above, further wherein the isolated polypeptide comprises an N-terminal Clostridium perfringens cell wall peptidoglycan catalytic domain polypeptide or fragment thereof, a C-terminal Clostridium perfringens cell wall binding domain polypeptide or fragment thereof and optionally a linker polypeptide or fragment thereof connecting the N- and C-terminal domains; wherein the isolated polypeptide comprises:
a) an amino acid sequence which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11, or an amino acid sequence which is a fragment of said N-terminal catalytic domain polypeptide and which is at least 80% identical to the amino acid sequence of the sequence set forth in SEQ ID NOs: 1, 2, 3, 4, 5, 6, 7, 8 or 11 which corresponds to the amino acid sequence of the fragment; and
b) an amino acid sequence which is at least 80% identical to an endolysin C-terminal Clostridium perfringens cell wall binding domain polypeptide, or a fragment of said polypeptide; or which is an amino acid sequence which is at least 80% identical to the amino acid sequence of the C-terminal cell wall binding domain of the Clostridium perfringens bacteriophage endolysin polypeptide set forth in SEQ ID NO: 1, 2, 3, 5, 6, or 8, or an amino acid sequence which is a fragment of said C-terminal binding domain polypeptide and which is at least 80% identical to the amino acid sequence of the sequence set forth in SEQ ID NOs: 1, 2, 3, 5, 6, or 8 which corresponds to the amino acid sequence of the fragment; and
c) an optional linker amino acid sequence which is at least 80% identical to the linker of an endolysin polypeptide, or a fragment of said polypeptide; or an optional amino acid sequence which is at least 80% identical to the amino acid sequence of a linker of the Clostridium perfringens bacteriophage endolysin polypeptide set forth in SEQ ID NO: 1, 2, 3, 5, 6, or 8, or an amino acid sequence which is a fragment of said linker and which is at least 80% identical to the amino acid sequence of the sequence set forth in SEQ ID NOs: 1, 2, 3, 5, 6, or 8, which corresponds to the amino acid sequence of the fragment; wherein the linker amino acid sequence connects the amino acid sequence of the N-terminal cell wall peptidoglycan catalytic domain of the Clostridium perfringens bacteriophage endolysin polypeptide or fragment thereof and the C-terminal cell wall binding domain of the Clostridium perfringens bacteriophage endolysin polypeptide or fragment thereof.

An endolysin polypeptide of the invention may be an isolated endolysin polypeptide which has antimicrobial activity as defined above, wherein the isolated polypeptide comprises an amino acid sequence comprising or consisting of an N-terminal Clostridium perfringens cell wall peptidoglycan catalytic domain polypeptide or fragment thereof, an amino acid sequence comprising or consisting of a C-terminal Clostridium perfringens cell wall binding domain polypeptide or fragment thereof and an amino acid sequence comprising or consisting of a linker polypeptide or fragment thereof connecting the N- and C-terminal domains; wherein the isolated polypeptide comprises:
a) an amino acid sequence which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11, or an amino acid sequence which is a fragment of said N-terminal catalytic domain polypeptide and which is at least 80% identical to the amino acid sequence of the sequence set forth in SEQ ID NOs: 1, 2, 3, 4, 5, 6, 7, 8 or 11 which corresponds to the amino acid sequence of the fragment; and
b) an amino acid sequence which is at least 80% identical to the amino acid sequence of the C-terminal Clostridium perfringens cell wall binding domain of the Clostridium perfringens bacteriophage endolysin polypeptide set forth in SEQ ID NO: 1, 2, 3, 5, 6, or 8, or an amino acid sequence which is a fragment of said C-terminal binding domain polypeptide and which is at least 80% identical to the amino acid sequence of the sequence set forth in SEQ ID NOs: 1, 2, 3, 5, 6, or 8 which corresponds to the amino acid sequence of the fragment; and
c) an amino acid sequence which is at least 80% identical to the amino acid sequence of a linker of the Clostridium perfringens bacteriophage endolysin polypeptide set forth in SEQ ID NO: 1, 2, 3, 5, 6, or 8, or an amino acid sequence which is a fragment of said linker and which is at least 80% identical to the amino acid sequence of the sequence set forth in SEQ ID NOs: 1, 2, 3, 5, 6, or 8 which corresponds to the amino acid sequence of the fragment.

An endolysin polypeptide of the invention may be an isolated endolysin polypeptide which has antimicrobial activity as defined above, wherein the isolated polypeptide consists of or comprises:
a) an amino acid sequence consisting of or comprising a fragment of said N-terminal catalytic domain and which is at least 80% identical to the amino acid sequence of the sequence set forth in SEQ ID NOs: 1, 2, 3, 4, 5, 6, 7, 8 or 11 which corresponds to the amino acid sequence of the fragment; and/or
b) an amino acid sequence consisting of or comprising a fragment of said C-terminal binding domain and which is at least 80% identical to the amino acid sequence of the sequence set forth in SEQ ID NOs: SEQ ID NO: 1, 2, 3, 5, 6, or 8 which corresponds to the amino acid sequence of the fragment; and/or
c) an amino acid sequence consisting of or comprising a fragment of said linker and which is at least 80% identical to the amino acid sequence of the sequence set forth in SEQ ID NOs: 1, 2, 3, 5, 6, or 8 which corresponds to the amino acid sequence of the fragment.

In any of the endolysin polypeptides of the invention described above which possess an N-terminal Clostridium perfringens cell wall peptidoglycan catalytic domain or an amino acid sequence which is fragment thereof, and a C-terminal Clostridium perfringens cell wall binding domain or an amino acid sequence which is a fragment thereof, and optionally a linker amino acid sequence or an amino acid sequence which is a fragment thereof, the percentage identity to the reference sequence may be 80% or higher. Accordingly, in any such isolated endolysin polypeptide, the endolysin polypeptide may comprise or consist of an amino acid sequence which is a N-terminal Clostridium perfringens cell wall peptidoglycan catalytic domain and which is at least 80%, 85%, 90%, 91%, 92%, 93%, 94%, 95%, 96%, 97%, 98%, 99% or 100% identical to the amino acid sequence of a known endolysin a N-terminal Clostridium perfringens cell wall peptidoglycan catalytic domain polypeptide. In the case of a fragment of said amino acid sequence, the endolysin polypeptide may comprise or consist of an amino acid sequence which is at least 80%, 85%, 90%, 91%, 92%, 93%, 94%, 95%, 96%, 97%, 98%, 99% or 100% identical to the amino acid sequence of the sequence of the known endolysin N-terminal Clostridium perfringens cell wall peptidoglycan catalytic domain polypeptide which corresponds to the amino acid sequence of the fragment. In any such isolated endolysin polypeptide, the endolysin polypeptide may comprise or consist of an amino acid sequence which is an N-terminal Clostridium perfringens cell wall peptidoglycan catalytic domain and which is at least 80%, 85%, 90%, 91%, 92%, 93%, 94%, 95%, 96%, 97%, 98%, 99% or 100% identical to the amino acid sequence of the C. perfringens bacteriophage endolysin polypeptide set forth in SEQ ID NOs: 1, 2, 3, 4, 5, 6, 7, 8 or 11. In the case of an amino acid sequence which is a fragment of said amino acid sequence, the endolysin polypeptide may comprise or consist of an amino acid sequence which is at least 80%, 85%, 90%, 91%, 92%, 93%, 94%, 95%, 96%, 97%, 98%, 99% or 100% identical to the amino acid sequence of the sequence set forth in SEQ ID NOs: 1, 2, 3, 4, 5, 6, 7, 8 or 11 which corresponds to the amino acid sequence of the fragment. In any such isolated endolysin polypeptide, the endolysin polypeptide may comprise or consist of an amino acid sequence which is a C-terminal Clostridium perfringens cell wall binding domain and which is at least 80%, 85%, 90%, 91%, 92%, 93%, 94%, 95%, 96%, 97%, 98%, 99% or 100% identical to the amino acid sequence of a known endolysin C-terminal Clostridium perfringens cell wall binding domain polypeptide. In the case of a fragment of said amino acid sequence, the endolysin polypeptide may comprise or consist of an amino acid sequence which is at least 80%, 85%, 90%, 91%, 92%, 93%, 94%, 95%, 96%, 97%, 98%, 99% or 100% identical to the amino acid sequence of the sequence of the known endolysin C-terminal Clostridium perfringens cell wall binding domain polypeptide which corresponds to the amino acid sequence of the fragment. Alternatively, in any such isolated endolysin polypeptide, the endolysin polypeptide may comprise or consist of an amino acid sequence which is a C-terminal Clostridium perfringens cell wall binding domain and which is at least 80%, 85%, 90%, 91%, 92%, 93%, 94%, 95%, 96%, 97%, 98%, 99% or 100% identical to the amino acid sequence of a C. perfringens bacteriophage endolysin polypeptide set forth in SEQ ID NOs: 1, 2, 3, 5, 6, or 8. In the case of a fragment of said amino acid sequence, the endolysin polypeptide may comprise or consist of an amino acid sequence which is at least 80%, 85%, 90%, 91%, 92%, 93%, 94%, 95%, 96%, 97%, 98%, 99% or 100% identical to the amino acid sequence of the sequence set forth in SEQ ID NOs: 1, 2, 3, 5, 6, or 8 which corresponds to the amino acid sequence of the fragment. In any such isolated endolysin polypeptide, the endolysin polypeptide may optionally comprise or consist of an amino acid sequence which is at least 80%, 85%, 90%, 91%, 92%, 93%, 94%, 95%, 96%, 97%, 98%, 99% or 100% identical to the amino acid sequence of a linker of a known endolysin polypeptide. In the case of a fragment of said amino acid sequence, the endolysin polypeptide may comprise or consist of an amino acid sequence which is at least 80%, 85%, 90%, 91%, 92%, 93%, 94%, 95%, 96%, 97%, 98%, 99% or 100% identical to the amino acid sequence of a linker of a known endolysin polypeptide which corresponds to the amino acid sequence of the fragment. Alternatively, in any such isolated endolysin polypeptide, the endolysin polypeptide may comprise or consist of an amino acid sequence which is at least 80%, 85%, 90%, 91%, 92%, 93%, 94%, 95%, 96%, 97%, 98%, 99% or 100% identical to the amino acid sequence of the linker of the Clostridium perfringens bacteriophage endolysin polypeptide set forth in SEQ ID NO: 1, 2, 3, 5, 6, or 8. In the case of a fragment of said amino acid sequence, the endolysin polypeptide may comprise or consist of an amino acid sequence which is at least 80%, 85%, 90%, 91%, 92%, 93%, 94%, 95%, 96%, 97%, 98%, 99% or 100% identical to the amino acid sequence of the sequence set forth in SEQ ID NOs: 1, 2, 3, 5, 6, or 8 which corresponds to the amino acid sequence of the fragment.

An endolysin polypeptide of the invention may be an isolated endolysin polypeptide which has antimicrobial activity as defined above, wherein the isolated polypeptide comprises: an amino acid sequence wherein the N-terminal catalytic domain, the C-terminal cell wall binding domain and the linker are all at least 80%, 85%, 90%, 91%, 92%, 93%, 94%, 95%, 96%, 97%, 98%, 99% or 100% identical to corresponding amino acid sequences respectively of the N-terminal domain, a C-terminal domain and a linker of the Clostridium perfringens bacteriophage endolysin polypeptide set forth in SEQ ID NOs: 1, 2, 3, 4, 5, 6, 7, 8 or 11.

Any of the endolysin polypeptides defined above may have any antimicrobial activity as defined herein.

An endolysin polypeptide of the invention may be an isolated endolysin polypeptide which has antimicrobial activity as defined above, wherein the polypeptide comprises or consists of:
a) an amino acid sequence comprising or consisting of all of the N-terminal domain, the C-terminal domain(s) and the linker(s) of the Clostridium perfringens bacteriophage endolysin polypeptide set forth in SEQ ID NOs: SEQ ID NO: 1, 2, 3, 6, or 8, and wherein the amino acid sequences are 100% identical to the sequences of the N-terminal domain, the C-terminal domain and the linker of the Clostridium perfringens bacteriophage endolysin polypeptide set forth in SEQ ID NOs: SEQ ID NO: 1, 2, 3, 5, 6, or 8; or
b) an amino acid sequence comprising or consisting of fragments of all of the N-terminal domain, the C-terminal domain and the linker of the Clostridium perfringens bacteriophage endolysin polypeptide set forth in SEQ ID NOs: 1, 2, 3, 5, 6, or 8, and wherein the amino acid sequences of each fragment are 100% identical to the sequences of the Clostridium perfringens bacteriophage endolysin polypeptide set forth in SEQ ID NOs: SEQ ID NO: 1, 2, 3, 5, 6, or 8 which correspond to the amino acid sequences of the fragment.

An endolysin polypeptide of the invention may be an isolated endolysin polypeptide which has antimicrobial activity as defined above, wherein the polypeptide comprises or consists of an amino acid sequence which is 80%, 85%, 90%, 91%, 92%, 93%, 94%, 95%, 96%, 97%, 98%, 99% or 100% identical to the amino acid sequence of the Clostridium perfringens bacteriophage endolysin polypeptide set forth in SEQ ID NO: 1, 2, 3, 4, 5, 6, 7 8 or 11.

An endolysin polypeptide of the invention may be an isolated endolysin polypeptide which has antimicrobial activity as defined above, wherein the polypeptide comprises or consists of an amino acid sequence which is 100% identical to the amino acid sequence of the Clostridium perfringens bacteriophage endolysin polypeptide set forth in SEQ ID NO: 1, 2, 3, 4, 5, 6, 7, 8 or 11.

An endolysin polypeptide of the invention may be an isolated endolysin polypeptide or fragment which has antimicrobial activity as defined above, wherein the antimicrobial activity is bacteriolytic activity and/or bacterial growth inhibitory activity.

An endolysin polypeptide of the invention may be an isolated endolysin polypeptide or fragment which has antimicrobial activity as defined above, wherein the isolated polypeptide or fragment has bacteriolytic activity and/or bacterial growth inhibitory activity against, Clostridium perfringens.

An endolysin polypeptide of the invention may be an isolated endolysin polypeptide or fragment which has antimicrobial activity as defined above, wherein the isolated polypeptide or fragment has bacteriolytic activity and/or bacterial growth inhibitory activity against Clostridium perfringens, preferably a Type A strain of Clostridium perfringens. Alternatively, or in addition, when any such polypeptide or fragment is tested in a cell viability assay, as described herein, at a concentration of 5 μg/ml against Clostridium perfringens strain NCTC8237 it may exhibit a Δ Log 10 value of approximately 0.40 or more, 2.00 or more or 3.70 or more. Alternatively, or in addition, when any such polypeptide or fragment is tested in a cell viability assay, as described herein, at a concentration of 5 μg/ml against Clostridium perfringens strain NCTC8237 it may exhibit a % reduction in cell viability of approximately 60% or more, 70% or more, 80% or more, 90% or more or 100%. Alternatively, or in addition, when any such polypeptide or fragment is tested in a cell turbidity reduction assay, as described herein, at a concentration of 5 μg/mL against Clostridium perfringens strain NCTC8237 it may exhibit a t50% lysis value of 7 minutes or less; or it exhibits a t50% lysis value of 3 minutes or less. Alternatively, or in addition, when any such polypeptide or fragment is tested in a minimum inhibitory concentration (MIC)/minimum bactericidal concentration (MBC) assay, as described herein, against Clostridium perfringens strain Cp6 at 2.1×10⁴ cells/mL it exhibits an MIC and/or an MBC value of 17 μg/mL or less, preferably 1.7 μg/mL or less; or it exhibits an MIC and/or an MBC value of 0.65 μM or less, preferably 0.05 μM or less.

Alternatively, or in addition, any such polypeptide or fragment may be tested for endolysin activity (i.e. hydrolytic activity), and therefore antimicrobial activity, by assessing the rate of degradation of purified peptidoglycan (PGN) in vitro, wherein the endolysin polypeptide or fragment may be determined to possess endolysin hydrolytic activity if it promotes a statistically significant increase in the rate of degradation of purified PGN in vitro. The rate of degradation of purified PGN in vitro may be assessed by reference to the background rate of PGN degradation, i.e. by reference to a control preparation which does not contain an endolysin polypeptide. The rate of degradation of purified PGN in vitro may be assessed by reference to the rate of PGN degradation by a reference or control polypeptide, wherein a reference or control polypeptide is a polypeptide which is not capable of promoting a statistically significant increase in the rate of degradation of purified PGN in vitro, such as a polypeptide which is a PGN-catalytically inactive endolysin, or any polypeptide which is not an endolysin, such as bovine serum albumin or lysozyme.

Alternatively, or in addition, any such polypeptide or fragment may be tested for endolysin activity (i.e. hydrolytic activity), and therefore antimicrobial activity, by assessing whether it promotes a statistically significant reduction in the absolute amount of purified PGN in vitro at the end-point of a PGN degradation assay. The amount of degradation (reduction in amount) of purified PGN in vitro at the end-point of a PGN degradation assay may be assessed by reference to the background amount of PGN degradation, i.e. by reference to a control preparation which does not contain an endolysin polypeptide. The amount of degradation (reduction in amount) of purified PGN in vitro at the end-point of a PGN degradation assay may be assessed by reference to the amount of PGN degradation by a reference or control polypeptide, wherein a reference or control polypeptide is a polypeptide which is not capable of promoting a statistically significant reduction in the amount of purified PGN in vitro, such as a polypeptide which is a PGN-catalytically inactive endolysin, or any polypeptide which is not an endolysin, such as bovine serum albumin or lysozyme.

The invention provides an isolated recombinant nucleic acid molecule comprising a first nucleic acid sequence encoding any endolysin polypeptide or fragment of the invention, optionally wherein the isolated recombinant nucleic acid molecule also comprises a second nucleic acid sequence encoding a promoter and wherein the first nucleic acid sequence is operably linked to the second nucleic acid sequence; optionally wherein the first nucleic acid sequence of the isolated recombinant nucleic acid molecule encodes a cDNA. The invention provides an isolated recombinant polynucleotide expression vector comprising the said nucleic acid molecule and comprising the first and second nucleic acid sequences. In any such isolated recombinant nucleic acid molecule comprising the first and second nucleic acid sequences or in any such isolated recombinant polynucleotide expression vector comprising the first and second nucleic acid sequences, the promoter may capable of promoting expression of the endolysin polypeptide or fragment which has antimicrobial activity in a cell of a unicellular microorganism, optionally a yeast cell, a bacterial cell such as Lactobacillus, a fungal cell, or an algal cell, preferably an algal cell. The nucleic acid sequence encoding the endolysin polypeptide or fragment may be codon optimised for expression in a host cell, optionally wherein the host cell is a cell of a unicellular microorganism, optionally a yeast cell, a bacterial cell such as Lactobacillus, a fungal cell, or an algal cell, preferably an algal cell.

The invention also provides a host cell comprising a population of nucleic acid molecules as defined above and encoding an endolysin polypeptide or fragment as defined above and/or a population of recombinant polynucleotide expression vectors as defined above encoding an endolysin polypeptide or fragment as defined above, optionally wherein the host cell additionally comprises an endolysin polypeptide or fragment as defined above and which has been expressed from said molecules or vectors, optionally wherein the host cell is a cell of a unicellular microorganism, optionally a yeast cell, a bacterial cell such as Lactobacillus, a fungal cell, or an algal cell, preferably an algal cell, or wherein the host cell is a cell of a plant. In any such host cell, each one of the molecules or vectors of the population may encode the same endolysin polypeptide or fragment as defined above, or molecules or vectors of the population may encode two or more different endolysin polypeptides or fragments as defined above, optionally wherein the host cell additionally comprises said endolysin polypeptides or fragments and which have been expressed from said molecules or vectors, optionally wherein the host cell is a cell of a unicellular microorganism, optionally a yeast cell, a bacterial cell such as Lactobacillus, a fungal cell, or an algal cell, preferably an algal cell, or wherein the host cell is a cell of a plant.

The invention also provides a cell lysate comprising a population of endolysin polypeptides or fragments as defined above and which have been expressed from a nucleic acid molecule as defined above and/or a recombinant polynucleotide expression vector as defined above, optionally wherein the population consists of the same endolysin polypeptide or fragment as defined above, or wherein the population consists of two or more different endolysin polypeptides or fragments as defined above, optionally wherein the lysate is produced following expression of the endolysin polypeptides or fragments in a host cell which is a cell of a unicellular microorganism, optionally a yeast cell, a bacterial cell such as Lactobacillus, a fungal cell, or an algal cell preferably an algal cell, or wherein the host cell is a cell of a plant.

The invention also provides a composition comprising a population of endolysin polypeptides or fragments which have antimicrobial activity as defined above, optionally wherein the population consists of the same endolysin polypeptide or fragment as defined above, or wherein the population consists of two or more different endolysin polypeptides or fragments thereof as defined above. In any such composition, the population of endolysin polypeptides or fragments which have antimicrobial activity may be: (a) added to the composition as purified polypeptides or fragments; or (b) added to the composition as components of one or more cell extracts; or (c) added to the composition as purified polypeptides or fragments and as components of one or more cell extracts; optionally wherein in (b) or (c) the cell extract is an extract of a unicellular microorganism, optionally a yeast cell, a bacterial cell such as Lactobacillus, a fungal cell, or an algal cell, preferably an algal cell, or wherein the cell is a cell of a plant. In any such composition comprising a population of host cells, host cells of the population may comprise an endolysin polypeptide or fragment which has antimicrobial activity as defined above expressed within the host cells, wherein the endolysin polypeptide or fragment consists of the same endolysin polypeptide or fragment as defined above, or wherein the endolysin polypeptide or fragment consists of two or more different endolysin polypeptides or fragments thereof as defined above.

The invention also provides a whole-cell composition comprising a population of whole host cells, wherein cells of the population comprise an endolysin polypeptide or fragment which has antimicrobial activity as defined above expressed within the host cells, wherein the endolysin polypeptide or fragment consists of the same endolysin polypeptide or fragment as defined above, or wherein the endolysin polypeptide or fragment consists of two or more different endolysin polypeptides or fragments thereof as defined above. Host cells of the population may be cells of a unicellular microorganism, optionally yeast cells, bacterial cells such as lactobacilli, fungal cells, or algal cells. Host cells of the population are preferably unicellular algal cells, more preferably Chlamydomonas Sp., yet more preferably Chlamydomonas reinhardtii cells.

Any of the above-described compositions or whole-cell compositions may comprise a dried biomass comprising the endolysin polypeptide or fragment which has antimicrobial activity expressed within algal cells, preferably wherein the composition or whole-cell composition is spray-dried.

The invention also provides a dried biomass composition comprising a cell lysate as defined above, and/or comprising a composition and/or whole-cell composition as defined above, wherein the dried biomass composition comprises a cell lysate comprising said endolysin polypeptide(s) or fragment(s) which has antimicrobial activity as defined above and/or host cells comprising said endolysin polypeptide(s) or fragment(s) which has antimicrobial activity as defined above and expressed within the host cells. Host cells of the population are algal cells, preferably unicellular algal cells, more preferably Chlamydomonas Sp., yet more preferably Chlamydomonas reinhardtii cells; preferably wherein the dried biomass composition is spray-dried.

The invention also provides an antimicrobial formulation comprising an endolysin polypeptide or fragment which has antimicrobial activity as defined above and a pharmaceutically acceptable carrier/excipient.

Any such antimicrobial formulation may comprise a population of host cells, wherein the host cells of the population are as defined above, or wherein the formulation comprises a cell lysate as defined above, or wherein the formulation comprises a composition as defined above, or wherein the formulation comprises a dried biomass composition as defined above.

The invention also provides an animal foodstuff comprising: (a) one or more foodstuffs; and (b) an isolated endolysin polypeptide or fragment which has antimicrobial activity as defined above.

The invention also provides an animal foodstuff comprising:
(a) one or more foodstuffs; and
(b) further comprising:
   (i) any host cell as defined above;
   (ii) any cell lysate as defined above;
   (iii) any composition as defined above;
   (iv) any whole cell composition as defined above;
   (v) any dried biomass composition as defined above; and/or
   (vi) any antimicrobial formulation as defined above.

The foodstuff may be suitable for consumption by poultry, optionally a broiler chicken, preferably Gallus gallus domesticus; or wherein the foodstuff is suitable for consumption by a pig, preferably Sus scrofa domesticus; or wherein the foodstuff is suitable for consumption by a rodent, optionally a mouse or rat.

Any of the above described host cells, cell lysates, compositions, whole-cell compositions, dried biomass compositions, antimicrobial formulations, or an animal foodstuffs may comprise two or more different endolysin polypeptides or fragments which have antimicrobial activity as defined above. The exhibited antimicrobial activity provided by the two or more different endolysin polypeptides or fragments may be a synergistic activity.

Any combination of two or more endolysin polypeptides or fragments may be provided. Combinations may comprise: an endolysin polypeptide having an amino acid sequence as set forth in SEQ ID NO: 1 in combination with an endolysin polypeptide having an amino acid sequence as set forth in any one of SEQ ID NOS: 2, 3, 4, 5, 6, 7, 8 or 11; an endolysin polypeptide having an amino acid sequence as set forth in SEQ ID NO: 2 in combination with an endolysin polypeptide having an amino acid sequence as set forth in any one of SEQ ID NOS: 3, 4, 5, 6, 7, 8 or 11; an endolysin polypeptide having an amino acid sequence as set forth in SEQ ID NO: 3 in combination with an endolysin polypeptide having an amino acid sequence as set forth in any one of SEQ ID NOS: 4, 5, 6, 7, 8 or 11; an endolysin polypeptide having an amino acid sequence as set forth in SEQ ID NO: 4 in combination with an endolysin polypeptide having an amino acid sequence as set forth in any one of SEQ ID NOS: 5, 6, 7, 8 or 11; an endolysin polypeptide having an amino acid sequence as set forth in SEQ ID NO: 5 in combination with an endolysin polypeptide having an amino acid sequence as set forth in any one of SEQ ID NOS: 6, 7, 8 or 11; an endolysin polypeptide having an amino acid sequence as set forth in SEQ ID NO: 6 in combination with an endolysin polypeptide having an amino acid sequence as set forth in any one of SEQ ID NOS: 7, 8 or 11; an endolysin polypeptide having an amino acid sequence as set forth in SEQ ID NO: 7 in combination with an endolysin polypeptide having an amino acid sequence as set forth in any one of SEQ ID NOS: 8 or 11; and an endolysin polypeptide having an amino acid sequence as set forth in SEQ ID NO: 8 in combination with an endolysin polypeptide having an amino acid sequence as set forth in SEQ ID NO: 11. It is to be understood that the above-noted combinations may include one or more further endolysin polypeptide. It is also to be understood that the above-noted combinations relate to combinations of fragments of the said endolysin polypeptides and combinations of the full-length endolysin polypeptides fragments of the said endolysin polypeptides.

Any of the above described host cells, cell lysates, compositions, whole-cell compositions, dried biomass compositions, antimicrobial formulations, or an animal foodstuffs may be spray-dried, lyophilized or in powdered form.

In any of the above described nucleic acids, expression vectors, host cells, cell lysates, compositions, whole-cell compositions, dried biomass compositions, antimicrobial formulations, or an animal foodstuffs of the invention, reference to endolysin polypeptide, fragment or variant encompasses combinations of one or more endolysin polypeptides and/or one or more fragments and/or one or more variants.

The invention also provides any of the above described nucleic acids, expression vectors, host cells, cell lysates, compositions, whole-cell compositions, dried biomass compositions, antimicrobial formulations, or an animal foodstuffs of the invention for use as a medicament and for use in the treatment of a bacterial infection in an animal. The bacterial infection may be a Clostridium perfringens infection, optionally an infection caused by a Type A strain of Clostridium perfringens.

The invention also provides any of the above described nucleic acids, expression vectors, host cells, cell lysates, compositions, whole-cell compositions, dried biomass compositions, antimicrobial formulations, or an animal foodstuffs of the invention for use in the treatment of a disease or disorder caused by a Clostridium perfringens infection, optionally an infection caused by a Type A strain of Clostridium perfringens. The disease or disorder caused by the Clostridium perfringens infection may be food poisoning, gas gangrene, necrotic enteritis, a gut lesion and/or a gastrointestinal infection.

The invention provides a method for the prevention or treatment of a disease or disorder in an animal, the method comprising administering to the animal a prophylactically or therapeutically effective amount of any isolated endolysin polypeptide or fragment which has antimicrobial activity disclosed herein, any host cell disclosed herein, any cell lysate disclosed herein, any composition disclosed herein, any whole-cell composition disclosed herein, any dried biomass composition disclosed herein, any antimicrobial formulation disclosed herein, or animal foodstuff disclosed herein and for any specific prevention or treatment of a disease or disorder in an animal disclosed herein. The animal may be a poultry animal, optionally a broiler chicken, preferably Gallus gallus domesticus; or the animal may be a pig, preferably Sus scrofa domesticus.

The invention also provides a method of obtaining any isolated polypeptide or fragment which has antimicrobial activity defined herein comprising:
(a) culturing a host cell comprising any nucleic acid molecule or any recombinant polynucleotide expression vector disclosed herein in a culture system;
(b) harvesting the cultured host cell; and
(c) isolating the polypeptide or fragment from the host cell and/or culture solution.

The invention also provides a method of obtaining a dried biomass composition comprising any isolated endolysin polypeptide or fragment which has antimicrobial activity defined above, the method comprising:
(a) culturing any of the host cells defined above in a culture system under conditions wherein the endolysin polypeptide or fragment which has antimicrobial activity is expressed;
(b) harvesting and isolating the host cells from the culture system;
(c) drying the host cells to form a dried biomass; and
(d) formulating the dried biomass into a composition.

The invention also provides a method of obtaining a dried biomass composition comprising any isolated endolysin polypeptide or fragment which has antimicrobial activity defined above, the method comprising:
(a) culturing any of the host cells defined above in a culture system under conditions wherein the endolysin polypeptide or fragment which has antimicrobial activity is expressed;
(b) creating a lysate from the cultured host cells;
(c) drying the lysate to form a dried biomass; and
(d) formulating the dried biomass into a composition;
   wherein the lysate is created by lysing host cells in situ in the culture system, or wherein the lysate is created by lysing host cells after the host cells have been harvested and isolated from the culture system.

In any such method of obtaining a dried biomass composition, the host cells may be algal cells, preferably unicellular algal cells, more preferably Chlamydomonas Sp., yet more preferably Chlamydomonas reinhardtii cells. In any such method of obtaining a dried biomass composition, the step of drying to form a dried biomass may comprise spray-drying.

### BRIEF DESCRIPTION OF THE FIGURES

FIG. 1: shows expression of a tRNA-Wtca molecule allowed translation of a RFP coding sequence with WTGG-TGA codons.

Cultures of strains transformed with plasmids ecAL002, ecAL003 and ecAL004 after 6 h of addition of IPTG to the media. (A) Cultures illuminated with white light; (B), cultures irradiated with UV. (C) SDS-PAGE gel stained with InstantBlue protein stain of protein extracts from cultures transformed with plasmids ecAL002, ecAL003 and ecAL004. Cells were collected 5 h after IPTG addition to the media, and disrupted by sonication. Total protein extract (T) and supernatant after centrifugation at 15,000 rpm for 5 min (S) were resolved in 4-15% polyacrylamide gels. Arrowhead indicates the protein band likely corresponding to RFP.

FIG. 2: shows SDS-PAGE analyses of bAL002 and bAL007 to bAL017 clarified lysates.

Protein gels confirmed that all endolysins except of AMI3phi24R (bAL014) were being expressed. Bands corresponding to endolysins are boxed in red.

FIG. 3: shows Example spot assay results from screening set 1.

C. perfringens strain (NCTC 8238) freshly-plated cells and cell lawns screened against E. coli-expressed endolysins, including various negative/positive controls. Denotations on the agar plates correspond to the legend presented. 20 μg/ml ampicillin was shown to inhibit Cp growth on freshly-plated cells but exhibited no bacteriolytic activity on cell lawns. At the concentration tested, lysozyme, disodium EDTA, and their combinations had no effect on both freshly-plated cells and cell lawns. In contrast, depending on the particular E. coli-expressed endolysin, bacteriolytic activities were observed on freshly-plated cells and cell lawns. For Example, the clarified lysate of bAL016 (GH25CPFORC3) was able to produce clearance zones on both cell-lawns and freshly-plated cells. The size of the clearance zones is not indicative of the specific activity of an endolysin as the clarified lysate spots were not normalized to the endolysin expression levels.

FIG. 4: shows Example spot assay results on C. perfringens strain (NCTC 8237) from screening set 2.

Freshly-plated cells and cell lawns screened against E. coli-expressed endolysins, including various negative/positive controls. Spot assay results were similar to screening set 1. Other antibiotics tested (chloramphenicol, kanamycin, and spectinomycin) showed no effect on both exponential and stationary phase cells. In contrast to screening set 1, a higher concentration of disodium EDTA (10 mM) showed inhibitory effect on freshly-plated cells, but not on cell lawns. Similarly to set 1, the clarified lysate of bAL016 (GH25CPFORC3) produced clearance zones on both cell lawns and freshly-plated cells. The size of the clearance zones is not indicative of the specific activity of an endolysin as the clarified lysate spots were not normalized to the endolysin expression levels.

FIG. 5: shows spot assay results on 4 non-C. perfringens species commonly found in the poultry gut (DSMZ 753, 1351, 6011, and 20083).

Freshly-plated cells and cell lawns screened against E. coli-expressed endolysins, including various negative/positive controls. Samples and spot layouts are similar to what's indicated in FIG. 4. 20 μg/ml ampicillin and 10 mM disodium EDTA exhibited inhibitory effects on freshly-plated cells depending on the species but had no effect on cell lawns. Chloramphenicol also exhibited some inhibitory effect on freshly-plated cells. In contrast, all E. coli-expressed endolysins showed no bacteriolytic activities on both freshly-plated cells and cell lawns.

FIG. 6: shows SDS-PAGE analysis and densitometry of purified GH25CPFORC3, AMI2phiCPV4 and AMI2phiZP2 endolysin.

Protein gel results show that the endolysins were appropriately purified from the corresponding E. coli strain with minimal contaminant protein carry-over. Densitometry analysis using the BSA standard curve determined that the purified endolysin stock concentrations for GH25CPFORC3, AMI2phiCPV4, and AMI2phiZP2 were approximately 2400 μg/mL, 750 μg/mL, and 1000 μg/mL, respectively.

FIG. 7: shows Example spot assay results to assess the inhibitory effects and bacteriolytic activity of purified endolysins on C. perfringens.

(A) freshly plated cells—Example spot assay to test the endolysins' inhibitory effects on C. perfringens (APHA B00976). (B) cell lawn 2—Example spot assay to test the endolysins' bacteriolytic activities on C. perfringens (APHA B00976). No visible growth detection is indicated by an −, whereas +/−, +, ++, and +++ correspond to increasingly clear and larger clearance zones.

FIG. 8: shows turbidity reduction assay plate format. Sample layout and effective loading concentrations of the enzybiotics on a 96-microwell plate with 100 μL final volume per well.

Control samples included PBS control (100p/well PBS, pH 7.0 only; 8× replicates) and the blank control (10 μL/well PBS, pH 7.0+90 μL/well resuspended cells; 8× replicates). Except for lysozyme, the GH25CPFORC3, AMI2phiCPV4, and AMI2phiZP2 endolysins were assayed at 3× replicates for each loading concentration.

FIG. 9: shows lysozyme (A) and endolysin (GH25CPFORC3 (B), AMI2phiCPV4 (C), and AMI2phiZP2 (D)) dose responses on NCTC 2837 turbidity reduction.

FIG. 10: shows ranking of endolysin bacteriolytic activity on NCTC 2837 at different loading concentrations (5, 2.5, 1.25, and 0.63 μg/mL—A to D respectively).

FIG. 11: shows lysozyme (A) and endolysin (GH25CPFORC3 (B), AMI2phiCPV4 (C), and AMI2phiZP2 (D)) dose responses on NCTC 8237 turbidity reduction.

FIG. 12: shows ranking endolysin of bacteriolytic activity on NCTC 8237 at different loading concentrations (5, 2.5, 1.25, and 0.63 μg/mL—A to D respectively).

FIG. 13: shows lysozyme (A) and endolysin (GH25CPFORC3 (B), AMI2phiCPV4 (C), and AMI2phiZP2 (D)) dose responses on NCTC 8238 turbidity reduction.

FIG. 14: shows ranking of endolysin bacteriolytic activity on NCTC 8238 at different loading concentrations (5, 2.5, 1.25, and 0.63 μg/mL—A to D respectively).

FIG. 15: shows lysozyme (A) and endolysin (GH25CPFORC3 (B), AMI2phiCPV4 (C), and AMI2phiZP2 (D)) dose responses on NCTC 8239 turbidity reduction.

FIG. 16: shows ranking of endolysin bacteriolytic activity on NCTC 8239 at different loading concentrations (5, 2.5, 1.25, and 0.63 μg/mL—A to D respectively).

FIG. 17: shows summary of an endolysin's bacteriolytic activity at different loading concentrations (5, 2.5, 1.25, and 0.63 μg/mL) and on different C. perfringens reference strains (NCTC 2837 (A), 8237 (B), 8238 (C), 8239 (D)).

Extracted ‘t_50% lysis’ values plotted on scatter plots allow more concise description of the ranking and dose responses of the endolysins, including on different strains. The scatter plots suggest that NCTC 8237 was the most susceptible to the endolysins tested, and that NCTC 8238 was the most resistant.

FIG. 18: shows summary of lysozyme and endolysin bacteriolytic activities on NCTC 8238.

Lysozyme ‘t_50% lysis’ values could only be determined from turbidity reduction assay results on NCTC 8238. While lysozyme showed higher bacteriolytic activities on NCTC 8238 than on the other 3 strains (NCTC 2837, 8237, 8239), the summary plot shows that its lytic activities are still much less compared to the endolysins.

FIG. 19: shows log 10 reductions of C. perfringens strains treated with endolysins at loading concentrations of 1 μg/mL and 5 μg/mL.

Δ log 10 values of four different C. perfringens (NCTC 2837, 8237, 8238, 8239) cells treated with (A) GH25CPFORC3, (B) AMI2phiCPV4 and (C) AMI2phiZP2 endolysins with initial bacterial loading of 15.3 million cells/mL are presented. The Δ log 10 values were calculated as the log 10 of the ratio between the untreated cell control reactions over the endolysin-treated cell reactions for each strain. Average Δ log 10 values and standard deviation from three biological replicates are shown. Assay results support endolysin performance rankings established from Example 12, from highest performance to the lowest: 1) GH25CPFORC3, 2) AMI2phiCPV4, 3) AMI2phiZP2.

FIG. 20: shows dose-response of C. perfringens viability against endolysins. Percentage of reduction in cell viability was calculated from the Δ log 10 values of FIG. 19, and plotted against the endolysin concentration used in the assay. Results showed that percentage of reduction in cell viability increased with increasing endolysins added.

The plots in FIG. 20 are alternative plots of the data shown in FIG. 19. These alternative plots show the percentage reduction in the strains' viabilities as a function of the endolysin loading concentrations. The plots show that increasing endolysin concentrations were able to reduce the viability of the cells. However, for all strains, the endolysin and cell viability dose responses did not vary linearly as the addition of 5 times more endolysin, from 1 μg/mL to 5 μg/mL, did not reduce cell viability by 5 times. It is possible that the endolysins' antimicrobial activities are being underestimated given the current assay conditions.

FIG. 21: shows example spot assay results to investigate synergistic interactions between GH25CPFORC3 ('FORC3) and AMI2phiCPV4 ('V4) endolysins and their bacteriolytic activities on C. perfringens. Individual or combined endolysins were spotted on NCTC 8238 cell lawns according to the sample layout. No visible growth detection is indicated by an −, whereas +/−, +, ++, and +++ correspond to increasingly clear and larger clearance zones. Synergies were observed as larger clearance zones were produced for when 100 ug/mL each of GH25CPFORC3 and AMI2phiCPV4 were spotted compared to 200 ug/mL GH25CPFORC3 or AMI2phiCPV4 spots. Similarly, larger clearance zones were produced for when 50 ug/mL each of GH25CPFORC3 and AMI2phiCPV4 were spotted compared to 100 ug/mL GH25CPFORC3 or AMI2phiCPV4 spots.

FIG. 22: Affinity purification of 6×His-tagged AMI2phiZP2, AMI2phiCPV4, and GH25CPF4969 endolysins. SDS-PAGE gel and densitometry analyses show that the endolysins were appropriately purified from the corresponding E. coli strain. Densitometry analyses using the BSA standard curve determined that the purified endolysin stock concentrations for AMI2phiZP2, AMI2phiCPV4, and GH25CPF4969 were approximately 1700 μg/mL, 1000 μg/mL, and 3600 μg/mL, respectively.

FIG. 23: Example checkerboard PGN assay plate layout and endolysin loading concentrations. Two checkerboard PGN assays were done in one 96-microwell plate with an example sample layout for an 1× endolysin starting concentration of 10 μg/mL 10× stocks of column endolysins (i.e. endolysin 1 or 3) were 2-fold serially-diluted 6 times to 1.6 μg/mL, whereas 10× stocks of row endolysins (i.e. endolysins 2 or 4) were 2-fold serially-diluted 4 times to 6 μg/mL. 10 μL/well of the corresponding endolysin 1 or 3 10× stocks were dispensed into the appropriate columns and similarly 10 μL/well of the corresponding endolysin 2 or 4 10× stocks were dispensed into the appropriate rows. 10 μL/well of PBS, pH 7.0 was added to endolysin 1 or 3 only control columns 6 and 12, respectively. Similarly, 10 μL/well of PBS, pH 7.0 was added to endolysin 2 or 4 only control row H. An additional 10 μL/well of PBS, pH 7.0 was added to well H6 and H12 for the 0 μg/mL loading well (i.e. PGN only control). The PGN assay was initiated by dispensing 80 μL/well of PGN assay stock (prepared as described in Example 20), thereby effectively diluting each endolysin 10-fold. Wells H6 and H12 are “PGN only” control wells with 0 μg/mL endolysin. Endolysin loadings in each well correspond to the row and column loading concentrations indicated.

FIG. 24: Synergistic effects of purified GH25CPFORC3 and AMI2phiZP2 endolysins on C. perfringens strain Cp6 PGN degradation. (A) PGN degradation profiles of 2.5 μg/mL GH25CPFORC3, 2.5 μg/mL AMI2phiZP2, and 2.5 μg/mL GH25CPFORC3+2.5 μg/mL AMI2phiZP2. (B) PGN degradation profiles of 2.5 μg/mL GH25CPFORC3, 0.63 μg/mL AMI2phiZP2, and 2.5 μg/mL GH25CPFORC3+0.63 μg/mL AMI2phiZP2. (C) PGN degradation profiles of 1.25 μg/mL GH25CPFORC3, 2.5 μg/mL AMI2phiZP2, and 1.25 μg/mL GH25CPFORC3+2.5 μg/mL AMI2phiZP2. (D) Summary of initial PGN degradation rates. Degradation profile labels correspond to the purified endolysin and its loading. Additionally, accompanying the label in parenthesis describes the initial degradation rate (OD/hr) of the PGN degradation profile. Synergistic effects were assessed to be present when an endolysin combination exhibited 1) higher PGN degradation rate compared to the sum of each endolysin's individual rates and/or 2) higher PGN degradation to lower ODs than when the endolysins are used on their own. Therefore, results show that the endolysin combination is synergistic.

FIG. 25: Synergistic effects of purified GH25CPFORC3 and AMI2phiCPV4 endolysins on C. perfringens strain Cp6 PGN degradation. (A) PGN degradation profiles of 2.5 μg/mL GH25CPFORC3, 2.5 μg/mL AMI2phiCPV4, and 2.5 μg/mL GH25CPFORC3+2.5 μg/mL AMI2phiCPV4. (B) PGN degradation profiles of 1.25 μg/mL GH25CPFORC3, 2.5 μg/mL AMI2phiCPV4, and 1.25 μg/mL GH25CPFORC3+2.5 μg/mL AMI2phiCPV4. (C) PGN degradation profiles of 0.16 μg/mL GH25CPFORC3, 5 μg/mL AMI2phiCPV4, and 0.16 μg/mL GH25CPFORC3+5 μg/mL AMI2phiCPV4. (D) Summary of initial PGN degradation rates. Degradation profile labels correspond to the purified endolysin and its loading. Additionally, accompanying the label in parenthesis describes the initial degradation rate (OD/hr) of the PGN degradation profile. Synergistic effects were assessed to be present when an endolysin combination exhibited 1) higher PGN degradation rate compared to the sum of each endolysin's individual rates and/or 2) higher PGN degradation to lower ODs than when the endolysins are used on their own. Therefore, results show that the endolysin combination is synergistic.

FIG. 26: Synergistic effects of purified GH25CPF4969 and AMI3CPF4969 endolysins on C. perfringens strain Cp6 PGN degradation. (A) PGN degradation profiles of 10 μg/mL GH25CPF4969, 5 μg/mL AMI3CPF4969, and 10 μg/mL GH25CPF4969+5 μg/mL AMI3CPF4969. (B) PGN degradation profiles of 10 μg/mL GH25CPF4969, 2.5 μg/mL AMI3CPF4969, and 10 μg/mL GH25CPF4969+2.5 μg/mL AMI3CPF4969. (C) PGN degradation profiles of 5 μg/mL GH25CPF4969, 10 μg/mL AMI3CPF4969, and 5 μg/mL GH25CPF4969+10 μg/mL AMI3CPF4969. (D) PGN degradation profiles of 0.16 μg/mL GH25CPF4969, 10 μg/mL AMI3CPF4969, and 0.16 μg/mL GH25CPF4969+10 μg/mL AMI3CPF4969. (E) Summary of initial PGN degradation rates. Degradation profile labels correspond to the purified endolysin and its loading. Additionally, accompanying the label in parenthesis describes the initial degradation rate (OD/hr) of the PGN degradation profile. Synergistic effects were assessed to be present when an endolysin combination exhibited 1) higher PGN degradation rate compared to the sum of each endolysin's individual rates and/or 2) higher PGN degradation to lower ODs than when the endolysins are used on their own. Therefore, results show that the endolysin combination is synergistic.

FIG. 27: Synergistic effects of purified GH25phiS63 and GH25CPF4969 endolysins on C. perfringens strain Cp6 PGN degradation. (A) PGN degradation profiles of 10 μg/mL GH25phiS63, 10 μg/mL GH25CPF4969, and 10 μg/mL GH25phiS63+10 μg/mL GH25CPF4969. (B) PGN degradation profiles of 5 μg/mL GH25phiS63, 10 μg/mL GH25CPF4969, and 5 μg/mL GH25phiS63+10 μg/mL GH25CPF4969. (C) Summary of initial PGN degradation rates. Degradation profile labels correspond to the purified endolysin and its loading. Additionally, accompanying the label in parenthesis describes the initial degradation rate (OD/hr) of the PGN degradation profile. Synergistic effects were assessed to be present when an endolysin combination exhibited 1) higher PGN degradation rate compared to the sum of each endolysin's individual rates and/or 2) higher PGN degradation to lower ODs than when the endolysins are used on their own. Therefore, results show that the endolysin combination is synergistic.

FIG. 28: Synergistic effects of purified GH25CPFORC3 and AMI3CPF4969 endolysins on C. perfringens strain Cp6 PGN degradation. (A) PGN degradation profiles of 1.25 μg/mL GH25CPFORC3, 2.5 μg/mL AMI3CPF4969, and 1.25 μg/mL GH25CPFORC3+2.5 μg/mL AMI3CPF4969. (B) PGN degradation profiles of 0.63 μg/mL GH25CPFORC3, 2.5 μg/mL AMI3CPF4969, and 0.63 μg/mL GH25CPFORC3+2.5 μg/mL AMI3CPF4969. (C) Summary of initial PGN degradation rates. Degradation profile labels correspond to the purified endolysin and its loading. Additionally, accompanying the label in parenthesis describes the initial degradation rate (OD/hr) of the PGN degradation profile. Synergistic effects were assessed to be present when an endolysin combination exhibited 1) higher PGN degradation rate compared to the sum of each endolysin's individual rates and/or 2) higher PGN degradation to lower ODs than when the endolysins are used on their own. Therefore, results show that the endolysin combination is synergistic.

FIG. 29: Synergistic effects of purified GH25CPF4969 and AMI2phiZP2 endolysins on C. perfringens strain Cp6 PGN degradation. (A) PGN degradation profiles of 2.5 μg/mL GH25CPF4969, 2.5 μg/mL AMI2phiZP2, and 2.5 μg/mL GH25CPF4969+2.5 μg/mL AMI2phiZP2. (B) PGN degradation profiles of 0.31 μg/mL GH25CPF4969, 10 μg/mL AMI2phiZP2, and 0.31 μg/mL GH25CPF4969+10 μg/mL AMI2phiZP2. (C) Summary of initial PGN degradation rates. Degradation profile labels correspond to the purified endolysin and its loading. Additionally, accompanying the label in parenthesis describes the initial degradation rate (OD/hr) of the PGN degradation profile. Synergistic effects were assessed to be present when an endolysin combination exhibited 1) higher PGN degradation rate compared to the sum of each endolysin's individual rates and/or 2) higher PGN degradation to lower ODs than when the endolysins are used on their own. Therefore, results show that the endolysin combination is synergistic.

FIG. 30: Plate layouts for PGN degradation assay (A) and MIC assay (B) used to evaluate lytic and antimicrobial activity of algal extracts.

FIG. 31: Bacteriolytic activities of endolysin-expressing algal extracts on Clostridium perfringens Cp6 cells (whole-cell Cp6 spot assay). Samples from shake flask cultures of Chlamydomonas strains crAL035, crAL038, crAL039, crAL045, crAL057, and crAL061 were collected to assay their lytic activities on C. perfringens Cp6 cells. Soluble extracts prepared from each strain were normalized to 10 mg TSP/mL and 10 μL were spotted on solid agar media embedded with C. perfringens Cp6 cells. Soluble extracts were spotted in duplicates. The number in the center of each spot indicates the Chlamydomonas strain from which the extract was prepared. Lytic activities were qualitatively assessed based on the size and clarity of the clearance zones around each spot. crAL035, a strain that was created by transformation with a vector without an endolysin gene, was used as a control.

FIG. 32: Lytic activity of endolysin-expressing algal extracts on Clostridium perfringens Cp6 cells (PGN degradation assay). (A) PGN degradation profiles of extracts from algal strains expressing GH25CPFORC3 only (crAL045 and crAL061). (B) PGN degradation profiles of extracts from algal strains expressing AMI2phiZP2 only (crAL038 and crAL039) (C) PGN degradation profiles of strains crAL057 (which co-expresses GH25CPFORC3 and AMI2phiZP2), crAL038 (AMI2phiZP2 only) and crAL061 (GH25CPFORC3 only). (D) Summary of the initial PGN degradation rates extracted from plots A to C.

FIG. 33: Minimal inhibitory concentration (MIC) of extracts from endolysin-expressing algal strains on C. perfringens Cp6 cells. The MIC values of soluble extracts prepared from endolysin-expressing Chlamydomonas strains. MIC is expressed as total soluble protein in microgram per millilitre (μg TSP/mL) of the soluble extract, which fully inhibits bacteria growth. Error bars correspond to 1 standard error.

FIG. 34: Plate layout for the PGN degradation assay used to evaluate the lytic activity of algal extracts. Algal extracts were prepared at 10 mg TSP/mL (10× stock) and then 2-fold serially-diluted up to 7 times to 78 μg TSP/mL. Extract from strain crAL035 was 2-fold serially diluted only 3 times to 1250 μg TSP/mL. 10 μL/well of extract dilutions were dispensed into a 96-microwell plate according to the plate layout shown. For “PGN only” control wells with no extract loaded, 10 μL/well PBS, pH 7.0, was dispensed. The PGN assay was initiated by dispensing 90 μL/well PGN assay stock

FIG. 35: Bacteriolytic activity determination of endolysin-expressing strains crAL075, crAL076, crAL077, crAL078, crAL079 and crAL080 on C. perfringens Cp6 cells (whole-cell Cp6 spot assay). (A) Spot assay plate layout. (B) Spot assay results of different algal extracts. The number in the center of each spot refers to the corresponding strain ID (for example, “58” refers to strain crAL058). This is followed by a number that refers to dilution factor (1, 2, and 4). All algal extracts were normalized to a 1× stock of 10 mg TSP/mL.

FIG. 36: PGN degradation profiles of algal extracts produced from Chlamydomonas strains crAL035, crAL038, crAL039, crAL041, crAL058, crAL075, crAL076, crAL077, crAL078, crAL079, and crAL080. (A) PGN degradation profiles of 125, 250, 500, and 1000 μg TSP/mL extract from crAL035, which does not carry any endolysin gene. (B) Background PGN degradation when mixed with buffer (PBS, pH 7.0) instead of algal extract (4× replicates). (C) PGN degradation profiles of extracts from strains expressing putative GH25 endolysins: crAL041 (GH25CPFORC3), crAL075 (GH25phiS63), and crAL079 (AMI3CPF4969). (D) PGN degradation profiles of extracts from strains expressing putative AMI2 endolysins: crAL038 (AMI2phiZP2) and crAL080 (GH25CPF4969). (E) PGN degradation profiles of extracts from strains expressing putative AMI3 endolysins: crAL076 (AMI3CPFORC25), crAL077 (AMI3CPJP55), and crAL078 (AMI3phi24R). (F) PGN degradation profiles of extracts from the negative control crAL035 strain which does not express an endolysin gene, and from inactive strains crAL075 and crAL077.

FIG. 37: Lytic activities of algal extracts produced from Chlamydomonas strains crAL035, crAL038, crAL039, crAL041, crAL045, crAL058, crAL075, crAL076, crAL077, crAL078, crAL079, and crAL080. (A) Dose response in PGN degradation initial rates by increasing amounts of extracts containing putative GH25 endolysins. (B) Dose response in PGN degradation initial rates by increasing amounts of extracts containing putative AMI2 endolysins (C) Dose response in PGN degradation initial rates by increasing amounts of extracts containing putative AMI3 endolysins (D) Summary of activities of extracts from endolysin-expressing C. reinhardtii strains, expressed as change in OD at 600 nm/(h×(μg TSP/ml)

FIG. 38: Growth profiles of Chlamydomonas reinhardtii strains crAL045 and crAL039 grown in a 5 L bioreactor. Optical density at 750 nm (OD750 nm) was used to follow the increase in cell density over time. Cultures were grown at 25° C. under dark conditions, with air bubbling and pH control. (A) shows growth profile of strain crAL045 and (B) shows the growth profile of strain crAL039. Strain crAL045 was harvested at approximately 41 OD after 6 days of growth. Cells were spray-dried, giving a total yield of 25.8 g of dried biomass. Similarly, strain crAL039 was harvested at approximately 116 OD after 5 days of growth and yielded 42 g of dried biomass after spray drying.

FIG. 39: MIC/MBC assay plate layout and effective extract loading concentrations. The MIC/MBC assay was initiated accordingly to indicated plate format. The TSP content of the soluble extracts of crAL045 and crAL039 used was 30 mg TSP/mL (10× stock). crAL045 and crAL039 soluble extracts were 2× serially-diluted using PBS, pH 7.0 buffer 11 times, or to effectively 2048× dilution. To initiate the MIC assay, 10 μL/well of 2-fold serially-diluted soluble extracts were dispensed according to the plate layout followed by the addition of 90 μL/well of ˜10\hat{ }5 cells/mL Cp6, giving the effective loading concentrations. Serial dilutions of crAL045 extracts were assayed in 3× replicates whereas crAL039 extracts were assayed in duplicates.

FIG. 40: Antimicrobial activities of crAL045 & crAL039 spray-dried biomass. Spray-dried biomass from crAL045 and crAL039 strains was used to produce soluble extracts for antimicrobial activity determination. Low MIC values using 2.5E5 cells/mL of Clostridium perfringens strain CP6 showed that both crAL045 and crAL039 dried biomass possessed high antimicrobial activity. MBC values increased slightly compared to MIC values. Antimicrobial activity ranking matched those observed using soluble extracts from wet shake flask pellets (Example 24), where crAL045 is more active than crAL039. Interestingly, extracts from spray-dried biomass were able to produce much lower MIC values compared to from shake flask extracts.

FIG. 41: Plate layouts for PGN degradation assays showing algal extract enhancement of representative endolysins. (A) PGN assay layout used to show 35D, 35 L, 53D, and 54D algal extract enhancements of GH25CPFORC3 and AMI2phiZP2 endolysins. 2× serial-dilutions of the endolysins diluted in either PBS, pH 7.0 buffer or the four algal extracts were assayed without replicates. (B) PGN assay layout used to show 54D algal extract enhancement of GH25CPFORC3, GH25CPF4969, AMI2phiZP2, and AMI3CPF4969 endolysins. 2× serial-dilutions of the endolysins diluted in either PBS, pH 7.0 buffer or the 54D algal extracts were assayed in 3× replicates. For each plate layout, 4× replicate “PGN only” control wells with 0 μg/mL endolysin loaded were also included for the diluents to determine their background degradation rates, if any. Effective extract concentrations noted only correspond to the wells diluted using extracts.

FIG. 42: Fold enhancement calculation of the endolysin in algal extract compared to in PBS, pH 7.0 buffer. The equation was used to calculate the fold enhancement of an algal extract on the PGN degradation activities of an endolysin compared to in PBS, pH 7.0 buffer. For example, ν_{2.5,ex} corresponds to the initial PGN degradation rate of an endolysin diluted in algal extract at a loading concentration of 2.5 μg/mL. Or, ν_{0,ex} corresponds to the initial PGN degradation rate of algal extract only with 0 μg/mL endolysin loading (i.e. background degradation rate).

FIG. 43: C. reinhardtii extracts enhance the PGN degradation activity of GH25CPFORC3 endolysin at 2.5 μg/mL concentration. PGN degradation profiles of 2.5 μg/mL of purified GH25CPFORC3 endolysin mixed with 35D (A), 35 L (B), 53D (C), and 54D (D) C. reinhardtii extracts are shown and compared with that of the purified endolysin mixed only with PBS, pH 7.0 buffer. Initial rates extracted from the PGN degradation profiles are summarized in the bar plot (E); error bars correspond to 1 standard error. Results show that all four C. reinhardtii extracts enhance the PGN degradation activity of GH25CPFORC3. Additionally, the bar plot shows that C. reinhardtii extracts on their own (0 μg/mL endolysin) cannot degrade PGN.

FIG. 44: C. reinhardtii extracts enhance the PGN degradation activity of GH25CPFORC3 endolysin at 1.25 μg/mL concentration. PGN degradation profiles of 1.25 μg/mL of purified GH25CPFORC3 endolysin mixed with 35D (A), 35 L (B), 53D (C), and 54D (D) C. reinhardtii extracts are shown and compared with that of the purified endolysin mixed only with PBS, pH 7.0 buffer. Initial rates extracted from the PGN degradation profiles are summarized in the bar plot (E); error bars correspond to 1 standard error. Results show that all four C. reinhardtii extracts enhance the PGN degradation activity of GH25CPFORC3. Additionally, the bar plot shows that C. reinhardtii extracts on their own (0 μg/mL endolysin) cannot degrade PGN.

FIG. 45: C. reinhardtii extracts enhance the PGN degradation activity of GH25CPFORC3 endolysin at 0.63 μg/mL concentration. PGN degradation profiles of 0.63 μg/mL of purified GH25CPFORC3 endolysin mixed with 35D (A), 35 L (B), 53D (C), and 54D (D) C. reinhardtii extracts are shown and compared with that of the purified endolysin mixed only with PBS, pH 7.0 buffer. Initial rates extracted from the PGN degradation profiles are summarized in the bar plot (E); error bars correspond to 1 standard error. Results show that all four C. reinhardtii extracts enhance the PGN degradation activity of GH25CPFORC3. Additionally, the bar plot shows that C. reinhardtii extracts on their own (0 μg/mL endolysin) cannot degrade PGN.

FIG. 46: Fold enhancement of 35D, 35 L, 53D, and 54D algal extracts on the PGN degradation activity of GH25CPFORC3 endolysin. Results from single replicates show that at the indicated loading concentrations, and depending on the algal extract, the GH25CPFORC3 endolysin is enhanced to up to approximately 2.5-fold compared to when mixed in PBS, pH 7.0 buffer.

FIG. 47: C. reinhardtii extracts enhance the PGN degradation activity of GH25CPFORC3 endolysin at 0.31, 0.16, and 0.08 μg/mL concentration. PGN degradation profiles of 0.31 (A), 0.16 (B) and 0.08 (C) μg/mL of purified GH25CPFORC3 endolysin mixed with 35D, 35 L, 53D and 54D C. reinhardtii extracts are shown and compared with that of the purified endolysin mixed only with PBS, pH 7.0 buffer. Comparison of the degradation profiles show that the endolysin's PGN degradation activities is enhanced in the presence of algal extracts compared to only in buffer.

FIG. 48: C. reinhardtii extracts enhance the PGN degradation activity of AMI2phiZP2 endolysin. PGN degradation profiles of 2.5 (A), 1.25 (B), and 0.63 (C) μg/mL of purified AMI2phiZP2 endolysin mixed with 54D C. reinhardtii extracts are shown and compared with that of the purified endolysin mixed only with PBS, pH 7.0 buffer. Initial rates extracted from the PGN degradation profiles are summarized in the bar plots (D−2.5 μg/mL) (E−1.25 μg/mL) and (F−0.63 μg/mL); error bars correspond to 1 standard error. Results show that all four C. reinhardtii extracts enhance the PGN degradation activity of AMI2phiZP2. Additionally, the bar plot shows that C. reinhardtii extracts on their own (0 μg/mL endolysin) cannot degrade PGN.

FIG. 49: Fold enhancement of 35D, 35 L, 53D, and 54D algal extracts on the PGN degradation activity of AMI2phiZP2 endolysin. Results from single replicates show that at the indicated loading concentrations, and depending on the algal extract, the AMI2phiZP2 endolysin is enhanced up to approximately 2.2-fold compared to when mixed in PBS, pH 7.0 buffer.

FIG. 50: 54D C. reinhardtii extract enhancement on the PGN degradation activity of GH25CPFORC3 endolysin is reproducible. Representative PGN degradation profiles of 2.5 (A), 1.25 (B), 0.63 (C), 0.31 (D), 0.16 (E), and 0 (F) μg/mL of purified GH25CPFORC3 endolysin mixed with 54D C. reinhardtii extract are shown and compared with that of the purified endolysin mixed only with PBS, pH 7.0 buffer. Initial rates extracted from the PGN degradation profiles are summarized in the bar plot (G); error bars correspond to 1 standard error. Results show that algal extract enhancement of the PGN degradation activity of GH25CPFORC3 endolysin is reproducible and consistent with 3× replicates. The enhancement is significant particularly at reduced loading concentrations when the endolysin diluted in buffer exhibits degradation activities almost as low as background (0 μg/mL endolysin), and can be seen for example at 0.16 μg/mL endolysin concentration.

FIG. 51: 54D C. reinhardtii extract enhancement on the PGN degradation activity of AMI2phiZP2 endolysin is reproducible. Representative PGN degradation profiles of 1.25 (A), 0.63 (B), 0.31 (C), 0.16 (D), and 0 (E) μg/mL of purified AMI2phiZP2 endolysin mixed with 54D C. reinhardtii extract are shown and compared with that of the purified endolysin mixed only with PBS, pH 7.0 buffer. Initial rates extracted from the PGN degradation profiles are summarized in the bar plot (F); error bars correspond to 1 standard error. Results show that algal extract enhancement of the PGN degradation activity of AMI2phiZP2 endolysin is reproducible and consistent with 3× replicates.

FIG. 52: 54D C. reinhardtii extract enhances the PGN degradation activity of AMI3CPF4969 endolysin. Representative PGN degradation profiles of 2.5 (A), 1.25 (B), 0.63 (C), 0.31 (D), 0.16 (E), and 0 (F) μg/mL of purified AMI3CPF4969 endolysin mixed with 54D C. reinhardtii extract are shown and compared with that of the purified endolysin mixed only with PBS, pH 7.0 buffer. Initial rates extracted from the PGN degradation profiles are summarized in the bar plot (G); error bars correspond to 1 standard error. Results were consistent with 3× replicates and show that algal extract enhancement also extends to AMI3CPF4969. The enhancement is significant particularly at reduced loading concentrations when the endolysin diluted in buffer exhibits degradation activities as near background levels (0 μg/mL endolysin), and can be seen for example at 1.25, 0.63, 0.31, and 0.16 μg/mL endolysin concentrations.

FIG. 53: 54D C. reinhardtii extract enhances the PGN degradation activity of GH25CPF4969 endolysin. Representative PGN degradation profiles of 1.25 (A), 0.63 (B), 0.31 (C), 0.16 (D), and 0 (E) μg/mL of purified GH25CPF4969 endolysin mixed with 54D C. reinhardtii extract are shown and compared with that of the purified endolysin mixed only with PBS, pH 7.0 buffer. Initial rates extracted from the PGN degradation profiles are summarized in the bar plot (F); error bars correspond to 1 standard error. Results were consistent with 3× replicates and show that algal extract enhancement also extends to GH25CPF4969.

FIG. 54: Fold enhancement of 54D algal extracts on the PGN degradation activities of endolysins. Algal extracts enhance the PGN degradation activities of endolysins GH25CPFORC3 and AMI3CPF4969, as well as endolysins AMI2phiZP2 and GH25CPF4969. Extract enhancement was significant for GH25CPFORC3 and AMI3CPF4969 endolysins, with up to approximately 69-fold and 44-fold enhancement for GH25CPFORC3 and AMI3CPF4969 endolysins, respectively. Extract enhancement for AMI3CPF4969 could also be said to be “infinitely” enhanced as the endolysin diluted in buffer exhibited degradation rates similar to background at concentrations below 0.63 μg/mL. Both AMI2phiZP2 and GH25CPF4969 endolysins were enhanced by algal extracts by up to approximately 2-fold.

FIG. 55: Algal extract enhances the antimicrobial activity of GH25CPFORC3 and AMI2phiZP2 endolysins. (A) Plate layout for the MIC determination of GH25CPFORC3 and AMI2phiZP2 diluted in PBS pH 7.0 or algal extract prepared from strain crAL054 grown in the dark (54D). (B) MIC values for GH25CPFORC3 and AMI2phiZP2 in PBS, pH 7.0 buffer or in 500 μg TSP/mL 54D extract. Results show that the extract enhances purified GH25CPFORC3 and AMI2phiZP2 endolysin activity approximately 2.5 and 4-fold, respectively, than when in the buffer. Error bars correspond to 1 standard error.

FIG. 56: Combinations of Chlamydomonas extracts tested to investigate synergistic effects. 9 different combinations of Chlamydomonas strains were tested to investigate their synergistic effects when used in combination. Combinations 8-9 use crAL058 extract, co-expressing GH25CPFORC3 and AMI2phiZP2 endolysins, and extract from a second strain expressing an endolysin not expressed in crAL058. The corresponding endolysin(s) expressed in each Chlamydomonas strain is also indicated.

FIG. 57: Example checkerboard PGN assay plate layout and extract loading concentrations. Two checkerboard PGN assays were initiated in one 96-microwell plate with an example sample layout for a 1× endolysin starting concentration of 500 μg/mL. 10× stocks of column extracts (i.e. extract 1 or 3) were 2× serially-diluted 6 times to 78.1 μg/mL, whereas 10× stocks of row extracts (i.e. extracts 2 or 4) were 2× serially-diluted 4 times to 313 μg/mL. 10 μL/well of the corresponding extract 1 or 3 dilutions were dispensed into the appropriate columns and similarly 10 μL/well of the corresponding extract 2 or 4 dilutions were dispensed into the appropriate rows. 10 μL/well of PBS, pH 7.0 was added to columns 6 and 12 (extract 1 or 3 dilutions only, respectively). Similarly, 10 μL/well of PBS, pH 7.0 was added to row H wells (extract 2 or 4 dilutions only). Well H6 and H12 (“PGN only” control, with 0 μg/mL endolysin) was ensured to contain a total of 20 μL/well PBS, pH 7.0. The PGN assay was initiated by dispensing 80 μL/well PGN assay stock, thereby effectively diluting each extract 10-fold. Extract loadings in each well correspond to the row and column loading concentrations indicated.

FIG. 58: Synergistic effects of Chlamydomonas extracts crAL041 (expressing GH25CPFORC3) and crAL038 (expressing AMI2phiZP2) on C. perfringens strain Cp6 PGN degradation. (A) PGN degradation profiles using 500 μg TSP/mL crAL041, 500 μg TSP/mL crAL038, and 500 μg TSP/mL crAL041+500 μg TSP/mL crAL038. (B) PGN degradation profiles using 250 μg TSP/mL crAL041, 250 μg TSP/mL crAL038, and 250 μg TSP/mL crAL041+250 μg TSP/mL crAL038. (C) PGN degradation profiles using 125 μg TSP/mL crAL041, 125 μg TSP/mL crAL038, and 125 μg TSP/mL crAL041+125 μg TSP/mL crAL038. (D) PGN degradation profiles using 62.5 μg TSP/mL crAL041, 62.5 μg TSP/mL crAL038, and 62.5 μg TSP/mL crAL041+62.5 μg TSP/mL crAL038. (E) Summary of initial PGN degradation rates. Degradation profile labels correspond to the Chlamydomonas extract and its loading. Additionally, accompanying the label in parenthesis describes the initial rate of the PGN degradation profile. Results show that crAL041 and crAL038 extracts are synergistic when used in combination. Initial rates summarized in the bar plot indicate that the combined rate, when the two extracts are used at the same time, is higher than when the sum of the rates of the extracts are used individually.

FIG. 59: Synergistic effects of Chlamydomonas extracts crAL041 (expressing GH25CPFORC3) and crAL080 (expressing GH25CPF4969) on C. perfringens strain Cp6 PGN degradation. (A) PGN degradation profiles using 250 μg TSP/mL crAL041, 250 μg TSP/mL crAL080, and 250 μg TSP/mL crAL041+250 μg TSP/mL crAL080. (B) PGN degradation profiles using 125 μg TSP/mL crAL041, 125 μg TSP/mL crAL080, and 125 μg TSP/mL crAL041+125 μg TSP/mL crAL080. (C) Summary of initial PGN degradation rates. Degradation profile labels correspond to the Chlamydomonas extract and its loading. Additionally, accompanying the label in parenthesis describes the initial rate of the PGN degradation profile. Results show that crAL041 and crAL080 extracts are synergistic when used in combination. Initial rates summarized in the bar plot indicate that the combined rate, when the two extracts are used at the same time, is higher than when the sum of the rates of the extracts are used individually.

FIG. 60: Synergistic effects of Chlamydomonas extracts crAL045 (expressing GH25CPFORC3) and crAL076 (expressing AMI3CPFORC25) on C. perfringens strain Cp6 PGN degradation. (A) PGN degradation profiles using 500 μg TSP/mL crAL045, 500 μg TSP/mL crAL076, and 500 μg TSP/mL crAL045+500 μg TSP/mL crAL076. (B) PGN degradation profiles using 250 μg TSP/mL crAL045, 250 μg TSP/mL crAL076, and 250 μg TSP/mL crAL045+250 μg TSP/mL crAL076. (C) PGN degradation profiles using 125 μg TSP/mL crAL045, 125 μg TSP/mL crAL076, and 125 μg TSP/mL crAL045+125 μg TSP/mL crAL076. (D) Summary of initial PGN degradation rates. Degradation profile labels correspond to the Chlamydomonas extract and its loading. Additionally, accompanying the label in parenthesis describes the initial rate of the PGN degradation profile. Results show that crAL045 and crAL076 extracts are synergistic when used in combination. Initial rates summarized in the bar plot indicate that the combined rate, when the two extracts are used at the same time, is higher than when the sum of the rates of the extracts are used individually.

FIG. 61: Synergistic effects of Chlamydomonas extracts crAL045 (expressing GH25CPFORC3) and crAL078 (expressing AMI3phi24R) on C. perfringens strain Cp6 PGN degradation. (A) PGN degradation profiles using 500 μg TSP/mL crAL045, 500 μg TSP/mL crAL078, and 500 μg TSP/mL crAL045+500 μg TSP/mL crAL078. (B) PGN degradation profiles using 250 μg TSP/mL crAL045, 250 μg TSP/mL crAL078, and 250 μg TSP/mL crAL045+250 μg TSP/mL crAL078. (C) PGN degradation profiles using 125 μg TSP/mL crAL045, 125 μg TSP/mL crAL078, and 125 μg TSP/mL crAL045+125 μg TSP/mL crAL078. (D) PGN degradation profiles using 62.5 μg TSP/mL crAL045, 62.5 μg TSP/mL crAL078, and 62.5 μg TSP/mL crAL045+62.5 μg TSP/mL crAL078. (E) Summary of initial PGN degradation rates. Degradation profile labels correspond to the Chlamydomonas extract and its loading. Additionally, accompanying the label in parenthesis describes the initial rate of the PGN degradation profile. Results show that crAL045 and crAL078 extracts are synergistic when used in combination. Initial rates summarized in the bar plot indicate that the combined rate, when the two extracts are used at the same time, is higher than when the sum of the rates of the extracts are used individually.

FIG. 62: Synergistic effects of Chlamydomonas extracts crAL079 (expressing AMI3CPF4969) and crAL076 (expressing AMI3CPFORC25) on C. perfringens strain Cp6 PGN degradation. (A) PGN degradation profiles using 500 μg TSP/mL crAL079, 500 μg TSP/mL crAL076, and 500 μg TSP/mL crAL079+500 μg TSP/mL crAL076. (B) PGN degradation profiles using 250 μg TSP/mL crAL079, 250 μg TSP/mL crAL076, and 250 μg TSP/mL crAL079+250 μg TSP/mL crAL076. (C) PGN degradation profiles using 125 μg TSP/mL crAL079, 125 μg TSP/mL crAL076, and 125 μg TSP/mL crAL079+125 μg TSP/mL crAL076. (D) PGN degradation profiles using 62.5 μg TSP/mL crAL079, 62.5 μg TSP/mL crAL076, and 62.5 μg TSP/mL crAL079+62.5 μg TSP/mL crAL076. (E) Summary of initial PGN degradation rates. Degradation profile labels correspond to the Chlamydomonas extract and its loading. Additionally, accompanying the label in parenthesis describes the initial rate of the PGN degradation profile. Results show that crAL079 and crAL076 extracts are synergistic when used in combination. Initial rates summarized in the bar plot indicate that the combined rate, when the two extracts are used at the same time, is higher than when the sum of the rates of the extracts are used individually.

FIG. 63: Synergistic effects of Chlamydomonas extracts crAL076 (expressing AMI3CPFORC25) and crAL038 (expressing AMI2phiZP2) on C. perfringens strain Cp6 PGN degradation. (A) PGN degradation profiles using 500 μg TSP/mL crAL076, 500 μg TSP/mL crAL038, and 500 μg TSP/mL crAL076+500 μg TSP/mL crAL038. (B) PGN degradation profiles using 250 μg TSP/mL crAL076, 250 μg TSP/mL crAL038, and 250 μg TSP/mL crAL076+250 μg TSP/mL crAL038. (C) PGN degradation profiles using 125 μg TSP/mL crAL076, 125 μg TSP/mL crAL038, and 125 μg TSP/mL crAL076+125 μg TSP/mL crAL038. (D) Summary of initial PGN degradation rates. Degradation profile labels correspond to the Chlamydomonas extract and its loading. Additionally, accompanying the label in parentheses describes the initial rate of the PGN degradation profile. Results show that crAL076 and crAL038 extracts are synergistic when used in combination. Initial rates summarized in the bar plot indicate that the combined rate, when the two extracts are used at the same time, is higher than when the sum of the rates of the extracts are used individually.

FIG. 64: Synergistic effects of Chlamydomonas extracts crAL080 (expressing GH25CPF4969) and crAL076 (expressing AMI3CPFORC25) on C. perfringens strain Cp6 PGN degradation. (A) PGN degradation profiles using 500 μg TSP/mL crAL080, 500 μg TSP/mL crAL076, and 500 μg TSP/mL crAL080+500 μg TSP/mL crAL076. (B) PGN degradation profiles using 500 μg TSP/mL crAL080, 250 μg TSP/mL crAL076, and 500 μg TSP/mL crAL080+250 μg TSP/mL crAL076. (C) PGN degradation profiles using 250 μg TSP/mL crAL080, 500 μg TSP/mL crAL076, and 250 μg TSP/mL crAL080+500 μg TSP/mL crAL076. (D) PGN degradation profiles using 250 μg TSP/mL crAL080, 250 μg TSP/mL crAL076, and 250 μg TSP/mL crAL080+250 μg TSP/mL crAL076. (E) Summary of initial PGN degradation rates. Degradation profile labels correspond to the Chlamydomonas extract and its loading. Additionally, accompanying the label in parenthesis describes the initial rate of the PGN degradation profile. Results show that crAL080 and crAL076 extracts are synergistic when used in combination. Initial rates summarized in the bar plot indicate that the combined rate, when the two extracts are used at the same time, is higher than when the sum of the rates of the extracts are used individually.

FIG. 65: Synergistic effects of Chlamydomonas extracts crAL058 (expressing both GH25CPFORC3 and AMIphiZP2) and crAL080 (expressing GH25CPF4969) on C. perfringens strain Cp6 PGN degradation. (A) PGN degradation profiles using 500 μg TSP/mL crAL058, 500 μg TSP/mL crAL080, and 500 μg TSP/mL crAL058+500 μg TSP/mL crAL080. (B) PGN degradation profiles using 250 μg TSP/mL crAL058, 250 μg TSP/mL crAL080, and 250 μg TSP/mL crAL058+250 μg TSP/mL crAL080. (C) PGN degradation profiles using 125 μg TSP/mL crAL058, 125 μg TSP/mL crAL080, and 125 μg TSP/mL crAL058+125 μg TSP/mL crAL080. (D) Summary of initial PGN degradation rates. Degradation profile labels correspond to the Chlamydomonas extract and its loading. Additionally, accompanying the label in parenthesis describes the initial rate of the PGN degradation profile. Results show that crAL058 and crAL080 extracts are synergistic when used in combination. Initial rates summarized in the bar plot indicate that the combined rate, when the two extracts are used at the same time, is higher than when the sum of the rates of the extracts are used individually. The presence of synergistic effects between crAL058 and crAL080 suggest that the three endolysins, GH25CPFORC3, AMI2phiZP2, and GH25CPF4969, are also synergistic when used in combination.

FIG. 66: Synergistic effects of Chlamydomonas extracts crAL058 (expressing both GH25CPFORC3 and AMIphiZP2) and crAL076 (expressing AMI3CPFORC25) on C. perfringens strain Cp6 PGN degradation. (A) PGN degradation profiles using 500 μg TSP/mL crAL058, 500 μg TSP/mL crAL076, and 500 μg TSP/mL crAL058+500 μg TSP/mL crAL076. (B) PGN degradation profiles using 250 μg TSP/mL crAL058, 250 μg TSP/mL crAL076, and 250 μg TSP/mL crAL058+250 μg TSP/mL crAL076. (C) Summary of initial PGN degradation rates. Degradation profile labels correspond to the Chlamydomonas extract and its loading. Additionally, accompanying the label in parenthesis describes the initial rate of the PGN degradation profile. Results show that crAL058 and crAL076 extracts are synergistic when used in combination. Initial rates summarized in the bar plot indicate that the combined rate, when the two extracts are used at the same time, is higher than when the sum of the rates of the extracts are used individually. The presence of synergistic effects between crAL058 and crAL076 also suggest that the three endolysins, GH25CPFORC3, AMI2phiZP2, and AMI3CPFORC25, are synergistic when used in combination.

FIG. 67: 96-microwell checkerboard setup and extract loading concentrations. The checkerboard MIC assay was repeated with modified extract loading concentrations and conditions. Similarly, a 20× concentrated stock of each extract was prepared and 2× serially-diluted. crAL045 extract was serially-diluted to ˜7.3 μg TSP/mL, whereas crAL039 extract was serially-diluted to ˜104.7 μg TSP/mL. 5 μL/well of the corresponding of crAL045 stock extract was dispensed into the appropriate columns and similarly 5 μL/well of the corresponding of crAL039 stock extract dispensed into the appropriate rows. The MIC assay was initiated by dispensing 90 μL/well of ˜10\hat{ }4 cells/mL of C. perfringens strain Cp6 in BHI+C medium into the microwell plate and effectively diluting each extract 20-fold. Row H and column 12 wells contain only 2-fold serial-diluted loading concentrations of crAL045 and crAL039 extract, respectively. Well H12 is a control growth well with no extract added. crAL045 and crAL039 extract loadings in each well correspond to the row and column loading concentrations indicated.

FIG. 68: Fractional Inhibition Concentration index value calculation. Fractional Inhibition Concentration (FIC) index calculation was used to determine synergistic effects between antimicrobials. A synergistic combination is when the combination results in a FIC value of ≤0.5. An additive effect is defined as 0.5<FIC≤4, whereas an antagonistic effect is when FIC>4.

FIG. 69: Synergistic effects of Chlamydomonas-expressed endolysin extracts, crAL045 and crAL039 (checkerboard MIC assay). Overnight incubation of ˜10\hat{ }4 cells/mL of C. perfringens strain Cp6 with different loading concentrations of crAL045 and crAL039 extracts confirm that they are synergistic in inhibiting C. perfringens cell growth (A). Well values greater than 0.10 OD620 nm correspond to cell growth with the growth control well (H12) having a 0.29 OD620 nm. Inhibited wells were assessed both on OD620 nm readings as well as visual inspections. Various levels of partial inhibition were also observed. The MIC of crAL045 extract alone was determined to be 23.4 μg TSP/mL (H5, *), whereas the MIC of crAL039 extract alone was determined to be 167.5 μg TSP/mL (B12, **). At a combined loading of 5.9 μg TSP/mL crAL045 extract and 41.9 μg TSP/mL crAL039 extract (D7, ***), the combination was calculated to be synergistic with a fractional inhibition concentration of 0.5 (calculation: 5.9/23.4+41.9/167.5=0.5). (B) Summary bar plot showing the significant drop of each extract, crAL045 and crAL039, required to inhibit Cp6 cell growth compared to when each extract is used on its own.

### DETAILED DESCRIPTION OF THE INVENTION

### Endolysin Polypeptides, Fragments and Variants Thereof

Novel bacteriophage-encoded endolysin polypeptides that can be employed as antimicrobial agents (“endolysins”) were identified by the inventors. The present invention embraces specific endolysins having amino acid sequences as set forth in SEQ ID NOs disclosed herein, each of which is identified herein as a polypeptide of the invention. These endolysins possess surprising and advantageous functional antimicrobial characteristics. These endolysins possess antibacterial activity. These endolysins possess antibacterial activity against species of Clostridium perfringens. Clostridium perfringens may also be referred to as “C. perfringens” or “Cp”. These endolysins may possess antibacterial activity against Type A species and strains of C. perfringens.

In addition, the invention also embraces variants of the specific endolysins having amino acid sequences as set forth in SEQ ID NOs disclosed herein and which are identified herein as a polypeptide of the invention. A “variant” may be a fragment/truncate, mutant or derivative of a specific endolysin having an amino acid sequence as set forth in SEQ ID NOs disclosed herein and which is identified herein as a polypeptide of the invention. For example, a variant may differ from a specific endolysin having an amino acid sequence as set forth in SEQ ID NOs disclosed herein by one or more amino acid substitutions. Thus the amino acid sequence of an endolysin variant may relate to the amino acid sequence of a particular SEQ ID NO by reference to a percentage identity, as defined in more detail herein. In all cases, a variant endolysin also possesses antimicrobial activity. All variant endolysins possess antibacterial activity, particularly against species of Clostridium perfringens. Variant endolysins may possess antibacterial activity against Type A species and strains of C. perfringens. Variant endolysins which do not possess antimicrobial and antibacterial activity as noted above are not within the scope of the present invention.

A “fragment” of an endolysin polypeptide, or “fragment” of a domain of an endolysin polypeptide, as described herein, is any polypeptide which has an amino acid sequence which is shorter than the full-length endolysin polypeptide or full-length domain of the endolysin polypeptide. Endolysins are well-characterised enzymes and the skilled person may readily delineate the sequence characteristics which define a full-length endolysin polypeptide or full-length domain of the endolysin polypeptide, such as by sequence alignment and analysis. Accordingly, a skilled person may readily delineate such a “fragment”.

The specific endolysins having amino acid sequences as set forth in SEQ ID NOs disclosed herein and which are identified herein as polypeptides of the invention as well as all variant endolysins may be collectively referred to herein as “endolysins” and/or “endolysins of the invention” and/or “endolysin polypeptides” and/or “endolysin polypeptides of the invention”.

Reference will be made herein to an “isolated” endolysin polypeptide or fragment thereof. This is merely to distinguish between the endolysin polypeptides or fragments thereof of the invention and endolysin polypeptides or fragments thereof which occur in nature. In the context of the present invention the term “isolated” may be used when referring to such endolysin polypeptides or fragments of the invention when expressed in host cells from nucleic acids and/or from expression vectors, or when present in lysates derived from such host cell and compositions derived from such host cells and lysates and so on. It is to be understood that in this context the term “isolated” is not to be interpreted literally, but is used synonymously with terms such as “recombinant” and “exogenous”, meaning that the endolysin polypeptide or fragment thereof of the invention has been introduced into the cell, lysate, composition etc. artificially by the hand of man.

### Endolysin Polypeptides of the Invention—General Structural Characteristics

The endolysins of the invention possess an N-terminal Clostridium perfringens cell wall peptidoglycan catalytic domain, also referred to herein as an N-terminal catalytic domain. The endolysins of the invention may possess one or more N-terminal Clostridium perfringens cell wall peptidoglycan catalytic domains.

The endolysins of the invention may additionally possess one or more C-terminal Clostridium perfringens cell wall binding domains, also referred to herein as C-terminal binding domain(s).

The endolysins of the invention may additionally possess one or more linkers connecting the N-terminal catalytic domain and the C-terminal binding domain, and/or one or more linkers connecting multiple C-terminal binding domains, if present, and/or one or more linkers connecting multiple N-terminal catalytic domains, if present.

### N-Terminal Catalytic Domain

The N-terminal domain of an endolysin of the invention as described herein is a catalytic domain responsible for the enzymatic cleavage of the peptidoglycan layer of the bacterial cell wall. The catalytic domains thus has an influence on the specificity of the endolysin against its target species, genera or strain.

The N-terminal catalytic domain may possess N-acetyl-β-D-glucosaminidase activity, N-acetyl-β-D-muramidase activity, N-acetylmuramoyl-L-alanine amidase activity and/or endopeptidase activity.

### C-Terminal Binding Domain

The C-terminal cell wall binding domain of an endolysin of the invention as described herein comprises the bacterial cell wall binding domain. The C-terminal cell wall binding domain is thus also responsible for the specificity of the endolysin against its target species, genera or strain. The domain binds noncovalently to the cell envelope, which can be part of the peptidoglycan or other cell wall associated molecules.

In certain polypeptides endolysins may comprise only the N-terminal catalytic domain and still have high specificity for its target bacterial species. The C-terminal binding domain may enhance the specificity of the endolysin but it may not be essential. An endolysin of the invention may also have more than one C-terminal binding domains in sequence, such as two C-terminal binding domains in sequence. It has been postulated that binding of the C-terminal binding domain(s) to the cell wall in the interior of the cell acts to prevent the endolysin from lysing other bacteria that the Clostridium perfringens bacteriophage has yet to infect. As such, the existence of the C-terminal binding domain(s) may be an evolutionary feature to ensure the survival of bacteriophage progeny.

### Linker

In certain polypeptides endolysins of the invention may comprise one or more linkers which connect the N- and C-terminal domains, and/or one or more linkers connecting multiple C-terminal binding domains, if present, and/or one or more linkers connecting multiple N-terminal catalytic domains, if present.

The linker which connects domains is preferably a flexible linker region.

Any suitable linker may be used to connect the domains. The linker may be a naturally-occurring linker, such as an endolysin linker defined by SEQ ID NO herein, or may be an artificial linker. Examples of linkers which may be employed are described in Chen et al. Adv Drug Deliv Rev. Oct. 15, 2013; 65(10): 1357-1369 and in Chichili et al. Protein Sci. 2013 February; 22(2): 153-167.

A linker may be a linear linker or a branched linker.

A linker may comprise a hydrocarbon chain. A hydrocarbon chain may comprise from 2 to about 2000 or more carbon atoms. The hydrocarbon chain may comprise an alkylene group, e.g. C2 to about 2000 or more alkylene groups. The hydrocarbon chain may have a general formula of —(CH2)n- wherein n is from 2 to about 2000 or more. The hydrocarbon chain may be optionally interrupted by one or more ester groups (i.e. —C(O)—O—) or one or more amide groups (i.e. —C(O)—N(H)—).

Any linker may be used selected from the group comprising PEG, polyacrylamide, poly(2-hydroxyethyl methacrylate), Poly-2-methyl-2-oxazoline (PMOXA), zwitterionic polymers, e.g. poly(carboxybetaine methacrylate) (PCBMA), poly[N-(3-sulfopropyl)-N-methacryloxyethyl-N, N dimethyl ammonium betaine](PSBMA), glycopolymers, and polypeptides.

A linker may comprise a polyethylene glycol (PEG) having a general formula of

—(CH2-CH2-O)n-, wherein n is from 1 to about 600 or more.

A linker may comprise oligoethylene glycol-phosphate units having a general formula of —[(CH2-CH2-O)n-PO2-O]m- where n is from 1 to about 600 or more and m could be 1-200 or more.

### Variation in Domain/Linker Architecture

As disclosed herein, the “full length” Clostridium perfringens bacteriophage endolysin polypeptides set forth in any one of SEQ ID NO: 1, 2, 3, 4, 5, 6, 7 and 8 all possess an N-terminal Clostridium perfringens cell wall peptidoglycan catalytic domain, as described and defined herein.

These sequences may additionally contain one C-terminal Clostridium perfringens cell wall binding domain, and/or they may also contain more than one C-terminal Clostridium perfringens cell wall binding domain, as described and defined herein. Further in addition, they may also contain one or more linker sequences as described and defined herein.

Any amino acid sequence which connects an N-terminal Clostridium perfringens cell wall peptidoglycan catalytic domain as defined herein and a C-terminal Clostridium perfringens cell wall binding domain as defined herein may be defined as a linker sequence herein.

Any amino acid sequence which connects one C-terminal Clostridium perfringens cell wall binding domain as defined herein with another C-terminal Clostridium perfringens cell wall binding domain as defined herein may be defined as a linker sequence herein.

In any of the endolysin polypeptides of the invention which comprise an N-terminal Clostridium perfringens cell wall peptidoglycan catalytic domain or fragment thereof as defined herein and one or more C-terminal Clostridium perfringens cell wall binding domain or fragment thereof as defined herein, and optionally one or more linker sequences or fragments thereof as defined herein, the N-terminal catalytic domain or fragment thereof, the C-terminal binding domain or fragment thereof (if present) and the linker or fragment thereof (if present) may be structured in any order.

Thus, for example, a Clostridium perfringens cell wall peptidoglycan catalytic domain (or fragment or variant thereof), which in a naturally-occurring endolysin polypeptide is positioned at the N-terminus of the polypeptide may instead be positioned at the C-terminus of a polypeptide of this invention.

Similarly, a Clostridium perfringens cell wall binding domain (or fragment or variant thereof), which in a naturally-occurring endolysin polypeptide is positioned at the C-terminus of the polypeptide may instead be positioned at the N-terminus of a polypeptide of this invention.

Any one or more linker sequences or fragments thereof in any order may connect any two or more N-terminal domain sequences or fragments thereof in any order and any two or more C-terminal domain sequences or fragments thereof in any order or any N-terminal domain sequences or fragments thereof and any C-terminal domain sequences or fragments thereof in any order.

### Endolysin Polypeptides of the Invention—Structural Characteristics

### Endolysins Polypeptides Comprising an N-Terminal Catalytic Domain

As discussed above, the endolysins of the invention possess an N-terminal Clostridium perfringens cell wall peptidoglycan catalytic domain, also referred to herein as an N-terminal catalytic domain, or a fragment thereof. Accordingly, an endolysin of the invention may be an isolated endolysin polypeptide which has antimicrobial activity, and wherein the isolated polypeptide comprises or consists of an amino acid sequence: which is an N-terminal Clostridium perfringens cell wall peptidoglycan catalytic domain polypeptide, and which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7 or 8; or which is a fragment of said N-terminal catalytic domain polypeptide, and which is at least 80% identical to the amino acid sequence of the sequence set forth in SEQ ID NOs: 1, 2, 3, 4, 5, 6, 7, or 8 which corresponds to the amino acid sequence of the fragment.

The isolated endolysin polypeptide may comprise or consist of an amino acid sequence which is an N-terminal Clostridium perfringens cell wall peptidoglycan catalytic domain and which is at least 85%, 90%, 91%, 92%, 93%, 94%, 95%, 96%, 97%, 98%, 99% or 100% identical to the amino acid sequence of the C. perfringens bacteriophage endolysin polypeptide set forth in SEQ ID NOs: 1, 2, 3, 4, 5, 6, 7, or 8. In the case of a fragment of said amino acid sequence, the endolysin polypeptide may comprise or consist of an amino acid sequence which is at least 85%, 90%, 91%, 92%, 93%, 94%, 95%, 96%, 97%, 98%, 99% or 100% identical to the amino acid sequence of the sequence set forth in SEQ ID NOs: 1, 2, 3, 4, 5, 6, 7, or 8 which corresponds to the amino acid sequence of the fragment.

The N-terminal Clostridium perfringens cell wall peptidoglycan catalytic domains of the endolysin polypeptides defined according to the amino acid sequences set forth in SEQ ID NOs: 1, 2, 3, 4, 5, 6, 7 or 8 are represented respectively by the amino acid sequence set forth in SEQ ID NOs: 12, 13, 14, 15, 16, 17, 19 and 20. In addition, the N-terminal Clostridium perfringens cell wall peptidoglycan catalytic domain of the endolysin polypeptide defined according to the amino acid sequences set forth in SEQ ID NO 6 may be represented by the amino acid sequence set forth in SEQ ID NO: 18.

Any of the endolysin polypeptides defined above may have any antimicrobial activity as defined herein.

### Endolysins Polypeptides Comprising an N-Terminal Catalytic Domain and a C-Terminal Binding Domain

As discussed above, the endolysins of the invention possess an N-terminal Clostridium perfringens cell wall peptidoglycan catalytic domain and additionally may possess one or more C-terminal Clostridium perfringens cell wall binding domains.

The endolysins of the invention may possess a “full length” N-terminal Clostridium perfringens cell wall peptidoglycan catalytic domain of the Clostridium perfringens bacteriophage endolysin polypeptide set forth in SEQ ID NO: 1, 2, 3, 4, 5, 6, 7 or 8 or a fragment thereof in combination with a “full length” C-terminal Clostridium perfringens cell wall binding domain of the Clostridium perfringens bacteriophage endolysin polypeptide set forth in SEQ ID NO: 1, 2, 3, 5, 6, or 8, or in combination with a fragment thereof, and optionally in combination with a “full length” linker of the Clostridium perfringens bacteriophage endolysin polypeptide set forth in SEQ ID NO: 1, 2, 3, 5, 6, or 8, or optionally in combination with a fragment thereof.

The C-terminal Clostridium perfringens cell wall binding domain of the endolysin polypeptides defined according to the amino acid sequences set forth in SEQ ID NOs: 1, 2, 3, 5, 6, or 8 are represented respectively by the amino acid sequence set forth in SEQ ID NOs: 33, 36, 37, 38, 41, and 43.

In addition, a C-terminal Clostridium perfringens cell wall binding domain of the endolysin polypeptide defined according to the amino acid sequences set forth in SEQ ID NO 1 may be represented by the amino acid sequence set forth in SEQ ID NO: 34. In addition, a C-terminal Clostridium perfringens cell wall binding domain of the endolysin polypeptide defined according to the amino acid sequences set forth in SEQ ID NO 1 may be represented by two C-terminal Clostridium perfringens cell wall binding domains as defined by the amino acid sequence set forth in SEQ ID NO: 35.

In addition, a C-terminal Clostridium perfringens cell wall binding domain of the endolysin polypeptide defined according to the amino acid sequences set forth in SEQ ID NO 5 may be represented by the amino acid sequence set forth in SEQ ID NO: 39. In addition, a C-terminal Clostridium perfringens cell wall binding domain of the endolysin polypeptide defined according to the amino acid sequences set forth in SEQ ID NO 1 may be represented by two C-terminal Clostridium perfringens cell wall binding domains as defined by the amino acid sequence set forth in SEQ ID NO: 40.

In addition, the C-terminal Clostridium perfringens cell wall binding domain of the endolysin polypeptide defined according to the amino acid sequences set forth in SEQ ID NO 6 may be represented by the amino acid sequence set forth in SEQ ID NO: 42.

A linker sequence of the endolysin polypeptide defined according to the amino acid sequence set forth in SEQ ID NO 1 may be represented by the amino acid sequence set forth in SEQ ID NO: 24 or 25. A linker sequence of the endolysin polypeptide defined according to the amino acid sequences set forth in SEQ ID NOs: 2 and 3 may be represented by the amino acid sequence set forth in SEQ ID NOs: 26 and 27 respectively. A linker sequence of the endolysin polypeptide defined according to the amino acid sequence set forth in SEQ ID NO 5 may be represented by the amino acid sequence set forth in SEQ ID NO: 28 or 29. A linker sequence of the endolysin polypeptide defined according to the amino acid sequences set forth in SEQ ID NOs: 6 and 8 may be represented by the amino acid sequence set forth in SEQ ID NOs: 30 and 31 respectively.

In the present disclosure, each one of the N-terminal Clostridium perfringens cell wall peptidoglycan catalytic domains, each one of the C-terminal Clostridium perfringens cell wall binding domains (if any) and each one of the linkers (if any) disclosed in any of the polypeptides set forth in SEQ ID NO: 1, 2, 3, 4, 5, 6, 7 or 8 are defined according to homology search using the Pfam software. In addition, any one of the N-terminal Clostridium perfringens cell wall peptidoglycan catalytic domains, any one of the C-terminal Clostridium perfringens cell wall binding domains (if any) and any one of the linkers (if any) disclosed in any of the polypeptides set forth in SEQ ID NO: 1, 2, 3, 4, 5, 6, 7 or 8 may alternatively be defined according to homology search using HHpred software, InterProScan software or the Structural Classification of Proteins (SCOP) database.

Accordingly, an endolysin of the invention may be an isolated endolysin polypeptide which has antimicrobial activity as defined above, further wherein the isolated polypeptide comprises an N-terminal Clostridium perfringens cell wall peptidoglycan catalytic domain polypeptide or fragment thereof, a C-terminal Clostridium perfringens cell wall binding domain polypeptide or fragment thereof and optionally a linker polypeptide or fragment thereof connecting the N- and C-terminal domains; wherein the isolated polypeptide comprises:
d) an amino acid sequence which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7 or 8, or an amino acid sequence which is a fragment of said N-terminal catalytic domain polypeptide and which is at least 80% identical to the amino acid sequence of the sequence set forth in SEQ ID NOs: 1, 2, 3, 4, 5, 6, 7, or 8 which corresponds to the amino acid sequence of the fragment; and
e) an amino acid sequence which is at least 80% identical to an endolysin C-terminal Clostridium perfringens cell wall binding domain polypeptide, or a fragment of said polypeptide; or an amino acid sequence which is at least 80% identical to the amino acid sequence of a C-terminal cell wall binding domain of the Clostridium perfringens bacteriophage endolysin polypeptide set forth in SEQ ID NO: 1, 2, 3, 5, 6, or 8, or an amino acid sequence which is a fragment of said C-terminal binding domain polypeptide and which is at least 80% identical to the amino acid sequence of the sequence set forth in SEQ ID NOs: 1, 2, 3, 5, 6, or 8 which corresponds to the amino acid sequence of the fragment; and
f) an optional amino acid sequence which is at least 80% identical to the linker of an endolysin polypeptide, or a fragment of said polypeptide; or an optional amino acid sequence which is at least 80% identical to the amino acid sequence of a linker of the Clostridium perfringens bacteriophage endolysin polypeptide set forth in SEQ ID NO: 1, 2, 3, 5, 6, or 8, or an amino acid sequence which is a fragment of said linker and which is at least 80% identical to the amino acid sequence of the sequence set forth in SEQ ID NOs: 1, 2, 3, 5, 6, or 8, which corresponds to the amino acid sequence of the fragment.

An endolysin of the invention may be an isolated endolysin polypeptide which has antimicrobial activity as defined above, wherein the isolated polypeptide comprises an N-terminal Clostridium perfringens cell wall peptidoglycan catalytic domain polypeptide or fragment thereof, a C-terminal Clostridium perfringens cell wall binding domain polypeptide or fragment thereof and a linker polypeptide or fragment thereof connecting the N- and C-terminal domains; wherein the isolated polypeptide comprises:
d) an amino acid sequence which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7 or 8, or an amino acid sequence which is a fragment of said N-terminal catalytic domain polypeptide and which is at least 80% identical to the amino acid sequence of the sequence set forth in SEQ ID NOs: 1, 2, 3, 4, 5, 6, 7, or 8 which corresponds to the amino acid sequence of the fragment; and
e) an amino acid sequence which is at least 80% identical to the amino acid sequence of the C-terminal Clostridium perfringens cell wall binding domain of the Clostridium perfringens bacteriophage endolysin polypeptide set forth in SEQ ID NO: 1, 2, 3, 5, 6, or 8, or an amino acid sequence which is a fragment of said C-terminal binding domain polypeptide and which is at least 80% identical to the amino acid sequence of the sequence set forth in SEQ ID NOs: 1, 2, 3, 5, 6, or 8 which corresponds to the amino acid sequence of the fragment; and
f) an amino acid sequence which is at least 80% identical to the amino acid sequence of a linker of the Clostridium perfringens bacteriophage endolysin polypeptide set forth in SEQ ID NO: 1, 2, 3, 5, 6, or 8, or an amino acid sequence which is a fragment of said linker and which is at least 80% identical to the amino acid sequence of the sequence set forth in SEQ ID NOs: 1, 2, 3, 5, 6, or 8 which corresponds to the amino acid sequence of the fragment.

An endolysin of the invention may be an isolated endolysin polypeptide which has antimicrobial activity as defined above, wherein the isolated polypeptide comprises: an amino acid sequence wherein the N-terminal catalytic domain, the C-terminal cell wall binding domain and the linker are all at least 80% identical to corresponding amino acid sequences respectively of the N-terminal domain, a C-terminal domain and a linker of the Clostridium perfringens bacteriophage endolysin polypeptide set forth in SEQ ID NOs: 1, 2, 3, 4, 5, 6, 7, or 8.

An endolysin of the invention may be an isolated endolysin polypeptide which has antimicrobial activity as defined above, wherein the isolated polypeptide comprises:
d) an amino acid sequence consisting of a fragment of said N-terminal catalytic domain and which is at least 80% identical to the amino acid sequence of the sequence set forth in SEQ ID NOs: 1, 2, 3, 4, 5, 6, 7, or 8 which corresponds to the amino acid sequence of the fragment; and/or
e) an amino acid sequence consisting of a fragment of said C-terminal binding domain and which is at least 80% identical to the amino acid sequence of the sequence set forth in SEQ ID NOs: SEQ ID NO: 1, 2, 3, 5, 6, or 8 which corresponds to the amino acid sequence of the fragment; and/or
f) an amino acid sequence consisting of a fragment of said linker and which is at least 80% identical to the amino acid sequence of the sequence set forth in SEQ ID NOs: 1, 2, 3, 5, 6, or 8 which corresponds to the amino acid sequence of the fragment.

In any of the endolysins of the invention described above which possess an N-terminal Clostridium perfringens cell wall peptidoglycan catalytic domain or an amino acid sequence which is fragment thereof and a C-terminal Clostridium perfringens cell wall binding domain or an amino acid sequence which is a fragment thereof and optionally a linker or an amino acid sequence which is a fragment thereof, the percentage identity to the reference sequence may be higher than 80%.

Accordingly, in any such isolated endolysin polypeptide, the endolysin polypeptide may comprise or consist of an amino acid sequence which is an N-terminal Clostridium perfringens cell wall peptidoglycan catalytic domain and which is at least 85%, 90%, 91%, 92%, 93%, 94%, 95%, 96%, 97%, 98%, 99% or 100% identical to the amino acid sequence of the C. perfringens bacteriophage endolysin polypeptide set forth in SEQ ID NOs: 1, 2, 3, 4, 5, 6, 7, or 8. In the case of an amino acid sequence which is a fragment of said amino acid sequence, the endolysin polypeptide may comprise or consist of an amino acid sequence which is at least 85%, 90%, 91%, 92%, 93%, 94%, 95%, 96%, 97%, 98%, 99% or 100% identical to the amino acid sequence of the sequence set forth in SEQ ID NOs: 1, 2, 3, 4, 5, 6, 7, or 8 which corresponds to the amino acid sequence of the fragment. In any such isolated endolysin polypeptide, the endolysin polypeptide may comprise or consist of an amino acid sequence which is a C-terminal Clostridium perfringens cell wall binding domain and which is at least 85%, 90%, 91%, 92%, 93%, 94%, 95%, 96%, 97%, 98%, 99% or 100% identical to the amino acid sequence of a known endolysin C-terminal Clostridium perfringens cell wall binding domain polypeptide. In the case of a fragment of said amino acid sequence, the endolysin polypeptide may comprise or consist of an amino acid sequence which is at least 85%, 90%, 91%, 92%, 93%, 94%, 95%, 96%, 97%, 98%, 99% or 100% identical to the amino acid sequence of the sequence of the known endolysin C-terminal Clostridium perfringens cell wall binding domain polypeptide which corresponds to the amino acid sequence of the fragment. Alternatively, in any such isolated endolysin polypeptide, the endolysin polypeptide may comprise or consist of an amino acid sequence which is a C-terminal Clostridium perfringens cell wall binding domain and which is at least 85%, 90%, 91%, 92%, 93%, 94%, 95%, 96%, 97%, 98%, 99% or 100% identical to the amino acid sequence of a C. perfringens bacteriophage endolysin polypeptide set forth in SEQ ID NOs: 1, 2, 3, 5, 6, or 8. In the case of a fragment of said amino acid sequence, the endolysin polypeptide may comprise or consist of an amino acid sequence which is at least 85%, 90%, 91%, 92%, 93%, 94%, 95%, 96%, 97%, 98%, 99% or 100% identical to the amino acid sequence of the sequence set forth in SEQ ID NOs: 1, 2, 3, 5, 6, or 8 which corresponds to the amino acid sequence of the fragment. In any such isolated endolysin polypeptide, the endolysin polypeptide may optionally comprise or consist of an amino acid sequence which is at least 85%, 90%, 91%, 92%, 93%, 94%, 95%, 96%, 97%, 98%, 99% or 100% identical to the amino acid sequence of a linker of a known endolysin polypeptide. In the case of a fragment of said amino acid sequence, the endolysin polypeptide may comprise or consist of an amino acid sequence which is at least 85%, 90%, 91%, 92%, 93%, 94%, 95%, 96%, 97%, 98%, 99% or 100% identical to the amino acid sequence of a linker of a known endolysin polypeptide which corresponds to the amino acid sequence of the fragment. Alternatively, in any such isolated endolysin polypeptide, the endolysin polypeptide may comprise or consist of an amino acid sequence which is at least 85%, 90%, 91%, 92%, 93%, 94%, 95%, 96%, 97%, 98%, 99% or 100% identical to the amino acid sequence of the linker of the Clostridium perfringens bacteriophage endolysin polypeptide set forth in SEQ ID NO: 1, 2, 3, 5, 6, or 8. In the case of a fragment of said amino acid sequence, the endolysin polypeptide may comprise or consist of an amino acid sequence which is at least 85%, 90%, 91%, 92%, 93%, 94%, 95%, 96%, 97%, 98%, 99% or 100% identical to the amino acid sequence of the sequence set forth in SEQ ID NOs: 1, 2, 3, 5, 6, or 8 which corresponds to the amino acid sequence of the fragment.

Any of the endolysin polypeptides defined above may have any antimicrobial activity as defined herein.

An endolysin of the invention may be an isolated endolysin polypeptide which has antimicrobial activity as defined above, wherein the polypeptide comprises:
c) an amino acid sequence comprising all of the N-terminal domain, the C-terminal domain(s) and the linker(s) of the Clostridium perfringens bacteriophage endolysin polypeptide set forth in SEQ ID NOs: 1, 2, 3, 6, or 8, and wherein the amino acid sequences are 100% identical to the sequences of the N-terminal domain, the C-terminal domain and the linker of the Clostridium perfringens bacteriophage endolysin polypeptide set forth in SEQ ID NOs: SEQ ID NO: 1, 2, 3, 5, 6, or 8; or
d) an amino acid sequence comprising fragments of all of the N-terminal domain, the C-terminal(s) domain and the linker(s) of the Clostridium perfringens bacteriophage endolysin polypeptide set forth in SEQ ID NOs: 1, 2, 3, 5, 6, or 8, and wherein the amino acid sequences of each fragment are 100% identical to the sequences of the Clostridium perfringens bacteriophage endolysin polypeptide set forth in SEQ ID NOs: 1, 2, 3, 5, 6, or 8 which correspond to the amino acid sequences of the fragment.

An endolysin of the invention may be an isolated endolysin polypeptide which has antimicrobial activity as defined above, wherein the polypeptide comprises an amino acid sequence which is 100% identical to the amino acid sequence of the Clostridium perfringens bacteriophage endolysin polypeptide set forth in SEQ ID NO: 1, 2, 3, 4, 5, 6, 7 or 8.

### Endolysins Polypeptides Comprising One or More Fragments and Variants

Any of the above-described fragments may be truncated with respect to the full length amino acid sequence set forth in SEQ ID NOs: 1, 2, 3, 4, 5, 6, 7, or 8 or with respect to domains/linkers defined in the amino acid sequences set forth in SEQ ID NOs: 1, 2, 3, 4, 5, 6, 7, or 8, provided that any such fragment has any antimicrobial activity as defined herein. Such a fragment may be truncated by any number of amino acids, at the N-terminal and/or C-terminal ends of the polypeptide.

An endolysin polypeptide according to the invention may be derived from any one of the C. perfringens bacteriophage endolysin polypeptides set forth in SEQ ID NOs: 1, 2, 3, 4, 5, 6, 7, or 8 by substituting, inserting, deleting, or adding any number of amino acids at any position, such as 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more or 50 or more amino acids, provided that any such fragment has any antimicrobial activity as defined herein. One more or all amino acid substitutions may be conservative amino acid substitutions.

An endolysin polypeptide according to the invention may be derived from one of the sequences as identified herein by adding one or more additional N- or C-terminal amino acids or chemical moieties to increase stability, solubility and activity.

An endolysin polypeptide according to the invention may have a length of at least 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 950 or more, 1000 or more, 1100 or more, 1200 or more, 1300 or more, 1400 or more, 1500 or more, or 2000 or more amino acids.

A variant endolysin polypeptide may be any non-naturally occurring or genetically engineered form of the endolysin polypeptide according to the invention, including any fragment or derivative thereof, provided that any such variant has any antimicrobial activity as defined herein. An endolysin polypeptide variant may differ in some engineered way from any of the polypeptides disclosed herein by reference to SEQ ID NOs. For example, a variant endolysin polypeptide according to the invention may be made by site-directed mutagenesis starting from the nucleotide sequence encoding a polypeptide defined by any one of SEQ ID NOs disclosed herein.

A fragment of variant endolysin polypeptide according to the invention may be defined according to percentage identity with respect to a reference amino acid sequence or a reference polynucleotide sequence. For example a fragment of variant endolysin polypeptide according to the invention may be defined according to percentage identity with respect to the amino acid sequence of an endolysin polypeptide defined by a sequence set forth in any one of SEQ ID NOs: 1 to 8 as the reference amino acid sequence, or by a sub-sequence set forth in any one of SEQ ID NOs: 1 to 8 as the reference amino acid sequence, such as the sequence of any domain or linker defined therein or a sequence portion of such a domain or linker.

Amino acid identity may be calculated using any suitable algorithm. For example the PILEUP and BLAST algorithms can be used to calculate identity or line up sequences (such as identifying equivalent or corresponding sequences (typically on their default settings), for example as described in Altschul S. F., 1993 J Mol Evol 36:290-300; Altschul, S, F et al, 1990 J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff, 1992 Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul, 1993 Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al, 1984 Nucleic Acids Research 12, 387-395).

As described herein, the amino acid sequence of a polypeptide of the invention may comprises the amino acid sequence of the whole or a portion of a reference sequence defined by SEQ ID NO in which modifications, such as amino acid additions, deletions or substitutions are made relative to the reference sequence or portion thereof. The modifications may be conservative or non-conservative amino acid substitutions. Where there are multiple modifications in a single polypeptide, the modifications in the polypeptide sequence may be a combination of conservative and non-conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in the Table below.

TABLE
Chemical properties of amino acids

| | |
|---|---|
| Ala (A) | aliphatic, hydrophobic, neutral |
| Cys (C) | polar, hydrophobic, neutral |
| Asp (D) | polar, hydrophilic, charged (−) |
| Glu (E) | polar, hydrophilic, charged (−) |
| Phe (F) | aromatic, hydrophobic, neutral |
| Gly (G) | aliphatic, neutral |
| His (H) | aromatic, polar, hydrophilic, charged (+) |
| Ile (I) | aliphatic, hydrophobic, neutral |
| Lys (K) | polar, hydrophilic, charged (+) |
| Leu (L) | aliphatic, hydrophobic, neutral |
| Met (M) | hydrophobic, neutral |
| Asn (N) | polar, hydrophilic, neutral |
| Pro (P) | hydrophobic, neutral |
| Gln (Q) | polar, hydrophilic, neutral |
| Arg (R) | polar, hydrophilic, charged (+) |
| Ser (S) | polar, hydrophilic, neutral |
| Thr (T) | polar, hydrophilic, neutral |
| Val (V) | aliphatic, hydrophobic, neutral |
| Trp (W) | aromatic, hydrophobic, neutral |
| Tyr (Y) | aromatic, polar, hydrophobic |

Where amino acids have similar polarity, this can be determined by reference to the hydropathy scale for amino acid side chains in the Table below.

TABLE
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One of skill in the art may determine whether any fragment or variant endolysin possesses antimicrobial activity by performing a functional test for antimicrobial activity as described further herein.

### Endolysin Polypeptides of the Invention—Functional Characteristics

An endolysin of the invention may provide an antimicrobial effect that can be defined by bacteriolytic activity. Bacteriolytic activity can be measured using a turbidity reduction assay, a t50% lysis value (time required for an endolysin to lyse 50% of its initial cell population) and/or a cell viability assay (log₁₀ values plotted against increasing concentrations of endolysin or ratio of control over endolysin-treated cell reactions to calculate the percentage reduction of viable bacteria for each endolysin). Bacteriolytic activity can be defined by minimum bactericidal concentration (MBC).

An endolysin of the invention may provide an antimicrobial effect that can be defined by bacteriostatic or growth inhibitory activity. Bacteriostatic or growth inhibitory activity can be defined by minimum inhibitory concentration (MIC).

An endolysin of the invention may provide an antimicrobial effect against C. perfringens bacteria that is higher compared to the effect observed against relevant control bacteria. A relevant control may be any bacterial strain that is not C. perfringens (herein referred to as a “non-Cp” strain). The non-Cp strain with its DSMZ (Deutsche Sammlung von Mikroorganismen und Zellkulturen) number in parentheses includes Clostridium colinum (DSMZ 6011), Clostridium leptum (DSMZ 753), Clostridium cellobioparum (DSMZ 1351), and Bifidobacterium adolescentis (DSMZ 20083) (see also Example 6). These are four non-Cp bacteria strains commonly found in poultry gut and may be used to determine whether endolysin activity and hence antimicrobial activity is specific to C. perfringens.

C. perfringens strains may be used to determine the antimicrobial activity of the endolysin of the invention. C. perfringens strains include NCTC (National Collection of Type Cultures) Type A strains procured from the Public Health England culture collection, with the accession numbers: 2837, 8235, 8237, 8238, 8239, 8359, 8678, 10578. These C. perfringens strains may be referred herein as “reference strains”. C. perfringens strains may also include Type A field isolates obtained procured from the United Kingdom Animal and Plant Health Agency (APHA) with the accession numbers: B00907, B00917, B00954, B00964, B00976, G00033, W000101, W00102. These C. perfringens Type A field isolates may be referred herein as “field isolates”.

An endolysin polypeptide of the invention may exhibit antimicrobial effects to one or more of the C. perfringens reference strains and the C. perfringens Type A field isolates disclosed herein. The endolysin of the invention may exhibit antimicrobial effects against NCTC strains: 2837, 8235, 2837, 8235, 8237, 8238, 8239, 8359, 8678 and/or 10578.

The antimicrobial activity may be determined by using a standard cell viability assay as disclosed herein to calculate a Δ log₁₀ value. The Δ log₁₀ value exhibited by an endolysin polypeptide or fragment may be determined using a concentration of 5 μg/ml of an endolysin or fragment of the invention against a C. perfringens reference strain, NCTC 8237. The Δ log₁₀ value may be approximately 0.40 or more, 2.00 or more or 3.70 or more. Approximately is taken to mean+/−10% or +/−5%.

The antimicrobial activity may be determined by using a standard cell viability assay as disclosed herein to calculate a % reduction in cell viability. The % reduction cell viability value exhibited by an endolysin polypeptide or fragment of the invention may be determined using a concentration of 5 μg/ml of an endolysin or fragment of the invention against a C. perfringens reference strain, NCTC 8237. The % reduction in cell viability may be approximately 60% or more, 70% or more, 80% or more, 90% or more or 100%. Approximately is taken to mean+/−10% or +/−5%.

The antimicrobial activity may be determined by using a standard cell turbidity reduction assay as disclosed herein, to calculate a t50 lysis value. The t50 lysis value exhibited by an endolysin polypeptide or fragment is determined using a concentration of 5 μg/ml of an endolysin of the invention or a fragment against a C. perfringens reference strain, NCTC 8237. The t50 lysis value provided may be approximately 7 minutes or less; or it may provide a t50% lysis value of 3 minutes or less. Approximately is taken to mean+/−10% or +/−5%.

The antimicrobial activity may be determined by using a standard minimum inhibitory concentration (MIC) assay and/or a minimum bactericidal concentration (MBC) assay. A standard minimum inhibitory concentration (MIC) assay or a minimum bactericidal concentration (MBC) assay may be performed against Clostridium perfringens strain Cp6 at 2.1×10⁴ cells/mL wherein an endolysin of the invention or a fragment exhibits an MIC value or an MBC value of 17 μg/mL or less, preferably 1.7 μg/mL or less; or it exhibits an MIC value or an MBC value of 0.65 μM or less, preferably 0.05 μM or less. Approximately is taken to mean+/−10% or +/−5%.

Endolysin activity (i.e. hydrolytic activity), and therefore antimicrobial activity, may be determined by assessing the rate of degradation of purified peptidoglycan (PGN) in vitro. This may be assessed by measuring the spontaneous rate of PGN degradation in any suitable buffer. The rate of degradation of purified PGN in vitro can be assessed by optical density. For use in such an assay, preferably the PGN is purified from a bacterial species, more preferably from a C. perfringens strain, such as the C. perfringens Cp6 strain. A protocol for the purification of PGN from a bacterial species is provided in Example 20 below.

Any endolysin polypeptide of the invention may be determined to possess endolysin hydrolytic activity, and therefore antimicrobial activity, if it promotes a statistically significant increase in the rate of degradation of purified PGN in vitro. The ability of a given endolysin polypeptide to promote a statistically significant increase in the rate of degradation of purified PGN in vitro can be assessed by reference to the background rate of PGN degradation, i.e. by reference to a control preparation which does not contain an endolysin polypeptide.

The ability of a given endolysin polypeptide to promote a statistically significant increase in the rate of degradation of purified PGN in vitro can be assessed by reference to the rate of PGN degradation by a reference or control polypeptide. A reference or control polypeptide should be a polypeptide which is not capable of promoting a statistically significant increase in the rate of degradation of purified PGN in vitro. A reference or control polypeptide can be a polypeptide which is a PGN-catalytically inactive endolysin, or any other suitable polypeptide which is not an endolysin, such as bovine serum albumin.

Any endolysin polypeptide of the invention may be determined to possess endolysin hydrolytic activity, and therefore antimicrobial activity, if it promotes a statistically significant reduction in the absolute amount of purified PGN in vitro (i.e. a statistically significant degradation of purified PGN in vitro) at the end-point (completion) of a PGN degradation assay.

The ability of a given endolysin polypeptide to promote a statistically significant reduction in the absolute amount of purified PGN in vitro can also be assessed by reference to the background reduction in the absolute amount of purified PGN, i.e. by reference to a control preparation which does not contain an endolysin polypeptide.

The ability of a given endolysin polypeptide to promote a statistically significant reduction in the absolute amount of purified PGN in vitro can also be assessed by reference to the reduction in the absolute amount of purified PGN degradation by a reference or control polypeptide. A reference or control polypeptide should be a polypeptide which is not capable of promoting a statistically significant reduction in the absolute amount of purified PGN in vitro. A reference or control polypeptide can be a polypeptide which is a PGN-catalytically inactive endolysin, or any other suitable polypeptide which is not an endolysin, such as bovine serum albumin.

Endolysin hydrolytic activity can be assessed in this way for purified endolysins, such as explained in the examples, including e.g. Example 21 below, as well as for endolysins comprised in cell extracts, as explained in the examples, including e.g. Example 24 below.

In the case of purified endolysins, activity may be assessed by reference to the background rate of degradation, or background reduction in the absolute amount, of purified PGN, i.e. by reference to a control preparation which does not contain a purified endolysin polypeptide. Alternatively, activity may be assessed by reference to a control preparation which contains a purified control polypeptide, which can be a polypeptide which is a PGN-catalytically inactive endolysin, or any other suitable polypeptide which is not an endolysin, such as bovine serum albumin. In the case of endolysins comprised in cell extracts, activity may be assessed by reference to the background rate of degradation, or background reduction in the absolute amount, of purified PGN, i.e. by reference to a control preparation which does not contain a cell extract comprising an endolysin polypeptide. Preferably the control contains a cell extract comparable with the test sample but minus the endolysin polypeptide. Alternatively, activity may be assessed by reference to a control preparation which contains a control polypeptide comprised in a comparable cell extract. A control polypeptide can be a polypeptide which is a PGN-catalytically inactive endolysin, or any other suitable polypeptide which is not an endolysin, such as bovine serum albumin.

Antimicrobial activity such as bacteriolytic activity and bacteriostatic activity, may be determined according to the assays described in the following section and in any of the relevant Examples.

Assays for bacterolytic activity include spot assays (see Example 8, 11, and 16) in which endolysin polypeptides of the invention and fragments thereof are added to C. perfringen cell lawns already established on the appropriate medium (e.g. agar plate). Any relevant negative control, such as PBS, may be used. Any positive control such as any antibiotic (e.g. ampicillin may be used) or any other product that exhibits an antimicrobial and/or bacteriolytic effect on C. perfringens may be used. Bacteriolytic activity is indicated by the presence of clearance zones and the extent of bacteriolytic activity can be determined by the size of the clearance zones.

Bacteriostatic (growth inhibitory) activity, may be determined as described herein and in Example 8, 11, and 16. Assays for bacteriostatic activity include spot assays in which C. perfringens strains are freshly-plated on the appropriate medium (e.g. agar plate) and varying concentrations of the endolysin polypeptide of the invention added to the freshly plated bacteria. The extent of the bacteriostatic activity can be determined by lack of growth of C. perfringen colonies compared with non-treated controls or controls treated with products that do not affect bacterial growth.

The relative increase in the bacteriolytic/log₁₀/t50% lysis value associated with any of the endolysin polypeptides of the invention, or the relative decrease in cell viability associated with any of the endolysin polypeptides of the invention compared with the reference endolysin polypeptide of a relevant control strain may be assessed by any suitable means. Any such relative increase or any such relative decrease associated with any of the endolysin polypeptides of the invention should be a statistically significant increase or decrease in the relevant property being assessed compared to a reference or control. A reference or control may be reference to the background, i.e. by reference to a control preparation which does not contain an endolysin polypeptide. A reference or control may be reference to a reference or control preparation comprising a reference or control polypeptide. A reference or control polypeptide should be a polypeptide which is not capable of increasing or decreasing the relevant property being assessed. A reference or control polypeptide can be a polypeptide which is a PGN-catalytically inactive endolysin, or any other suitable polypeptide which is not an endolysin, such as bovine serum albumin.

The relative increase may be assessed by comparing the endolytic/bacteriolytic/log₁₀/t50% lysis value/cell viability activity of purified proteins of the present invention against any suitable control protein. For example, the enzyme, lysozyme or bovine serum albumin may be employed as a control protein.

Preferably, the relative increase may be assessed by comparing the endolytic/bacteriolytic/log₁₀/t50% lysis value/cell viability activity in clarified lysates when polypeptides are separately expressed in a host cell, preferably in an E. coli or an algal host cell.

More specifically, the antimicrobial activities of the endolysin polypeptides of the invention can be determined using the assays outlined in the following section and in the Examples:

### Assays for Antimicrobial Effect (Δ Log 10 and Cell Viability) of Endolysins:

Δ Log 10, which is the log₁₀ of the ratio of untreated cell control reactions over endolysin-treated cell reactions, may be calculated according to the following method (see also Example 13):
1. inoculate Clostridium perfringens strain NCTC 8237 into BHI+C media and culture under anaerobic conditions at 37° C. overnight to stationary phase;
2. inoculate the overnight culture into fresh BHI+C media and culture under anaerobic conditions at 37° C. to OD 600 nm approximately 0.6;
3. centrifuge 1.8 mL culture at 16600× g for 5 minutes at room temperature under anaerobic conditions;
4. remove supernatant and resuspend cell pellet in 0.9 mL of resuspension buffer (NaCl 127 mM, Na₂HPO₄ 70 mM, NaH₂PO₄ 30 mM pH 7.0) under anaerobic conditions;
5. dilute cell suspension with resuspension buffer to OD 600 nm 0.6 under anaerobic conditions;
6. add purified endolysin polypeptide or fragment to a concentration of 5 μg/mL under anaerobic conditions;
7. incubate at 25° C. for 1 hour under anaerobic conditions;
8. determine viable cell count by serial dilution, plating cells on RCM solid media and incubating at 37° C. until colonies form under anaerobic conditions; and
9. calculate log 10 reduction value as the log 10 of the ratio of untreated cells control over cells treated with the endolysin polypeptide or fragment.

Cell viability may be calculated according to the following method (see also Example 13 and FIG. 20):
1. inoculate Clostridium perfringens strain NCTC 8237 into BHI+C media and culture under anaerobic conditions at 37° C. overnight to stationary phase;
2. inoculate the overnight culture into fresh BHI+C media and culture under anaerobic conditions at 37° C. to OD 600 nm approximately 0.6;
3. centrifuge 1.8 mL culture at 16600× g for 5 minutes at room temperature under anaerobic conditions;
4. remove supernatant and resuspend cell pellet in 0.9 mL of resuspension buffer (NaCl 127 mM, Na₂HPO₄ 70 mM, NaH₂PO₄ 30 mM pH 7.0) under anaerobic conditions;
5. dilute cell suspension with resuspension buffer to OD 600 nm 0.6 under anaerobic conditions;
6. add purified endolysin polypeptide or fragment to a concentration of 5 μg/mL under anaerobic conditions;
7. incubate at 25° C. for 1 hour under anaerobic conditions;
8. determine viable cell count by serial dilution, plating cells on RCM solid media and incubating at 37° C. until colonies form under anaerobic conditions;
9. calculate log 10 reduction value as the log 10 of the ratio of untreated cells control over cells treated with the endolysin polypeptide or fragment; and
10. use log 10 reduction value to calculate the percentage reduction of viable bacteria.

The t50% lysis value, which is defined as the time required for an endolysin to lyse 50% of its initial cell suspension may be calculated according to the following method (see also Example 12):
1. inoculate Clostridium perfringens strain NCTC 8237 in BHI+C media and culture under anaerobic conditions at 37° C. overnight to stationary phase;
2. inoculate the overnight culture into fresh BHI+C media and culture under anaerobic conditions at 37° C. until the culture reaches exponential phase (OD 620 nm approximately 1.0);
3. centrifuge a volume of the exponential phase culture at 3800× g for 30 minutes at 10° C. and then remove the supernatant;
4. resuspend the cells in PBS, pH 7.0, in a volume which is half of the initial pre-centrifuged culture volume in order to concentrate the cells;
5. transfer 90 μL of the resuspended cells into a well of a 96-microwell plate and add 10 μL of 10× concentrated endolysin polypeptide or fragment in PBS, pH 7.0, to a final concentration of 5 μg/mL;
6. monitor the reduction in turbidity by performing an OD 620 nm kinetic read with suitable intervals on a plate reader at room temperature;
7. monitor turbidity for 30-60 minutes, or until the OD 620 nm reduces such that it begins to plateau;
8. from the turbidity reduction profile, calculate t50% lysis (minutes), i.e. the time for 50% of cells to be lysed, using the following formula:

$$50 \% = \frac{{OE}_{{E \_ t}_{50 \% lysis}} - 0.03}{{OD}_{b \_ t = 0} - 0.03} \times 100 \%$$

wherein ODb_t=0 is the optical density of the initial cell suspension at the beginning of the test, prior to addition of the endolysin polypeptide or fragment; and
wherein ODE_t50% lysis is the optical density of the cell suspension corresponding to 50% reduction in the initial cell density following addition of the endolysin polypeptide or fragment.

### Assays for Minimum Inhibitory Concentration (MIC)/Minimum Bactericidal Concentration (MBC) of Endolysins:

The MIC/MBC value, which is defined as the minimum concentration of purified endolysin which leads to cell growth arrest for a given Clostridium perfringens cell density, may be calculated according to the following method (see also Example 15):
1. inoculate Clostridium perfringens strain Cp6 in BHI+C media and culture under anaerobic conditions at 37° C. overnight to stationary phase;
2. dilute stationary phase culture in fresh LB media under anaerobic conditions;
3. prepare stocks of a serial dilution of purified endolysin to 10× more concentrated than the final desired protein loading in PBS, pH 7.0 under anaerobic conditions;
4. mix a serial dilution of purified endolysin with the diluted stationary phase culture across the wells of a microtitre plate wherein for each well 45 μL of diluted stationary phase culture is mixed with 5 μL of the 10× concentrated and purified endolysin to a final cell loading of approximately 10⁴ cells/mL under anaerobic conditions;
5. incubate cells overnight for 12-20 hrs at 41° C. under anaerobic conditions;
6. determine the extent of growth inhibition by measuring the change in OD on a plate reader and compare to both the media and growth controls;
7. determine the minimum inhibitory concentration (MIC) for the endolysin, wherein MIC is the lowest concentration of endolysin which arrests cell growth and no visible growth is observed;
8. transfer 5 μL of culture from each well into a separate well and make up to 100 μL with BHI+C medium under anaerobic conditions;
9. incubate the transferred cultures overnight for 12-20 hrs at 41° C. under anaerobic conditions;
10. determine the extent of growth, and therefore the extent of residual viable Cp cells following exposure to endolysin, by measuring the change in OD on a plate reader following incubation and compare to both the media and growth controls;
11. determine the minimum bactericidal concentration (MBC) for the endolysin, wherein MBC is the lowest concentration of endolysin which kills all of the initial cell loading and no visible growth is observed.

### Synergy Between Endolysin Polypeptides

Endolysin variants of the invention may also be combined to yield synergistic antimicrobial effects (Examples 16 to 18, Example 21, Example 30). Accordingly, the present invention provides for a combination of two or more endolysin polypeptides of the invention to be used together wherein the exhibited antimicrobial activity is a synergistic activity.

Synergistic activity of endolysin polypeptides of the invention used together may be assessed according to any suitable assay described herein. The PGN degradation assay described herein is a particularly suitable assay. Synergistic activities of multiple endolysin polypeptides of the invention used together have been demonstrated in PGN degradation assays described herein, both as purified proteins (e.g. Example 21), and as components of cell extracts (e.g. Example 30).

The invention provides any host cell, cell lysate, composition, whole-cell composition, dried biomass composition or antimicrobial formulation as described and defined herein, wherein the exhibited antimicrobial activity is a synergistic activity.

The invention also provides any host cell, cell lysate, composition, whole-cell composition or antimicrobial formulation as described and defined herein, wherein the exhibited antimicrobial activity is a synergistic activity, comprising two endolysin polypeptides which are defined according to the amino acid sequences set forth in SEQ ID NO: 1 and SEQ ID NO: 2 and which exhibit synergistic antimicrobial activity, or any combination of fragments and variants of two endolysin polypeptides which are defined according to the amino acid sequences set forth in SEQ ID NO: 1 and SEQ ID NO: 2 and which exhibit synergistic antimicrobial activity.

### Polynucleotides and Vectors Encoding Endolysin Polypeptides of the Invention

The invention further provides nucleic acid molecules and vectors which encode a polypeptide of the invention. Exemplary polynucleotide molecules encoding polypeptides disclosed herein are provided as SEQ ID NOs: 1 to 8. Each of these sequences may include at the 5′ end a codon for the N terminal methionine (ATG) and, prior to the stop codon (TAA) at the 3′ end, codons for a 3× gly linker and a 6× his histidine tag, which may optionally be excluded.

The invention also provides for a polynucleotide encoding an endolysin polypeptide, such that the polynucleotide is codon optimized for efficient expression in a specific host. Codon optimisation may be carried out as according to Example 2 in which endogenous tryptophan “TGG” codons are replaced with “TGA” stop codons to minimise potentially toxic effects to E. coli cloning strains. The person skilled in the art also has knowledge of how to optimise codons and/or codon pairs.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al, 1989, Molecular Cloning—a laboratory manual; Cold Spring Harbor Press. The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

An expression vector to be used for expression of the polypeptide of the invention may comprise two multiple cloning sites (MCSs) which position DNA sequences of choice under the control of two independent T7 polymerase promoters, in which the promoters are engineered to respond to the presence of IPTG in the media, allowing control of gene expression. Such a vector may be the pETDuet (Merck Millipore) expression vector. The vector may comprise additional or alternative means of controlling gene expression. Furthermore, the expression vector is modified in accordance with Example 2 such that the first tryptophan codon “TGG”, or both the first and second tryptophan codons present in the nucleotide sequence encoding the endolysin of the invention is mutated to “TGA” stop codons to prevent toxicity to the E. coli cloning strain arising from undesirable expression of the endolysin of the invention. In such cases, co-expression of a t-RNA molecule in which the anti-codon is “TCA” is desired when expressing said mutated endolysin polypeptide in a host cell. The t-RNA molecule may be one identified in E. coli strains. The t-RNA molecule may be as described in Example 2, such as that identified from the E. coli k-12 strain (GenBank accession no. CP028306) in which the anti-codon, “CCA” is replaced with “TCA”. Any polynucleotide sequences such as the endolysin and the t-RNA molecule disclosed herein may further comprise additional sequences homologous to the MCSs to facilitate cloning into the MCSs.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al. Depending on the host/vector system utilized, any of a number of suitable transcription and translation control elements, including constitutive and inducible promoters, transcription enhancer elements, transcription terminators, etc. may be used in the expression vector (e.g., U6 promoter, HI promoter, etc.).

Thus, the present invention may relate to an expression vector comprising a nucleic acid construct or a polynucleotide according to the invention. An expression vector according to the invention may be a recombinant expression vector. Such vector may constitute a plasmid, a cosmid, a bacteriophage or a virus, or a part thereof, which is transformed by introducing a nucleic acid construct or a polynucleotide according to the invention. Such transformation vectors specific to the host organism to be transformed are well known to those skilled in the art and widely described in the literature. In order to produce a polynucleotide or endolysin polypeptide according to the invention in a host, a process for the transformation of a host organism, and integration of a polynucleotide, nucleic acid construct or expression vector according to the invention may be appropriate. Such transformation may be carried out by any suitable known means which have been widely described in the specialist literature and are well-known to the person skilled in the art.

As used herein, a “promoter” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Non-limiting examples of suitable promoters include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of skilled person. The expression vector may also contain a ribosome binding site for translation initiation and a transcription terminator.

The expression vector may also include appropriate sequences for amplifying expression. The expression vector may also include nucleotide sequences encoding protein tags (e.g., 6×His tag, hemagglutinin tag, green fluorescent protein, thioredoxin, etc.) that are fused to the site—directed modifying polypeptide, thus resulting in a chimeric polypeptide. Such tags may also be cleavable, for example using a TEV protease.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.)(e.g., sub-tissue or tissue specific promoter, cell type specific promoter, etc.), and it may be a temporally restricted promoter (i.e., the promoter is in the “ON” state or “OFF” state during specific stages of development or during specific stages of a biological process, e.g., of the cell cycle.

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g. a promoter) or a coding sequence and/or regulate translation of an encoded polypeptide.

A “vector” or “expression vector” is a replicon, such as plasmid, phage, virus, or cosmid, to which another DNA segment, i.e. an “insert”, may be attached so as to bring about the replication of the attached segment in a cell. An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

### Affinity Tags and Signal Peptide Sequences

An endolysin polypeptide of the invention may further comprise the endolysin sequence together with an affinity tag at the N terminus and/or at the C terminus to further assist with isolation from standard expression systems such as those described herein. Any suitable affinity tag may be used.

Suitable affinity tags may be joined directly to the N or C terminus of a polypeptide or joined indirectly by any suitable linker sequence, such as 3, 4 or 5 glycine residues. One such suitable affinity tag is a histidine tag. The histidine tag typically consists of six histidine residues, although it can be longer than this, for example up to 7, 8, 9, 10 or 20 amino acids or shorter, for example 5, 4, 3, 2 or 1 amino acids. Alternative affinity tags include an AviTag, a FLAG-tag, a HA-tag, a Myc-tag, a Strep-tag, a V5-tag, a Poly-arg, a HAT-tag, a calmodulin-binding peptide tag, a GST tag, a MBP tag, a Fh8 tag, a Strep-II tag, a cellulose binding peptide tag, a chitin-binding peptide tag, a protein A tag, a ubiquitin tag, a DHFR tag, a SBR tag.

Each endolysin of the invention may further comprise the endolysin sequence together with an expression-enhancing peptide sequence to further assist with expression. By assisting with expression it is meant that the total amount of endolysin polypeptide produced by a host cell may be increased, or the proportion of soluble endolysin polypeptide produced by the host cell may be increased. Thus an expression-enhancing peptide sequence may be any peptide sequence which can facilitate an increase in the amount of endolysin secreted into the culture medium by the host cell, such as an E. coli or an algal host cell. Any suitable expression-enhancing peptide sequence may be used with any of the endolysins of the invention, including any of the expression-enhancing peptide sequences described herein. The expression-enhancing peptide may be selected from secretion-enhancing peptides including OmpA, e mal, gIII, pelB, phoA, ompC, ompT, dsbA, torT, sufI, torA, STII, EOX, lamb, MglB, SfmC, TolB and MmAp. The expression-enhancing peptide may be selected from solubility-enhancing peptides PDI, GST, Trx, MBP, NusA-tag, SUMO, DsbC, Skp, Fh8, ZZ, GB1, T7PK, DsbA and SET.

Any of the endolysins of the invention may comprise the endolysin sequence together with any expression-enhancing peptide sequence and additionally any affinity tag.

Affinity purification of the endolysins of the invention may be employed as a means to produce purified and concentrated endolysin stocks without contaminant native E. coli lysate proteins.

### Host Cells and Expression Systems

The invention also provides a host cell comprising a polynucleotide according to the invention, a nucleic acid construct according to the invention or an expression construct according to the invention. A host cell according to the invention may be any microbial, prokaryotic or eukaryotic, cell which is suitable for expression of the polypeptide of the invention. Preferably, said cell is an E. coli or an algal cell.

The E. coli cell may be from the expression strain, Rosetta™ (DE3) pLysS. Thus, the E. coli cell used for expression may be a BL21 E. coli derivatives designed to enhance the expression of eukaryotic proteins that contain codons rarely used in E. coli and one that is suitable for production of protein from target genes cloned in pET vectors by induction with IPTG as well as express T7 lysozyme, which further suppresses basal expression of T7 RNA polymerase prior to induction. Examples of the host cell of the invention are those of the following strains: DH5alpha, HB101, JM 109 BL21 strains, C41 (DE3), C43 (DE3), DH10B, W3110, CyDisCo, K12 strains, Lemo21 (DE3), T7Express strains, Rosetta strains, SHuffle strains, Tuner strains, Origami strains, ArticExpress strains, AD494, BL21 and trxB.

An endolysin polypeptide of the invention is capable of being directly expressed in its soluble and functional (catalytically active) form in a host cell, preferably in an E. coli or algal host cell. Thus, steps of solubilisation and/or refolding of expressed proteins may be avoided. The capability of expressing soluble and functional endolysin polypeptides of the invention is revealed when a polypeptide of the invention is comprised in any suitable expression vector, when the vector is transformed into a host cell, when transformed host cells are subsequently cultured under suitable conditions to promote the expression of the polypeptide and when the activity of the polypeptide is assessed.

The endolysin polypeptides of the invention may also be expressed in an algal expression system. For example, the algal expression system may use an algal host cell for expression such as Chlamydomonas reinhardtii or Synechococcus elongatus. Such algal systems including the associated vectors are known in the art. The polypeptides of the invention may remain within the algal host cell, such as to produce whole-cell algal compositions comprising the said polypeptides. Alternatively the polypeptides may be purified following expression in algal cells. Such algal systems also allow for affinity purification of the endolysin polypeptide of the invention using known tags, such as those disclosed herein.

Algae may be utilised as host cells and/or expression systems for the expression of any of the polypeptides of the invention disclosed herein, and thus for use in any of the compositions or formulations disclosed herein. Such algae encompass both prokaryotic and eukaryotic algae, which preferably are microscopic algae and more preferably unicellular. Unicellular algae are also known as microalgae. Thus microalgae may be utilised as host cells and/or expression systems for the expression of any of the polypeptides of the invention disclosed herein and thus for use in any of the compositions or formulations disclosed herein. In certain preferred embodiments, the algae is a green algae (Chlorophyta), a brown algae (Phaeophyta), or diatoms (Bacillariophyta).

Examples of green algae, which are especially well-suited for use include members of the Chlamydomonas species, particularly Chlamydomonas reinhardtii; the Chlorella species, the Volvox species, and some marine macrophytes.

Overview of genetically transformed algal species, any of which may be used.

Species
Chlorophyta
Chlamydomonas
reinhardtii
Volvox carteri
Dunaliella salina
Dunaliella viridis
Haematococcus pluvialis
Chlorella sorokiniana
Chlorella kessleri (Parachlorella kessleri)
Chlorella ellipsoidea
Chlorella vulgaris
Ulva lactuca
Ostreococcus tauri
Rhodophyta
Cyanidioschyzon merolae
Porphyra yezoensis
Porphyra miniata
Kappaphycus alvarezii
Gracilaria changii
Porphyridium sp
Gracilaria
Heterokontophyta
Laminaria japonica
Undaria pinnatifida
Phaeodactylum tricornutum
Navicula saprophila (Fistulifera saprophila)
Cylindrotheca fusiformis
Thalassiosira weissflogii
Dinoflagellates
Amphidinium sp.
Symbiodinium microadriaticum
Cyanobacteria
Spirulina platensis (Arthrospira platensis)
Anabaena sp
Synechocystis sp.
Synechococcus
Nosctoc muscorum
Euglenids
Euglena gracilis

An example of an algal expression system for the expression of endolysin polypeptides of the invention is provided in Example 14. Further information concerning the use of algae for recombinant polypeptide expression can be found in the following additional references:

Dyo, Y. M., & Purton, S. (2018). The algal chloroplast as a synthetic biology platform for production of therapeutic proteins. Microbiology (Reading, England). doi:10.1099/mic.0.000599.
Spicer, A., & Purton, S. (2017). Genetic engineering of microalgae: Current status and future prospects. Microalgal Production for Biomass and High-Value Products (pp. 139-164). doi:10.1201/b19464.
Wannathong, T., Waterhouse, J. C., Young, R. E. B., Economou, C. K., & Purton, S. (2016). New tools for chloroplast genetic engineering allow the synthesis of human growth hormone in the green alga Chlamydomonas reinhardtii. APPLIED MICROBIOLOGY AND BIOTECHNOLOGY, 100 (12), 5467-5477. doi:10.1007/s00253-016-7354-6.
Purton, S. (2015). Algal chloroplast engineering: new tools, technologies and applications. European Journal of Phycology, 50, 94-95.
Gangl, D., Zedler, J. A. Z., Rajakumar, P. D., Martinez, E. M. R., Riseley, A., Wlodarczyk, A., . . . Robinson, C. (2015). Biotechnological exploitation of microalgae. JOURNAL OF EXPERIMENTAL BOTANY, 66 (22), 6975-6990. doi:10.1093/jxb/erv426.
Production of therapeutic proteins in algae, analysis of expression of seven human proteins in the chloroplast of Chlamydomonas reinhardtii. B A Rasala, M Muto, P A Lee, M Jager, R M F Cardoso, C A Behnke, P Kirk, Plant biotechnology journal 8 (6), 719-733.
Micro-algae come of age as a platform for recombinant protein production E Specht, S Miyake-Stoner, S Mayfield Biotechnology letters 32 (10), 1373-1383.
Regulation of chloroplast gene expression S P Mayfield, C B Yohn, A Cohen, A Danon. Annual review of plant biology 46 (1), 147-166.
Chlamydomonas reinhardtii chloroplasts as protein factories. S P Mayfield, A L Manuell, S Chen, J Wu, M Tran, D Siefker, M Muto Current opinion in biotechnology 18 (2), 126-133.

Accordingly, the invention provides a host cell comprising a population of nucleic acid molecules encoding any endolysin polypeptide of the invention, optionally wherein the host cell is a cell of a unicellular microorganism, optionally a yeast cell, a bacterial cell such as Lactobacillus, a fungal cell, or an algal cell. The invention additionally provides a host cell comprising a population of nucleic acid molecules encoding any endolysin polypeptide of the invention, optionally wherein the host cell is a plant cell.

The host cell may additionally comprise any of the endolysin polypeptides or fragments thereof defined herein and which have been expressed from said vectors. Each one of the molecules or vectors of the population may encode the same endolysin polypeptide or fragment as defined herein, or molecules or vectors of the population may encode two or more different endolysin polypeptides or fragments as defined herein.

The invention additionally provides a cell lysate comprising a population of any of the endolysin polypeptides or fragments thereof as defined herein, optionally wherein the population consists of the same endolysin polypeptide or fragment, or wherein the population consists of two or more different endolysin polypeptides or fragments. The lysate may be produced following expression of the endolysin polypeptides or fragments thereof in a host cell as defined herein, which may be a cell of a unicellular microorganism, optionally a yeast cell, a bacterial cell such as Lactobacillus, a fungal cell, or an algal cell, or the cell of a plant cell.

Reagents and systems for expressing proteins in yeast are described in: Protein expression-yeast, Nielsen K. H., Methods Enzymol. 2014; 536:133-47.

Reagents and systems for expressing proteins in fungi are described in: Gene expression in fungi, Kalkanci, A et al. IMA Fungus. 2011 June; 2(1): 29-32.

Reagents and systems for expressing proteins in yeast in particular are described in: Micro-algae come of age as a platform for recombinant protein production, Specht, E. et al. Biotechnol Lett. 2010 October; 32(10): 1373-1383.

Reagents and systems for expressing proteins in plants are described in: Plants as Expression Systems for Recombinant Proteins, Kawaka et al., Asian Journal of Biology 3(3): 1-8, 2017.

A host cell may be a cell of a host organism that is a GRAS organism (“Generally Regarded as Safe), or of a host organism that is listed on the Qualified Presumption of safety (QPS) list published by the European Food Safety Authority, including any organism routinely included in poultry diets.

### Compositions and Formulations Comprising Endolysins of the Invention

### Compositions and Formulations

In another aspect, the present invention provides compositions comprising any of the endolysin polypeptides of the invention.

The composition may comprise a population of any of the endolysin polypeptides or fragments defined herein. The population may consist of the same endolysin polypeptide or fragment defined herein. The population may consist of two or more different endolysin polypeptides or fragments as defined herein, in which case the exhibited antimicrobial activity may be a synergistic activity.

The composition may comprise a population of host cells, wherein host cells of the population comprise any of the endolysin polypeptides or fragments as defined herein.

The composition may comprise a whole-cell composition comprising a population of whole cells, wherein cells of the population comprise any of the endolysin polypeptides or fragments as defined herein. Cells of the population may be cells of a unicellular microorganism, optionally yeast cells, bacterial cells such as lactobacilli, fungal cells, or algal cells, or may be plant cells and which cells comprise any of the endolysin polypeptides or fragments as defined herein, e.g. by expression of said polypeptides or fragments.

Any of the above-described compositions may comprise the polypeptides or fragments, host cells and whole cells together with a suitable carrier or excipient, such as water or a physiological acceptable buffer.

In another aspect, the present invention provides formulations comprising any of the endolysin polypeptides of the invention. Preferably the formulations are antimicrobial formulations, antibacterial formulations and/or pharmaceutical formulations.

A formulation may comprise a population of host cells, wherein the host cells of the population comprise any of the endolysin polypeptides of the invention. A formulation may comprise a cell lysate, wherein the lysate comprises any of the endolysin polypeptides of the invention.

Any of the formulations may consist of the same endolysin polypeptide or fragment as defined herein, or may consist of two or more different endolysin polypeptides or fragments as defined herein. The exhibited antimicrobial activity may be a synergistic activity.

Any such formulation may comprise any of the endolysin polypeptides of the invention and at least one pharmaceutically acceptable carrier, diluent, vehicle or excipient. The carrier, diluent, vehicle or excipient must be ‘acceptable’ in the sense of being compatible with the other ingredients of the composition and not deleterious to a subject to which the composition is administered. Typically, carriers and the final composition, are sterile and pyrogen free.

Formulation of a suitable composition can be carried out using standard pharmaceutical formulation chemistries and methodologies all of which are readily available to the reasonably skilled artisan. For example, the agent can be combined with one or more pharmaceutically acceptable excipients or vehicles. Auxiliary substances, such as wetting or emulsifying agents, pH buffering substances, reducing agents and the like, may be present in the excipient or vehicle. Suitable reducing agents include cysteine, thioglycerol, thioreducin, glutathione and the like. Excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J., 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e. g., sterile pyrogen-free water) prior to parenteral administration of the reconstituted composition. The compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. The compositions may be suitable for administration by any suitable route including, for example, intradermal, subcutaneous, percutaneous, intramuscular, intra-arterial, intraperitoneal, intraarticular, intraosseous or other appropriate administration routes. Preferred compositions are suitable for administration by intravenous infusion.

Examples of formulations include topical lotions, creams, soaps, wipes, and the like. They may be formulated into liposomes, to reduce toxicity or increase bioavailability. Other methods for delivery include oral methods that entail encapsulation of the endolysins in microspheres or proteinoids, aerosol delivery (e.g., to the lungs), or transdermal delivery (e.g., by iontophoresis or transdermal electroporation). Other routine methods of administration will be known to those skilled in the art.

Pharmaceutical formulations suitable for oral administration may be provided in convenient unit forms including capsules, tablets, gels, pastes, ointments etc.

Formulations and compositions may comprise a thickener. Suitable thickeners include synthetic hectorite, Irish moss, iota carrageenan, gum tragacanth, starch, polyvinylpyrrolidone, hydroxyethylpropylcellulose, hydroxybutyl methyl cellulose, hydroxypropyl methyl cellulose, hydroxyethyl cellulose, sodium carboxymethyl cellulose, and colloidal silica.

Formulations and compositions may comprise a solubilising agent. Solubilising agents may include agents such as humectant polyols such propylene glycol, dipropylene glycol and hexylene glycol, cellosolves such as methyl cellosolve and ethyl cellosolve, vegetable oils and waxes containing at least about 12 carbons in a straight chain such as olive oil, castor oil and petrolatum and esters such as amyl acetate, ethyl acetate and benzyl benzoate.

Pharmaceutically acceptable excipients which are suitable for use in tablet formulations include, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. Tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

For hard gelatin capsule formulations, the active ingredient can be mixed with an inert solid diluent, for example, calcium carbonate, calcium phosphate or kaolin. For soft gelatin capsule formulations the active ingredient can be mixed with water or an oil medium, for example peanut oil, liquid paraffin or olive oil.

Excipients suitable for the manufacture of aqueous suspensions include suspending agents, for example sodium carboxymethylcellulose, methylcellulose, hydropropyl methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

Aqueous suspensions may also contain one or more preservatives, for example benzoates, such as ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil, for example Arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavouring agents may be added. These compositions may be preserved by the addition of an antioxidant such as ascorbic acid.

Any of the compositions or formulations described herein may also be formulated for parenteral administration, such as by injection, for example bolus injection or continuous infusion, and may be provided in unit dose form in ampules, pre-filled syringes, small volume infusion or in multi-dose containers, e.g. with an added preservative.

Preparations for parenteral administration of formulations and compositions described herein include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

For topical administration to the epidermis, any of the formulations and compositions described herein may be formulated as an ointment, cream, or lotion. Ointments and creams, may, for example, be formulated with an aqueous or oily base with the addition of suitable thickening and/or gelling agents. Lotions may be formulated with an aqueous or oily base and will in general also contain one or more emulsifying agents, stabilizing agents, suspending agents, thickening agents, or coloring agents. Formulations suitable for topical administration in the mouth include lozenges, e.g. in a flavoured base, usually sucrose and acacia or tragacanth; pastilles comprising the active ingredient in an inert base such as gelatin and glycerin or sucrose and acacia; and mouth washes comprising the active ingredient in a suitable liquid carrier.

For topical administration to the eye, any of the formulations and compositions described herein can be made up in solution or suspension in a suitable sterile aqueous or non-aqueous vehicle. Additives such as buffers (e.g. sodium metabisulphite or disodium edeate) and thickening agents such as hypromellose can also be included.

For intra-nasal administration, any of the formulations and compositions described herein can be provided in a liquid spray or dispersible powder or in the form of drops. Drops may be formulated with an aqueous or non-aqueous base also comprising one or more dispersing agents, solubilising agents, or suspending agents.

For administration by inhalation, any of the formulations and compositions described herein can be delivered by insufflator, e.g. a nebulizer or a pressurized pack or other convenient means of delivering an aerosol spray. Pressurised packs may comprise a suitable propellant. In the case of a pressurised aerosol the dosage unit may be determined by providing a value to deliver a metered amount.

Any of the formulations and compositions described herein can take the form of a dry powder composition, for example a powder mix of the active component and a suitable powder base such as lactose or starch. The powder composition may be presented in unit dosage form in, for example, capsules, cartridges or blister packs of gelatins, from which the powder can be administered with the aid of an inhalator or insufflator.

Any of the formulations and compositions described herein can be incorporated into a liquid disinfecting solution. Such solutions may further comprise antimicrobials or antifungals such as alcohol, providone-iodine solution and antibiotics as well as preservatives. These solutions can be used, e.g., as disinfectants of the skin or surrounding area, e.g. prior to insertion or implantation of a device such as a catheter, as catheter lock and/or flush solutions, and as antiseptic rinses for any medical device including, but not limited to catheter components such as needles, Luer-Lok connectors, needleless connectors and hubs as well as other implantable devices. These solutions can also be used to coat or disinfect surgical instruments.

The amount of endolysin polypeptide required for use in treatment will of course vary not only with the particular polypeptide but also with the route and form of administration, the nature and severity of the condition being treated, and the type, age and condition of the organism. Thus, appropriate concentrations of the active agent(s) to be incorporated into compositions and formulations can be routinely determined by those skilled in the art in accordance with standard practices.

### Additional Agents

Any of the above-described compositions, lysates or formulations may further comprise one or more additional agents having antimicrobial activity and wherein the additional agent(s) is not an endolysin polypeptide or fragment. The one or more additional agents may be selected form the group consisting of antibiotic agents, biofilm-degrading agents, biofilm-suppressing agents, sequestering agents such as chitosan, EDTA and citric acid, bacteriostatic agents such as glycerol. The one or more additional agents may be selected form the group consisting of a stabilising agent, an anti-clumping agent, a prebiotic agent, a probiotic agent and an edible gel such as congealed water nutrient matrix.

Any of the formulations and compositions described herein can additionally include, together with the endolysin(s), an antimicrobial agent which is not an endolysin, such as detergents and antibiotics. Suitable antibiotics include aminoglycosides (e.g., gentamicin, kanamycin and streptomycin), beta-lactams (e.g., penicillin, ampicillin, imipenem and cephalosporins such as Ceftazidime), quinolones (e.g., ciprofloxacin), macrolides such as azithromycin, clarithromycin, dirithromycin, erythromycin, roxithromycin and telithromycin, oxazolidinones such as linezolid, ansamycins such as rifamycin, sulfonamides, tetracyclines such as Doxycycline. Additional antibiotics include glycopeptides such as vancomycin, sulfisoxazole, trimethoprim, novobiocin, daptomycin and linezolid.

Generally, the antimicrobial is administered in a microbicidal amount. However, the antimicrobial can also be administered in microbistatic amount.

Antiparasitic compounds which can be included in the formulations and compositions described herein include the benzazoles (albendazole, mebendazole, thiabendazole, etc.), the azoles (metronidazole, tinidazole, etc.), macrocycles (amphotericin B, rifampin, ivermectin etc.) and others such as pyrantel pamoate, diethylcarbamazine, niclosamide, praziquantel, melarsoprol and eflornithine.

Antiviral compounds which can be included in the formulations and compositions described herein include the nucleoside analog reverse transcriptase inhibitors (acyclovir, didanosine, stavudine, zidovudine, lamivudine, abacavir, emtricitabine, entecavir etc.), uncoating inhibitors (amantadine, rimantadine, pleconaril etc.), protease inhibitors (saquinavir, ritonavir, indinavir, nelfinavir, amprenavir, etc.) and others such as zanamivir, oseltamivir, rifampin.

Antiviral compounds which can be included in the formulations and compositions described herein include an azole, such as miconazole, ketoconazole, clotrimazole, econazole, omoconazole, bifonazole, butoconazole, fenticonazole, isoconazole, sertaconazole, sulconazole, tioconazole, fluconazole, itraconazole, isavuconazole, ravuconazole, posaconazole, voriconazole, terconazole and albofungin; a macrocycle, such as natamycin, rimocidin, filipin, nystatin, amphotericin B, candicin, hamycin; an allyl amine such as terbinafine, naftifine and butenafine; an echinocandin such as anidulafungin, caspofungin and micafungin; or others such as polygodial, ciclopirox, tolnaftate, benzoic acid, undecylenic acid, flucytosine and griseofulvin.

Antifungal compounds which can be included in the formulations and compositions described herein include an azole, such as miconazole, ketoconazole, clotrimazole, econazole, omoconazole, bifonazole, butoconazole, fenticonazole, isoconazole, sertaconazole, sulconazole, tioconazole, fluconazole, itraconazole, isavuconazole, ravuconazole, posaconazole, voriconazole, terconazole and albofungin; a macrocycle, such as natamycin, rimocidin, filipin, nystatin, amphotericin B, candicin, hamycin; an allyl amine such as terbinafine, naftifine and butenafine; an echinocandin such as anidulafungin, caspofungin and micafungin; or others such as polygodial, ciclopirox, tolnaftate, benzoic acid, undecylenic acid, flucytosine and griseofulvin.

### Biomass Compositions

Any of the endolysin polypeptides of the invention may be comprised in a biomass composition.

Endolysins may be expressed in host cells, the host cells harvested and biomass compositions created. For example, host cells expressing endolysin polypeptides of the invention as described and defined herein may be harvested and used to form biomass compositions comprising host cells.

Host cells may be lysed and lysates used to form biomass compositions comprising lysates, for example the lysates may be dried to form dried biomass compositions comprising dried lysates. Lysates may include host cell debris following lysis. Alternatively, lysates may comprise host cell extracts wherein host cell debris is removed, for example host cell debris may be removed by centrifugation and the supernatants collected and used whilst the pelleted host cell debris is discarded.

Biomass compositions may comprise lysed and partially-lysed host cell debris. Biomass compositions may comprise host cell extract wherein host cell debris has been removed or substantially removed. Biomass compositions may comprise mixtures of lysed host cell debris, partially-lysed host cell debris and host cell extract in any combination.

In the methods of the invention algal cells, as described further herein, are preferred host cells. Any of the endolysin polypeptides of the invention may be comprised in an algal host cell following expression in the algal host cell, or in an algal host cell lysate/extract following expression in the algal host cell. Such cells and lysates/extracts may be used to create biomass compositions, as described above.

Methods of cultivating algal biomass include photoautotrophic production, heterotrophic production and mixotrophic production. Cultivation systems used for production of algal biomass include open ponds such as unmixed open ponds, circular ponds and open raceway ponds. Other cultivation systems include closed photobioreactors (PBR) such as tubular PBR, flat-plate PBR, Column PBR, continuously stirred tank reactors and fed-batch bioreactors. Cultivation systems also include hybrid production systems.

Following cultivation, algal biomass can be harvested using methods such as flocculation, chemical coagulation, combined flocculation, centrifugation and gravity sedimentation.

Any of the above-described biomass compositions may be dried. Dried biomass can be obtained by known methods such as spray-drying and freeze-drying. For example, dried biomass such as that of Chlamydomonas reinhardtii can be obtained by firstly culturing algae, for example in a fermentator, followed by harvesting of algae, pelleting by centrifugation and spray-drying of biomass (see for example, Kightlinger et al. Elec. J. Biotech. vol 17, 2014). Algae cells can optionally be lysed, for example, by autolysis. The lysed calls can then be dried, for example in a rotary evaporator under vacuum. The resulting product can then be dried overnight using a vacuum pump and ground to create an algae extract powder. Algal extracts can also be prepared from spray-dried biomass, by grinding in a bead mill or sonication. The algal extract powder can be used appropriately, for example, added to animal foodstuffs.

### Animal Foodstuffs

The present invention also provides foodstuffs, preferably animal foodstuffs, comprising one or more foodstuffs and a composition comprising a population of host cells. The host cells may comprise an endolysin polypeptide or fragment as described herein. The foodstuff may also comprise any host cell, cell lysate, composition or whole-cell composition as described herein. The foodstuff may also comprise an antimicrobial formulation as described herein.

A foodstuff of the invention is suitable for consumption by animals, including poultry, optionally a broiler chicken, preferably Gallus gallus domesticus a pig, preferably Sus scrofa domesticus or wherein the foodstuff is suitable for consumption by a rodent, optionally a mouse or rat. Consumption of the foodstuff may have a prophylactic or therapeutic effect on the animal. For example, consumption may reduce populations of C. perfringens that thrive within the animal thus treating the animal or an infection caused by C. perfringens. Consumption by an animal of the foodstuff may also prevent infection of C. perfringens or if the animal previously suffered from infection by C. perfringens, will have the effect of preventing further infection as well as curing infection.

A foodstuff comprising an endolysin of the invention may contain preservatives or any additives normally used in the preparation of foodstuffs for consumption and/or to prolong shelf-life.

A foodstuff comprising an endolysin of the invention may be provided in any suitable edible form, such as in powdered or ground form, animal feed, animal mash or pelleted form. The foodstuff comprising an endolysin of the invention may be provided in soluble form for drinking, such as a soluble aqueous composition comprising an endolysin of the invention.

### Methods of Using the Endolysin Polypeptides of the Invention

The invention provides for the use of polypeptides of the invention in various methods. For example, the polypeptides in accordance with the present invention may also be used in therapy or prophylaxis. In therapeutic applications, polypeptides or compositions are administered to a subject already suffering from a disorder or condition, in an amount sufficient to cure, alleviate or partially arrest the condition or one or more of its symptoms. Accordingly, the present invention may have bacterial static and/or bacteriocidal properties.

Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means. Thus the invention also provides a polypeptide of the invention for use in the treatment of the human or animal body.

Also provided herein is a method of prevention or treatment of disease or condition in a subject, which method comprises administering a polypeptide of the invention to the subject in a prophylactically or therapeutically effective amount. The polypeptide may be co-administered with another agent. The polypeptide is preferably administered orally but may be administered by any suitable route including, for example, intravenous infusion, intradermal, subcutaneous, percutaneous, intramuscular, intra-arterial, intraperitoneal, intraarticular, intraosseous or other appropriate administration routes. The amount of said polypeptide that is administered may be between 0.01 mg/kg BW and 2 mg/kg BW, between 0.04 and 2 mg/kg BW, between 0.12 mg/kg BW and 2 mg/kg BW, preferably between 0.24 mg/kg and 2 mg/kg BW and most preferably between 1 mg/kg and 2 mg/kg BW. The polypeptide may be administered on multiple occasions to the same subject.

Polypeptides of the invention may be particularly useful in the treatment or prevention of a disease or condition mediated by bacterial infection such as bacterial infection by C. perfringens, for example caused by colonisation of C. perfringens in the gastrointestinal tract. Accordingly, the invention provides a polypeptide of the invention for use in the treatment or prevention of a disease or condition characterised by presence of C. perfringens, for example in the gastrointestinal tract.

The invention also provides a method of treating or preventing a disease or disorder mediated by C. perfringens comprising administering to an individual a polypeptide of the invention. The disease or disorder may be a disease or disorder caused by a Clostridium perfringens infection, optionally an infection caused by a Type A strain of Clostridium perfringens, such as food poisoning, gas gangrene, necrotic enteritis, a gut lesion and/or a gastrointestinal infection.

The method may comprise repeat administration of the said polypeptide. The invention also provides a polypeptide of the invention for use in the manufacture of a medicament for the treatment or prevention of a disease or condition mediated by C. perfringens.

The polypeptide of the invention may be administered via one or more routes of administration using one or more of a variety of methods known in the art. The route and/or mode of administration will vary depending upon the desired results. Preferred routes of administration include oral administration. Other routes of administration include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection. Alternatively, a polypeptide can be administered via a non-parenteral route, such as a topical, epidermal or mucosal route of administration. Local administration is also preferred, including peritumoral, juxtatumoral, intratumoral, intralesional, perilesional, intra cavity infusion, intravesical administration, and inhalation. In a preferred embodiment, the invention is administered orally in the form of a foodstuff. Other routes that are useful are those that could be used to deliver the endolysin of the invention into the subject, preferably into the gut.

A suitable dosage of a peptide of the invention may be determined by a skilled medical practitioner. Actual dosage levels of a peptide may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular antibody employed, the route of administration, the time of administration, the rate of excretion of the antibody, the duration of the treatment, other drugs, compounds and/or materials used in combination with the particular compositions employed, the age, sex, weight, condition, general health and prior medical history of the patient being treated, and like factors well known in the medical arts.

A suitable dose of a peptide may be, for example, in the range of from about 0.1 μg/kg to about 100 mg/kg body weight of the patient to be treated. For example, a suitable dosage may be from about 1 μg/kg to about 10 mg/kg body weight per day or from about 10 g g/kg to about 5 mg/kg body weight per day.

Dosage regimens may be adjusted to provide the optimum desired response (e.g., a therapeutic response). For example, a single bolus may be administered, or the method may comprise several divided doses administered over time or the dose may be proportionally reduced or increased as indicated by the exigencies of the therapeutic situation, provided the required interval. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Any of the formulations and compositions described herein can be formulated as a vaccine, to be administered for prophylaxis. Any such vaccine formulation may be administered directly to the animal for prophylaxis. Any such vaccine formulation may be administered to an animal egg for prophylaxis. For example, the vaccine formulation may be administered, e.g. injected, into the egg within the region defined by either the amnion or yolk sac. The vaccine formulation may be administered, e.g. injected, into the amniotic fluid. Any such vaccine formulation may be in the form of a composition comprising the endolysin polypeptide of the invention expressed in an algal host cell.

### Endolysin/Composition Comprising Endolysin for Treatment

A composition according to the invention can be used to treat animals, including humans, infected with C. perfringens, preferably broiler chickens such as Gallus gallus domesticus. Any suitable route of administration can be used to administer said composition including but not limited to: oral, aerosol or other device for delivery to the lungs, nasal spray, intravenous, intramuscular, intraperitoneal, intrathecal, vaginal, rectal, topical, lumbar puncture, intrathecal, and direct application to the brain and/or meninges.

A composition according to the invention comprising a polynucleotide or a nucleic acid construct or an endolysin polypeptide or a vector or a cell according to the invention is preferably said to be active, functional or therapeutically active or able to treat, prevent and/or delay an infectious disease when it decreases the amount of a C. perfringens species present in a patient or in a cell of said patient or in a cell line or in a cell free in vitro system and preferably means that 99%, 90%, 80%, 70%, 60%, 50%, 40%, 30%, 20%, 10%, 5% or less of the initial amount of a C. perfringens species, is still detectable after treatment. Preferably no C. perfringens species is detectable after treatment. In this paragraph, the expression “amount of C. perfringens” preferably means alive C. perfringens genera. C. perfringens species may be detected using standard techniques known by the skilled person such as immunohistochemical techniques using C. perfringens-specific antibodies. C. perfringens species (alive and/or dead) may be detected using standard techniques known by the skilled person such as microbiological bacterial culture techniques and/or real-time quantitative reverse transcription polymerase chain reaction to assay for bacterial mRNA. Examples of how to detect C. perfringens include enumeration of C. perfringens from the intestinal contents and faces of the subject using standard plate count methodologies, or PCR methods such as real-time PCT or conventional PCR. For PCR-based methods any probes specific for C. perfringens may be used. Types of probes may include fluorogenic and hydrolysis-type (5′ nuclease) probes (see e.g. https://aem.asm.org/content/71/7/3911].

Effectiveness of C. perfringens treatment is preferably assessed in a sample from the subject such as a faecal sample, tissues or cells from a subject by comparison to the amount present in said subject before treatment with said composition or polypeptide according to the invention. Alternatively, the comparison can be made with a sample, tissues or cells of said individual or patient which has not yet been treated with said composition or polypeptide in case the treatment is local.

A composition comprising a polynucleotide or a nucleic acid construct or a polypeptide or a vector or a cell according to the invention may be administered to a subject in need thereof or of a cell, tissue or organ or said patient at least one week, one month, six month, one year or more.

Accordingly, the invention provides for a composition according to the invention, for use as a medicament for the treatment of a subject in need thereof Preferably, said composition is for use as a medicament in the treatment of a condition associated with infection of a subject with a C. perfringens, preferably a bacterium of the Type A strain.

The invention further provides for a method of treatment, delay and/or prevention of a condition associated with infection of a subject with a C. perfringens, preferably a bacterium of the Type A strain comprising administration an endolysin polypeptide according to the invention, or a polynucleotide according to the invention, or a nucleic acid construct according to the invention, or an expression construct according to the invention, or a host cell according to the invention, or a composition according to the invention. Accordingly, treatment may include the treatment of food poisoning in a subject.

The medical use herein described may be formulated as a product according to the invention for use as a medicament for treatment of the stated diseases but could equally be formulated as a method of treatment of the stated diseases using a product according to the invention, a product according to the invention for use in the preparation of a medicament to treat the stated diseases and use of a product according to the invention for the treatment of the stated diseases. Such medical uses are all envisaged by the present invention. The subject in need of treatment, delay and/or prevention of a condition associated with infection may by any animal subject, preferably a mammal, more a preferably broiler chicken such as Gallus gallus domesticus. Accordingly, subjects may include livestock such as a pig (e.g. Sus scrofa). Examples of subjects also include a dog or a cat, or a human subject.

The compound specifically targeting a bacterial cell can be an endolysin polypeptide according to the invention, or a polynucleotide according to the invention, or a nucleic acid construct according to the invention, or an expression construct according to the invention, or a host cell according to the invention, or a composition according to the invention.

The endolysin polypeptide according the invention, or a polynucleotide according to the invention, or a nucleic acid construct according to the invention, or an expression construct according to the invention, or a host cell according to the invention, or a composition according to the invention may conveniently be used in a method of treatment of an intracellular bacterial infection in a subject in need thereof, comprising:

administration of an effective amount of an agent that increases the intracellular pH of a host cell and/or of an intracellular compartment of a host cell, and administration of an effective amount of a antimicrobial agent. Said method is herein referred to as a method according to the invention.

The Inventors have surprisingly identified that treatment of certain diseases or disorders in animals may be effected by an antimicrobial formulation comprising a whole-cell composition, wherein whole-cells of the composition are microalgal host cells comprising an endolysin polypeptide having antimicrobial activity, or comprising a fragment or variant of an endolysin polypeptide wherein the fragment or variant has antimicrobial activity. The endolysin polypeptide, or the fragment or the variant of an endolysin polypeptide may be any of the endolysin polypeptides, or the fragments or the variants of an endolysin polypeptide disclosed herein. Alternatively, the endolysin polypeptide, or the fragment or the variant of an endolysin polypeptide may be any endolysin polypeptide, or any fragment or any variant of an endolysin polypeptide which possesses any of the surprisingly advantageous functional antimicrobial activities described and defined herein. Any such endolysin polypeptide, fragment or variant may therefore be regarded as functionally equivalent to specific polypeptides disclosed herein. In any such antimicrobial formulation, reference to endolysin polypeptide, fragment or variant encompasses combinations of one or more endolysin polypeptides and/or one or more fragments and/or one or more variants.

Accordingly, the invention also provides an antimicrobial formulation for use as a medicament, the formulation comprising a whole-cell composition, wherein whole-cells of the composition are microalgal host cells comprising an endolysin polypeptide having antimicrobial activity, or comprising a fragment or variant of an endolysin polypeptide wherein the fragment or variant has antimicrobial activity.

Any such antimicrobial formulation may be for use in the treatment of a bacterial infection in an animal. Any such antimicrobial formulation may be for use in the treatment of a bacterial infection which is a Clostridium perfringens infection, optionally an infection caused by a Type A strain of Clostridium perfringens. Any such antimicrobial formulation may be for use in the treatment of a disease or disorder caused by a Clostridium perfringens infection, optionally an infection caused by a Type A strain of Clostridium perfringens. The disease or disorder caused by the Clostridium perfringens infection may be food poisoning, gas gangrene, necrotic enteritis, a gut lesion and/or a gastrointestinal infection.

The invention also provides a method for the prevention or treatment of a disease or disorder in an animal, the method comprising administering to the animal a prophylactically or therapeutically effective amount of an antimicrobial formulation, the formulation comprising a whole-cell composition, wherein whole-cells of the composition are microalgal host cells comprising an endolysin polypeptide having antimicrobial activity, or comprising a fragment or variant of an endolysin polypeptide wherein the fragment or variant has antimicrobial activity.

In any such method the disease or disorder may be a bacterial infection in an animal. In any such method the bacterial infection may be a Clostridium perfringens infection, optionally an infection caused by a Type A strain of Clostridium perfringens. In any such method the disease or disorder may be caused by a Clostridium perfringens infection, optionally an infection caused by a Type A strain of Clostridium perfringens. The disease or disorder caused by the Clostridium perfringens infection may be food poisoning, gas gangrene, necrotic enteritis, a gut lesion and/or a gastrointestinal infection.

In any of the above-described antimicrobial formulations or methods the animal may be a poultry animal, optionally a broiler chicken, preferably Gallus gallus domesticus; or wherein the animal is a pig, preferably Sus scrofa domesticus.

In any of the above-described antimicrobial formulations or methods the antimicrobial activity of the endolysin polypeptide, fragment or variant may be bacteriolytic activity and/or bacterial growth inhibitory activity. The bacteriolytic activity and/or bacterial growth inhibitory activity of the endolysin polypeptide, fragment or variant may be bacteriolytic activity and/or bacterial growth inhibitory activity against, Clostridium perfringens, preferably a Type A strain of Clostridium perfringens.

In any of the above-described antimicrobial formulations or methods, when the endolysin polypeptide, fragment or variant is tested in a standard cell viability assay at a concentration of 5 μg/ml against Clostridium perfringens strain NCTC8237 it may exhibit a Δ Log 10 value of approximately 0.40 or more, 2.00 or more or 3.70 or more. In any of the above-described antimicrobial formulations or methods, when the endolysin polypeptide, fragment or variant is tested in a standard cell viability assay at a concentration of 5 μg/ml against Clostridium perfringens strain NCTC8237 it may exhibit a % reduction in cell viability of approximately 60% or more, 70% or more, 80% or more, 90% or more or 100%. In any of the above-described antimicrobial formulations or methods, when the endolysin polypeptide, fragment or variant is tested in a standard cell turbidity reduction assay at a concentration of 5 μg/mL against Clostridium perfringens strain NCTC8237 it may exhibit a t50% lysis value of 7 minutes or less; or it may exhibit a t50% lysis value of 3 minutes or less. In any of the above-described antimicrobial formulations or methods, when the endolysin polypeptide, fragment or variant is tested in a standard minimum inhibitory concentration (MIC)/minimum bactericidal concentration (MBC) assay against Clostridium perfringens strain Cp6 at 2.1×10⁴ cells/mL it may exhibit an MIC/MBC value of 17 μg/mL or less, preferably 1.7 μg/mL or less; or it may exhibit an MIC/MBC value of 0.65 μM or less, preferably 0.05 μM or less.

### Methods of Obtaining Endolysins of the Invention

The invention further provides for a method for the production of an endolysin polypeptide according to the invention, comprising:

culturing a host cell according to the invention under conditions conducive to the production of the endolysin polypeptide, optionally isolating and purifying the endolysin polypeptide from the culture broth, and optionally freeze-drying the endolysin polypeptide.

A polypeptide as disclosed herein may be produced by any suitable means. For example, the polypeptide may be synthesised directly using standard techniques known in the art, such as Fmoc solid phase chemistry, Boc solid phase chemistry or by solution phase peptide synthesis. Alternatively, a polypeptide may be produced by transforming a host cell, typically a bacterial e.g. E. coli cell, with a nucleic acid molecule or vector which encodes said polypeptide. Production of polypeptides by expression in bacterial host cells is described herein and is outlined in the Examples (see Example 2). A polypeptide of the invention may also be produced by transforming into an algal host cell.

A host cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes (e.g. E. coli), yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. The choice of method of genetic modification is generally dependent on the type of cell being transformed and the circumstances under which the transformation is taking place, and would be apparent to the skilled person.

The invention also includes cells that have been modified to express a polypeptide of the invention. Such cells may be modified to carry an expression vector encoding a polypeptide of the invention. Such cells typically include prokaryotic cells such as bacterial cells, for example E. coli. Such cells may be cultured using routine methods to produce a polypeptide of the invention. Preferably, a cell of the invention is that of an algal cell.

A polypeptide may be derivatised or modified to assist with their production, isolation or purification. For example, where a polypeptide of the invention is produced by recombinant expression in a bacterial host cell, the sequence of the polypeptide may include an additional methionine (M) residue at the N terminus to improve expression.

As another example, the polypeptide of the invention may be derivatised or modified by addition of a ligand which is capable of binding directly and specifically to a separation means. Alternatively, the polypeptide may be derivatised or modified by addition of one member of a binding pair and the separation means comprises a reagent that is derivatised or modified by addition of the other member of a binding pair. Any suitable binding pair can be used. In a preferred embodiment where the polypeptide for use in the invention is derivatised or modified by addition of one member of a binding pair, the polypeptide is preferably histidine-tagged or biotin-tagged. Typically the amino acid coding sequence of the histidine or biotin tag is included at the gene level and the polypeptide is expressed recombinantly in E. coli. The histidine or biotin tag is typically present at either end of the polypeptide, preferably at the C-terminus. It may be joined directly to the polypeptide or joined indirectly by any suitable linker sequence, such as 3, 4 or 5 glycine residues. The histidine tag typically consists of six histidine residues, although it can be longer than this, typically up to 7, 8, 9, 10 or 20 amino acids or shorter, for example 5, 4, 3, 2 or 1 amino acids.

The amino acid sequence of a polypeptide may be modified to include non-naturally occurring amino acids, for example to increase stability. When the polypeptides are produced by synthetic means, such amino acids may be introduced during production. The polypeptides may also be modified following either synthetic or recombinant production. Polypeptides may also be produced using D-amino acids. In such cases the amino acids will be linked in reverse sequence in the C to N orientation. This is conventional in the art for producing such polypeptides.

A number of side chain modifications are known in the art and may be made to the side chains of the polypeptides, subject to the polypeptides retaining any further required activity or characteristic as may be specified herein. It will also be understood that polypeptides may be chemically modified, e.g. post-translationally modified. For example, they may be glycosylated, phosphorylated or comprise modified amino acid residues.

The polypeptide may be PEGylated. The polypeptide of the invention may be in a substantially isolated form. It may be mixed with carriers or diluents (as discussed below) which will not interfere with the intended use and still be regarded as substantially isolated. It may also be in a substantially purified form, in which case it will generally comprise at least 90%, e.g. at least 95%, 98% or 99%, of the protein in the preparation.

### EXAMPLES

The following Examples are provided to illustrate the invention, but not to limit the invention.

### Example 1: Bioinformatics Search

There are a number of available genome sequences of Clostridium perfringens strains, as well as of C. perfringens bacteriophages. C. perfringens genomes NC_008261.1, NC_003366.1, CP000312.1, NZ_CP009557.1, NZ_CP010993.1, NZ_CP010994.1, NZ_AP017630.1, NZ_CP013101.1, NZ_CP023410.1, and bacteriophage genomes NC_003524, NC_019506, NC_019496, NC_019508, NC_011318, NC_017980, NC_018083, NC_017978, NC_018084, NC_021325, JF767210, NC_008265 were selected to carry out a sequence similarity search for endolysin genes. The BLAST tool of The National Center for Biotechnology Information (NCBI) was used to identify sequences of similarity at the amino acid level, but with less than 75% identity, to the following endolysin genes: Ply3626, CP51 L, PlyCP26F, Ply390 and plyS9. In addition, C. perfringens genome sequences were analysed using the PHASTER software (phaster.ca) to search for potential prophage regions. The prophage regions identified were subsequently screened to uncover potential endolysin sequences. Endolysin sequences of interest (SEQ ID NOs: 1 to 11) are listed in Table 1 below.

### Example 2: Construction of an E. coli Expression Vector that Co-Expresses a Synthetic tRNA

Endolysins are proteins with antimicrobial activity and could be potentially toxic to E. coli cloning strains. A cloning strategy was therefore implemented to minimize undesirable recombinant endolysin expression. Endolysin coding sequences were engineered to substitute endogenous tryptophan “TGG” codons with that of “TGA” stop codons. This was done for the first or first and second Trp codon of the endolysin coding sequences. Therefore, accumulation of recombinant endolysins should not be observed unless a tryptophan tRNA with anti-codon sequence mutated to “TCA” (i.e. tRNA-Wtca) is co-expressed with the endolysin gene.

After the successful cloning of a potentially toxic endolysin gene, an E. coli expression strain capable of producing the endolysin was also necessary. The expression vector system pETDuet (Merck Millipore) was chosen to be used for endolysin recombinant expressions in E. coli. This system provides two advantages: (i) the pETDuet vector has two multiple cloning sites (MCSs) which position DNA sequences of choice under the control of two independent T7 polymerase promoters, and (ii) both T7 polymerase promoters have been engineered to respond to the presence of IPTG in the media, allowing strict control of gene expression. The latter feature is important as 36% of stop codons in E. coli are TGA, and the production of tRNA-Wtca could cause additional toxic effects if the expression is not controlled. The mutant tRNA-Wtca DNA sequence was cloned into one of the MCSs, while endolysin coding sequences with mutated tryptophan codons (TGG->TGA) were cloned into the second MCS.

For the cloning of the tRNA-Wtca coding sequence, a 179-bp DNA sequence that encodes for the native tryptophan tRNA was identified in the genome of E. coli k-12 (GenBank accession no. CP028306). The anti-codon of this tRNA (“CCA”) was replaced by the triplet “TCA”. In addition, two 18-bp sequences were added to either end of the DNA sequence in order to facilitate its cloning into the pETDuet vector by recombination methods, where the whole sequence was externally synthesised (Eurofins Genomics). The 5′ and 3′ end 18-bp sequences were homologous to the sequence of the MCS1 of pETDuet, which allowed directional cloning of the synthesized gene using the Gibson assembly method (New England Biolabs). The resultant plasmid was sequence-verified and named ecAL002.

To test that the cloned tRNA-Wtca was functional, a Red Fluorescent Protein (RFP) gene containing three WTGG-TGA codons in its coding sequence was cloned into the empty MCS2 of ecAL002, resulting in plasmid ecAL004. The RFP sequence was synthesized externally (Eurofins Genomics), and included 18-bp flanking sequences at both ends. The gene's directional cloning into MCS2 of the pETDuet vector was achieved by the Gibson Assembly method. As an additional control, another plasmid was constructed wherein the tRNA-Wtca sequence was removed from MCS1 of plasmid ecAL004. This plasmid was named ecAL003. Plasmids ecAL002 (tRNA-Wtca only), ecAL003 (RFP only) and ecAL004 (RFP+tRNA-Wtca) were transformed into the E. coli expression strain Rosetta™ (DE3) pLysS (Merck). Transformants of which were selected based on ampicillin resistance conferred by the bla gene present on the backbone of the pETDuet-derivative plasmids. To induce RFP expression, the three strains were grown overnight at 37° C. in 5 ml of Luria broth (LB) media supplemented with 100 μg/ml ampicillin and 25 μg/ml chloramphenicol. 1 ml of the overnight cultures was used to inoculate 100 ml of LB-ampicillin-chloramphenicol media. The cultures were grown at 37° C. with shaking for 2.5 h before IPTG addition to the media at a final concentration of 1 mM to induce expression from the T7 polymerase promoters. Cultures were incubated at 30° C. with shaking, and cells harvested after 5 h by centrifugation.

Determination of the successful expression of RFP was done by visual inspection and SDS-PAGE. Briefly, protein extracts were obtained from each cell culture. A cell pellet from 25 mL of culture was resuspended in 1 mL phosphate buffered saline, pH 7.4 (PBS, pH 7.4), and cells were lysed after 10 cycles of sonication (10 s on/off at 10 μm amplitude for 10 cycles), using a Soniprep 150 (MSE). Cells were incubated on an ice bath during sonication to prevent sample overheating. The crude lysates were centrifuged for 5 minutes at 16600×g at 4° C., after which the clarified lysate (supernatant) was collected. Clarified lysate samples were analysed via SDS-PAGE using a mini-PROTEAN®3 electrophoresis system (Bio-rad) to assess RFP expression levels. Clarified lysate samples were mixed 1:1 with 2× Laemmli buffer (Bio-rad) and 100 mM DTT, and subsequently denatured at 100° C. for 5 minutes. The denatured Laemmli samples were then loaded and resolved in 4-15% precast polyacrylamide, 15-well Mini-Protean TGX gels (Bio-rad).

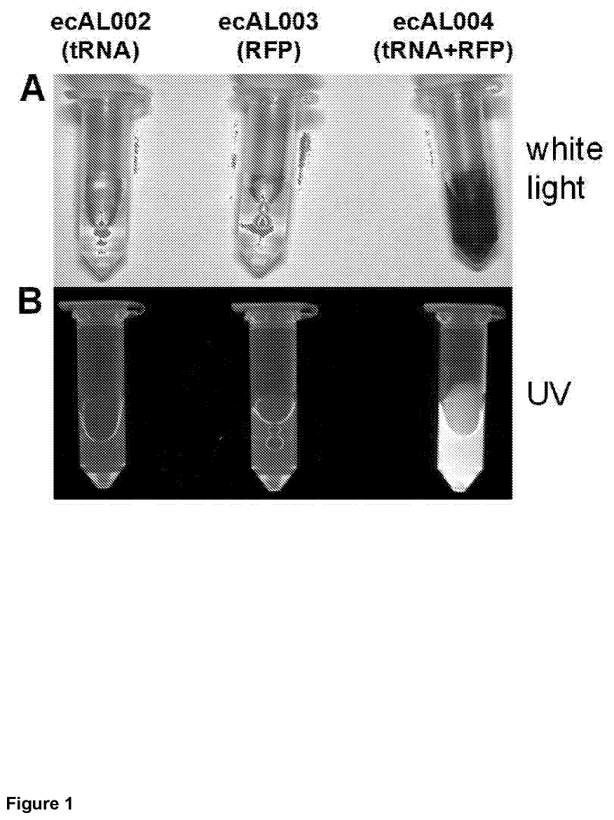

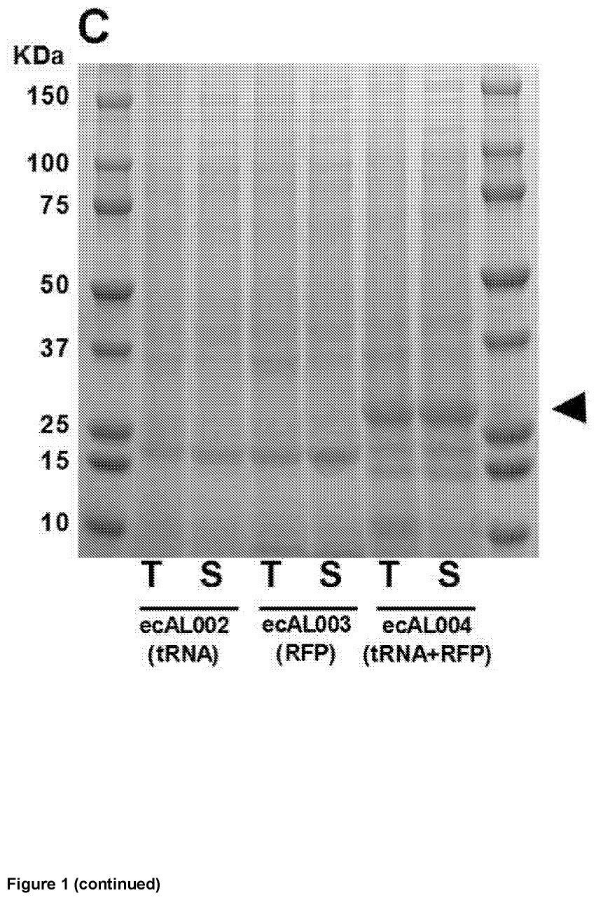

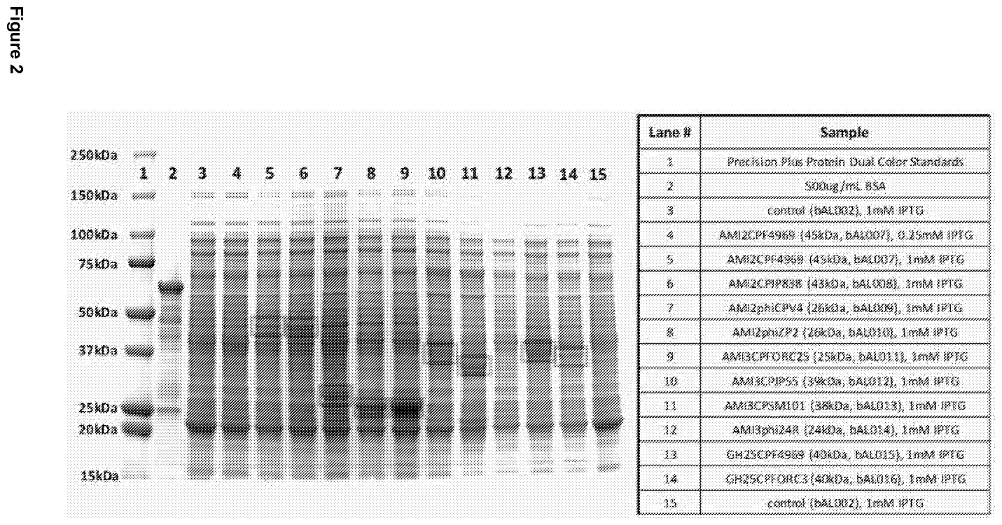

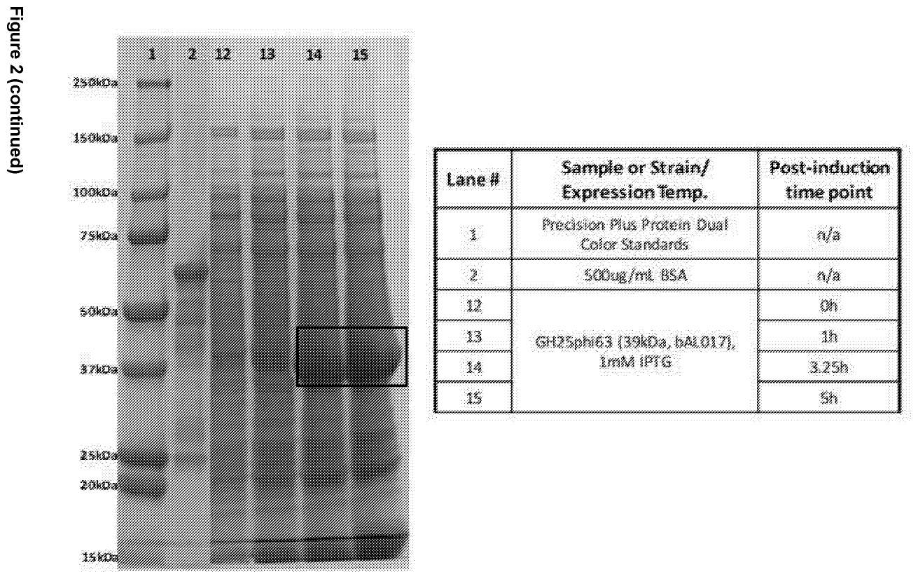

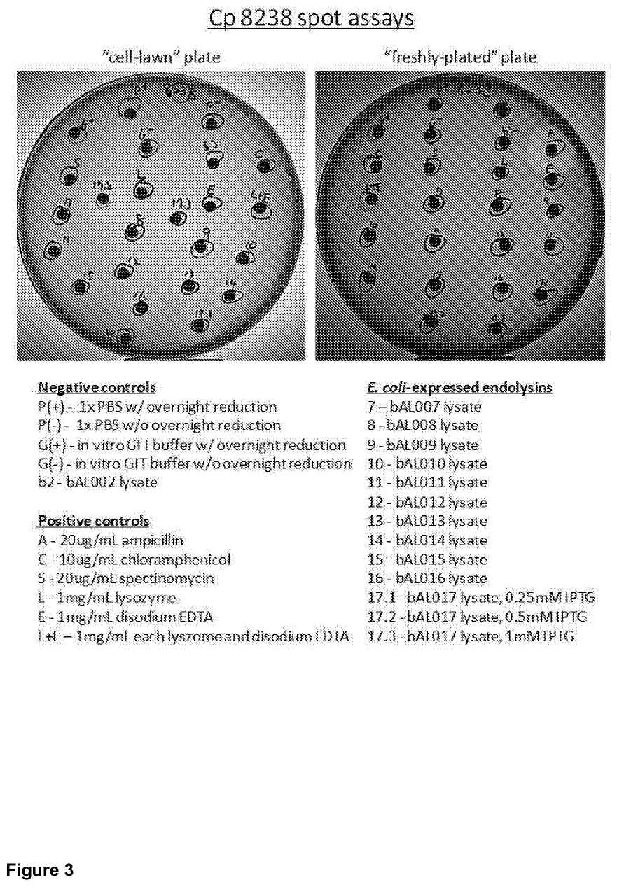

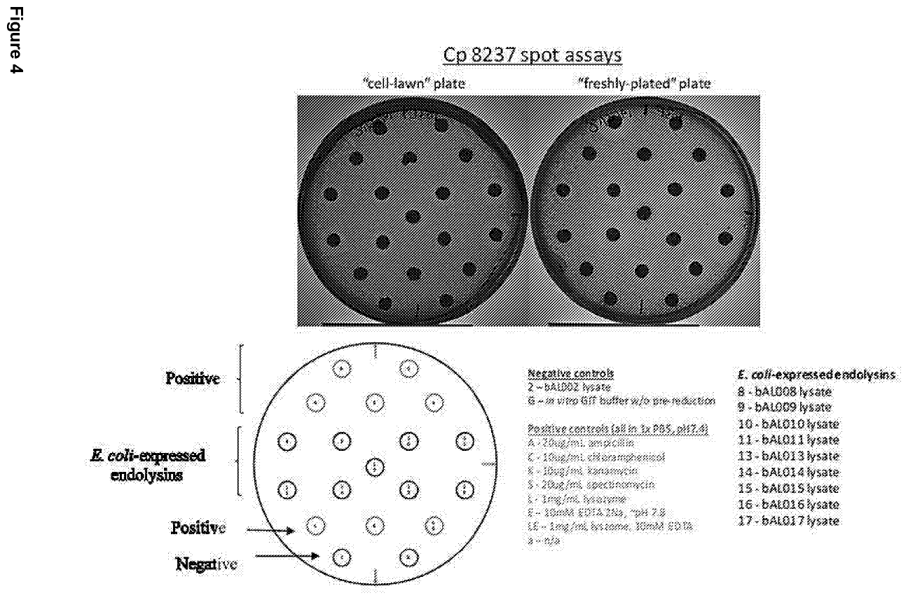

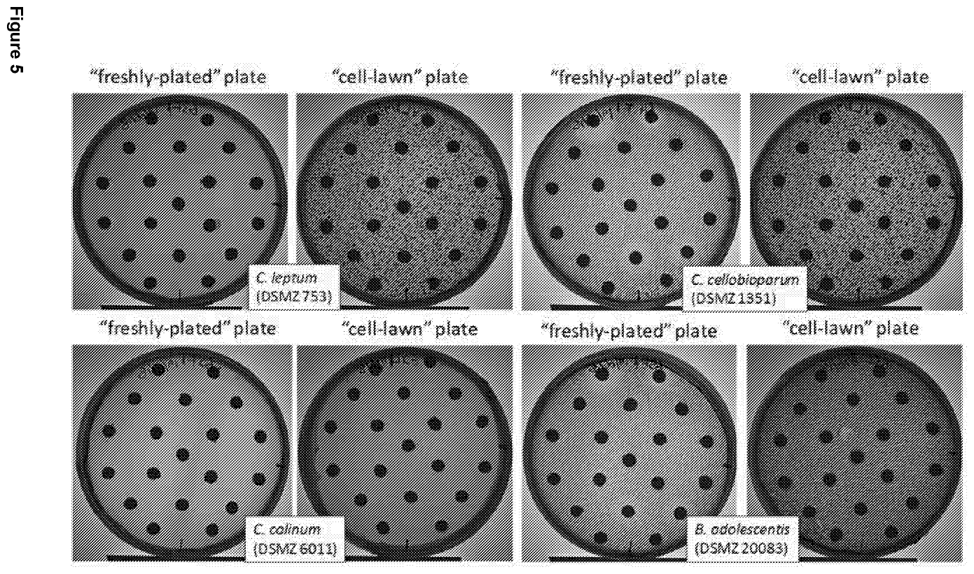

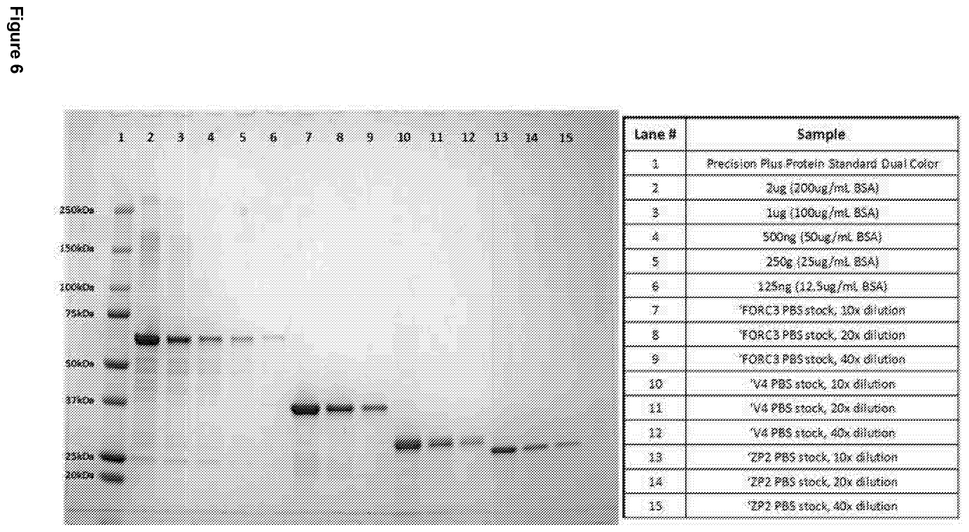

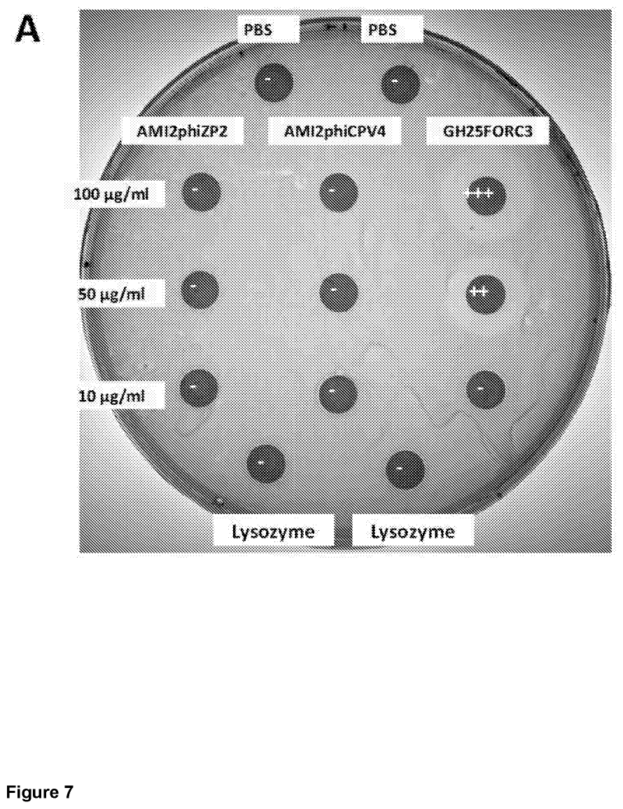

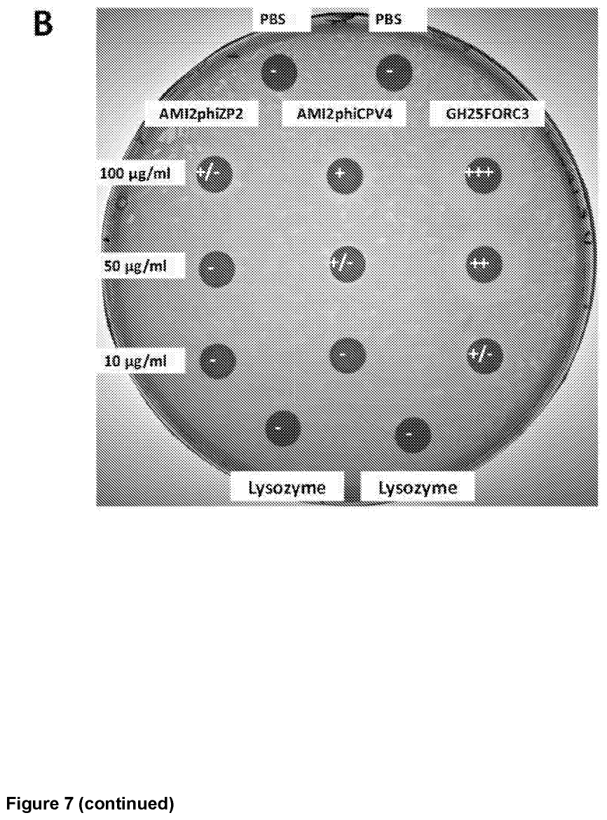

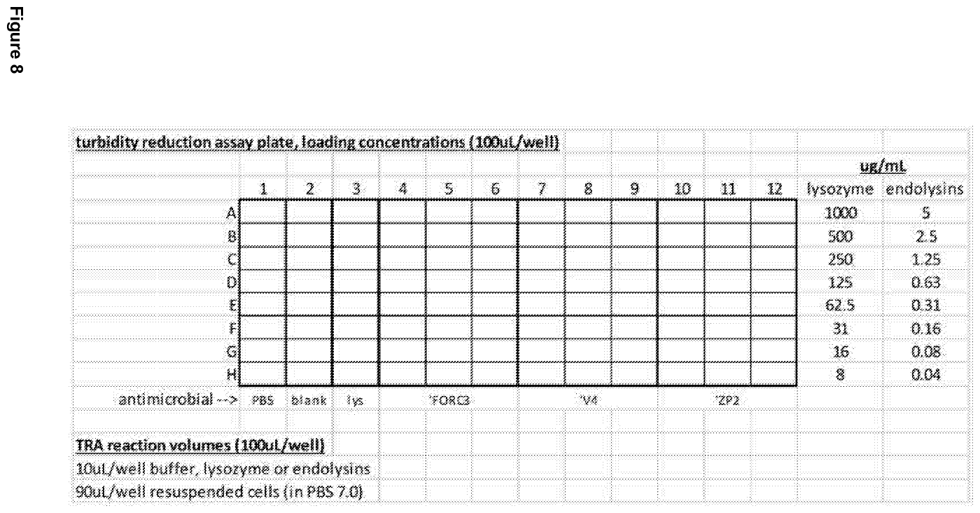

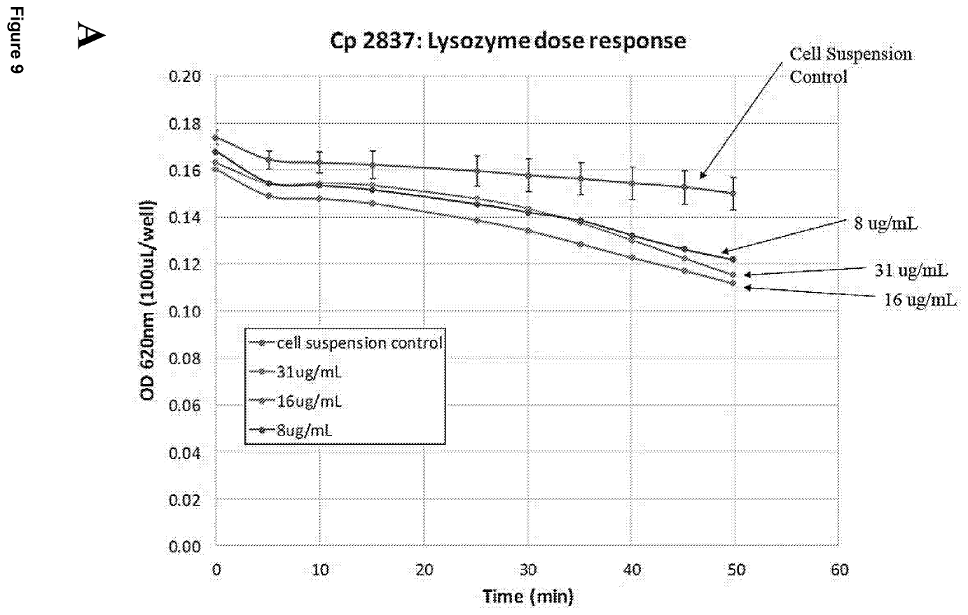

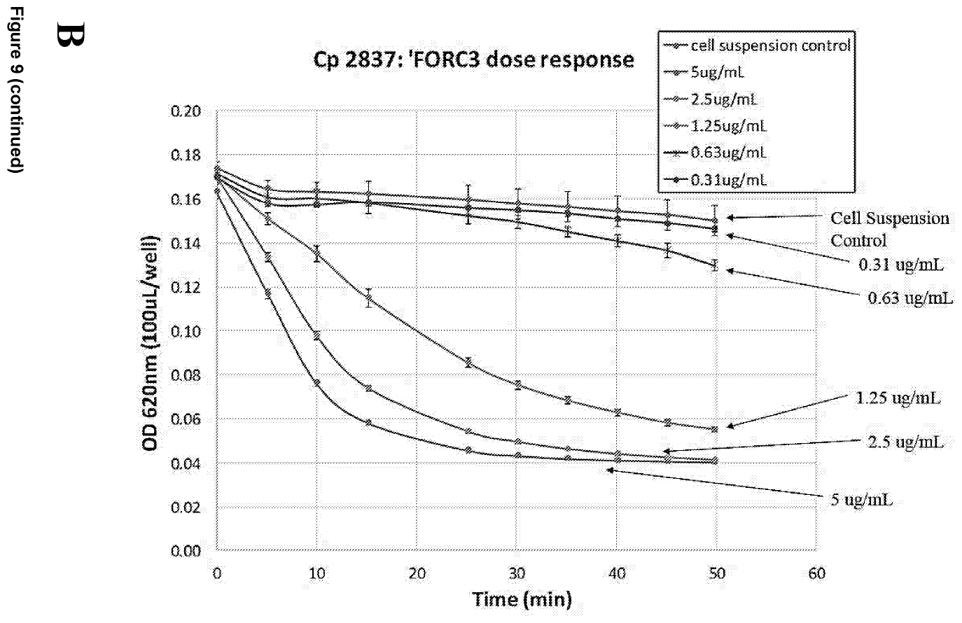

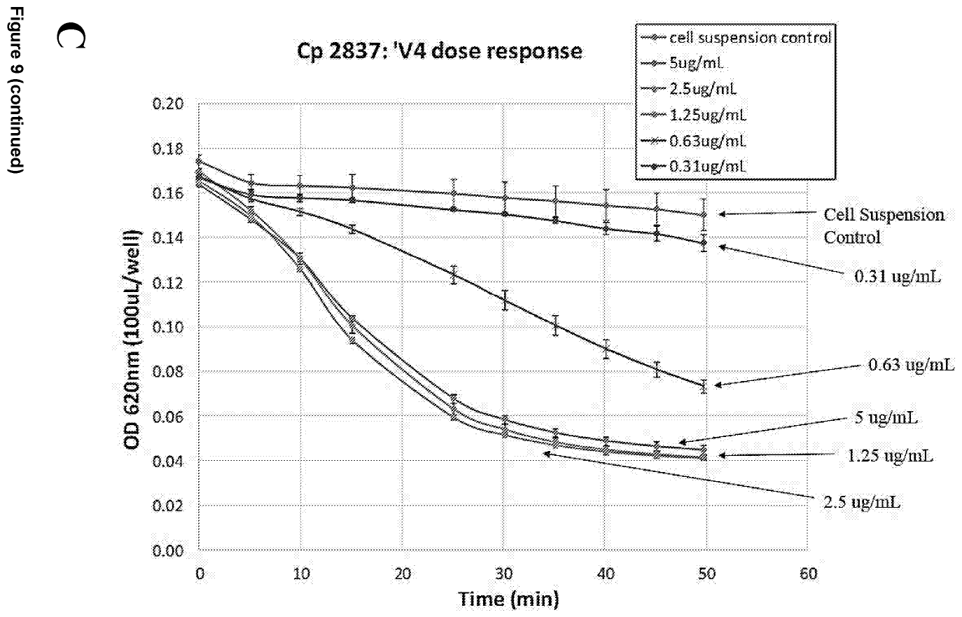

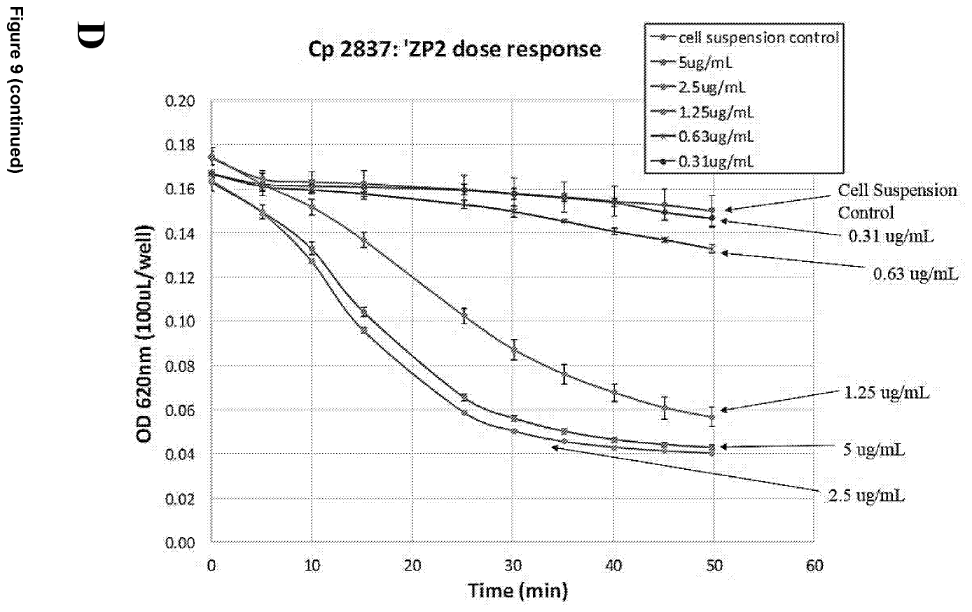

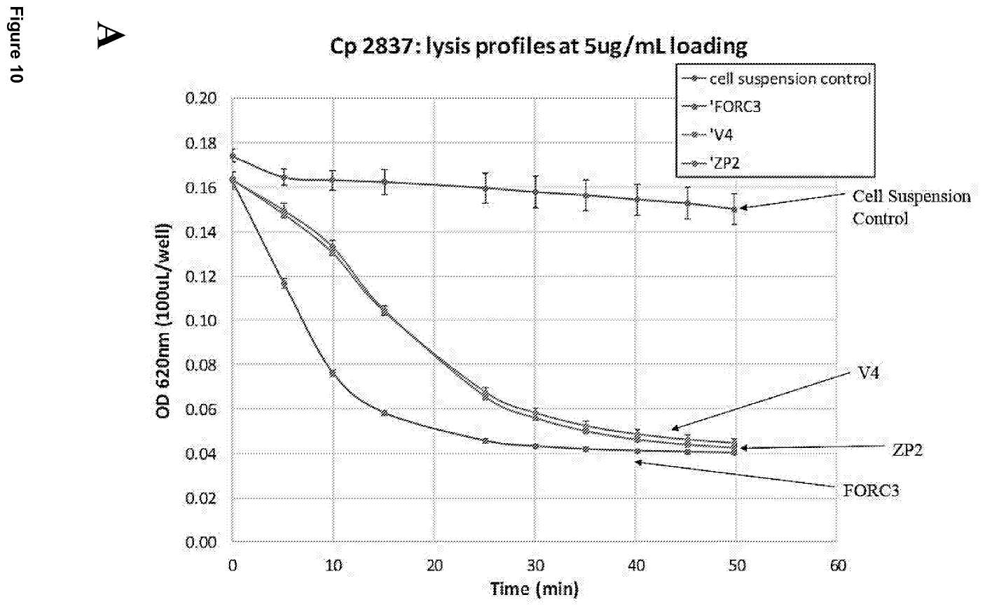

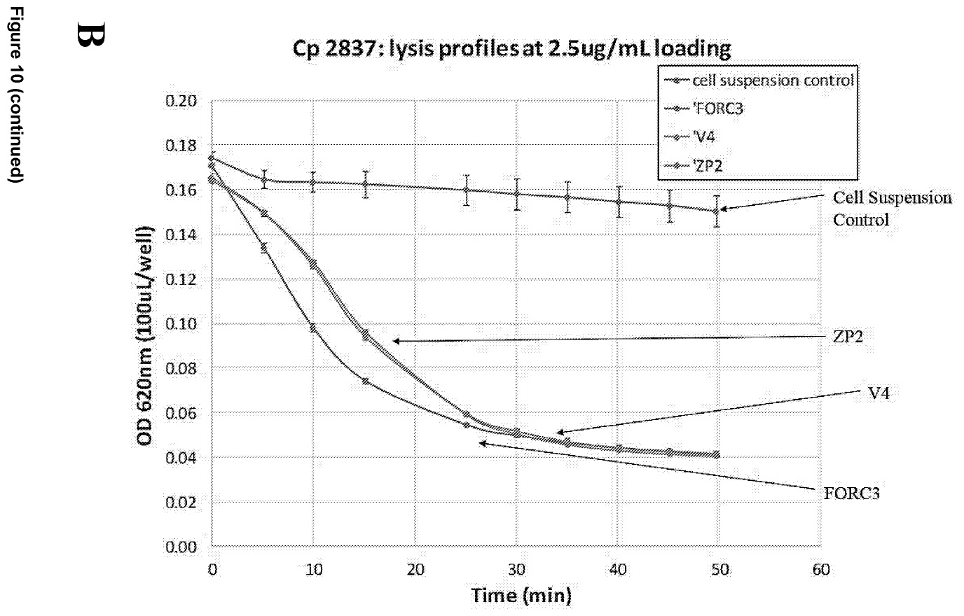

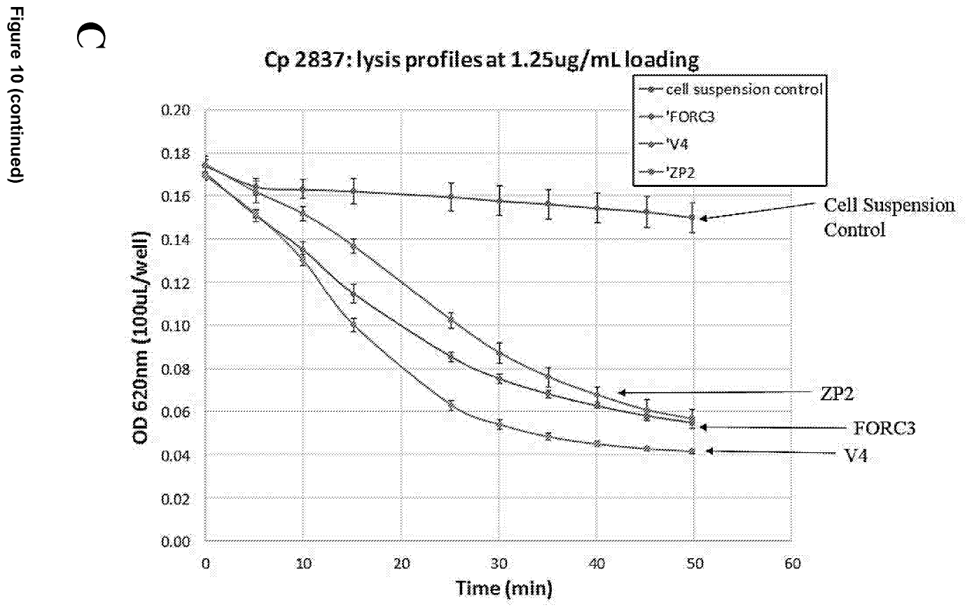

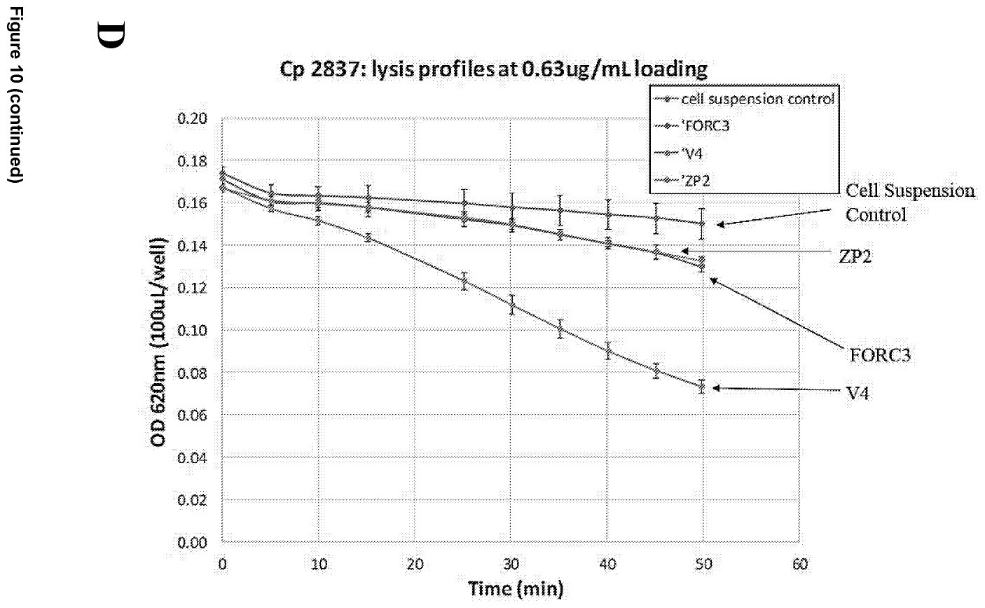

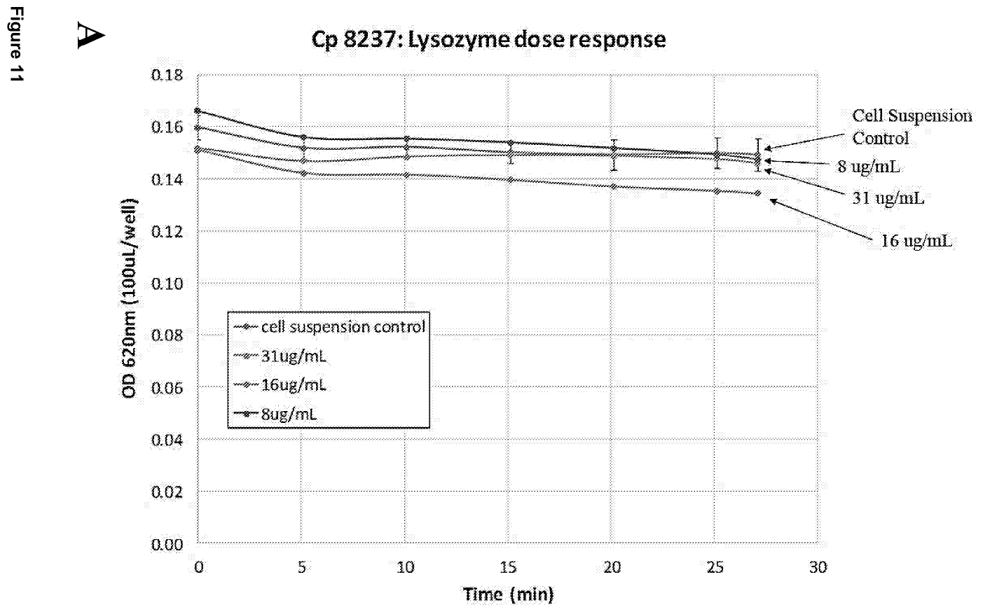

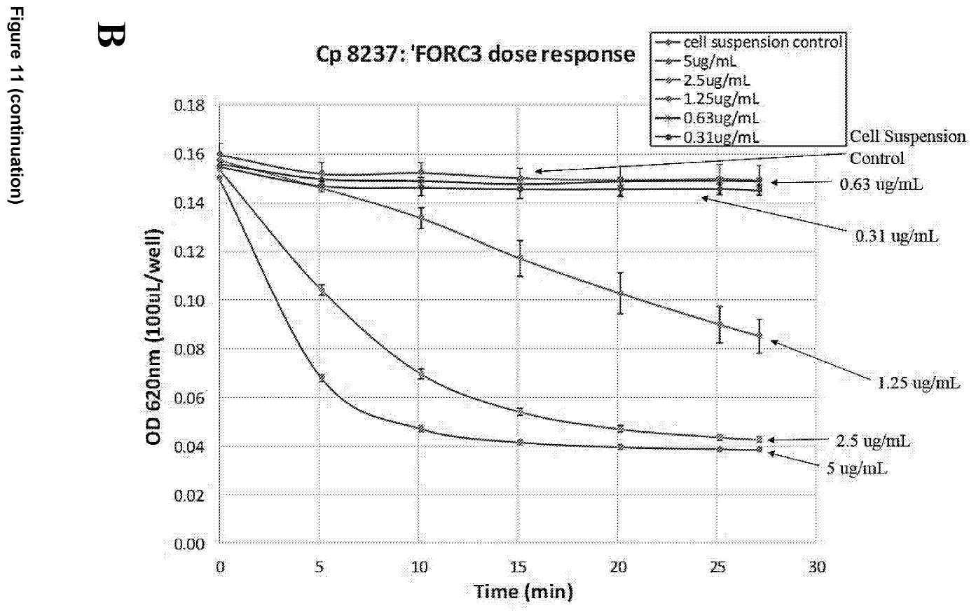

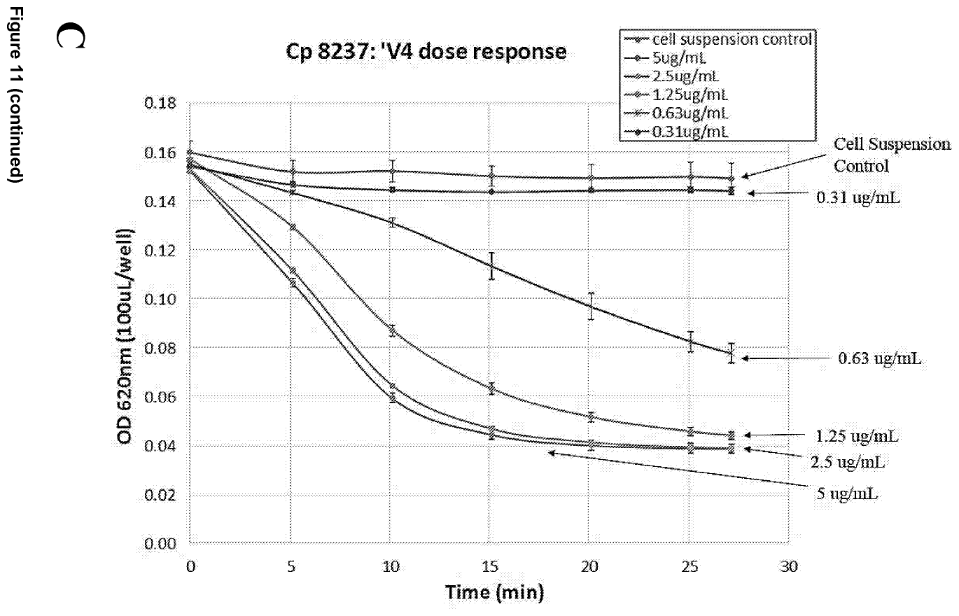

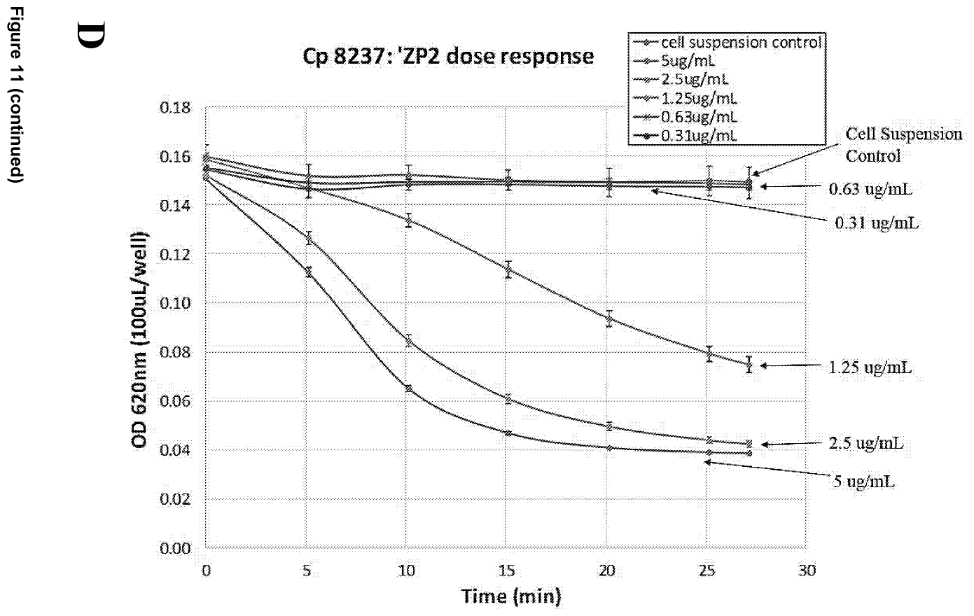

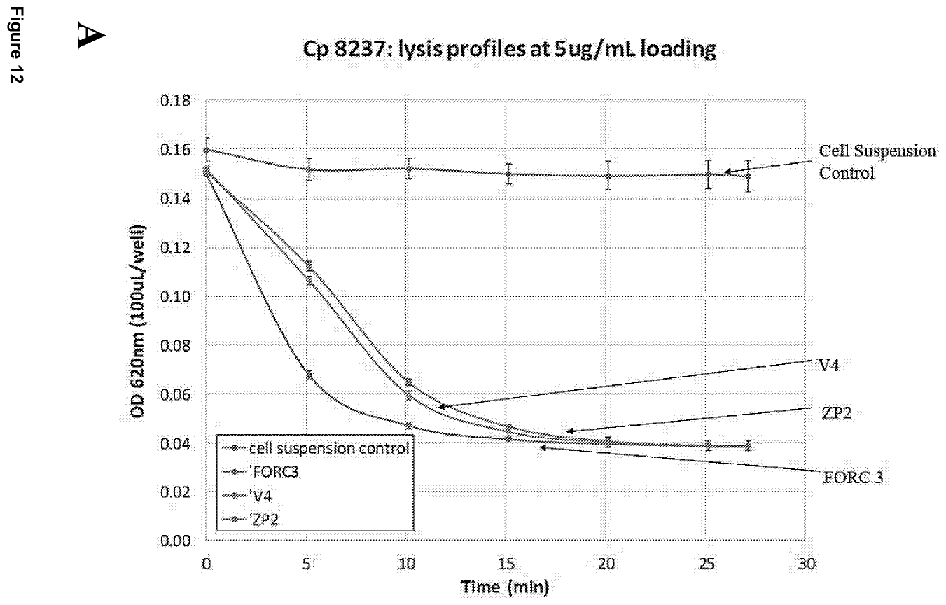

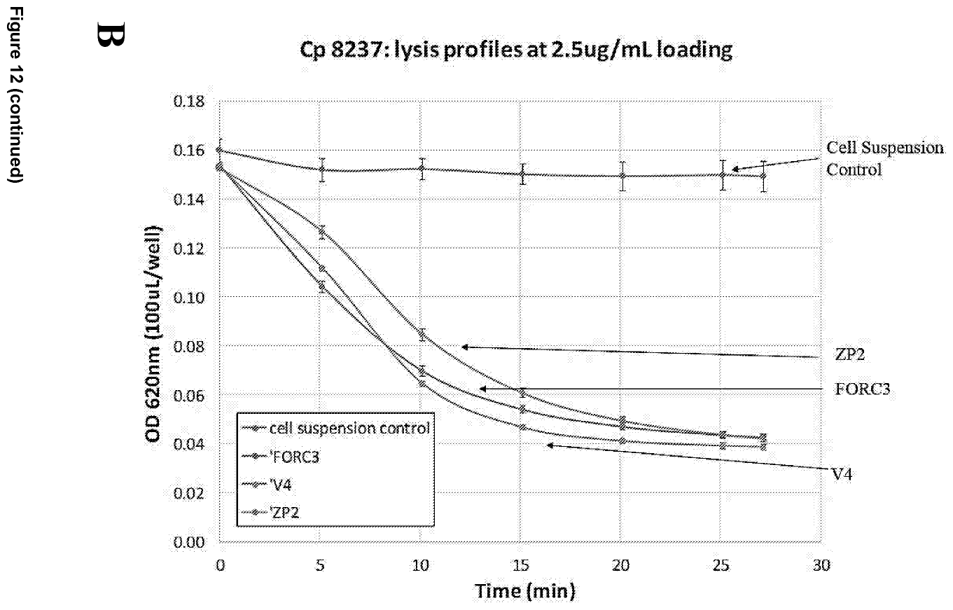

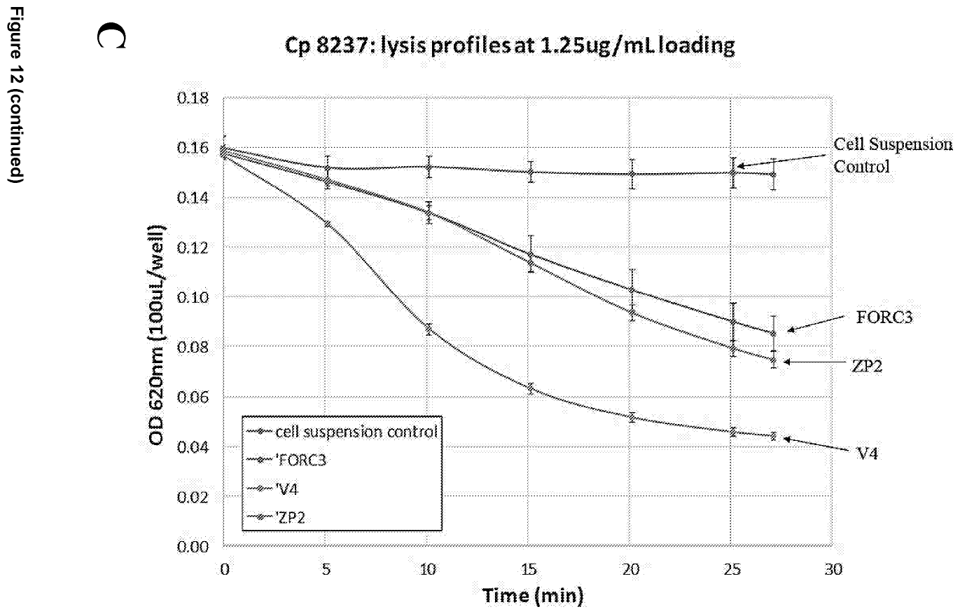

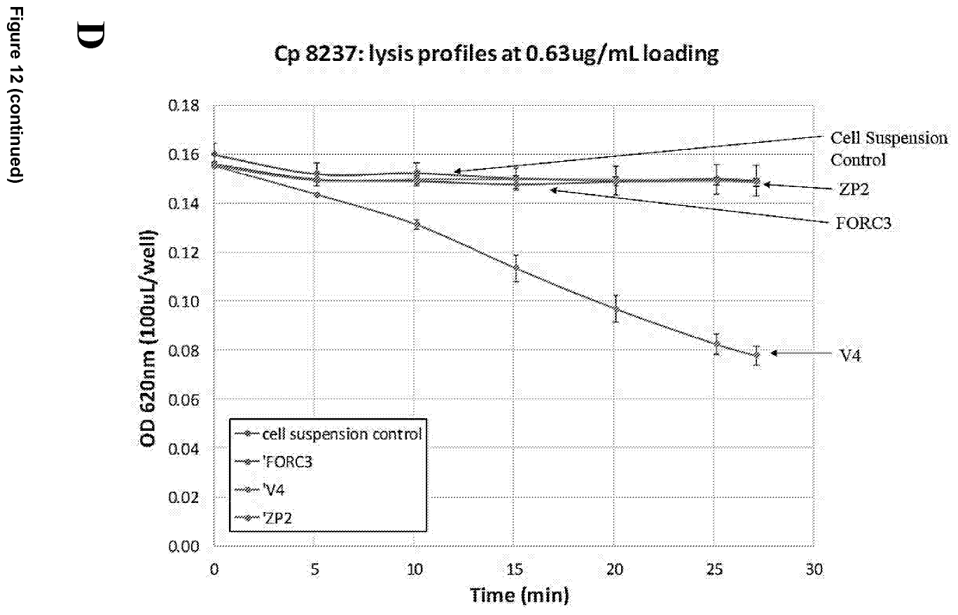

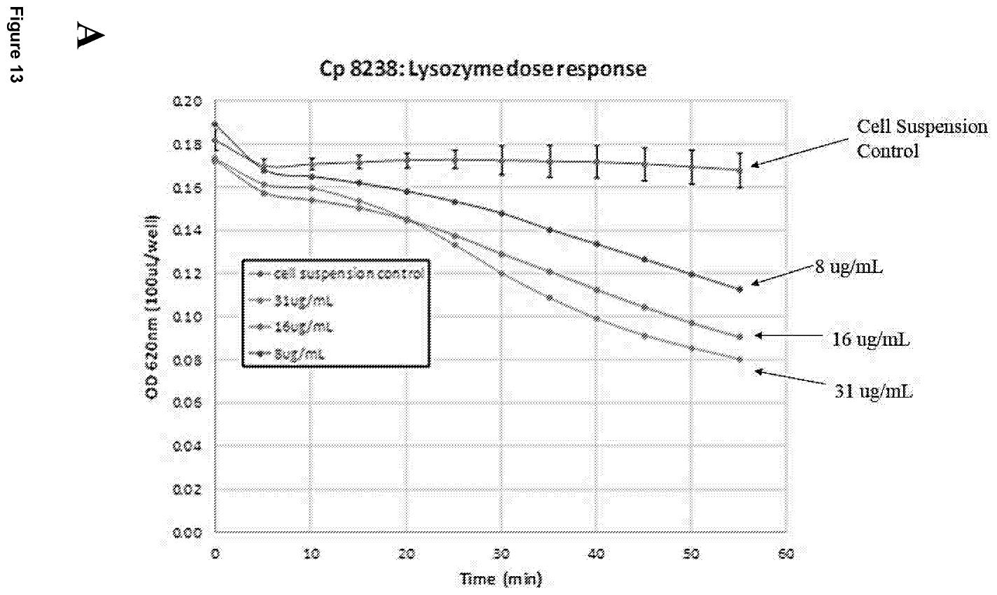

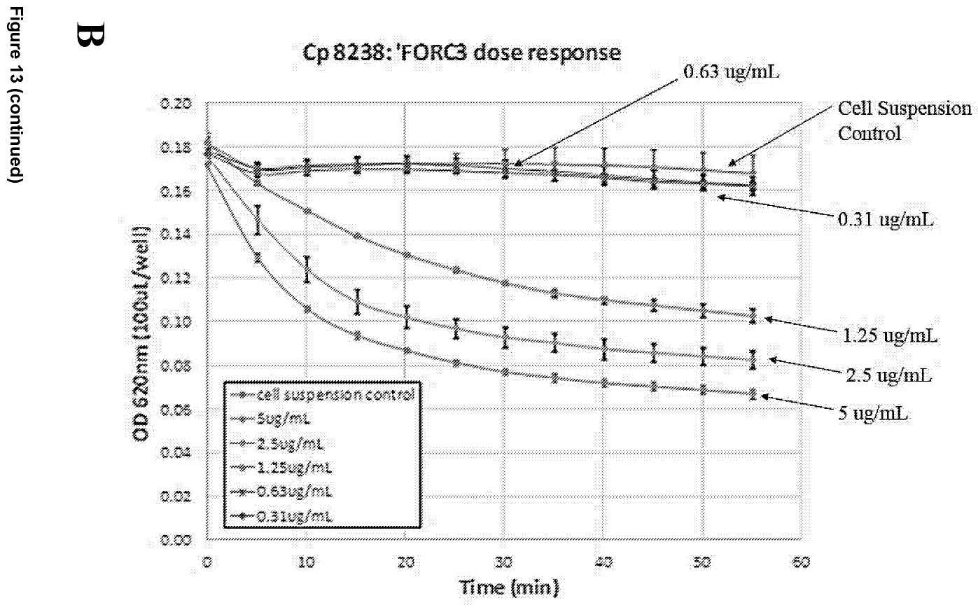

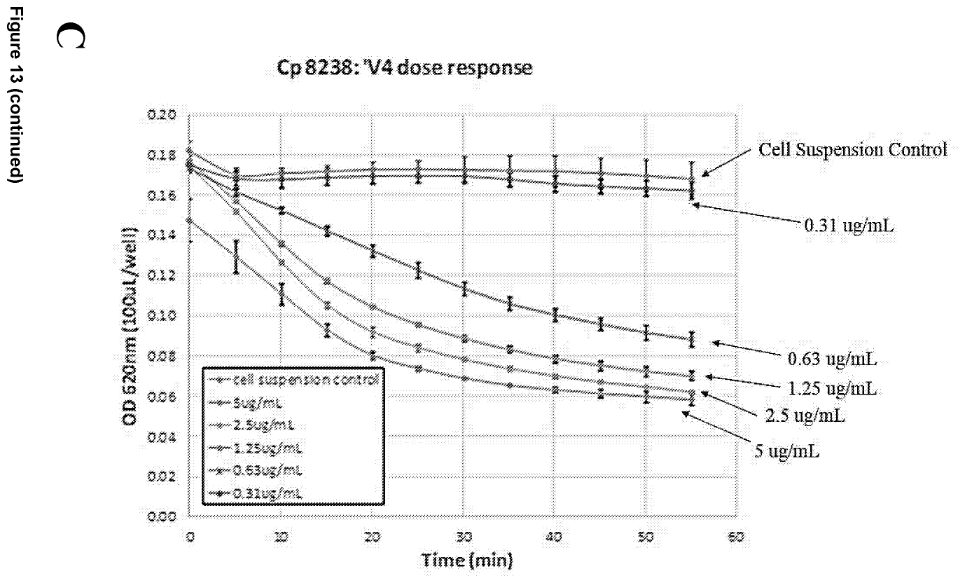

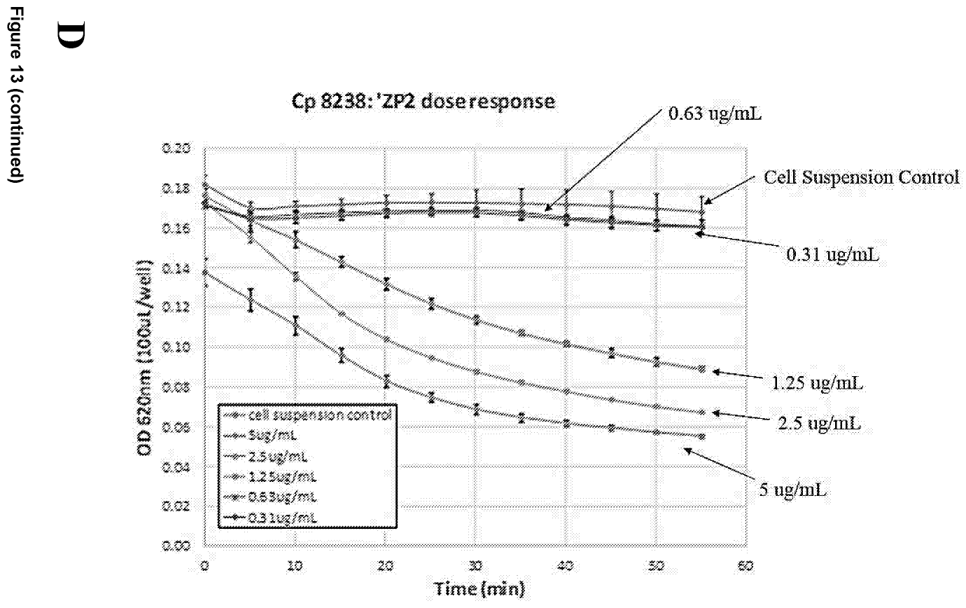

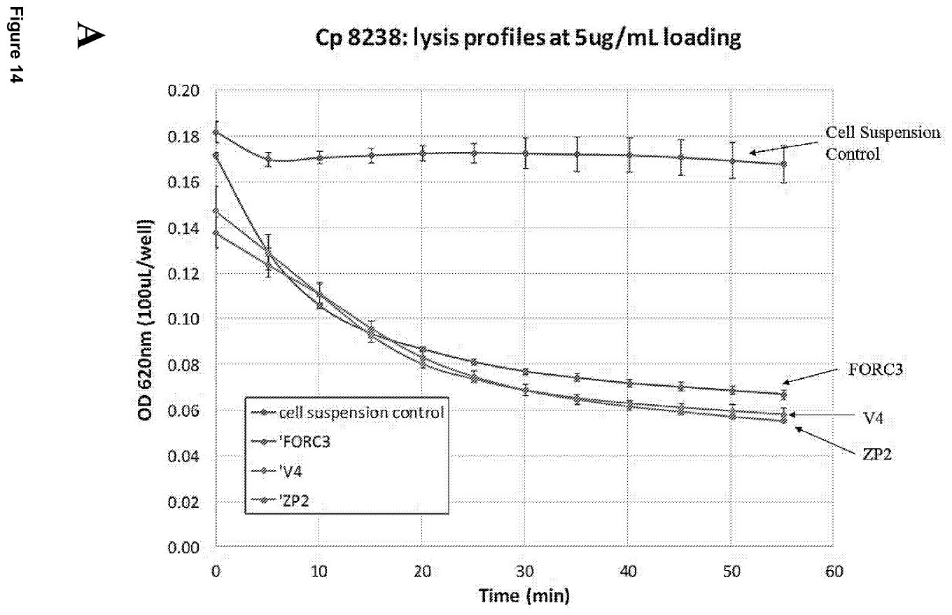

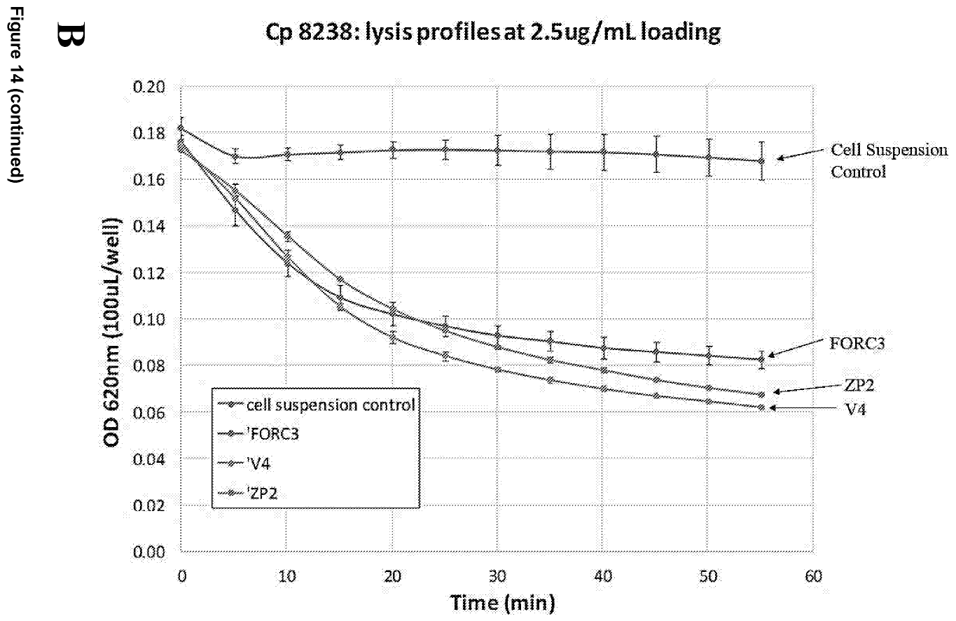

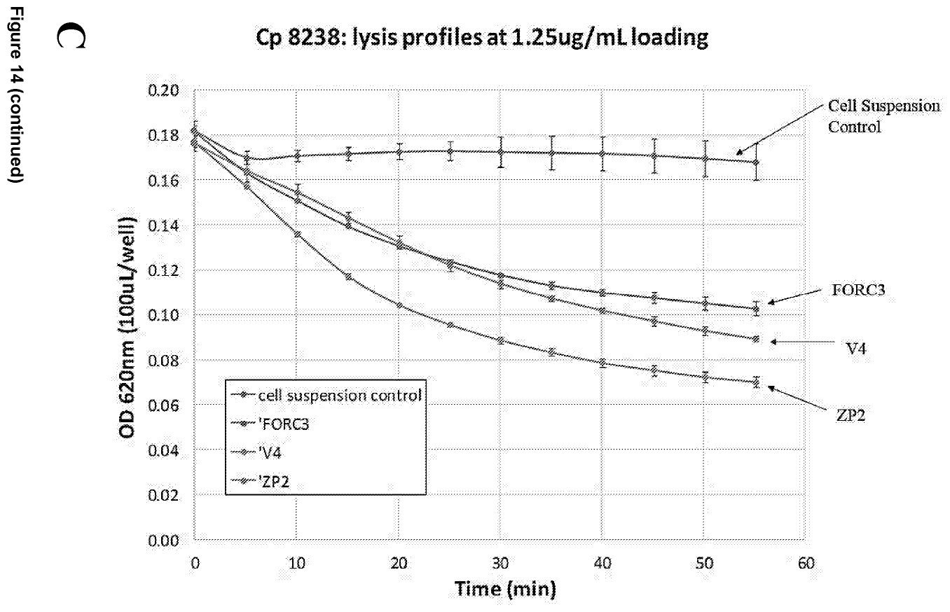

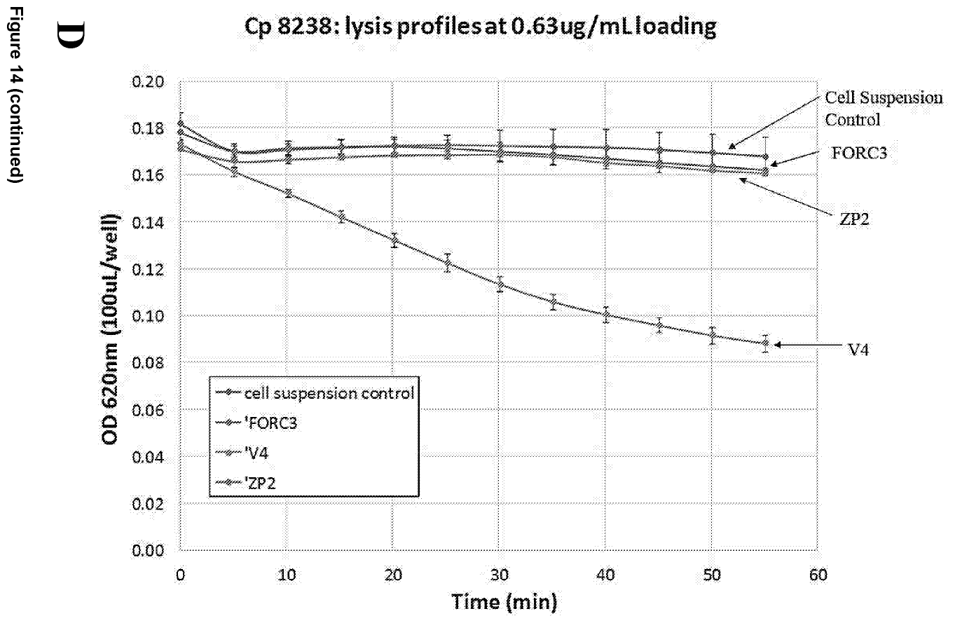

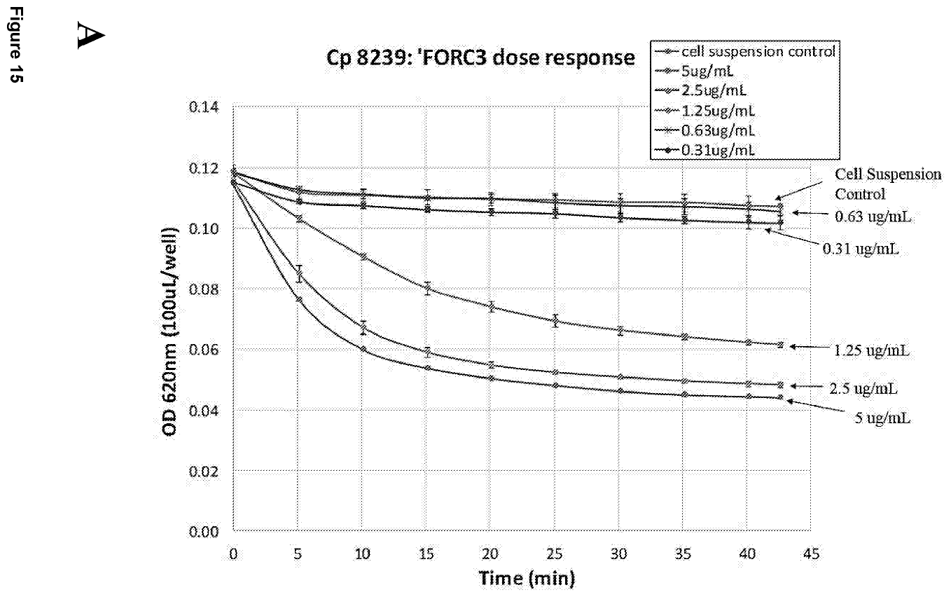

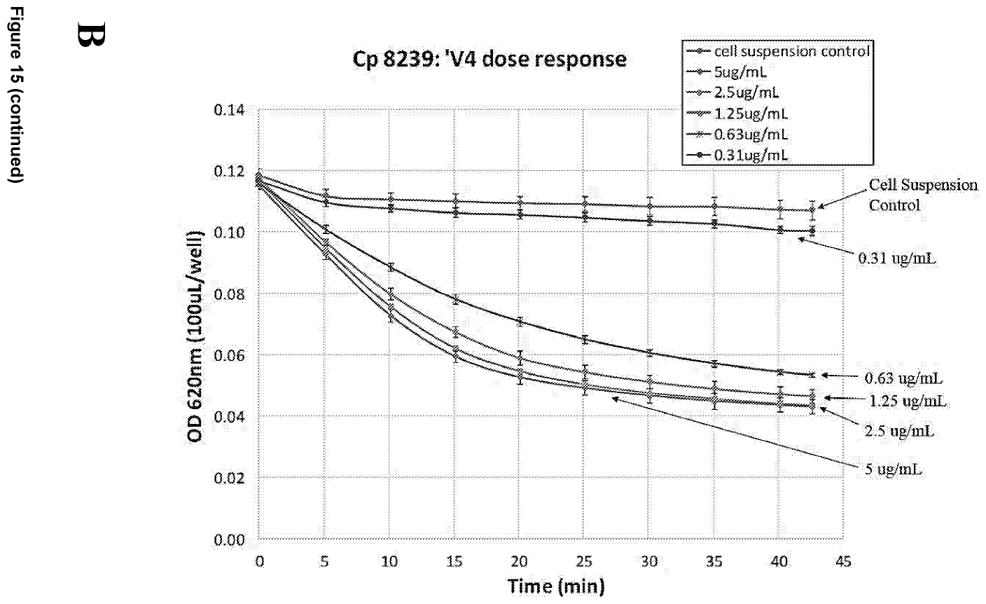

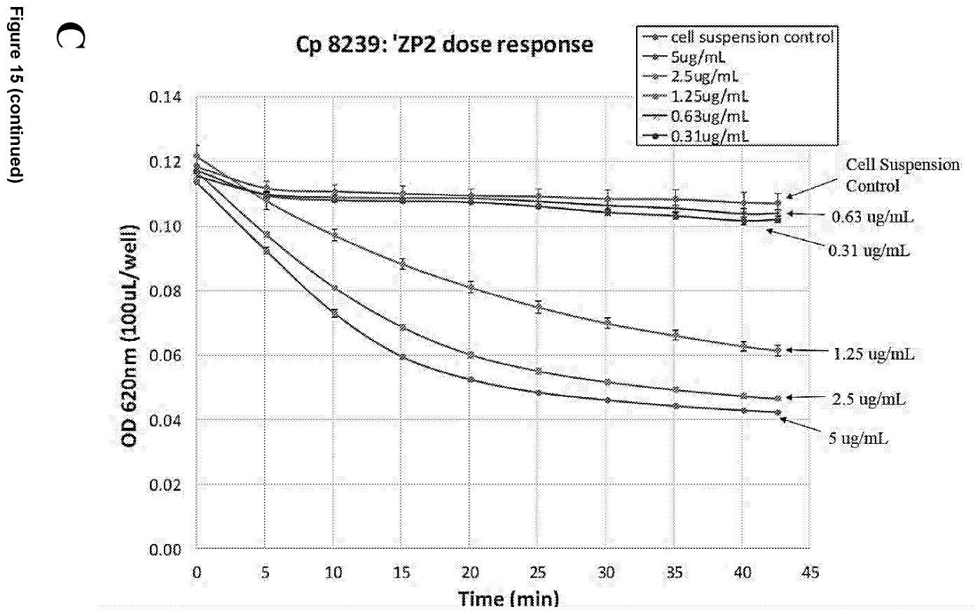

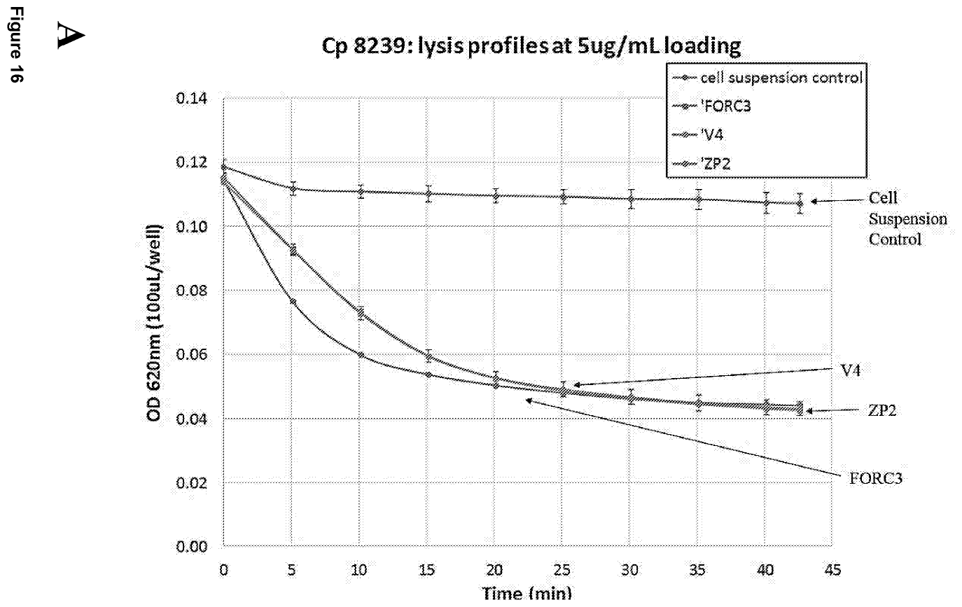

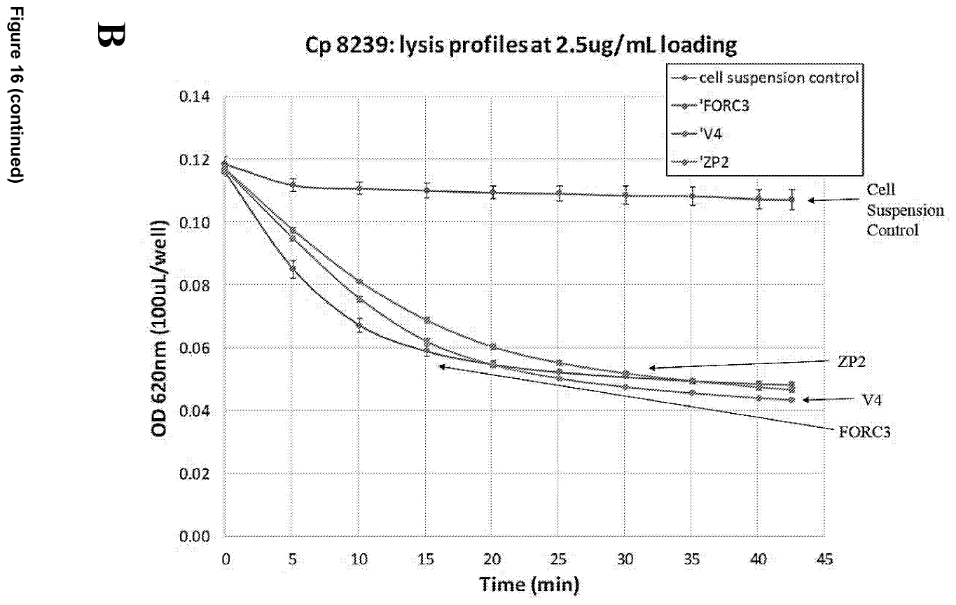

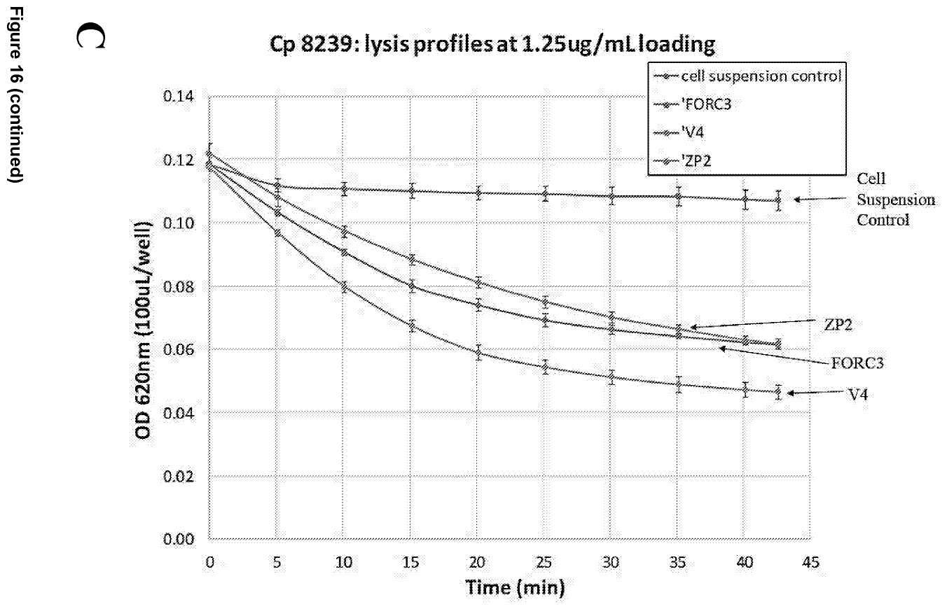

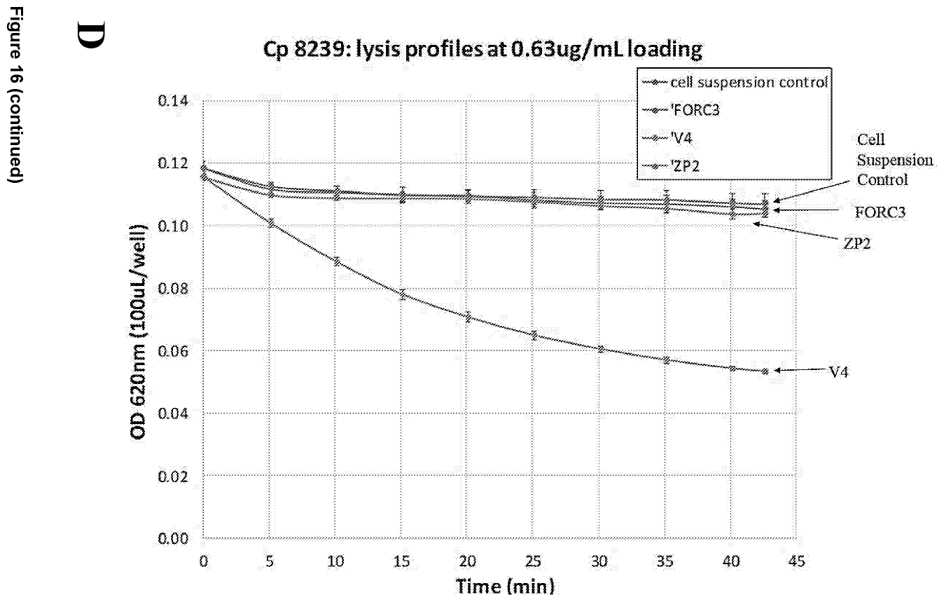

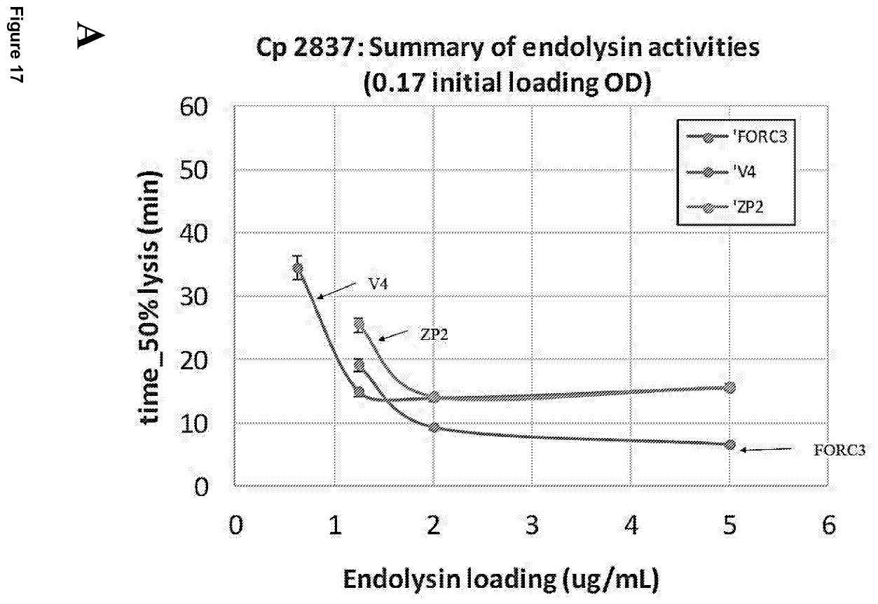

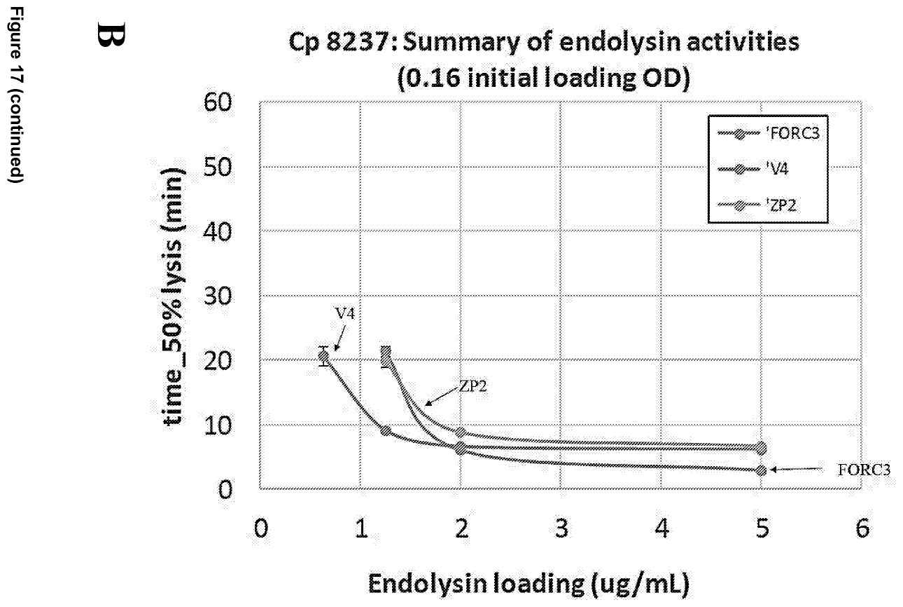

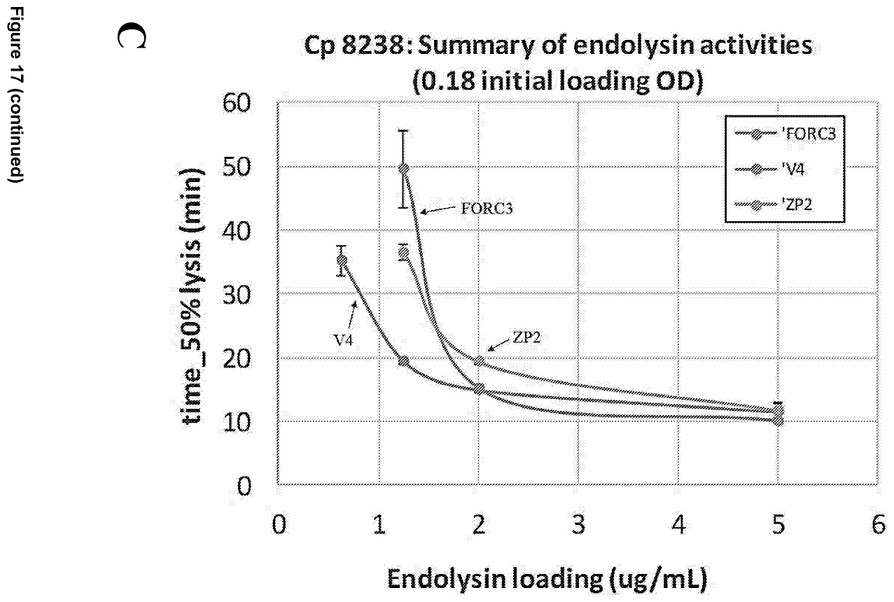

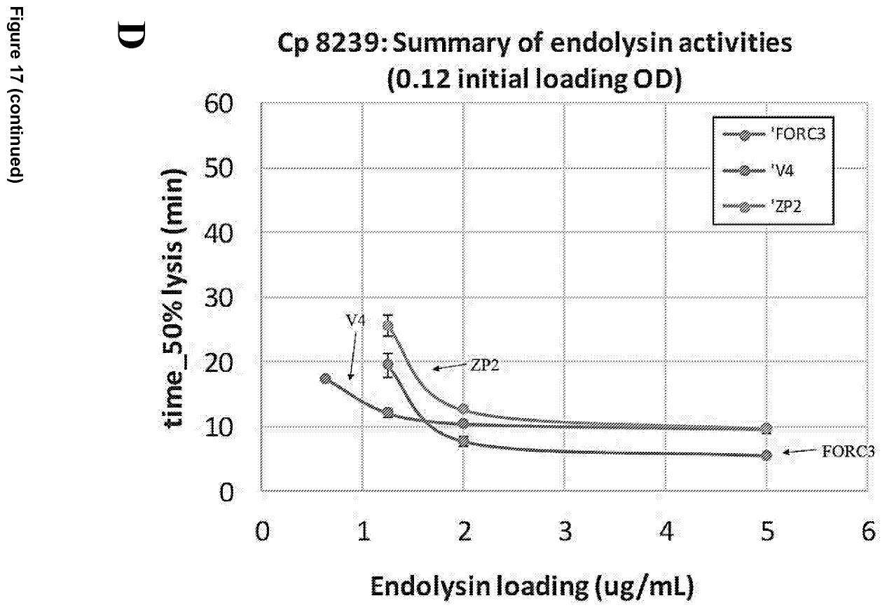

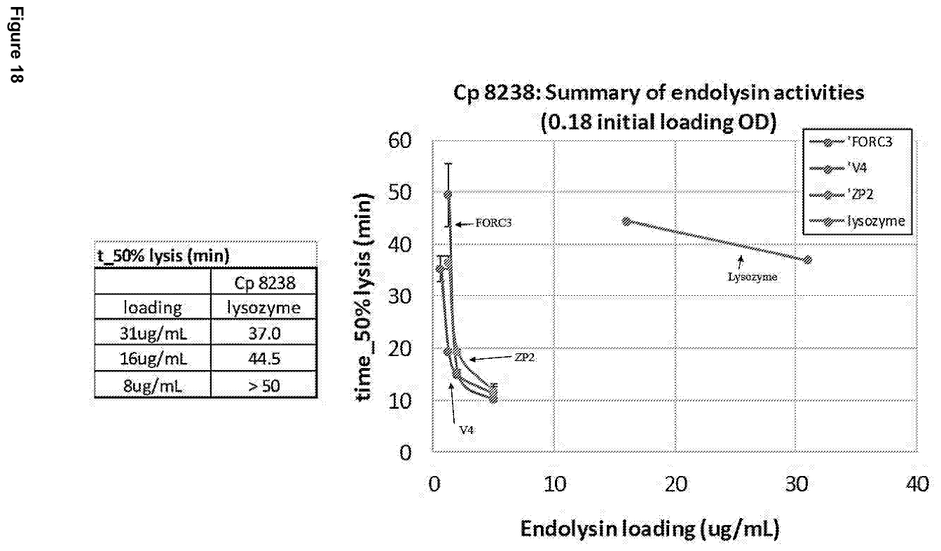

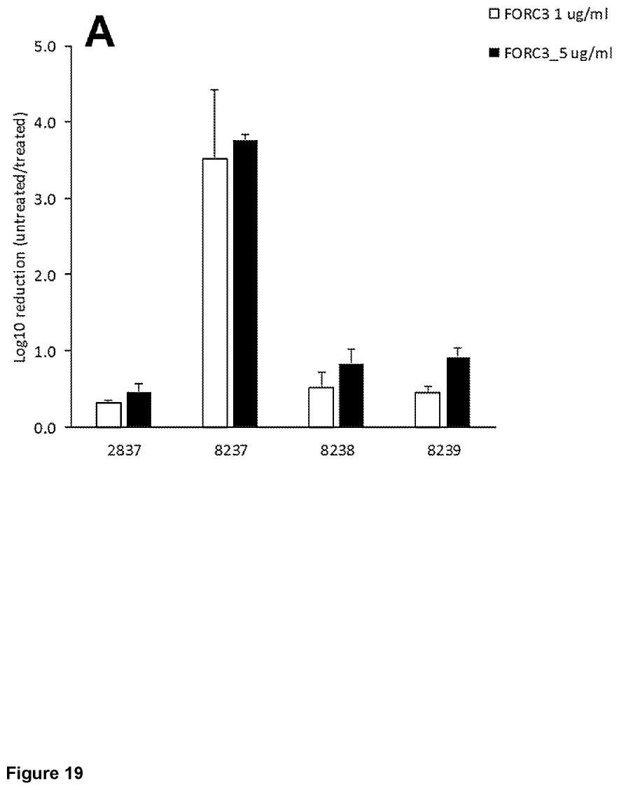

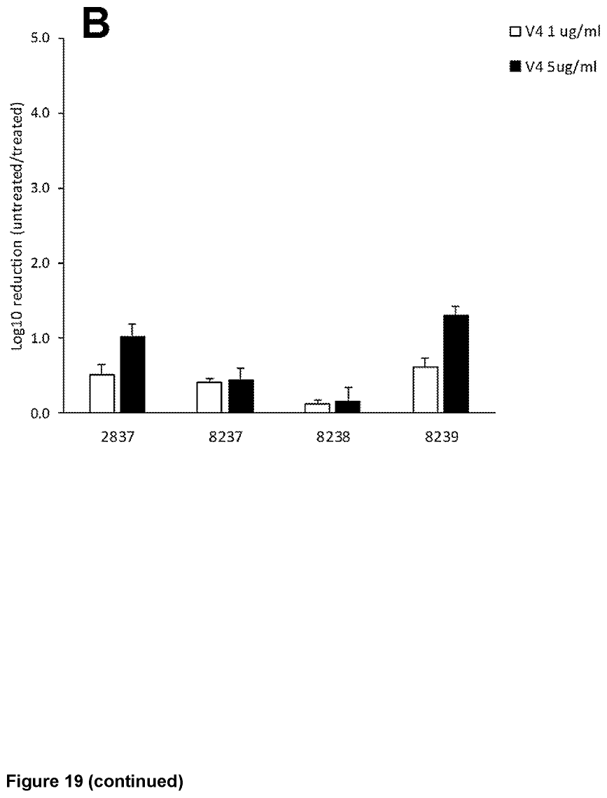

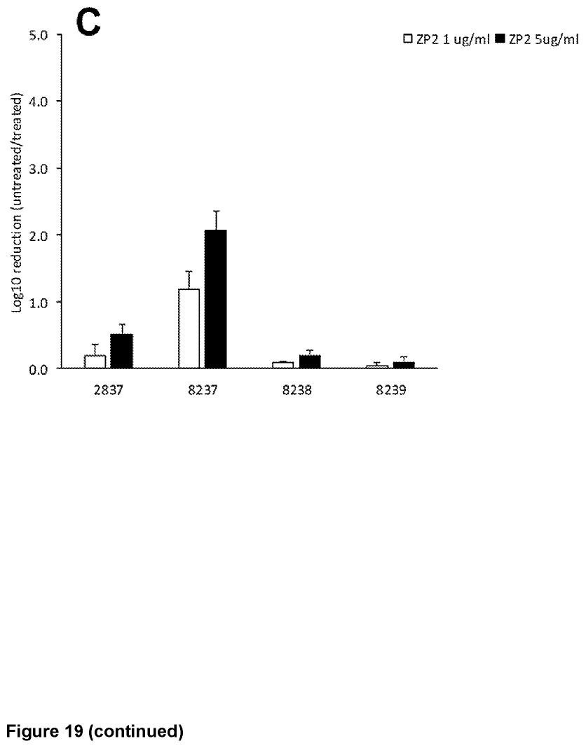

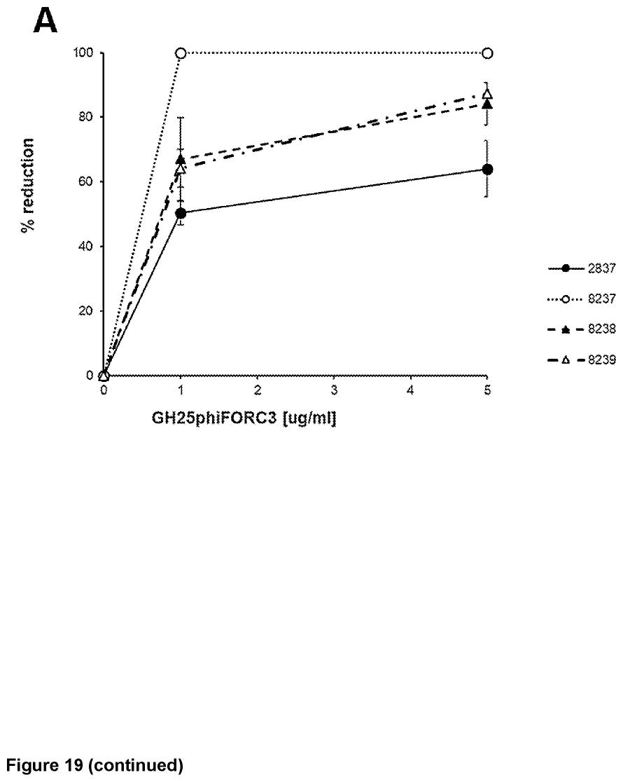

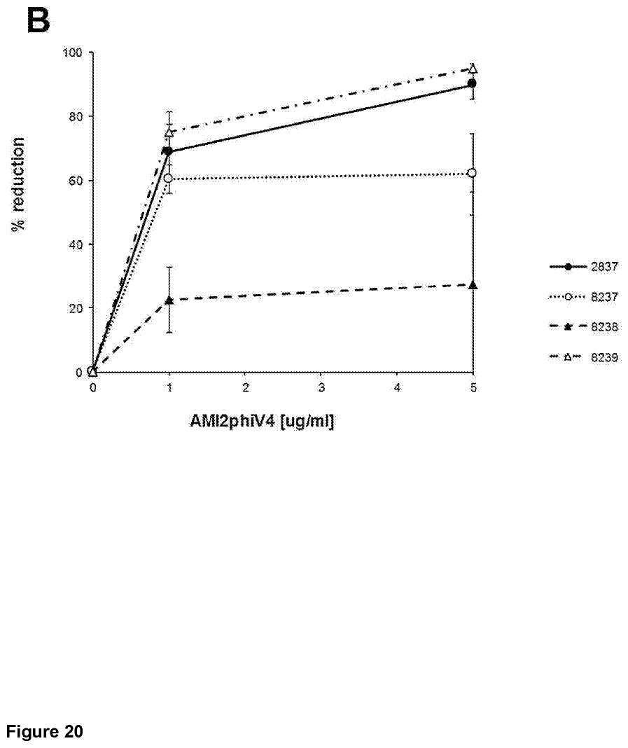

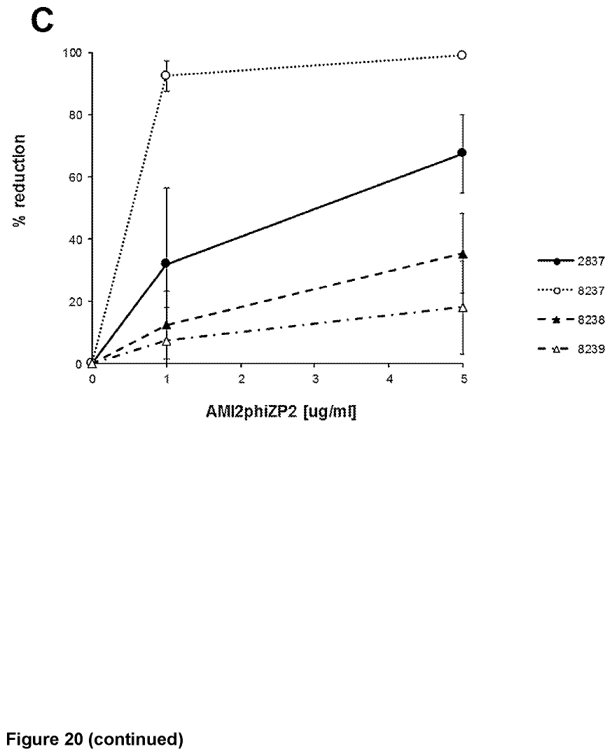

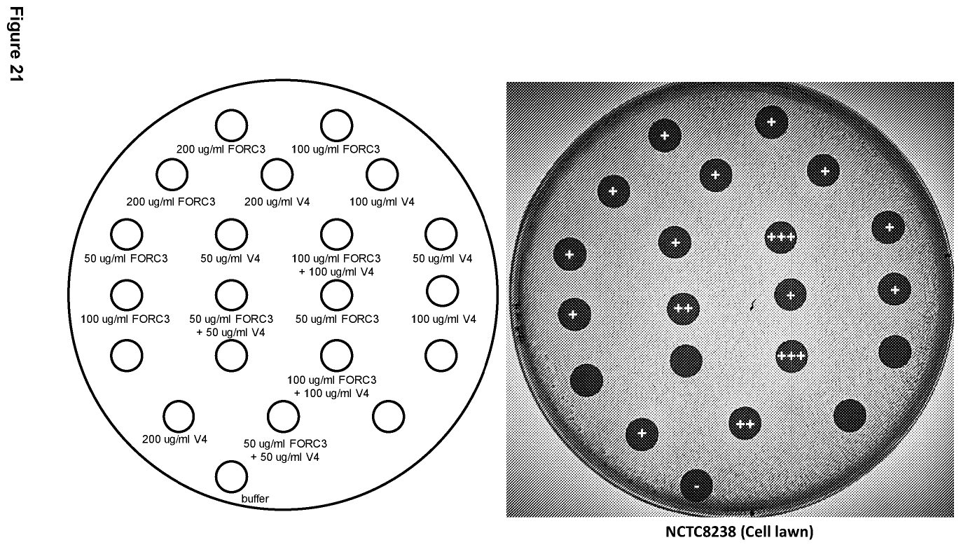

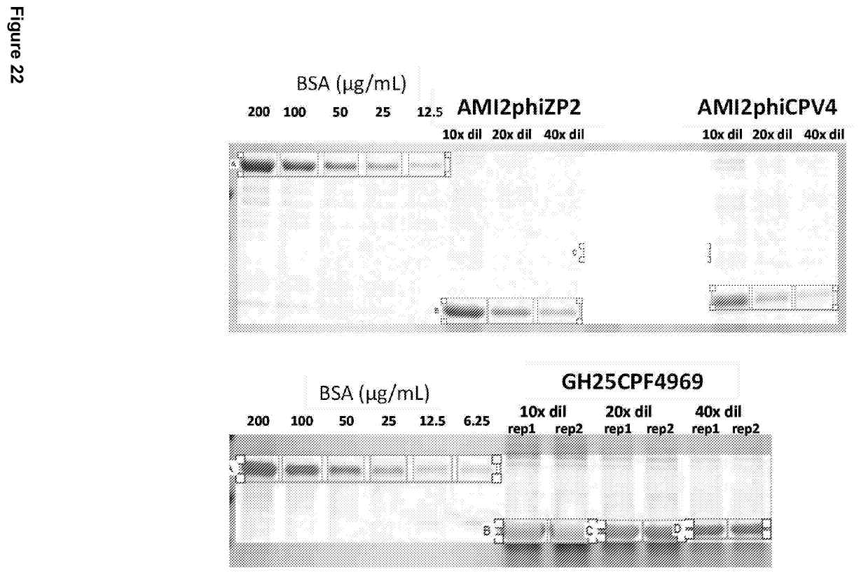

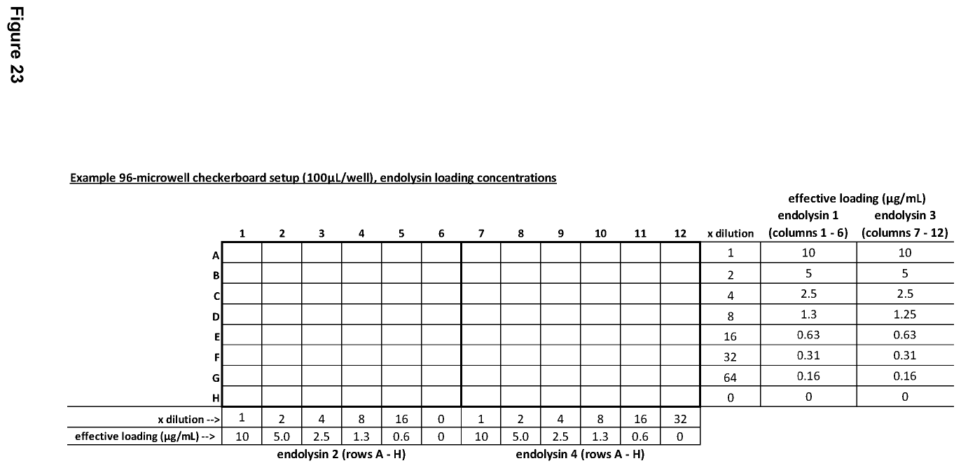

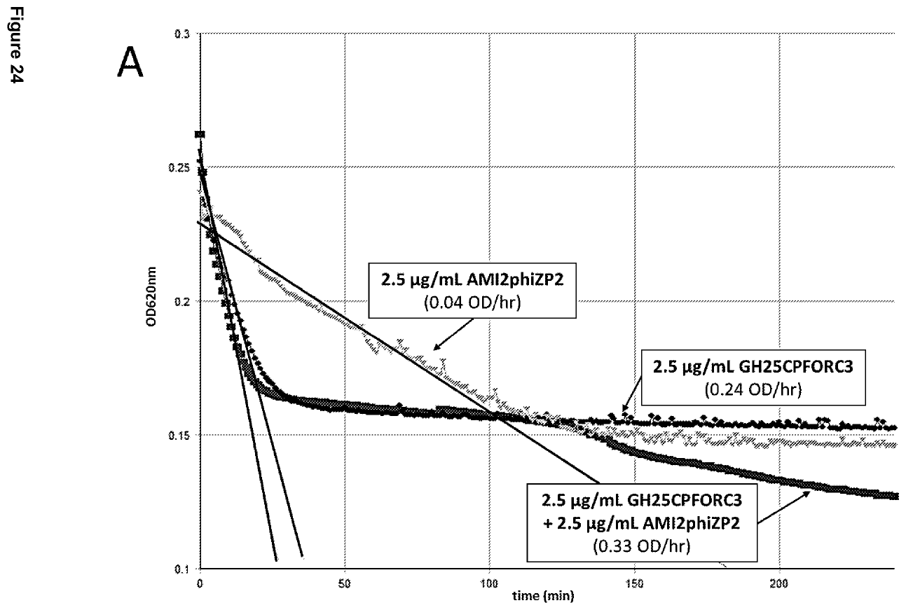

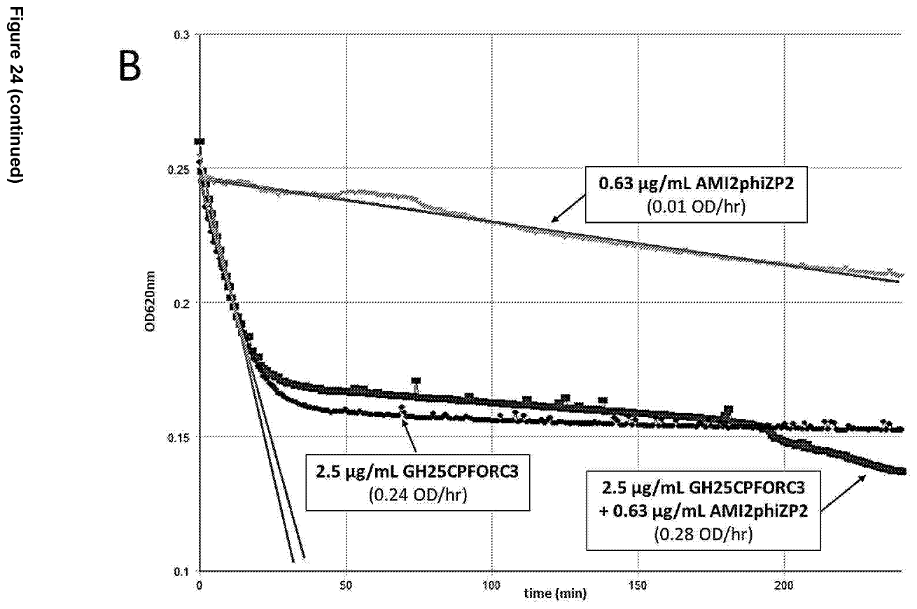

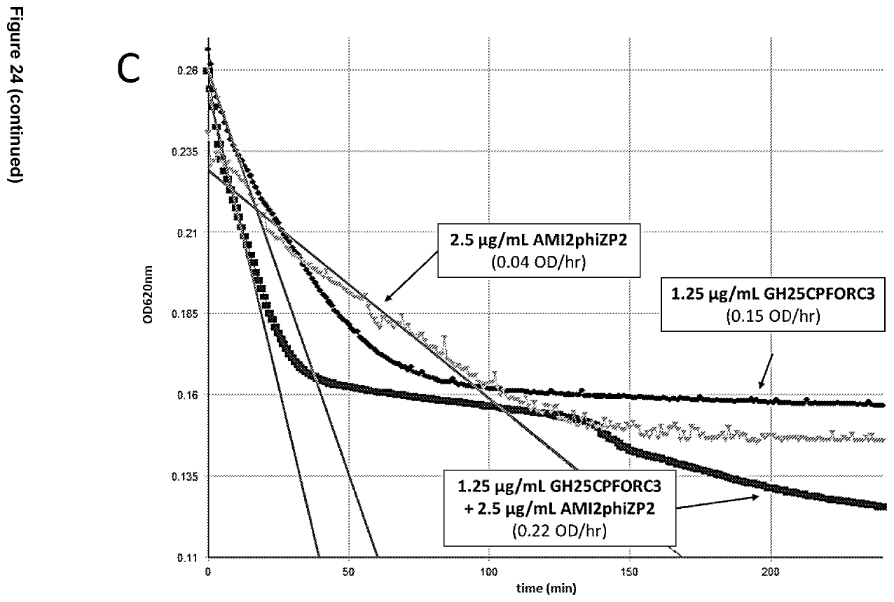

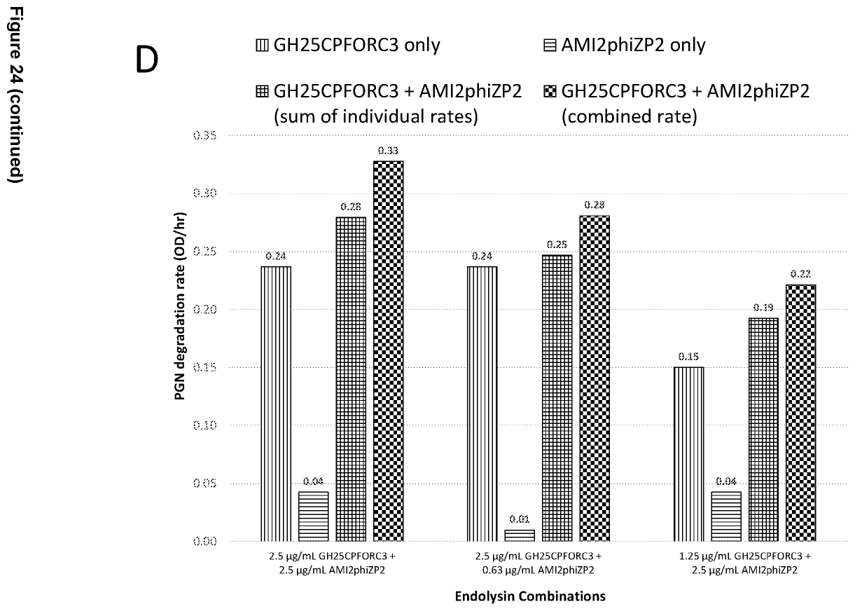

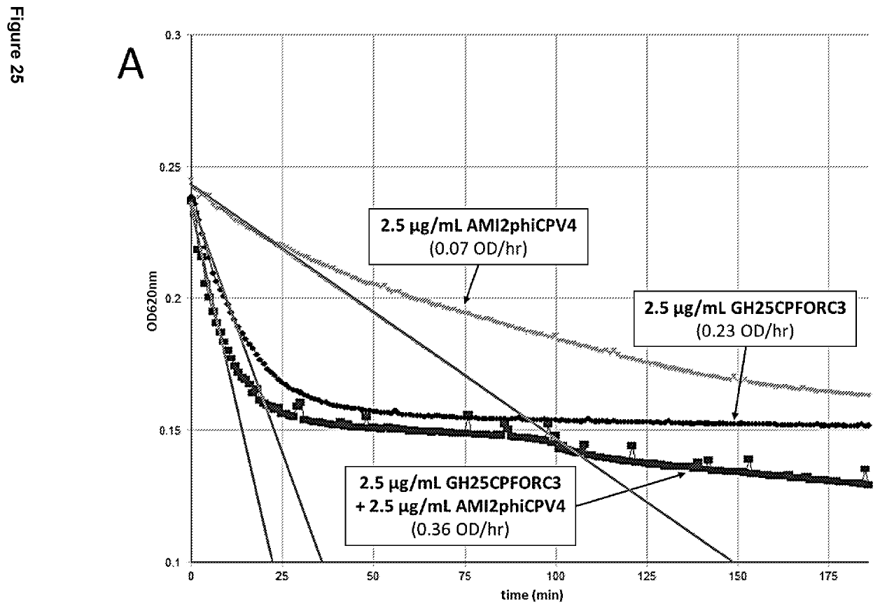

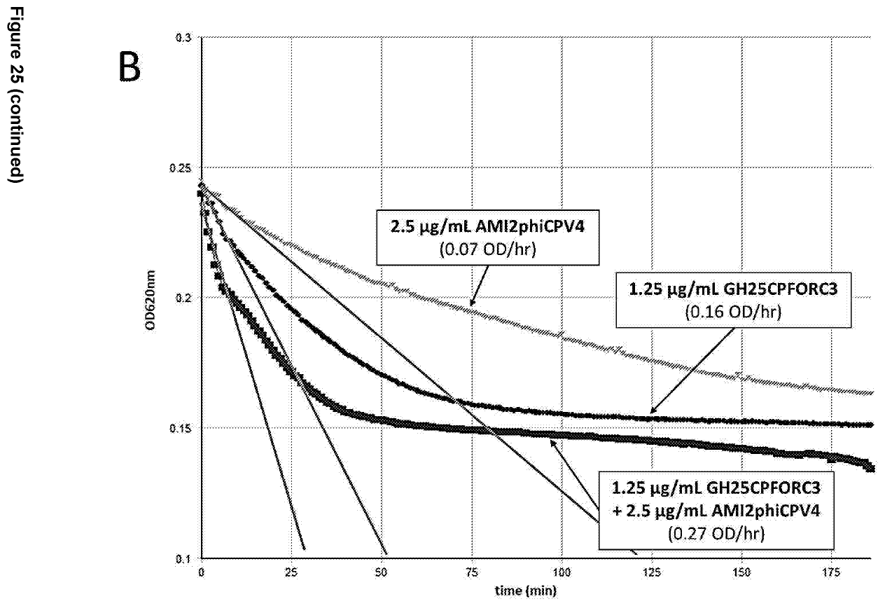

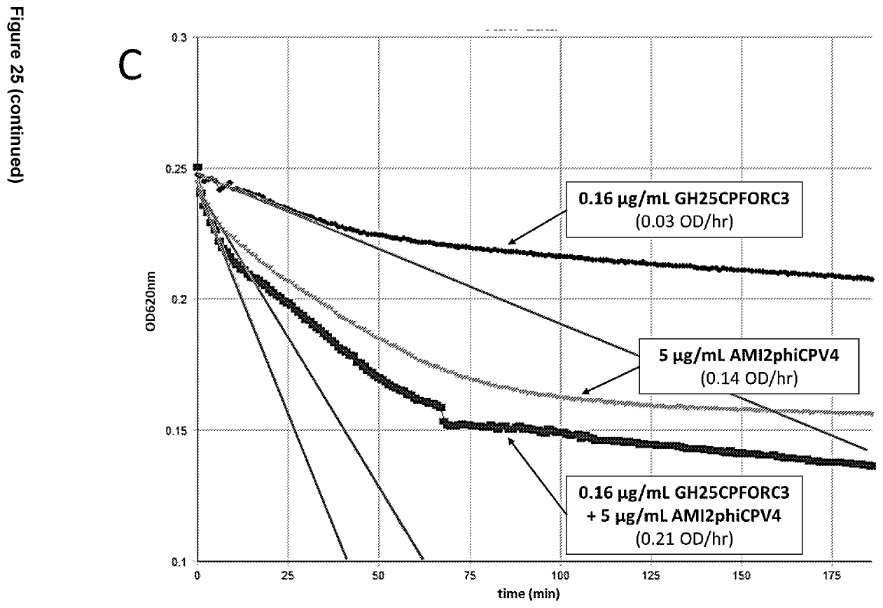

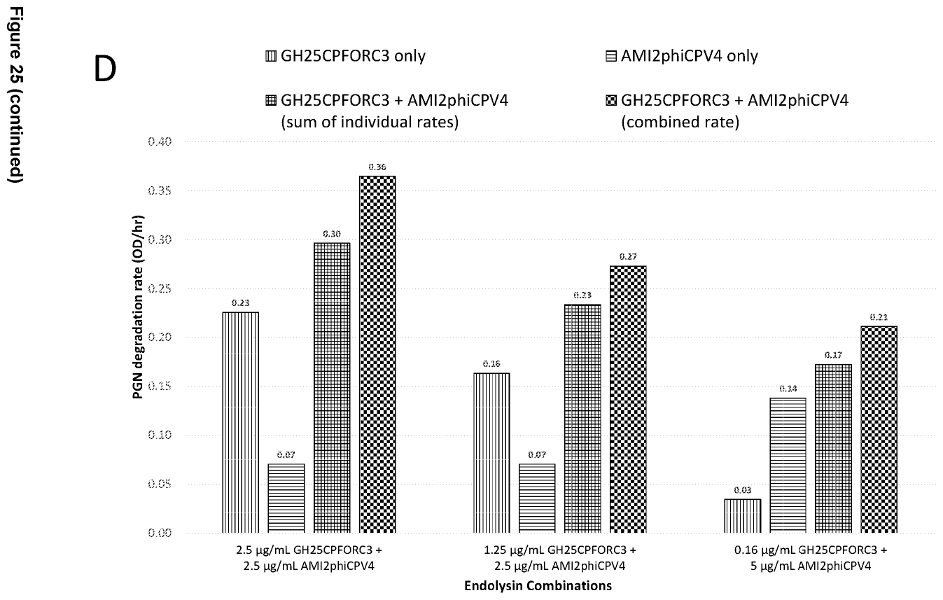

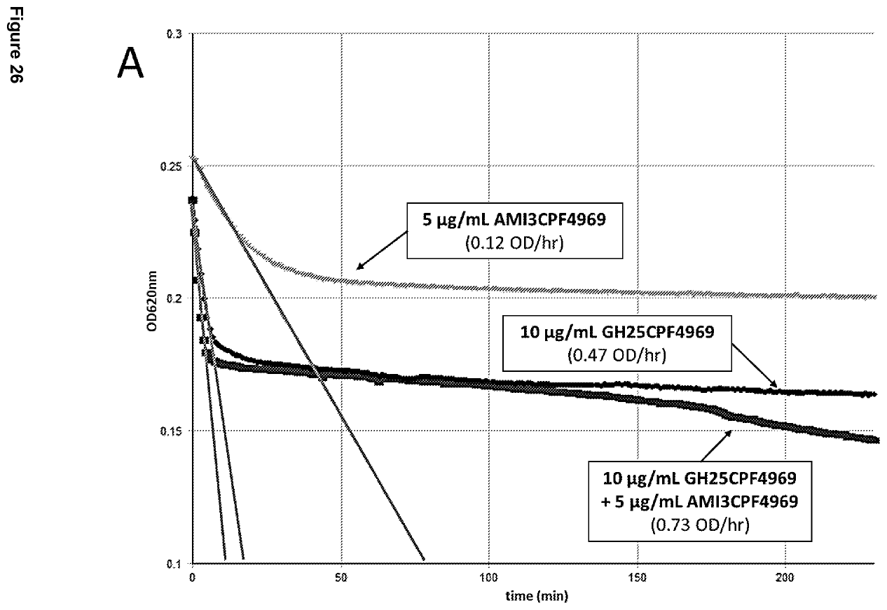

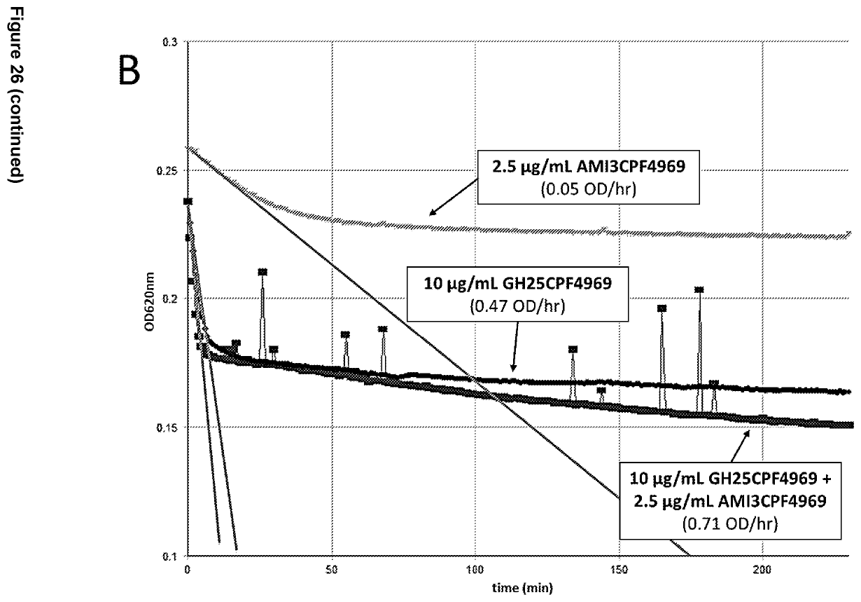

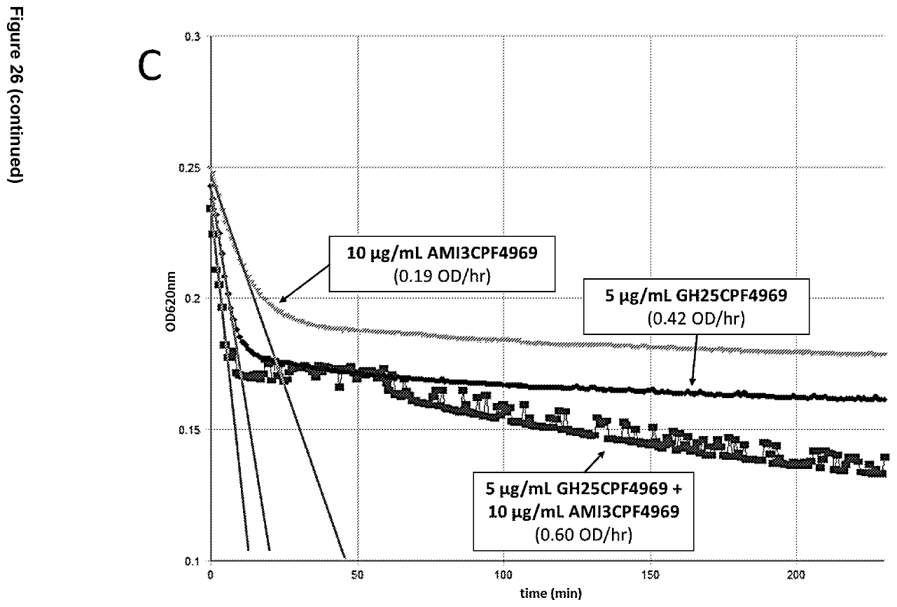

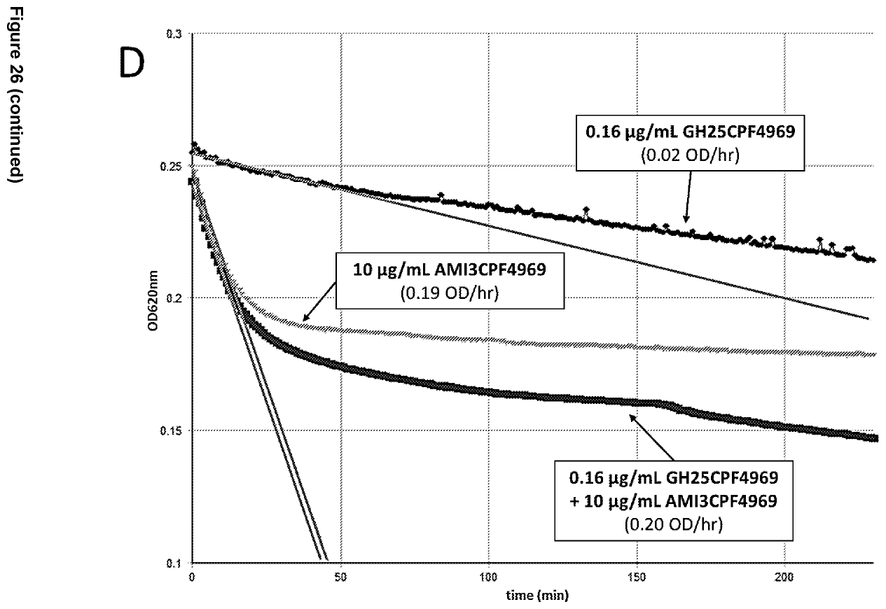

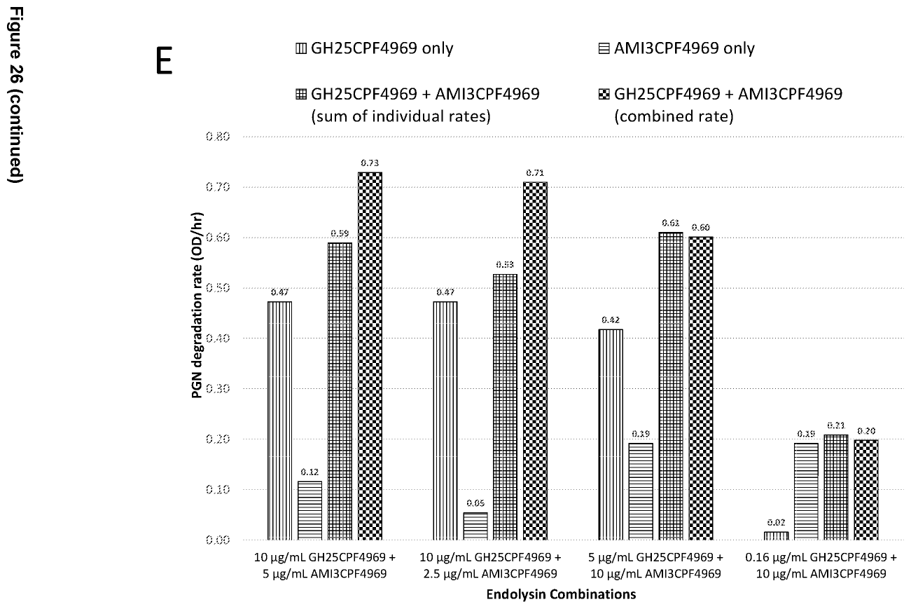

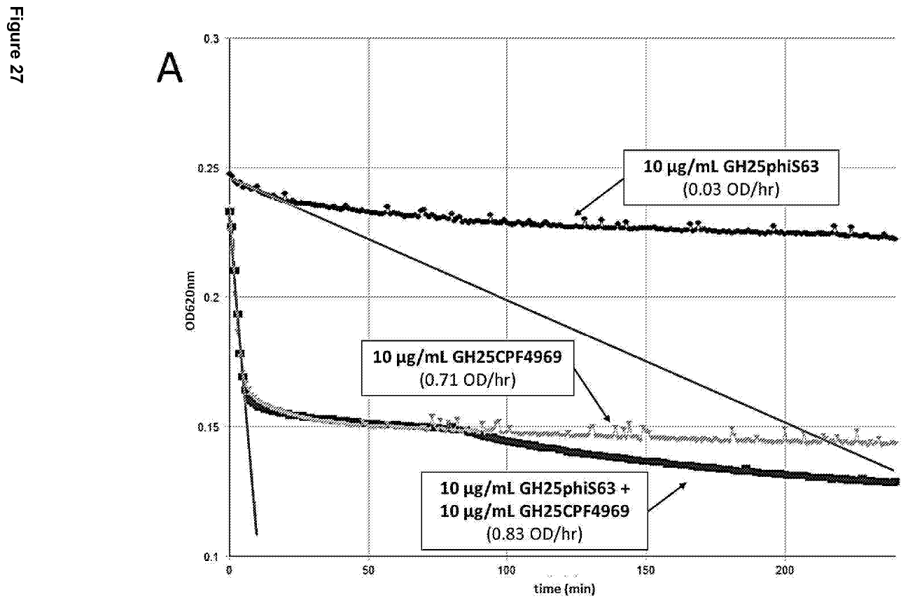

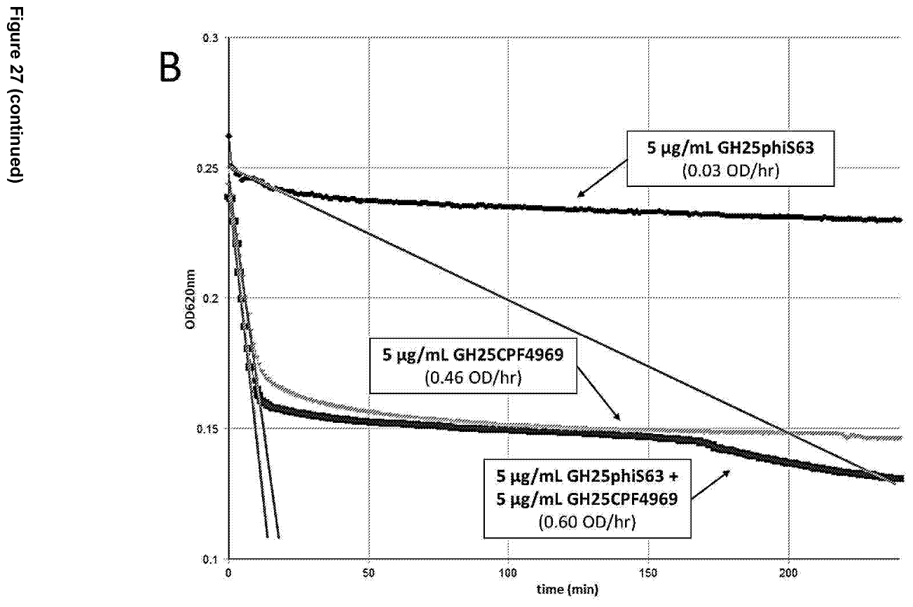

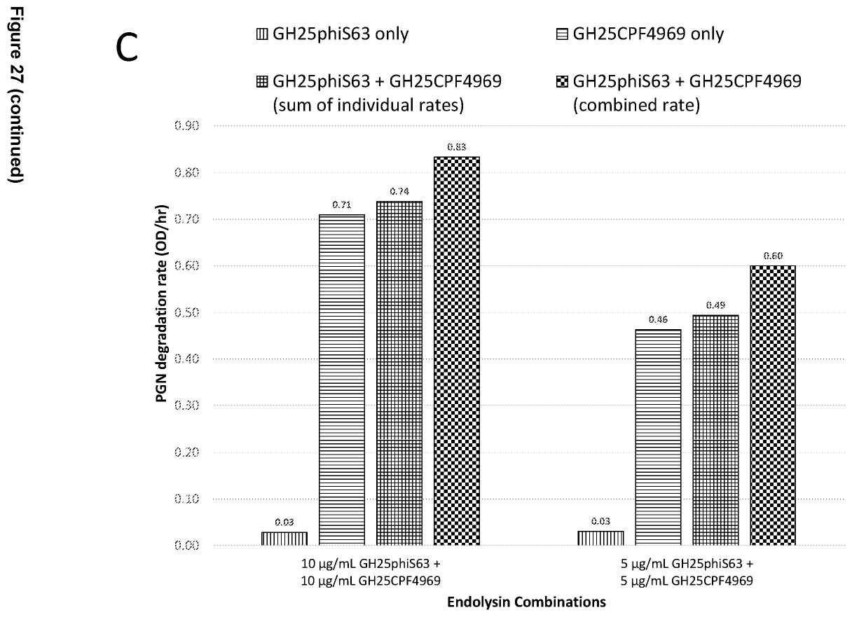

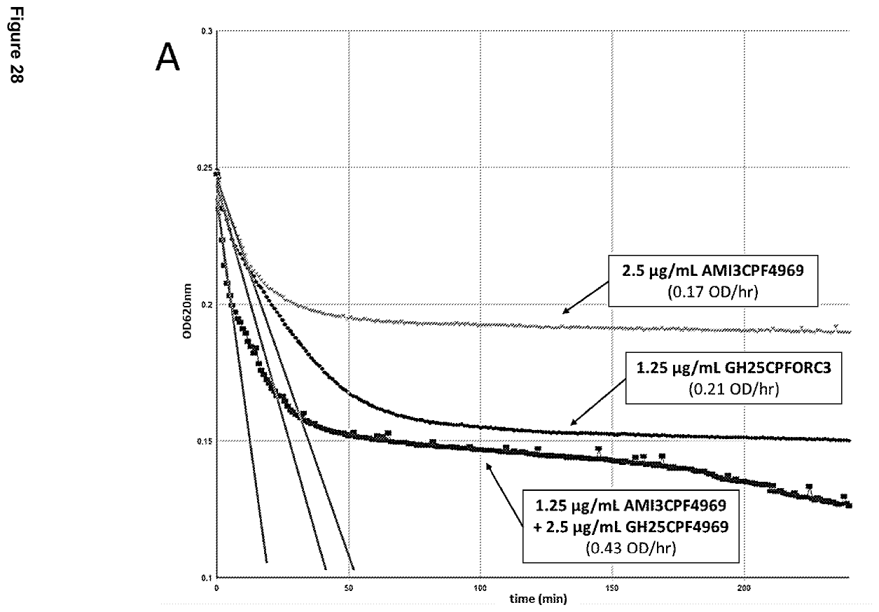

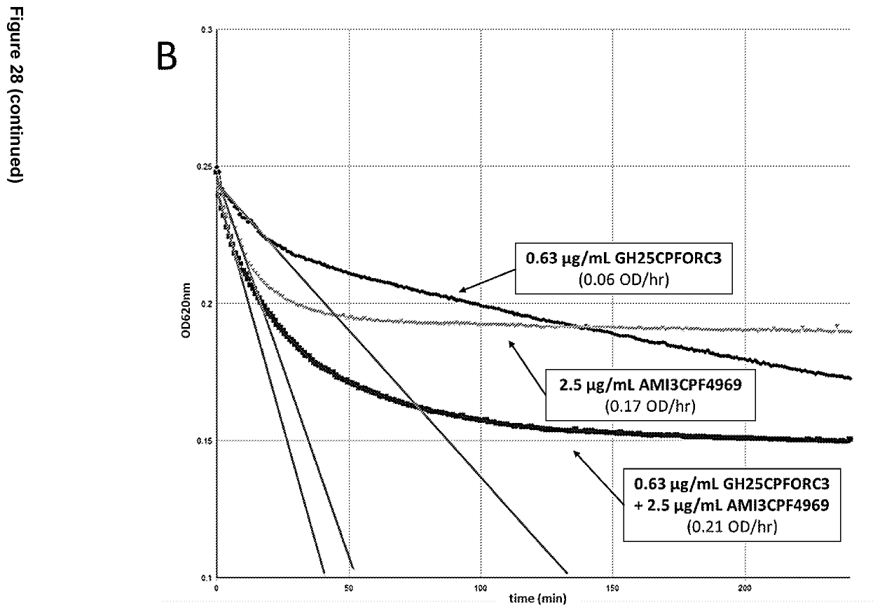

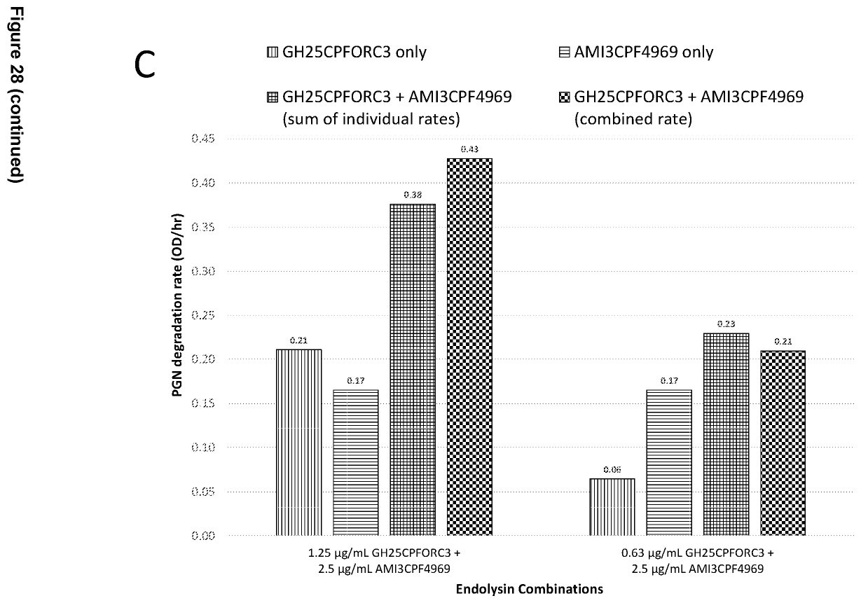

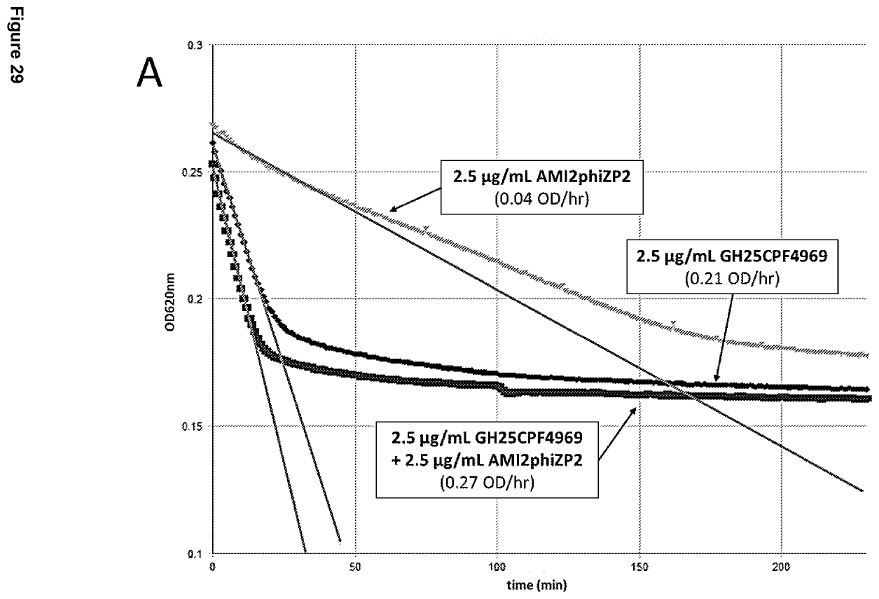

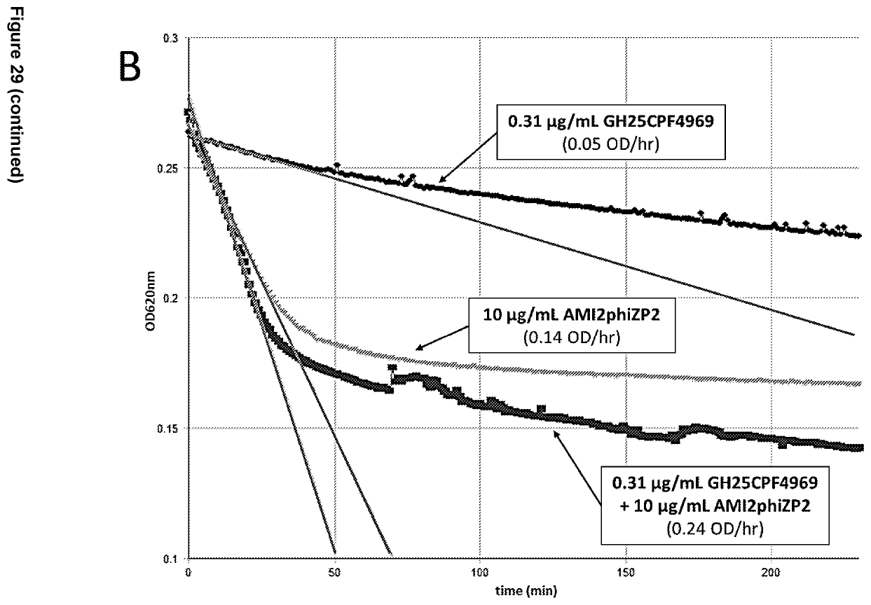

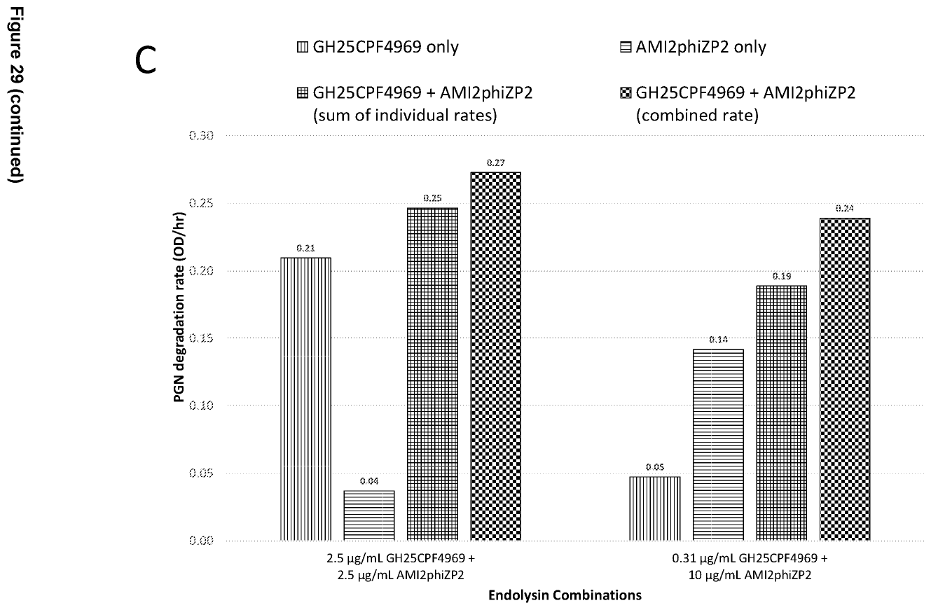

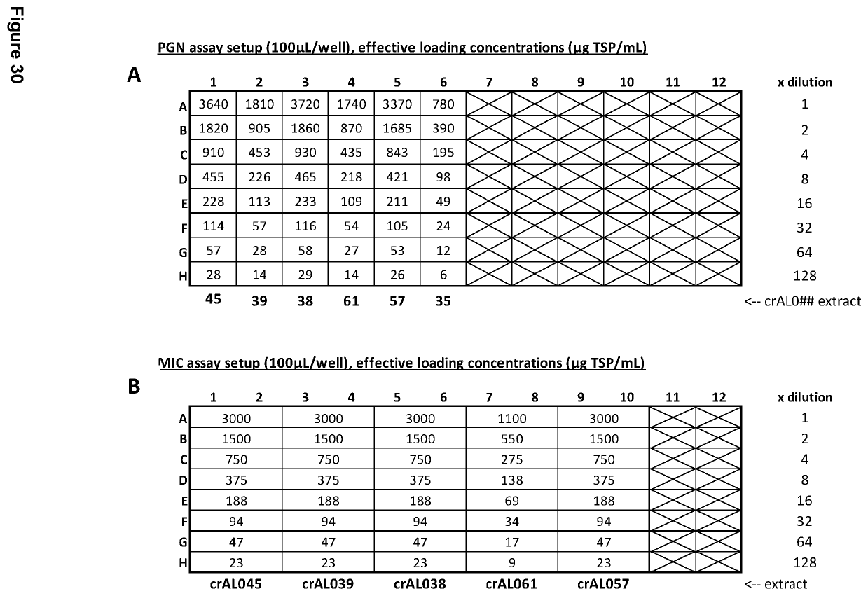

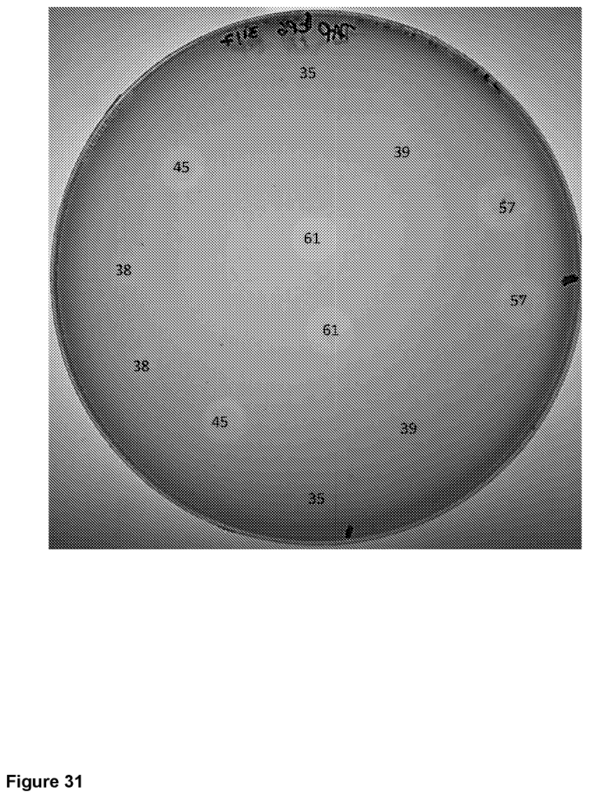

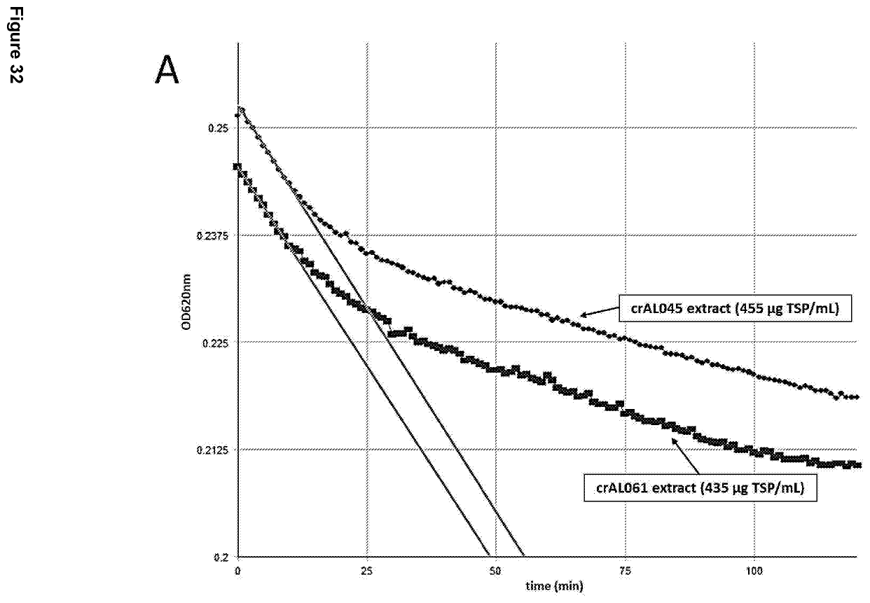

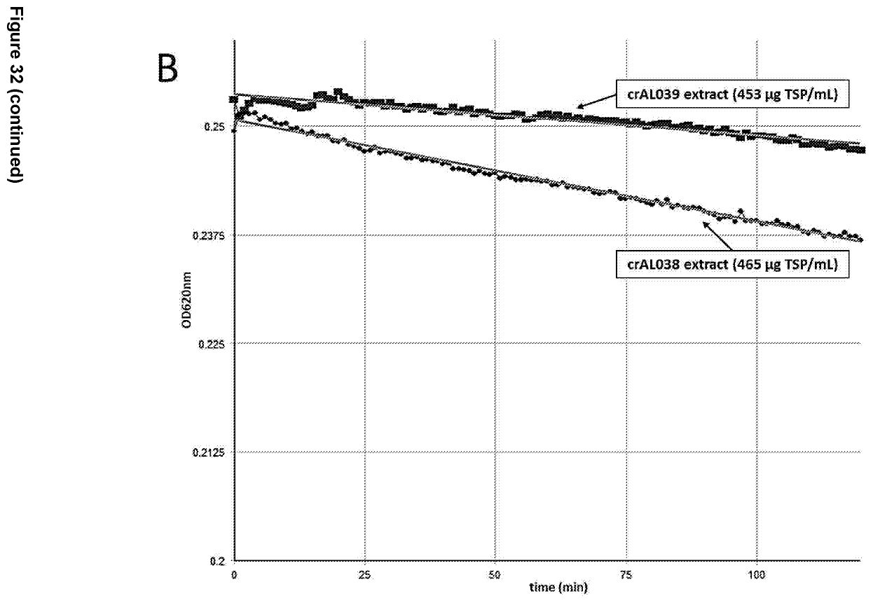

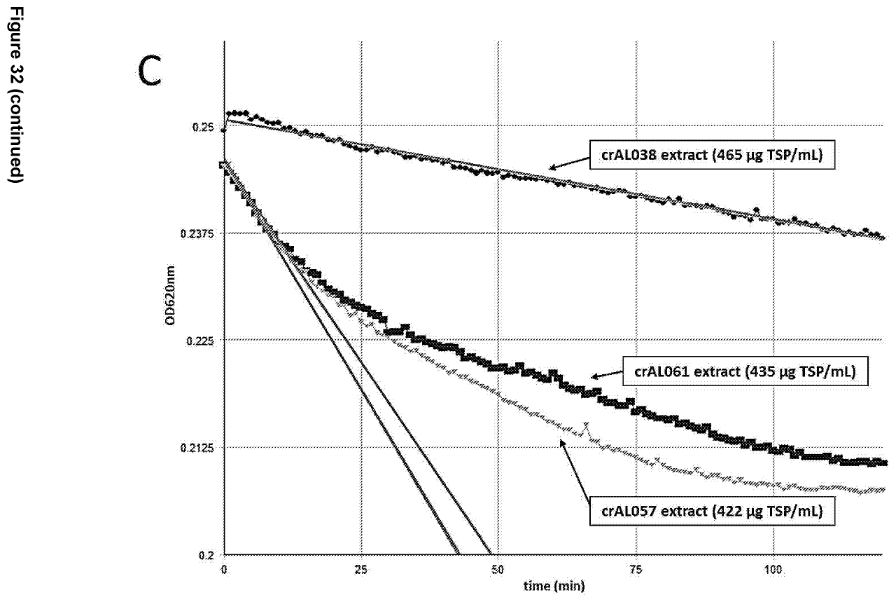

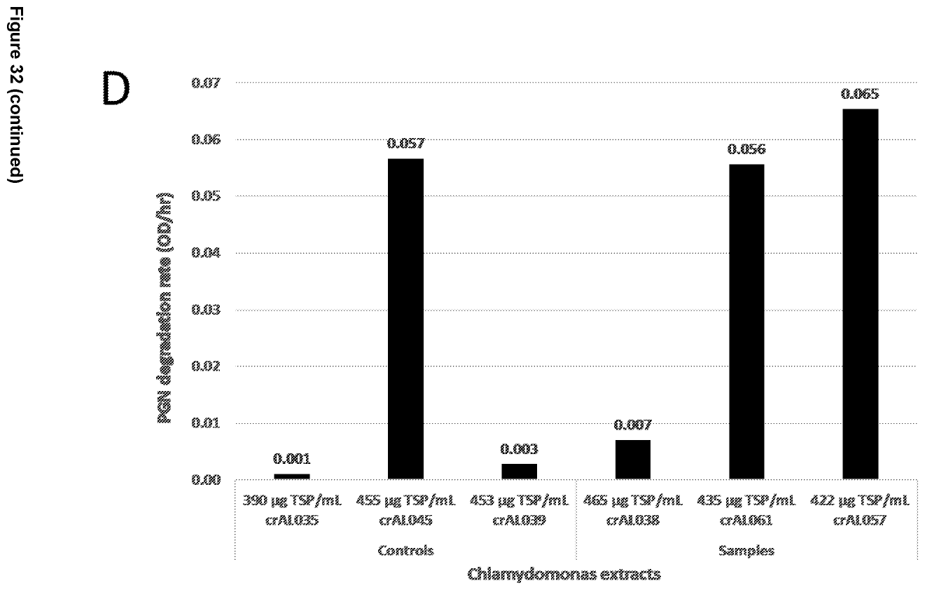

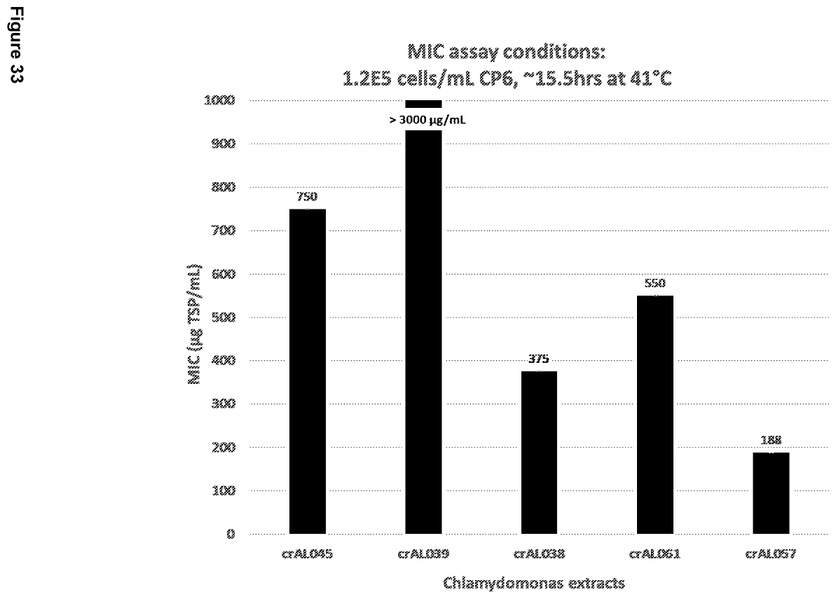

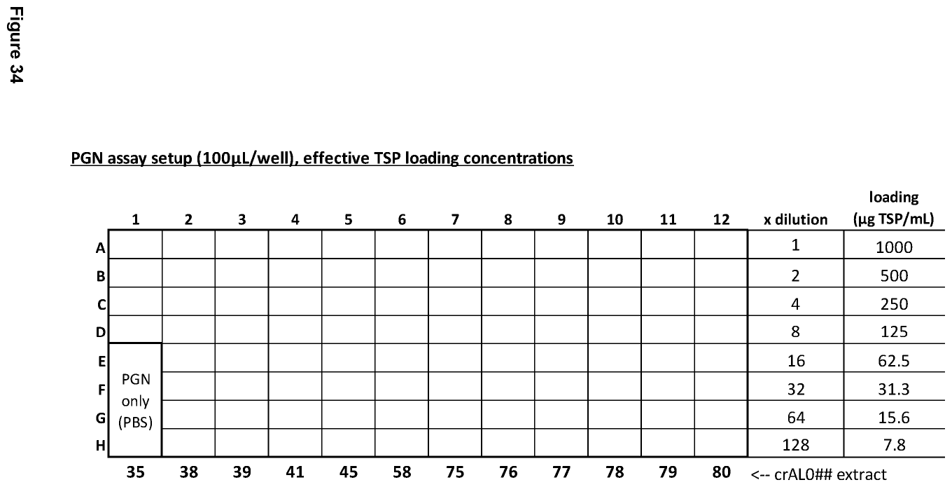

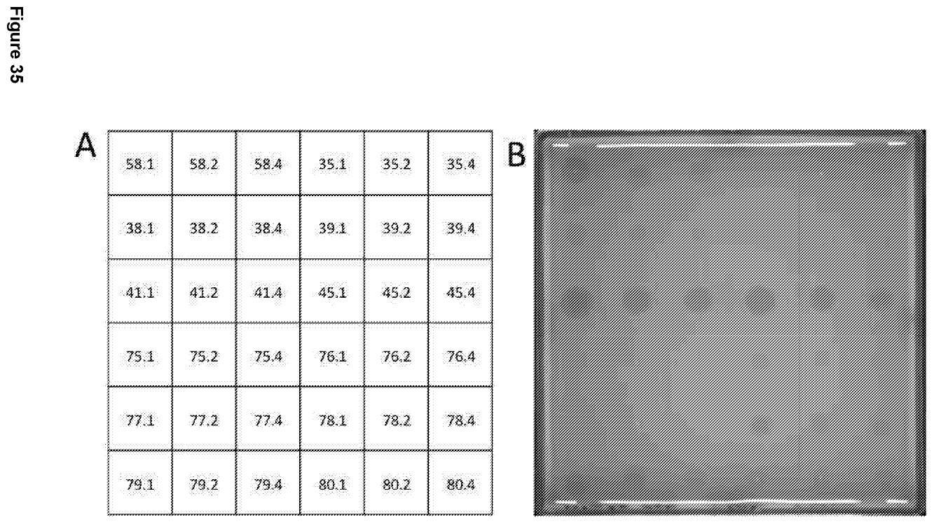

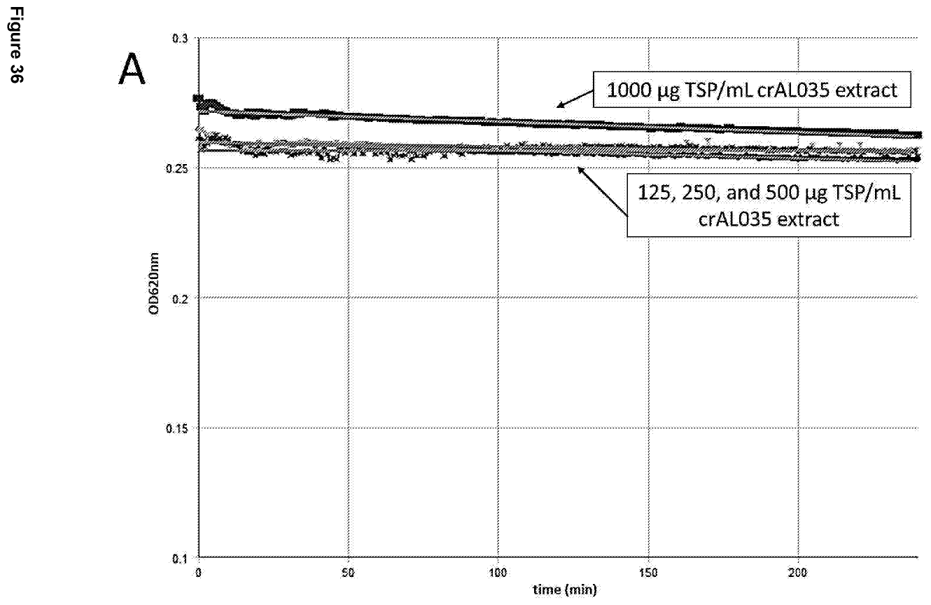

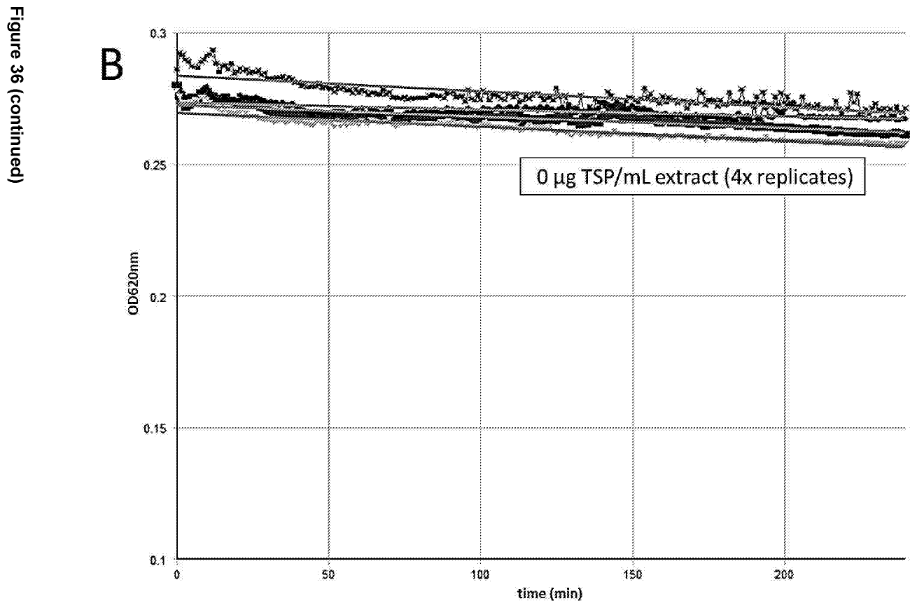

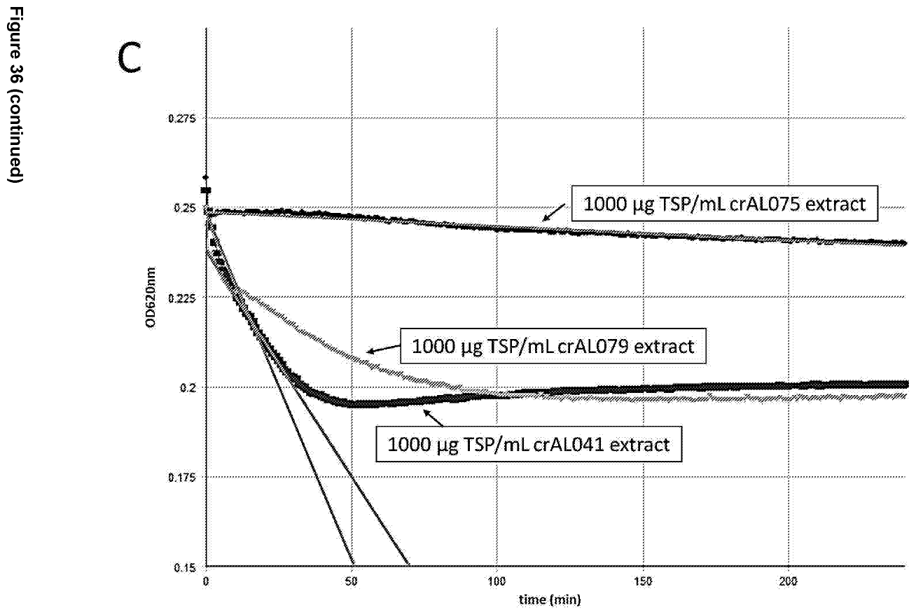

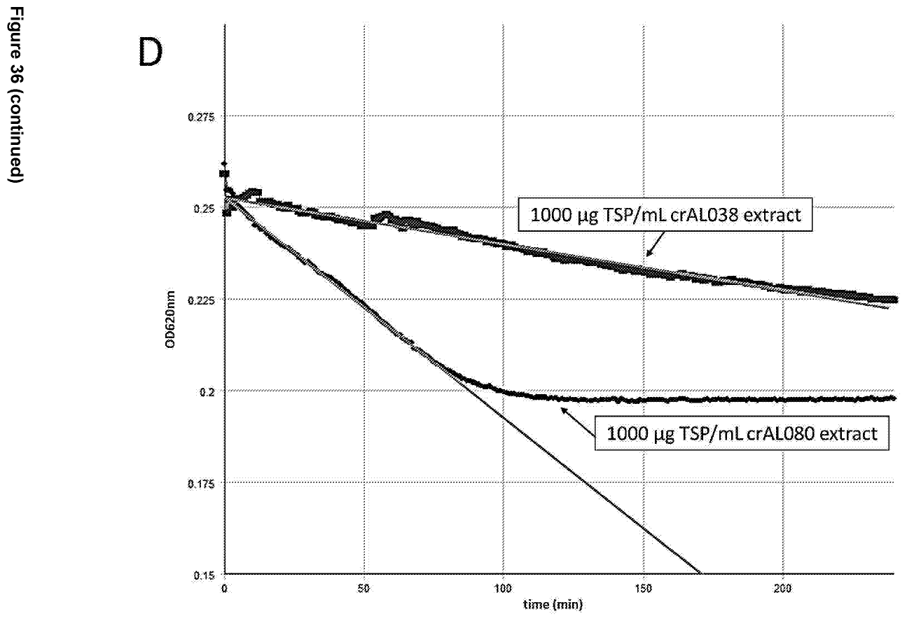

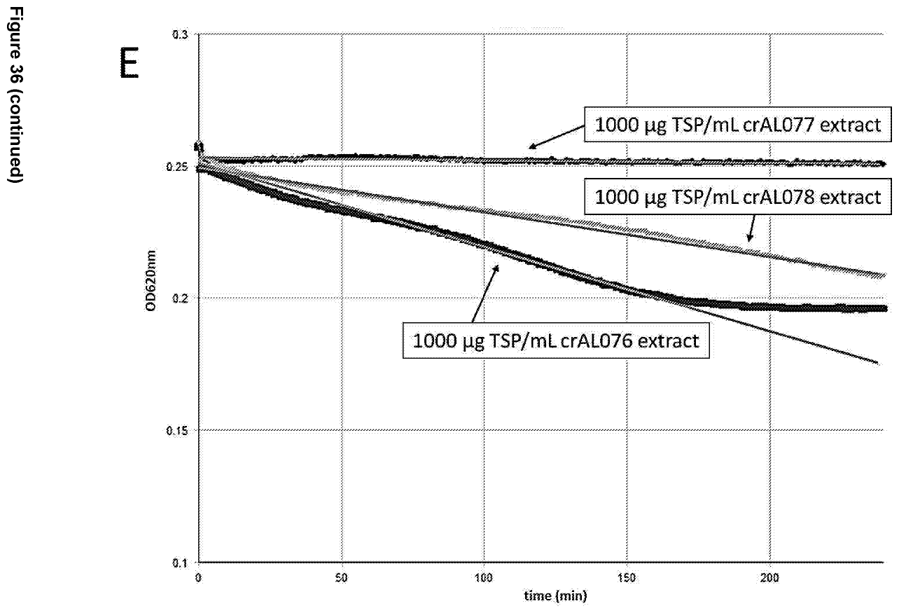

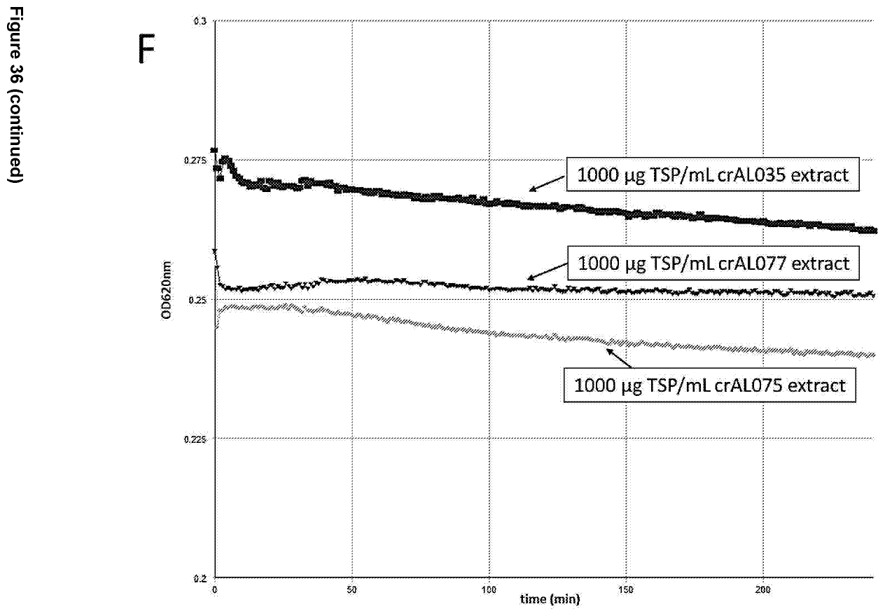

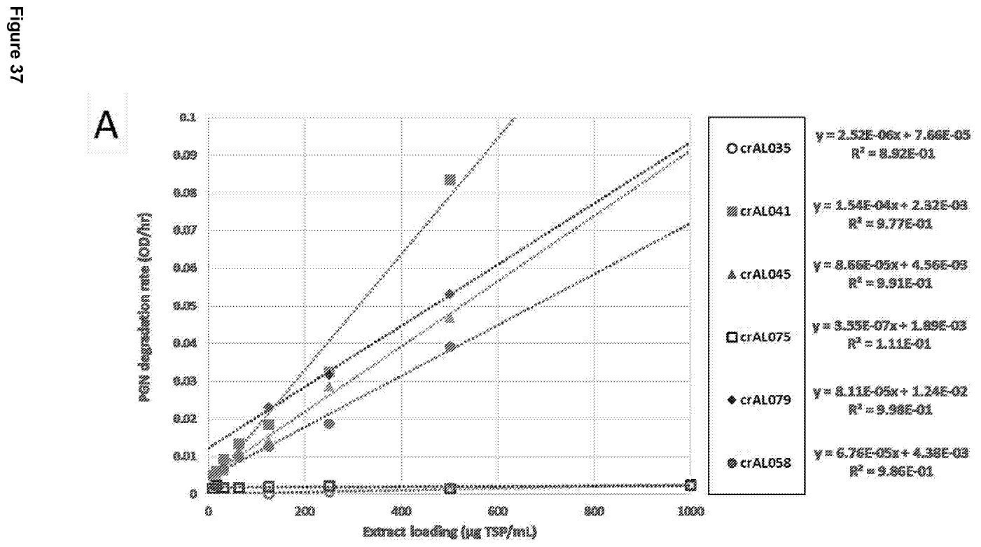

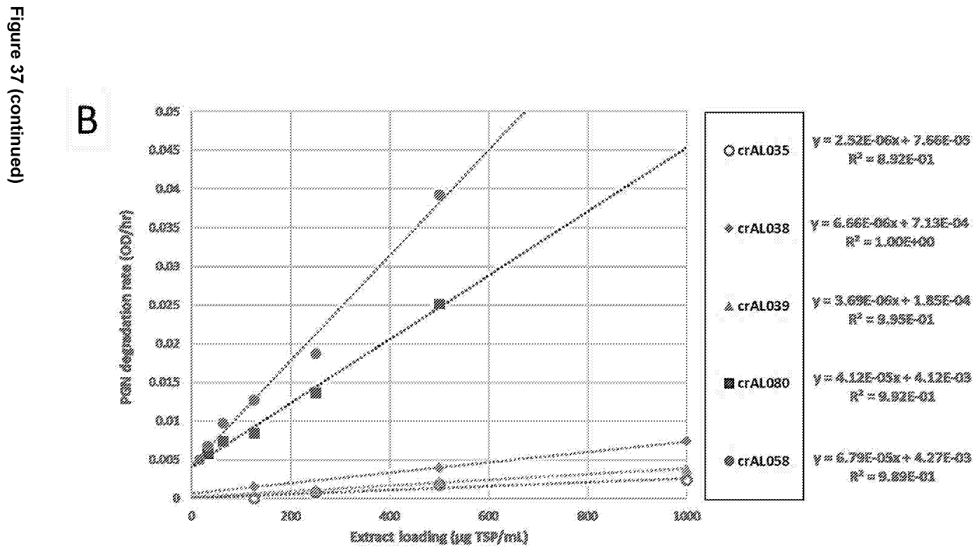

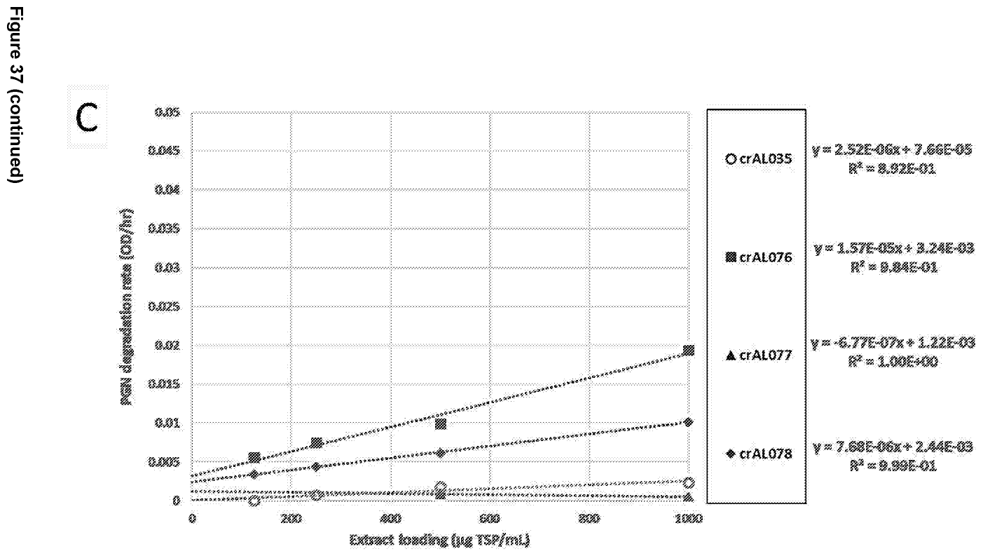

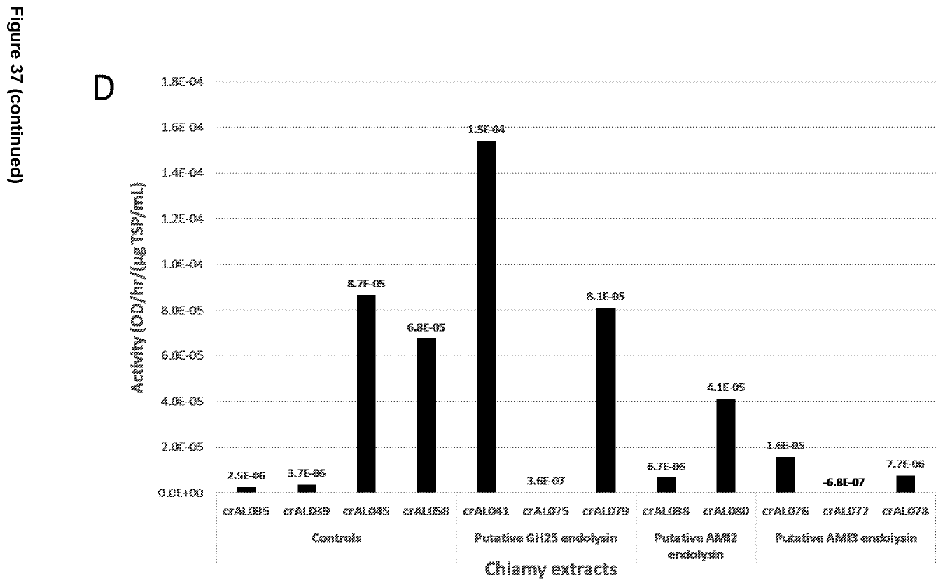

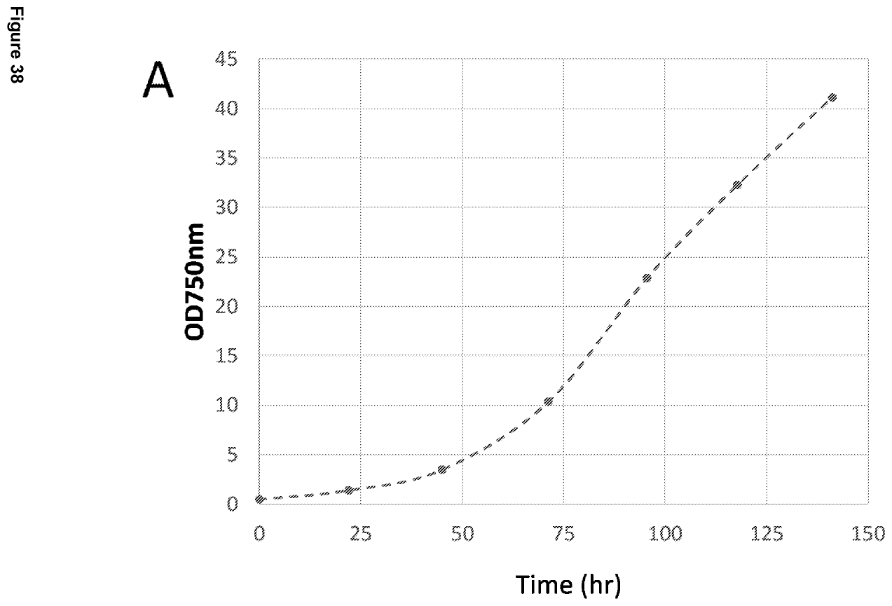

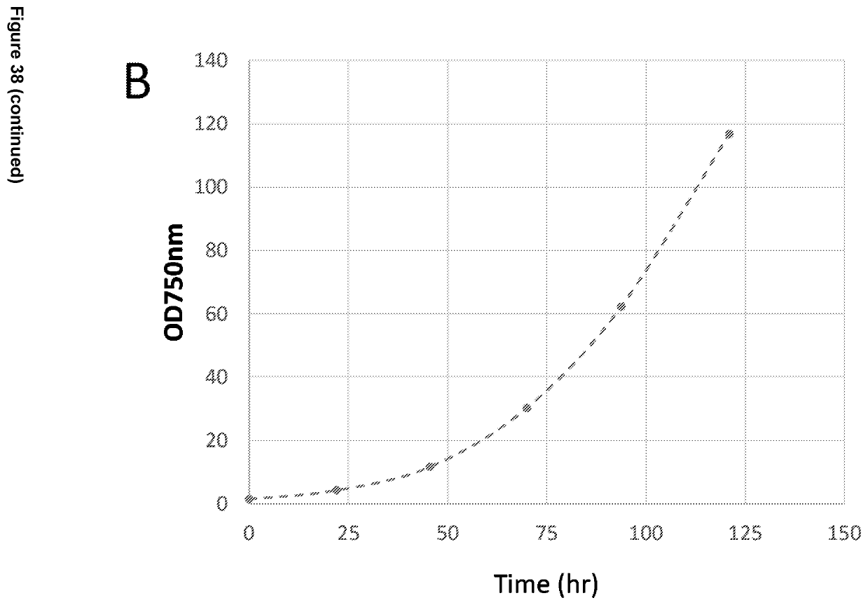

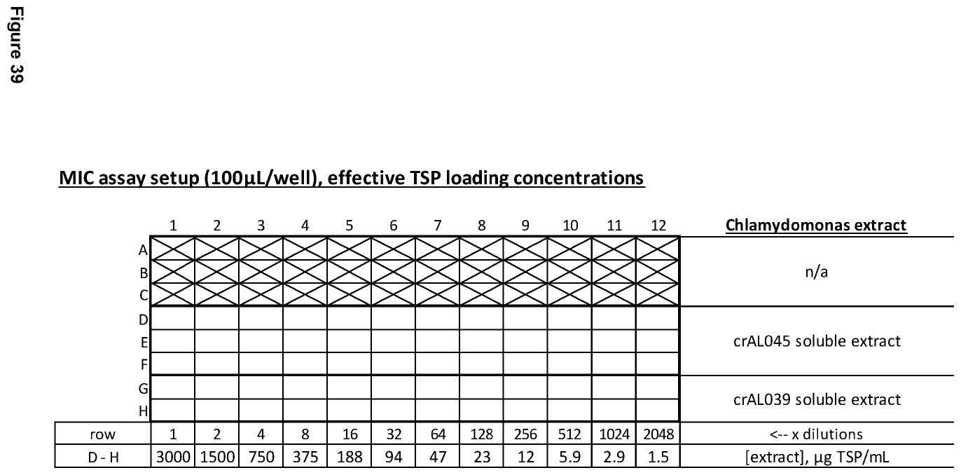

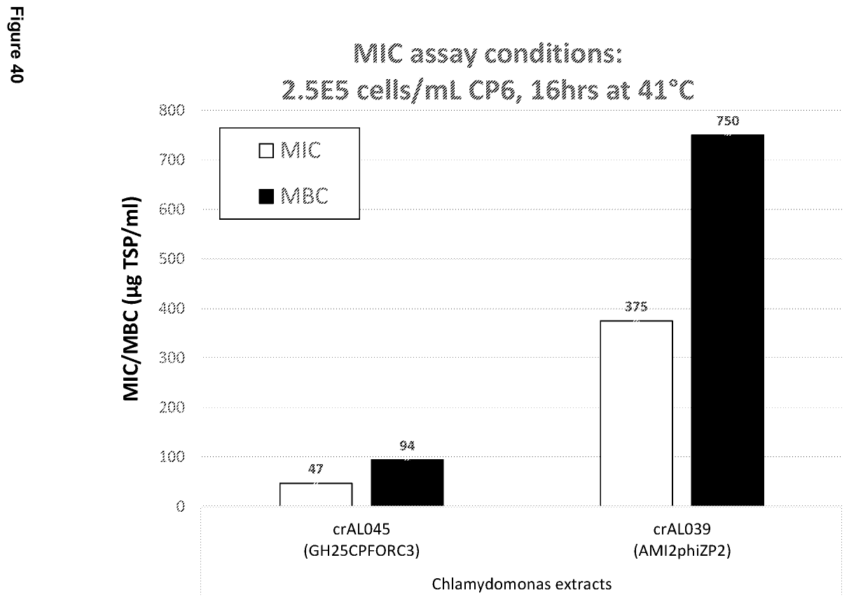

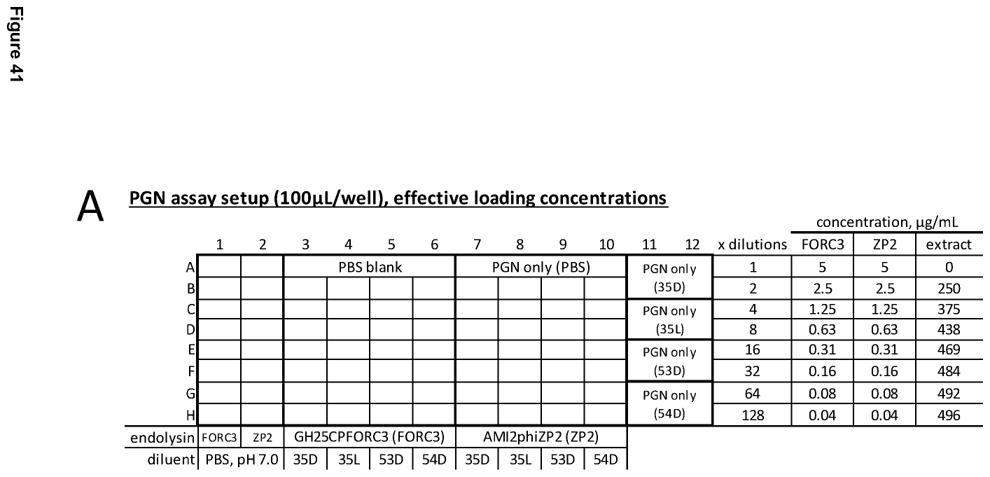

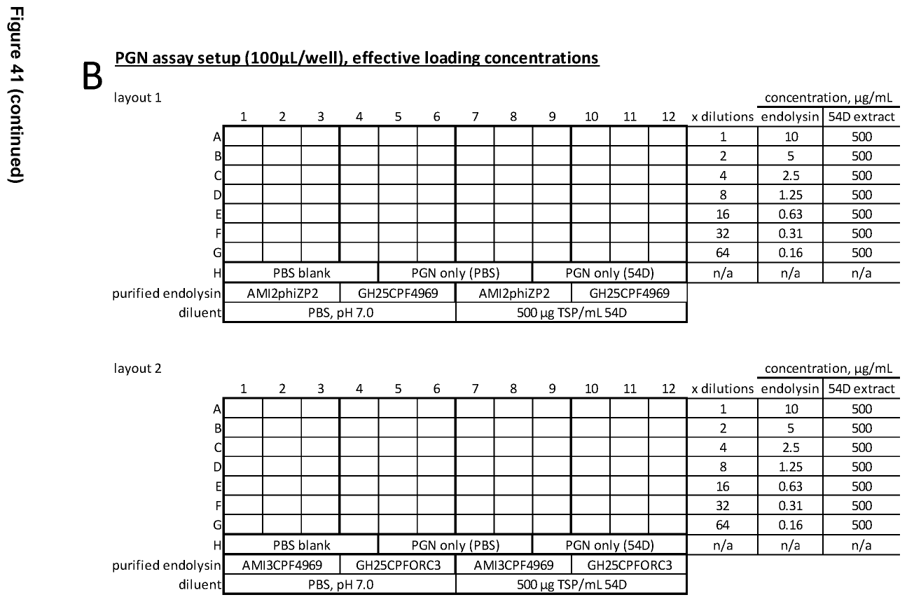

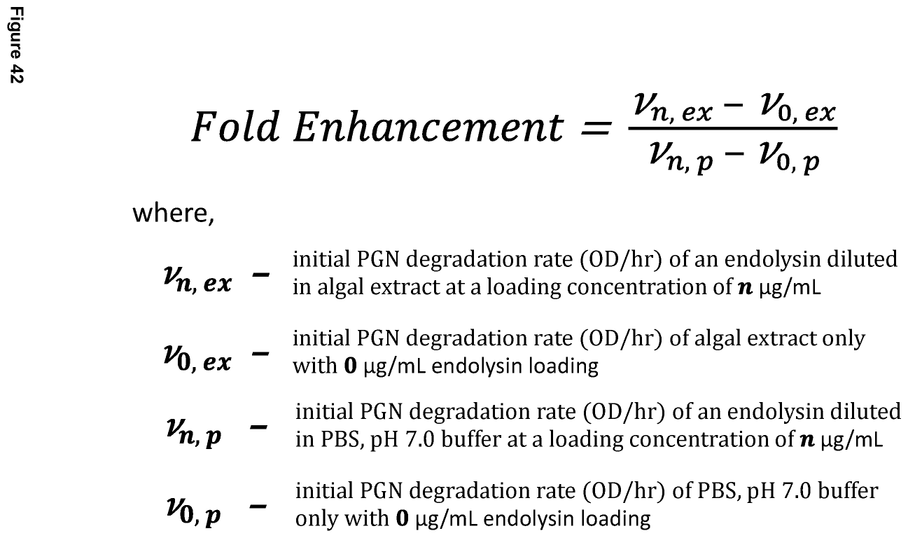

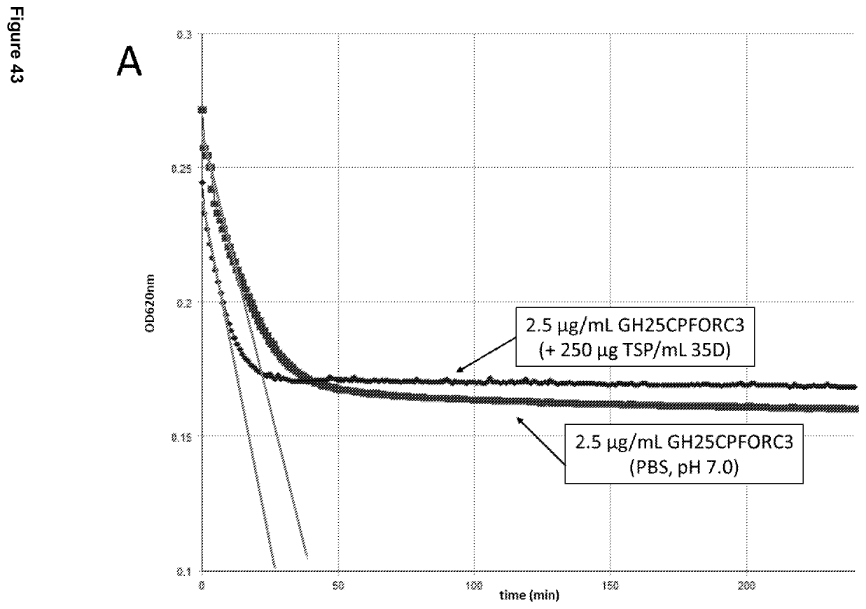

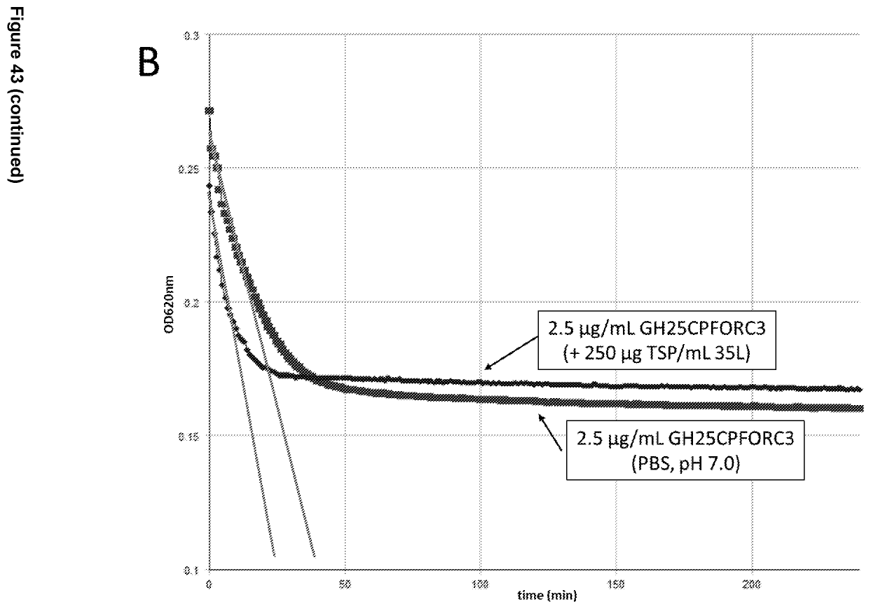

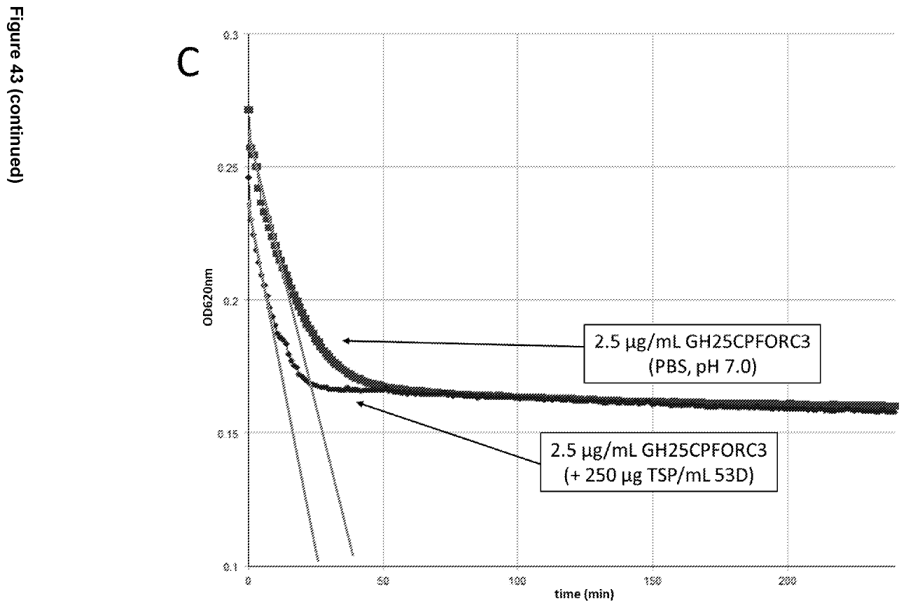

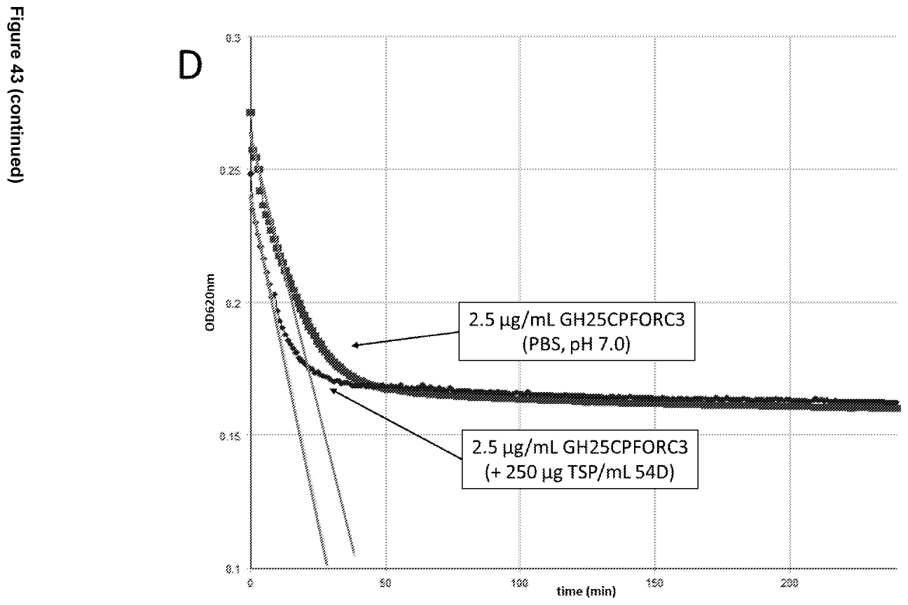

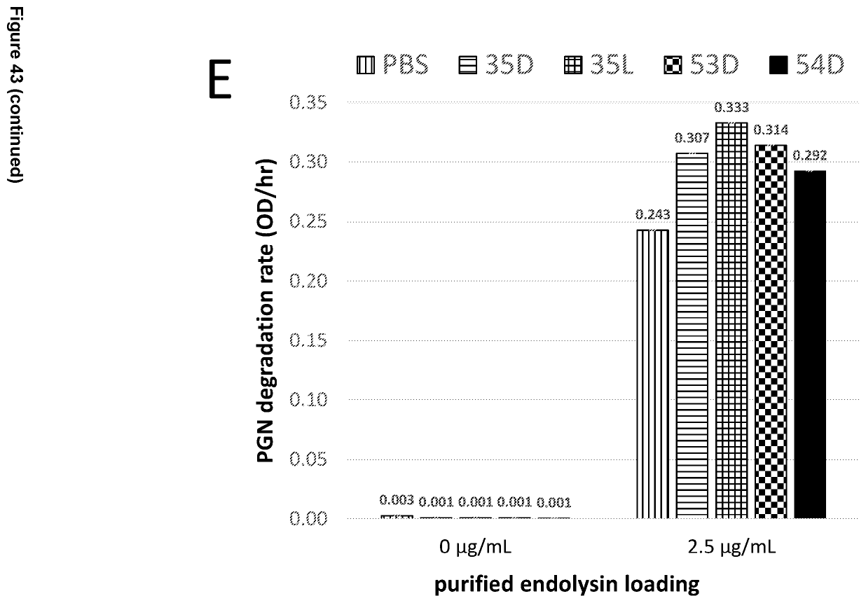

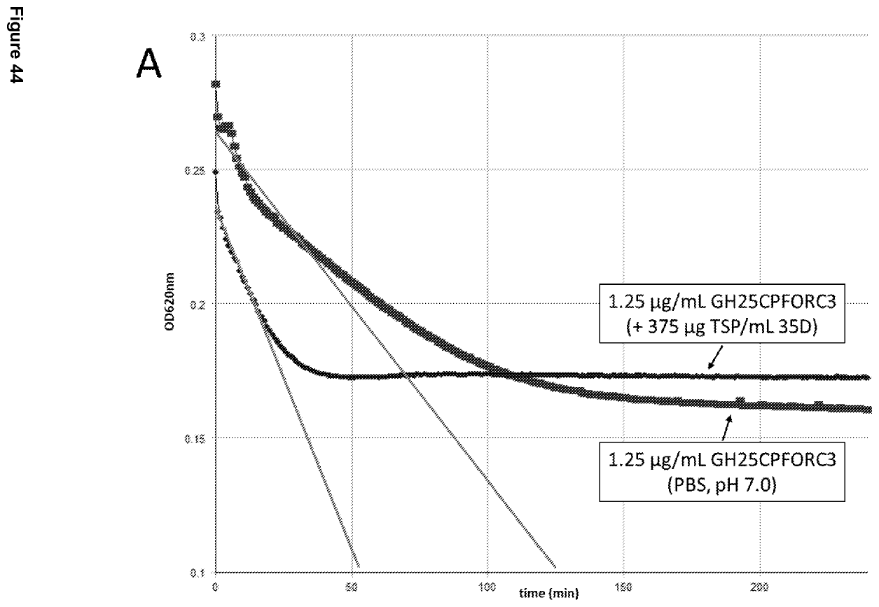

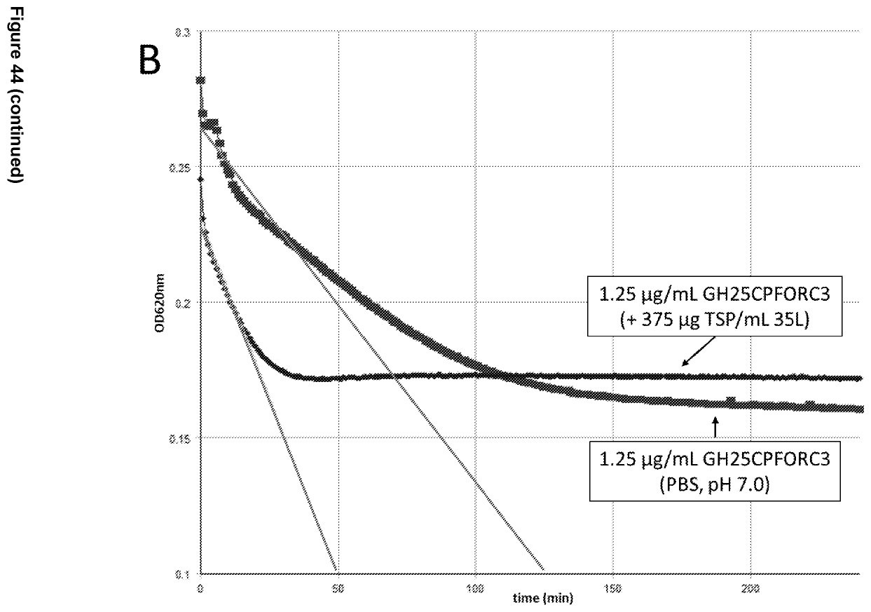

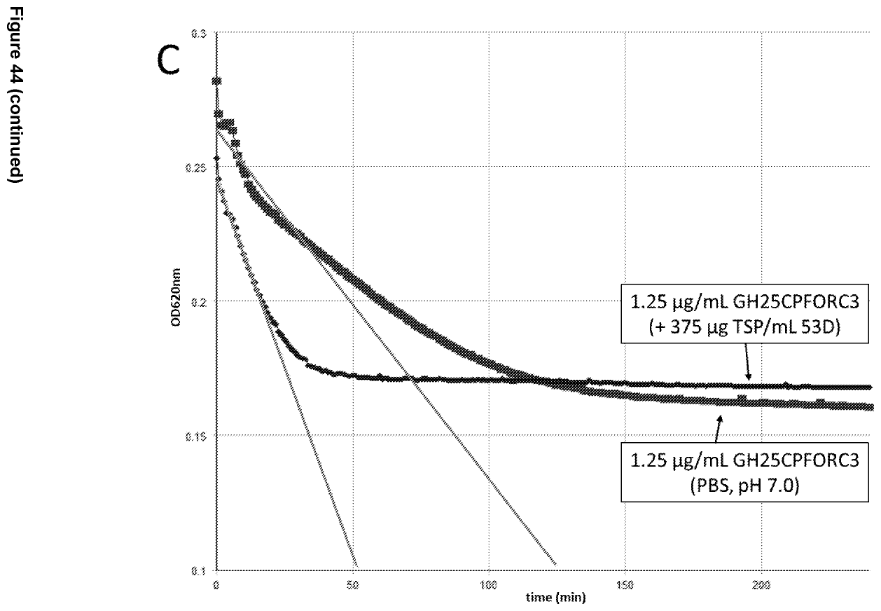

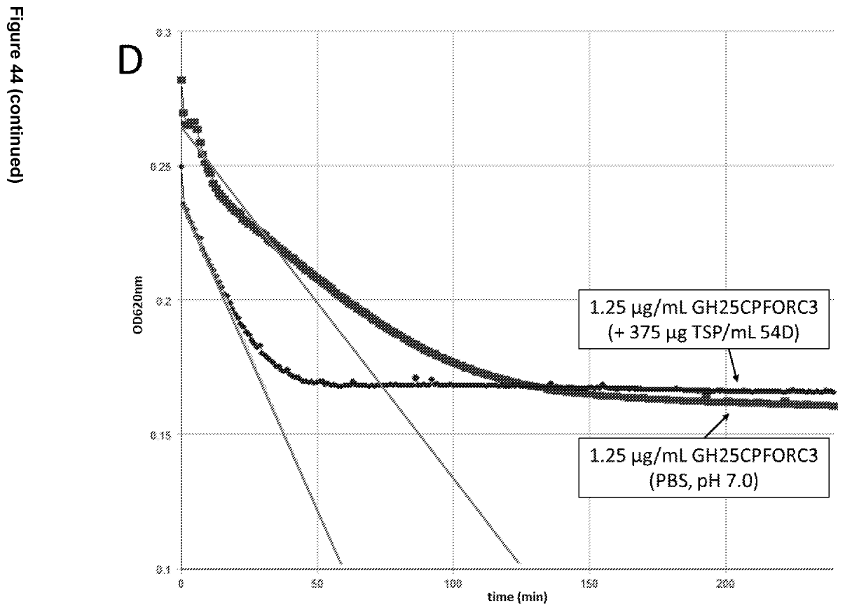

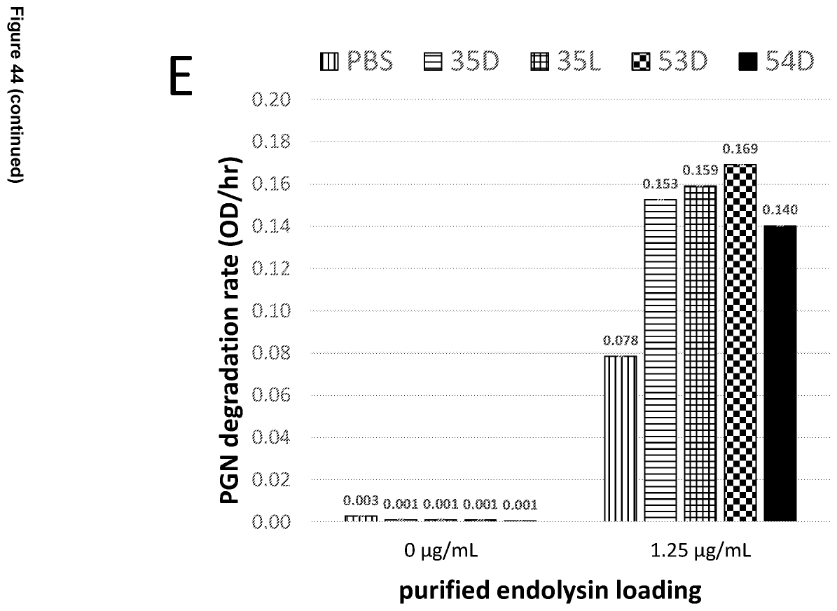

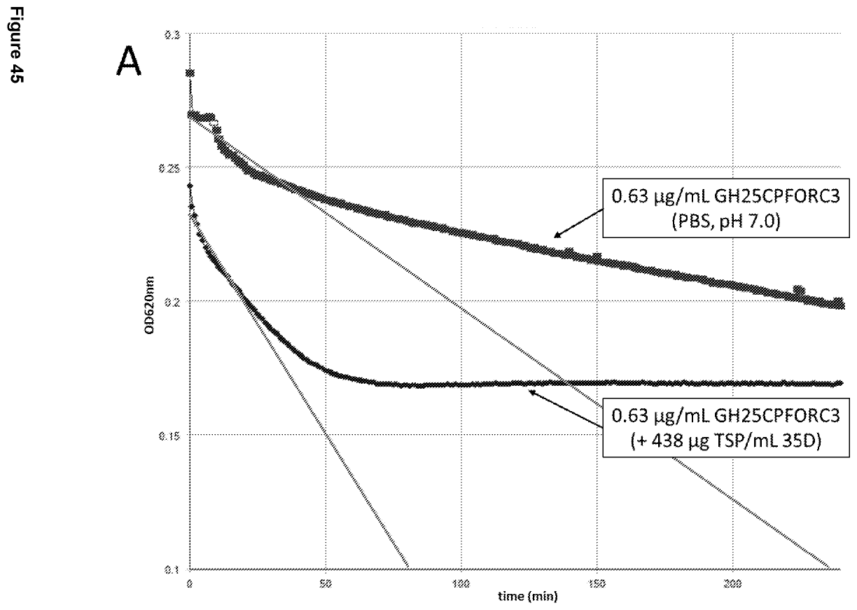

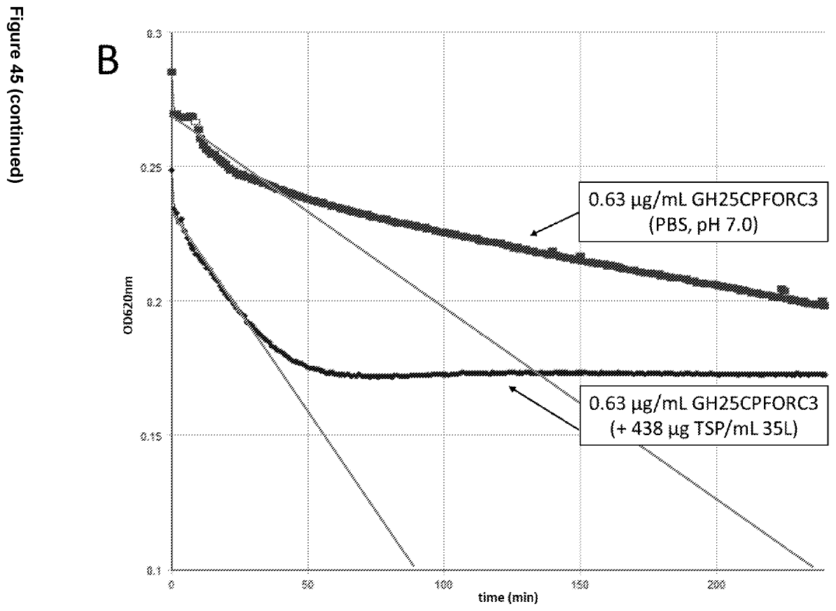

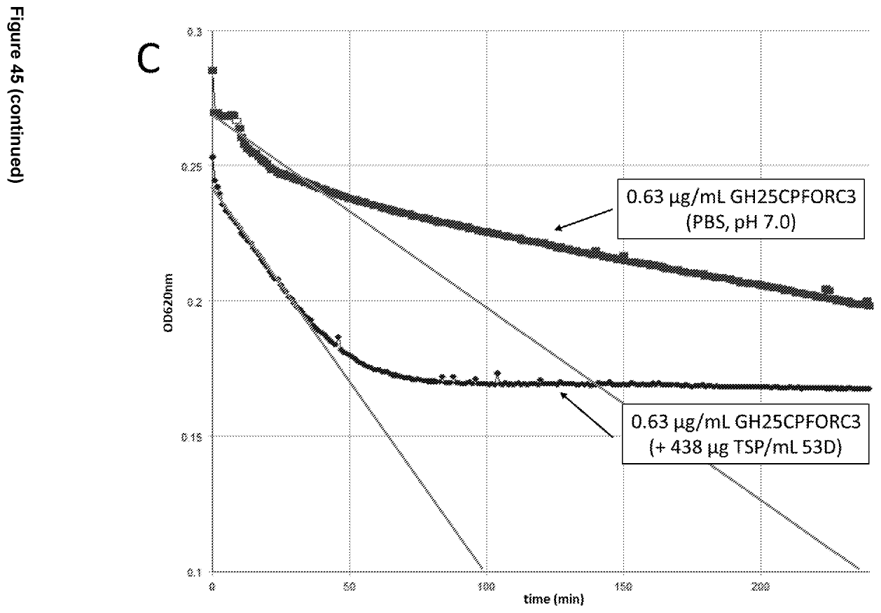

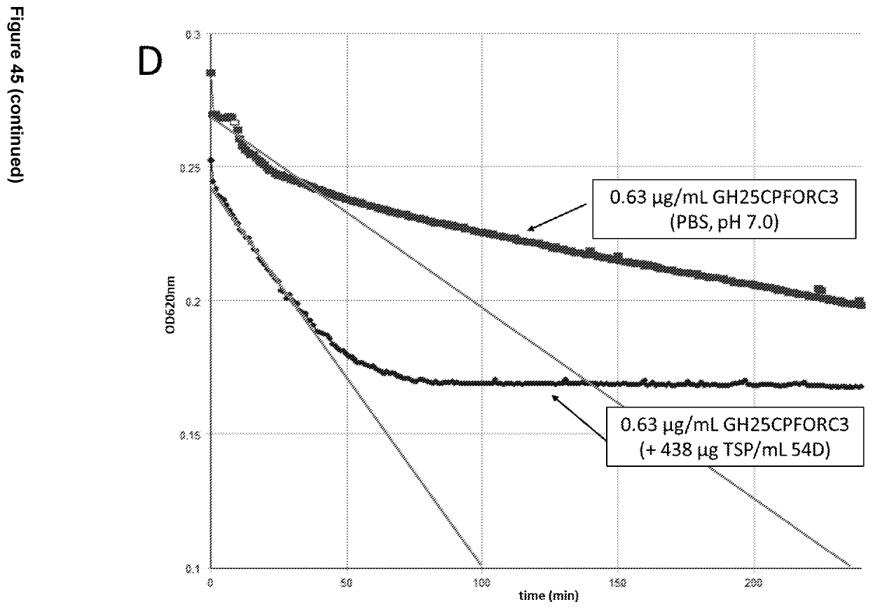

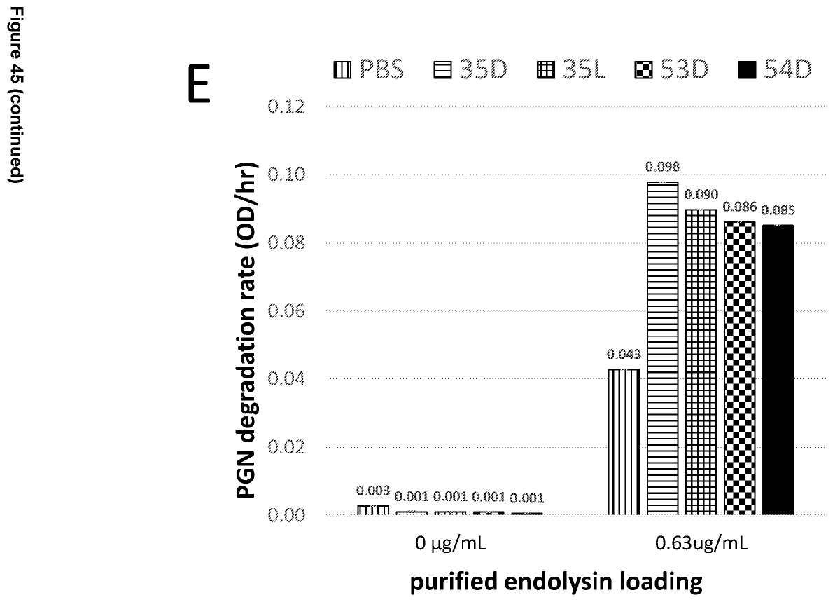

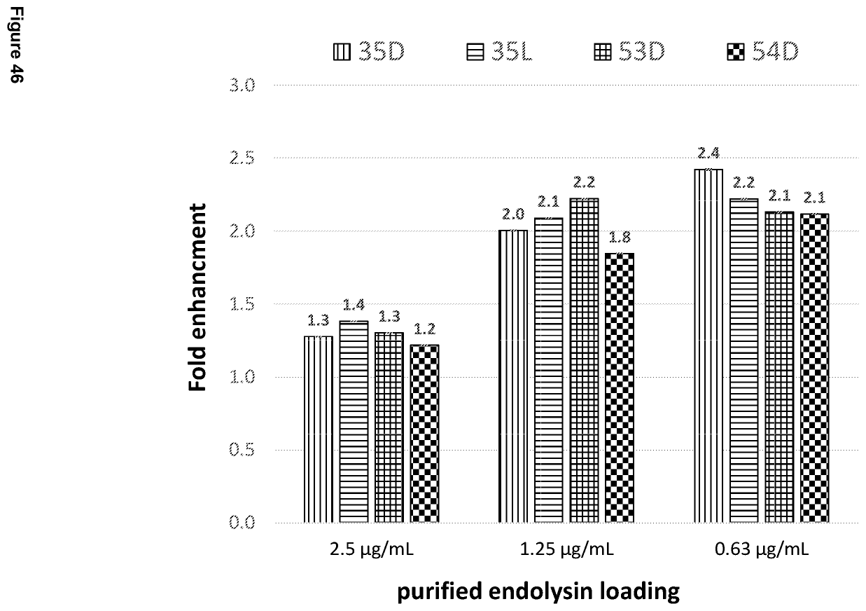

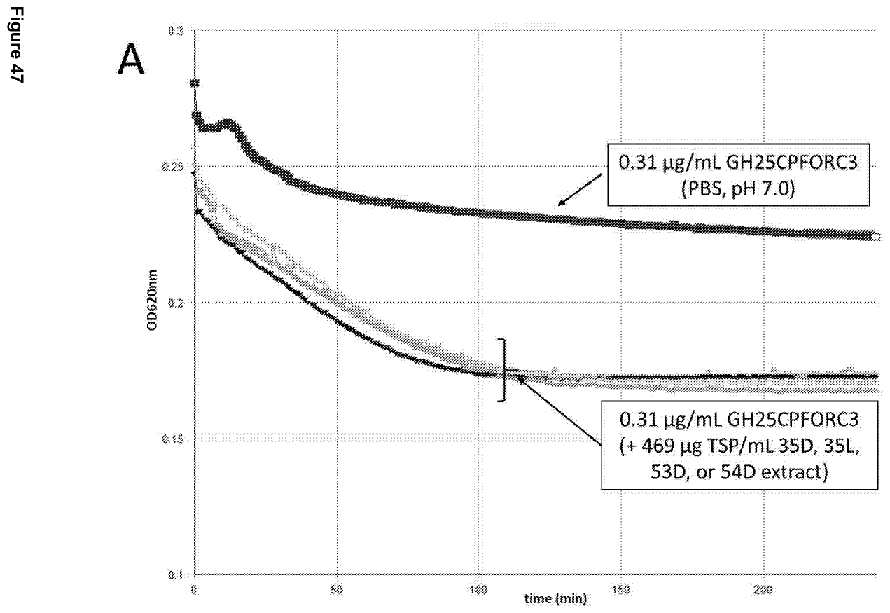

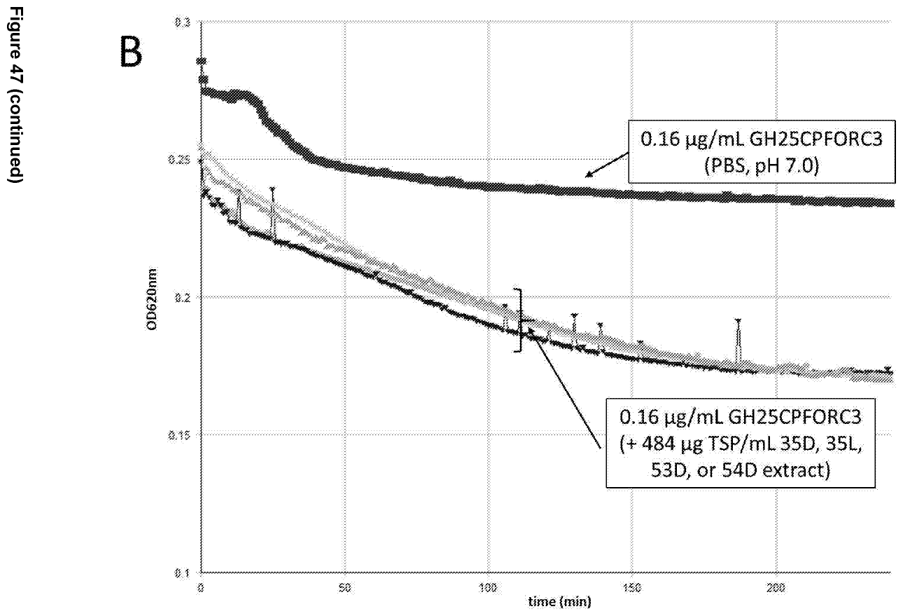

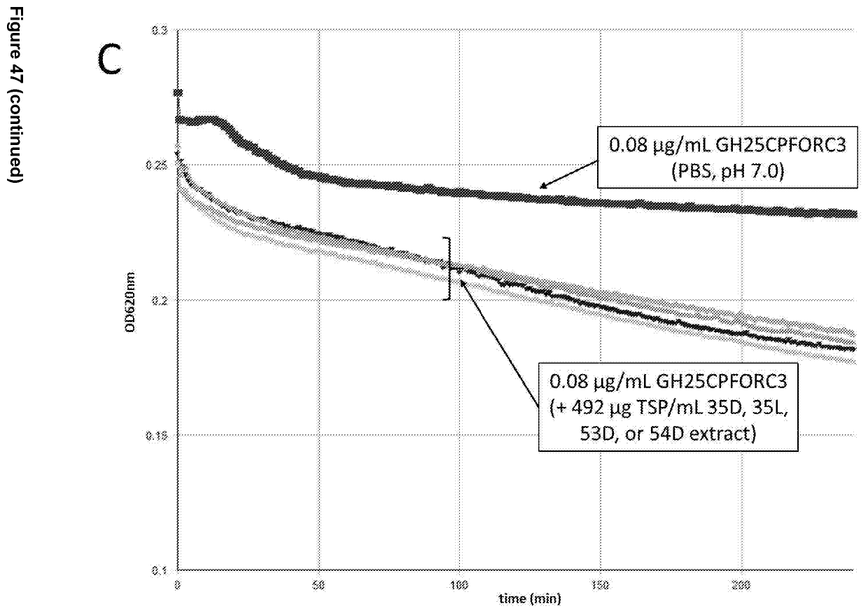

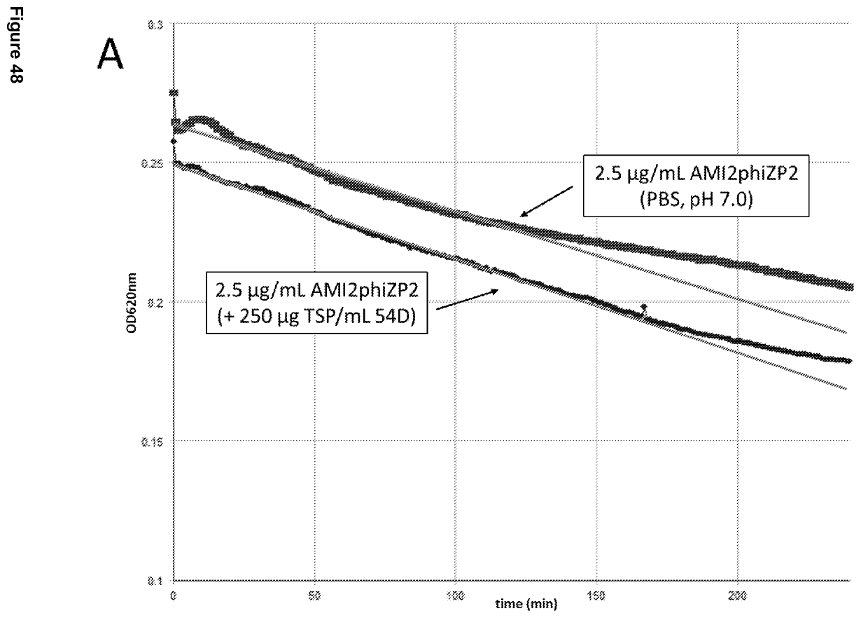

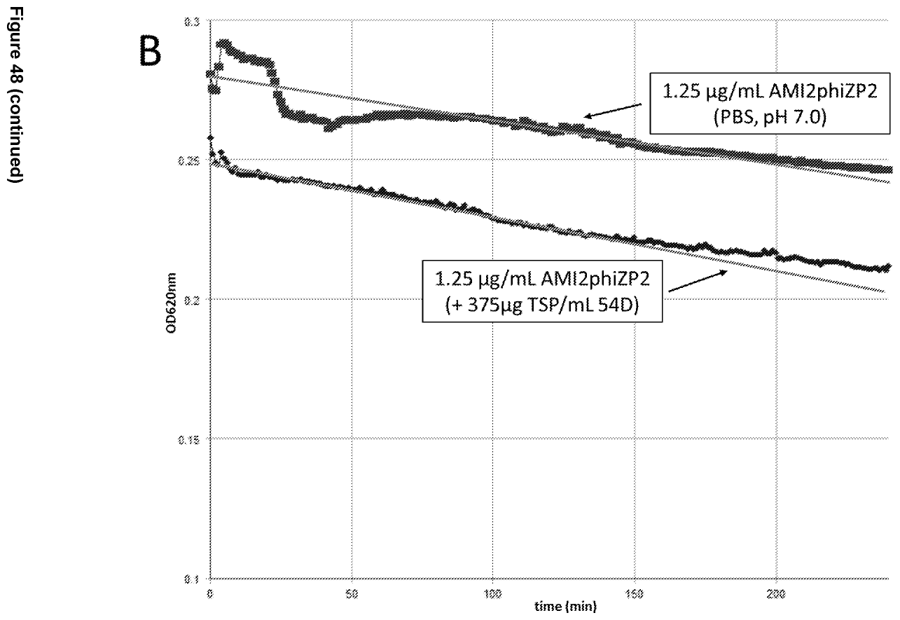

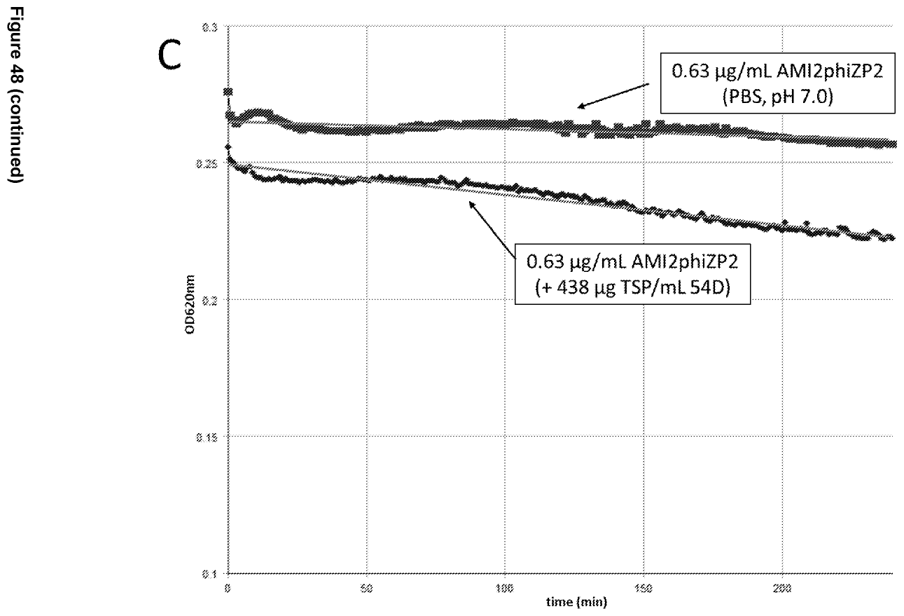

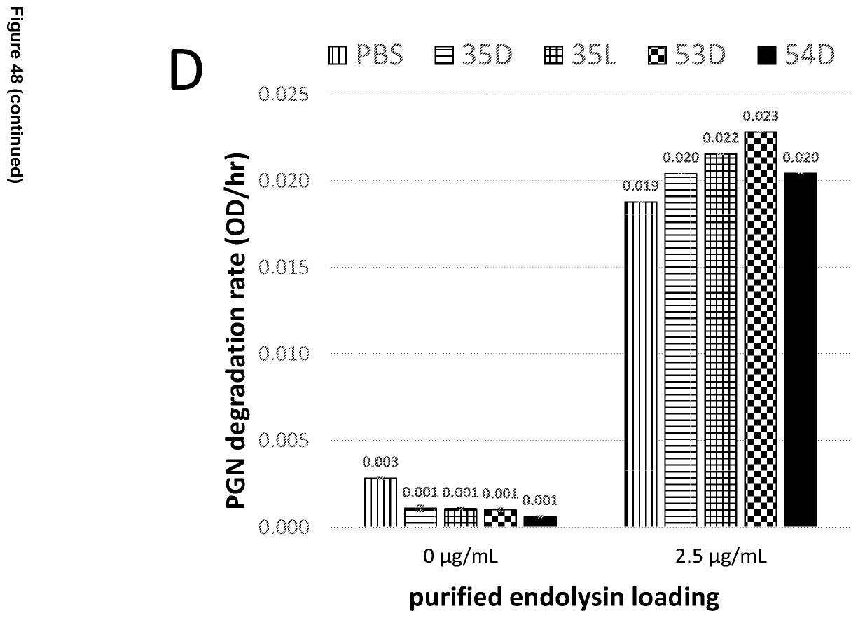

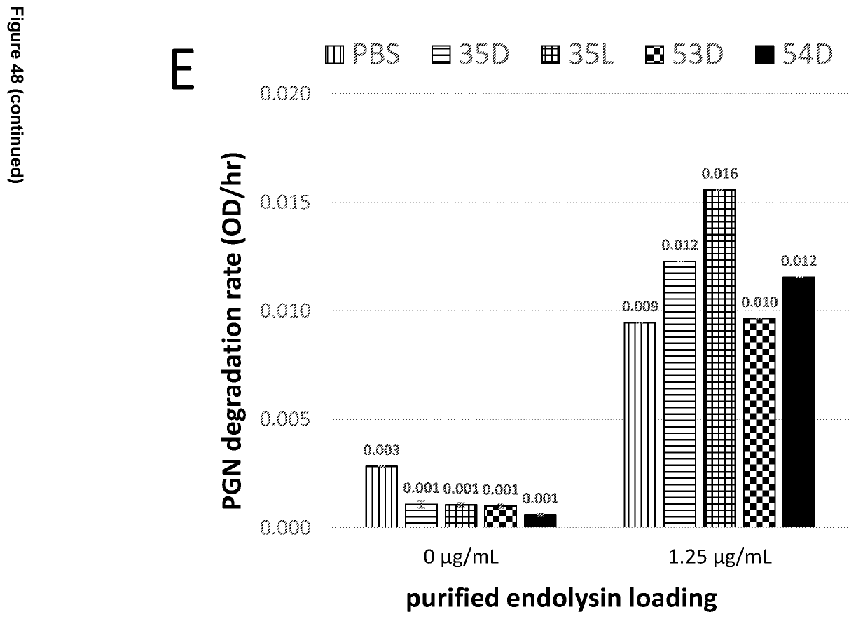

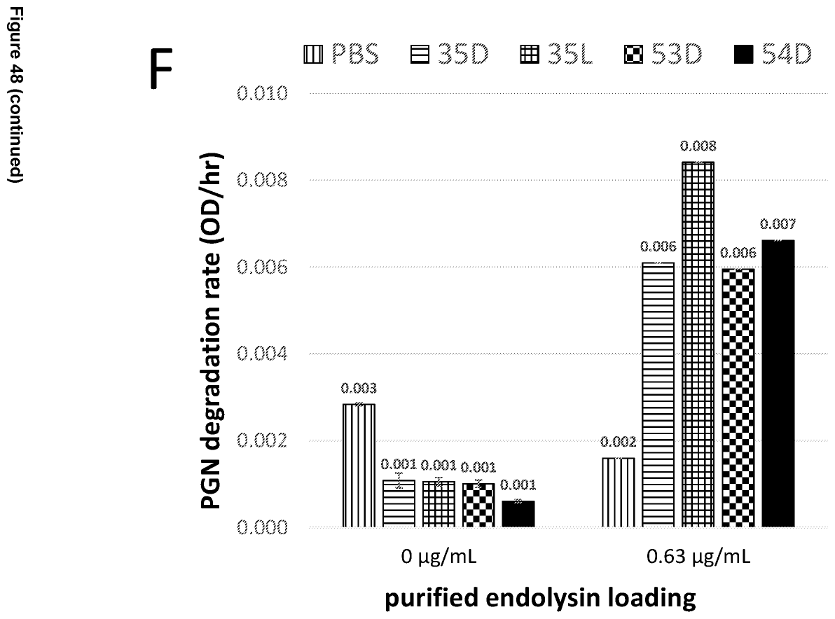

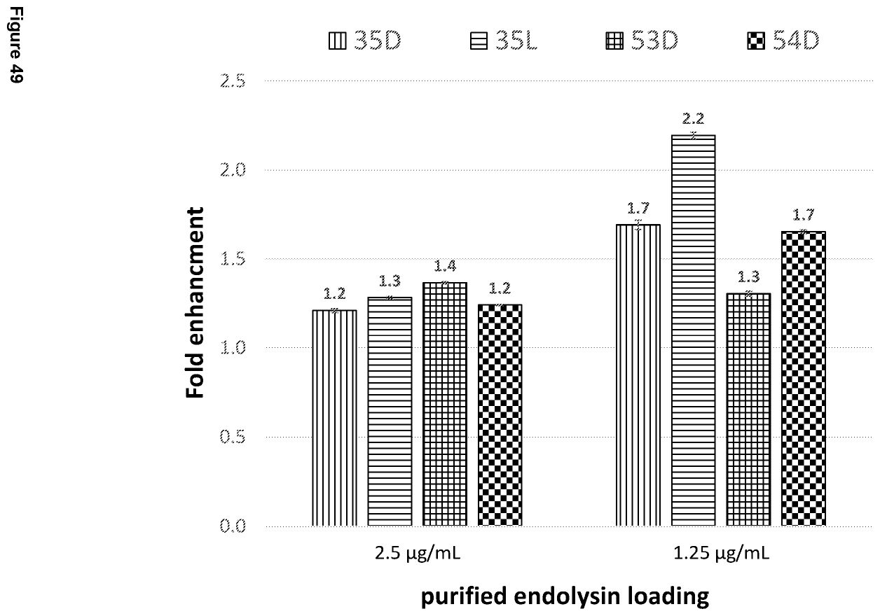

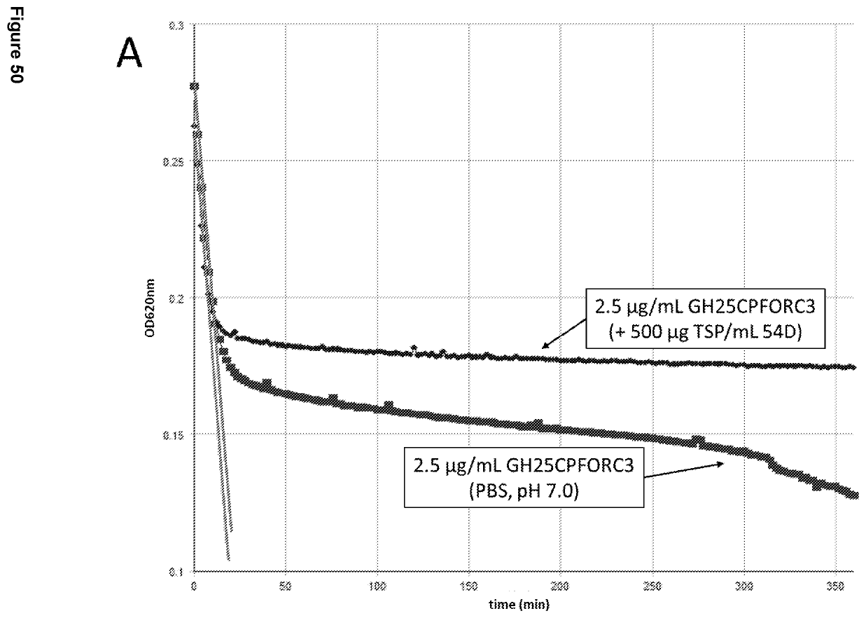

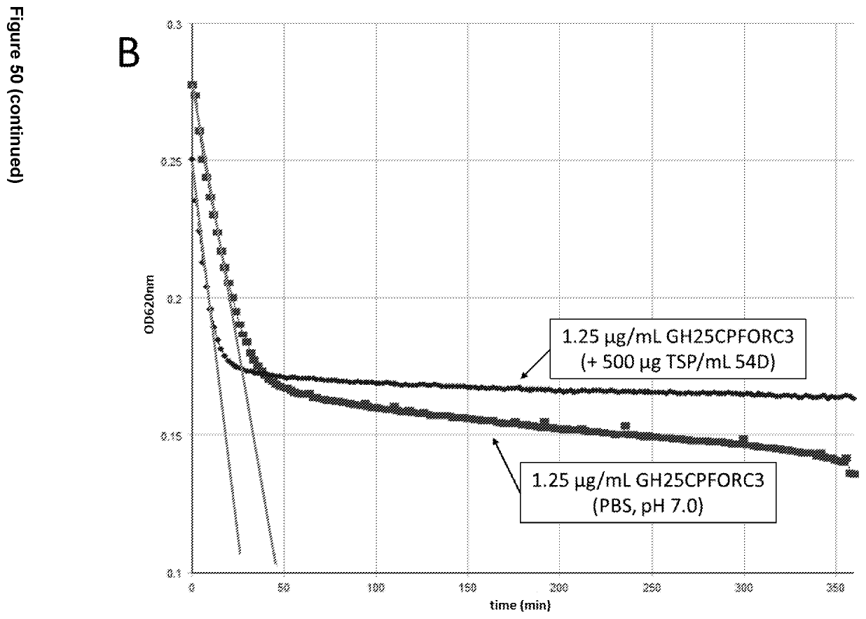

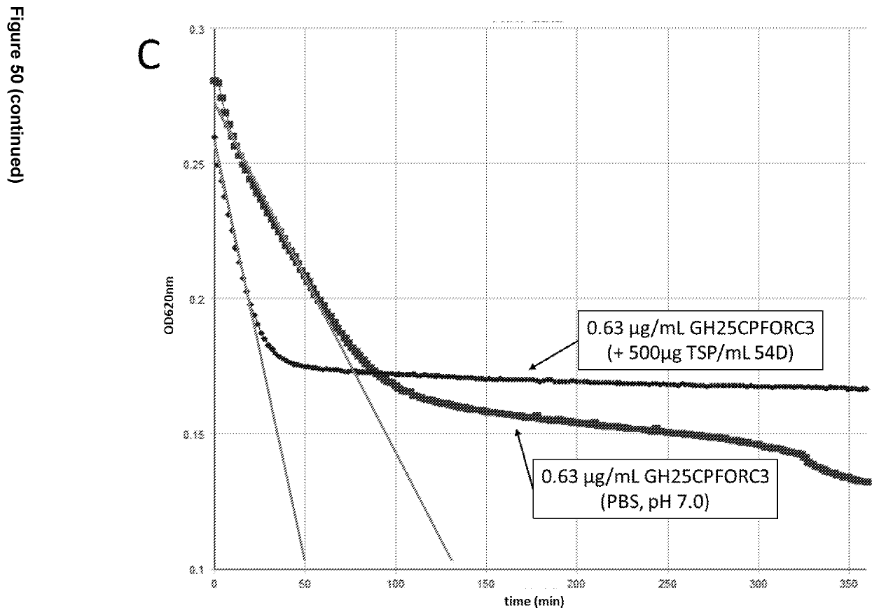

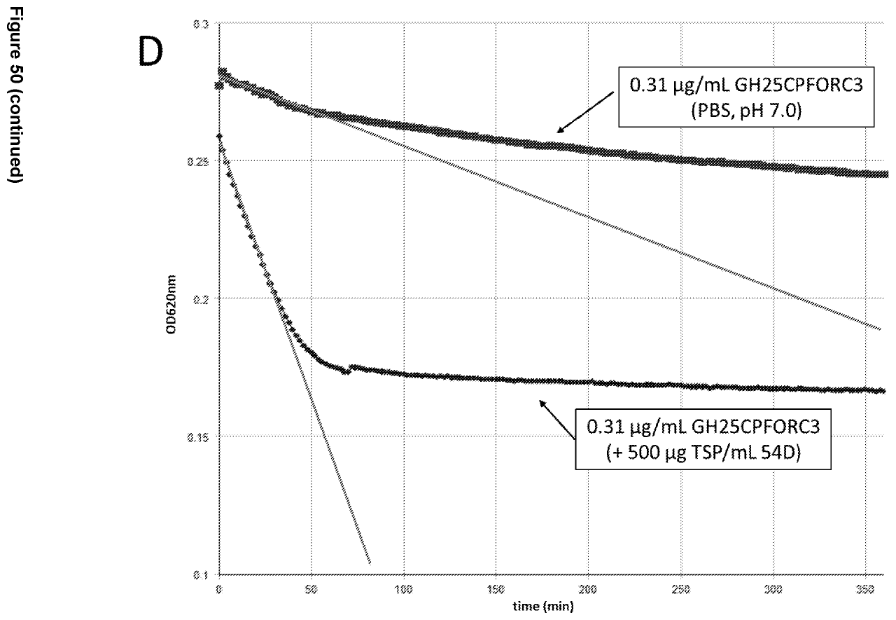

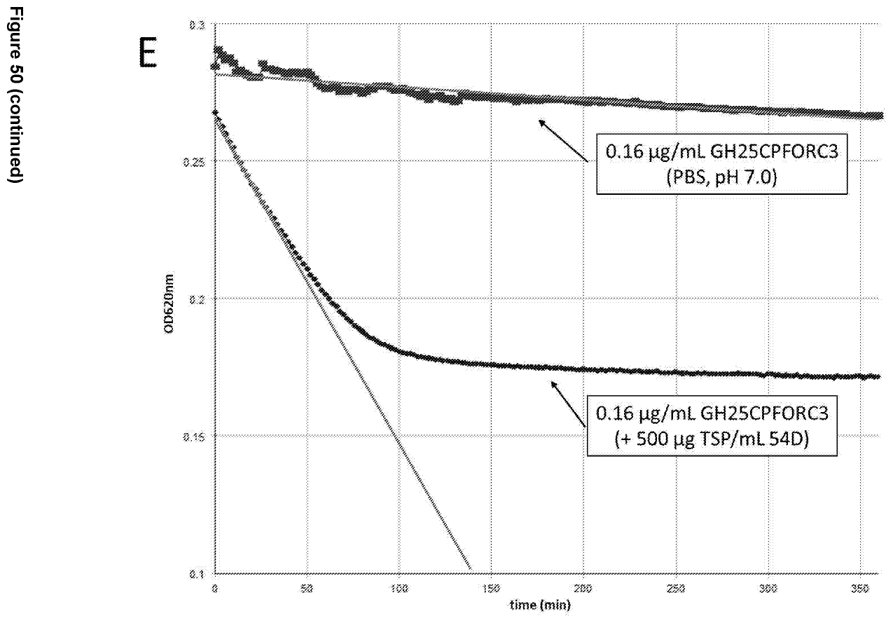

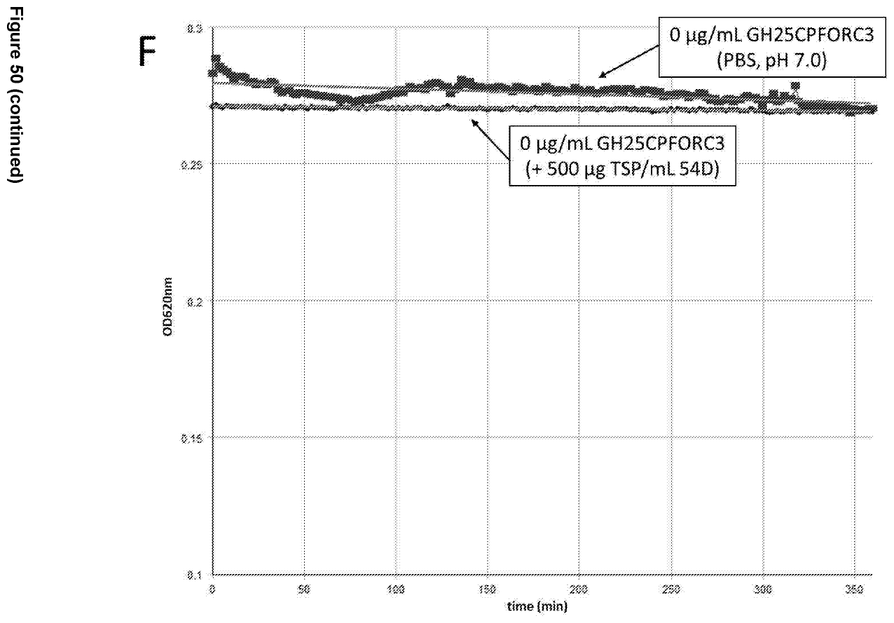

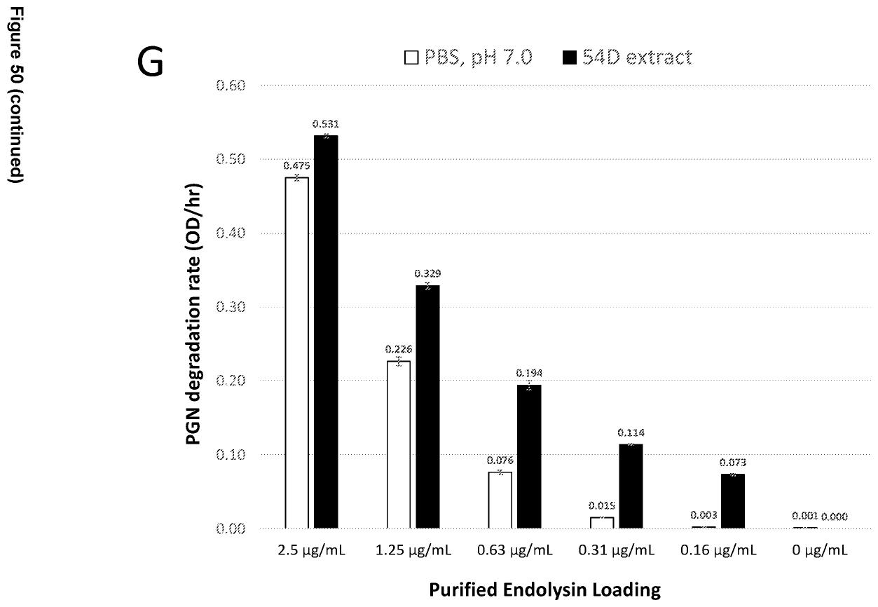

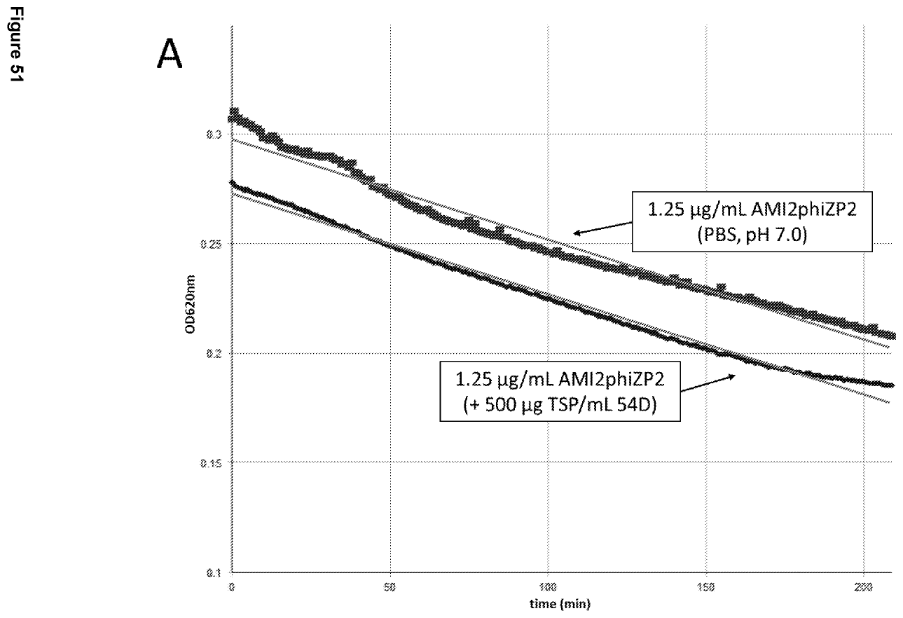

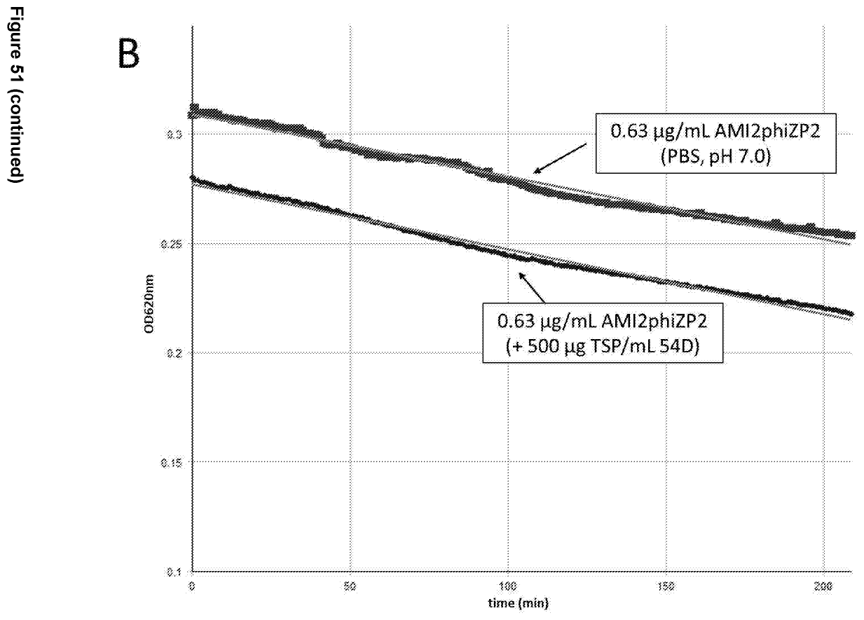

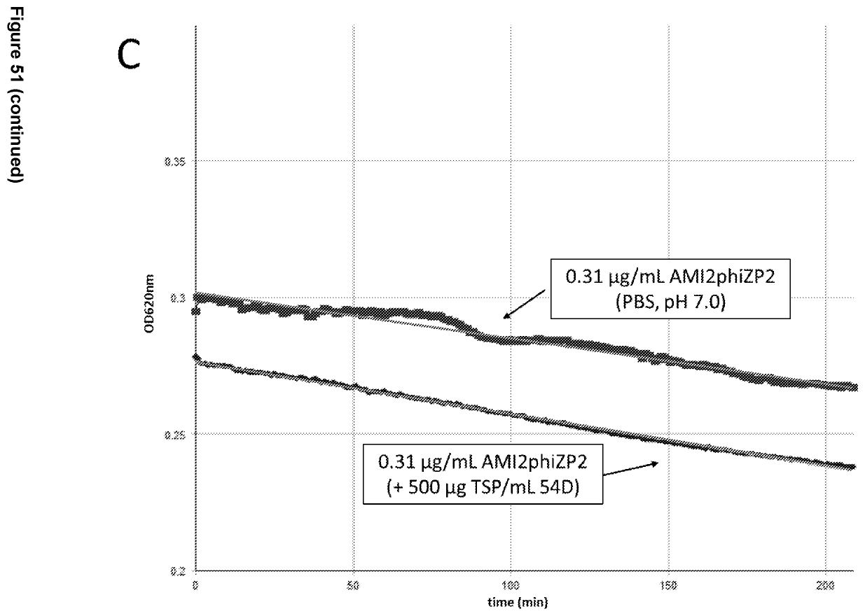

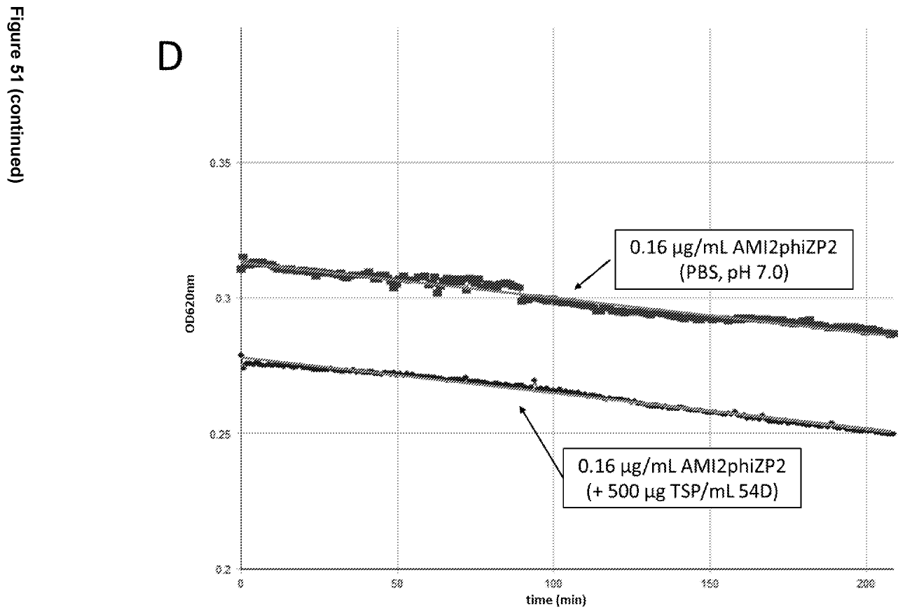

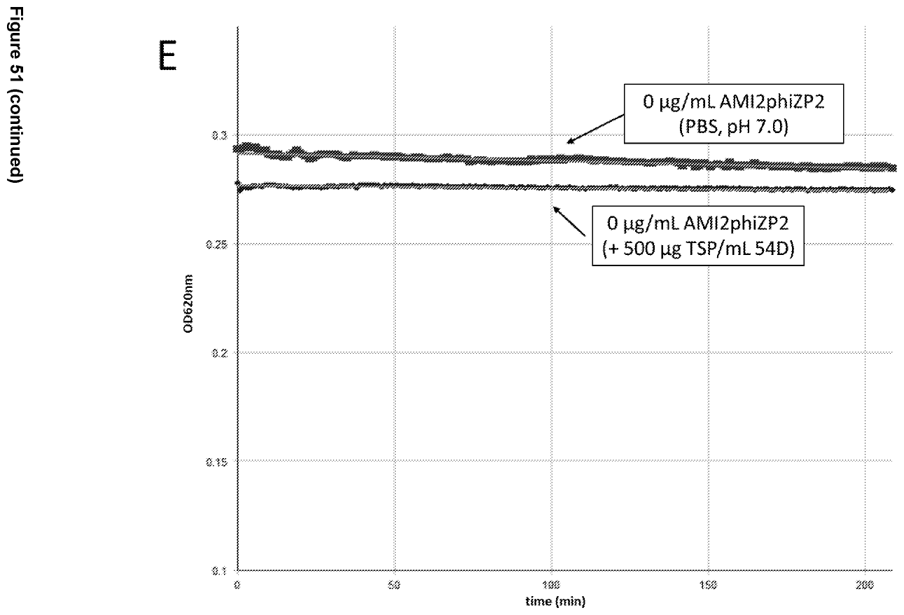

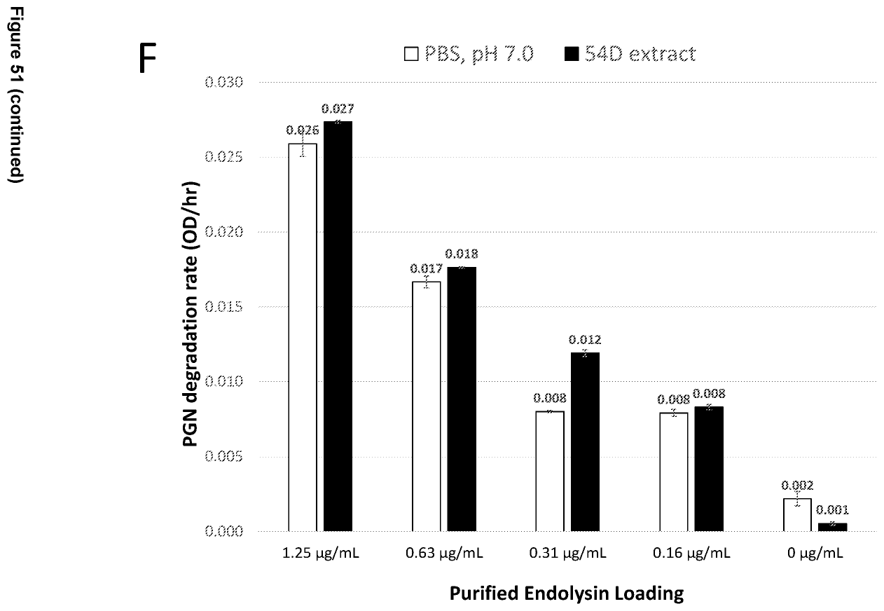

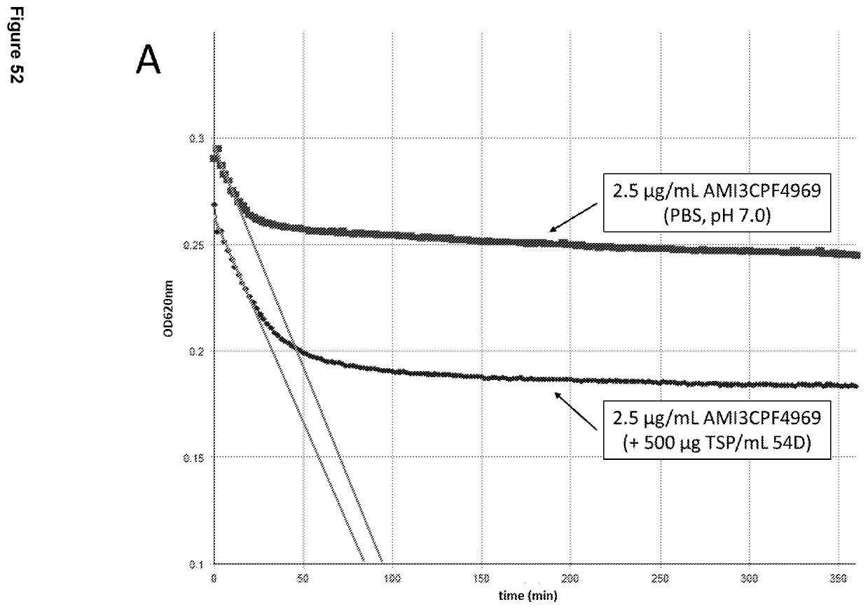

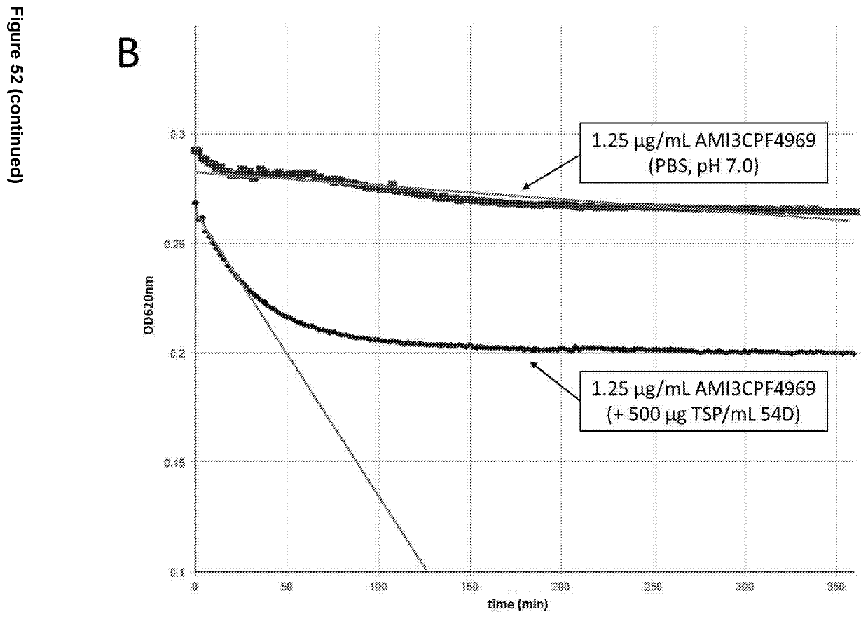

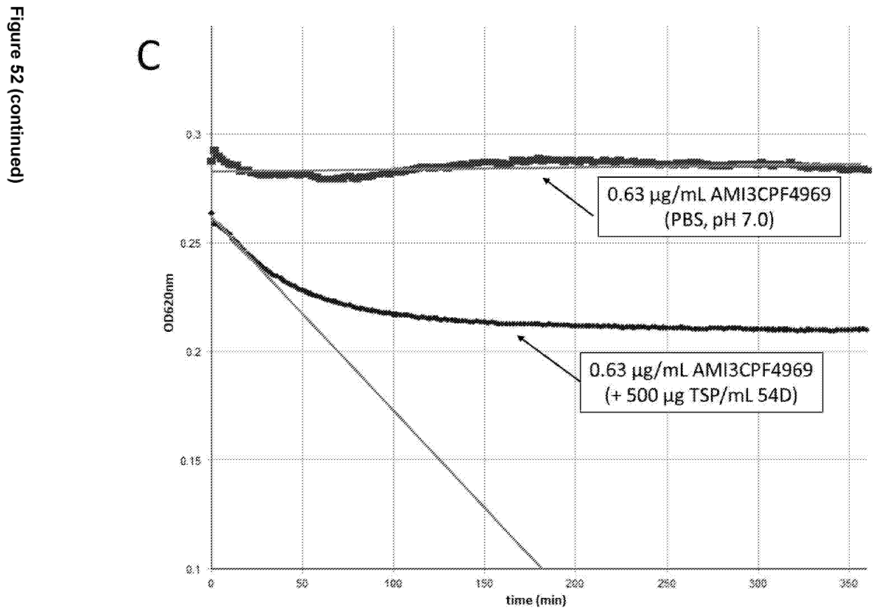

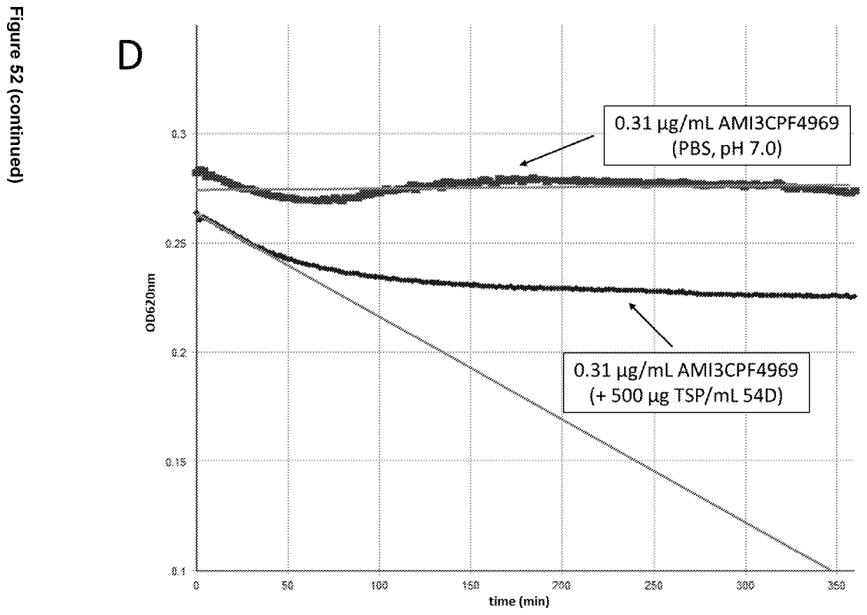

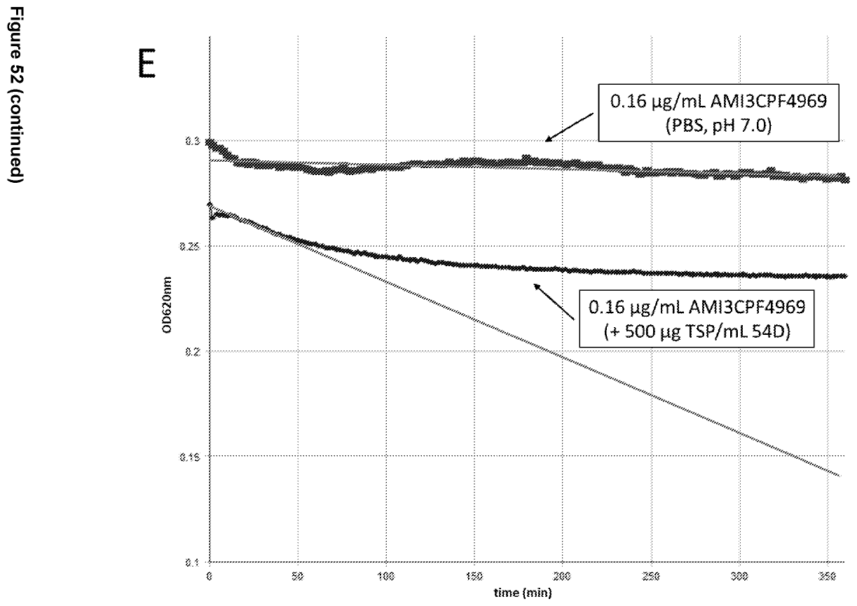

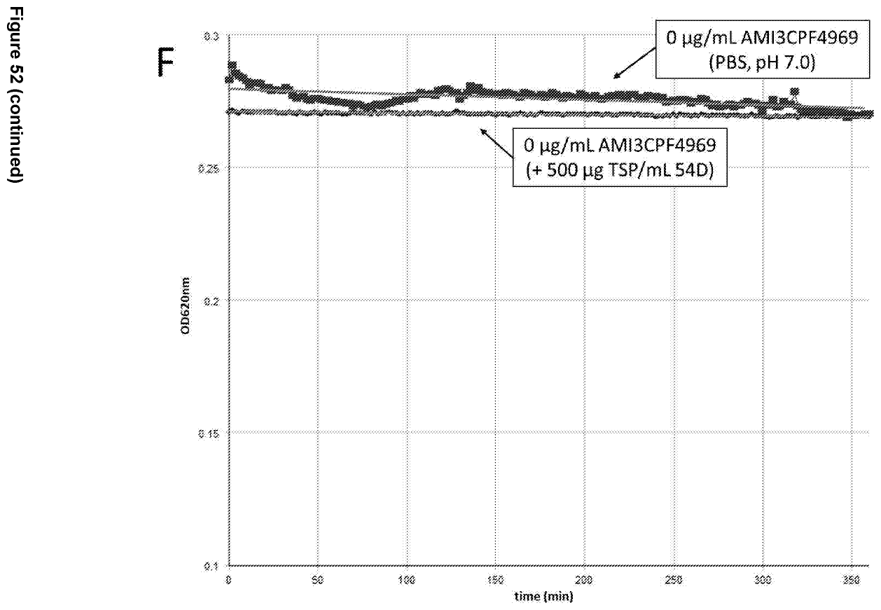

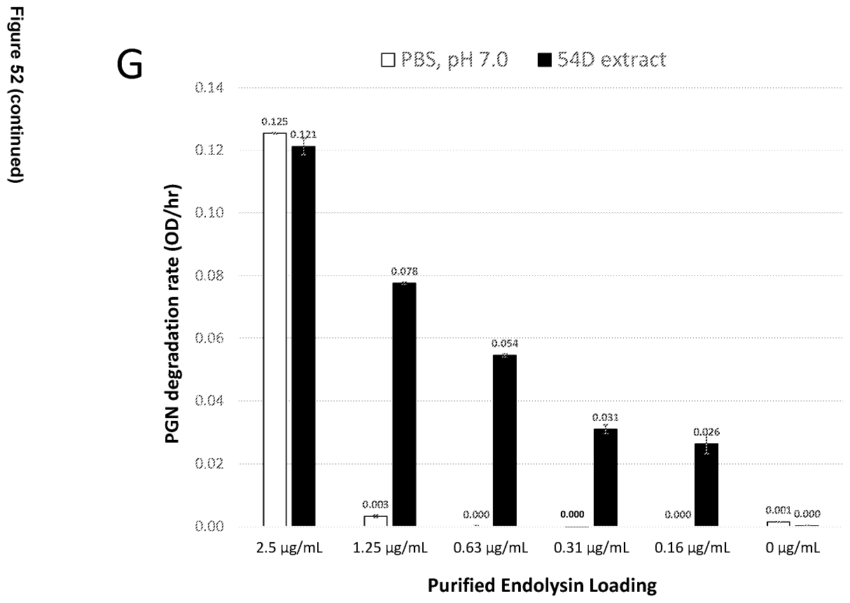

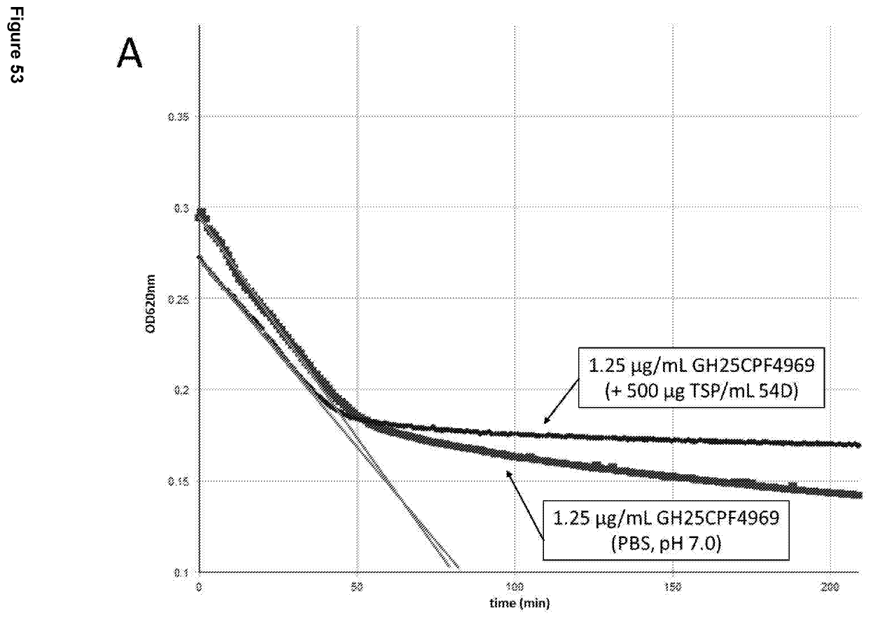

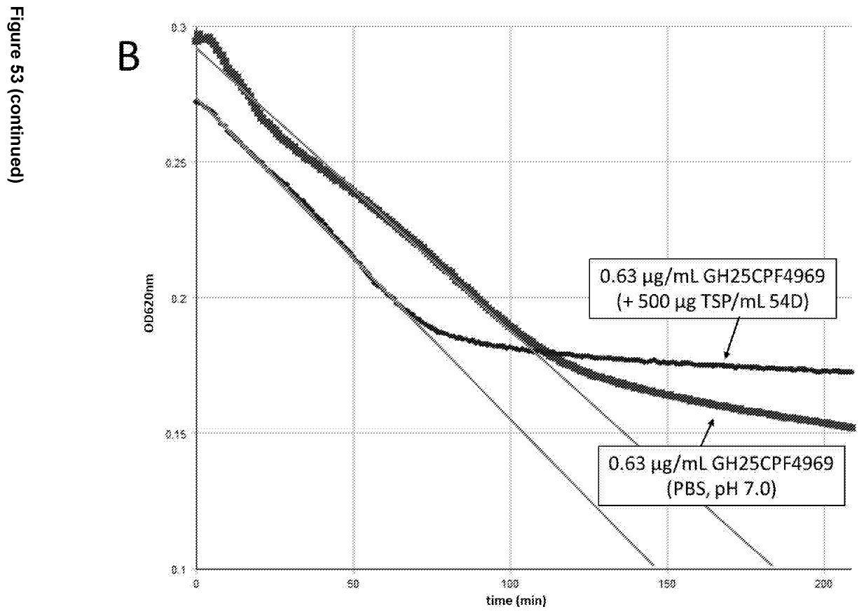

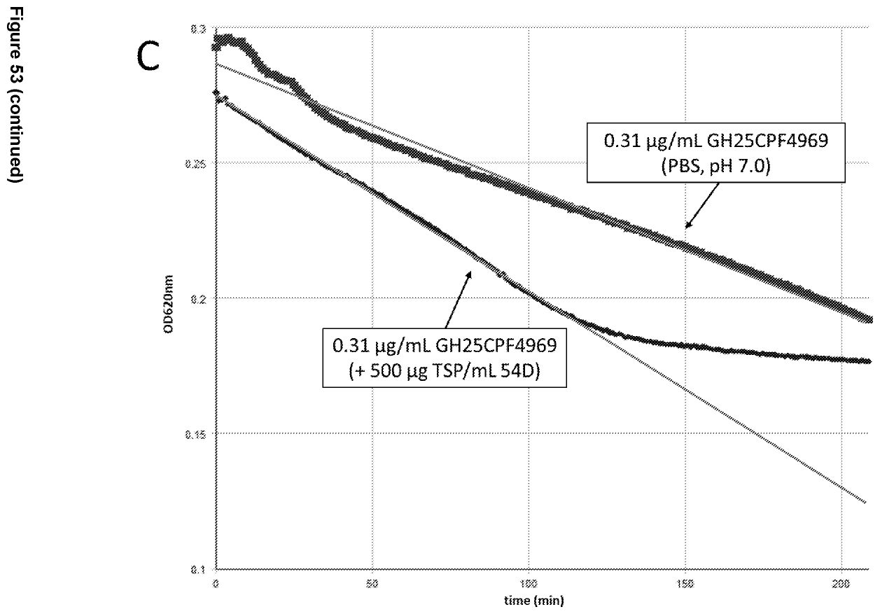

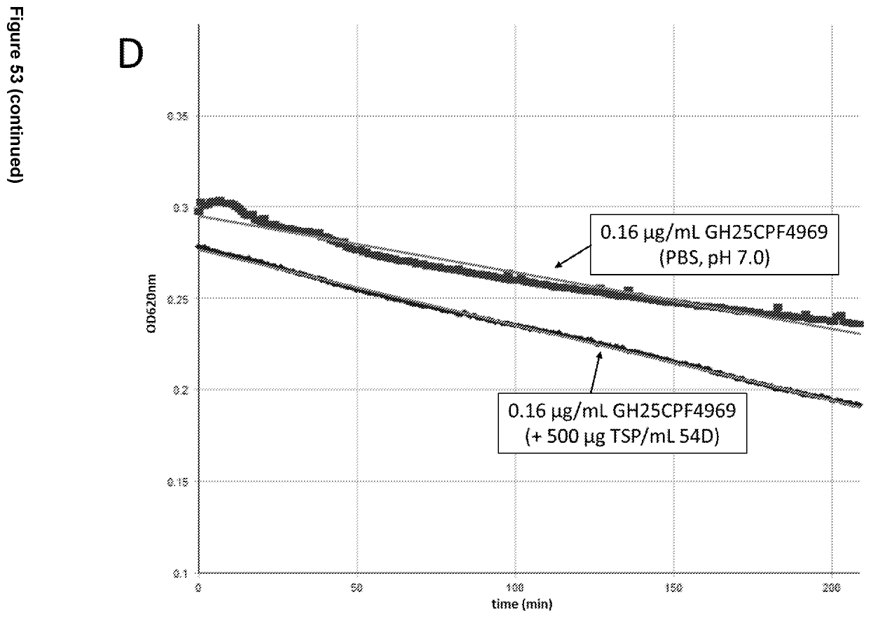

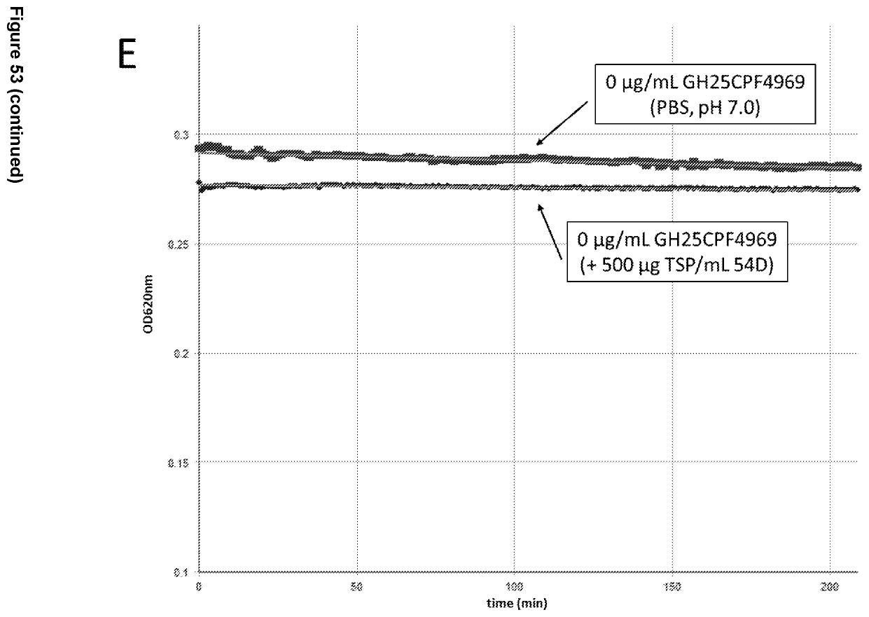

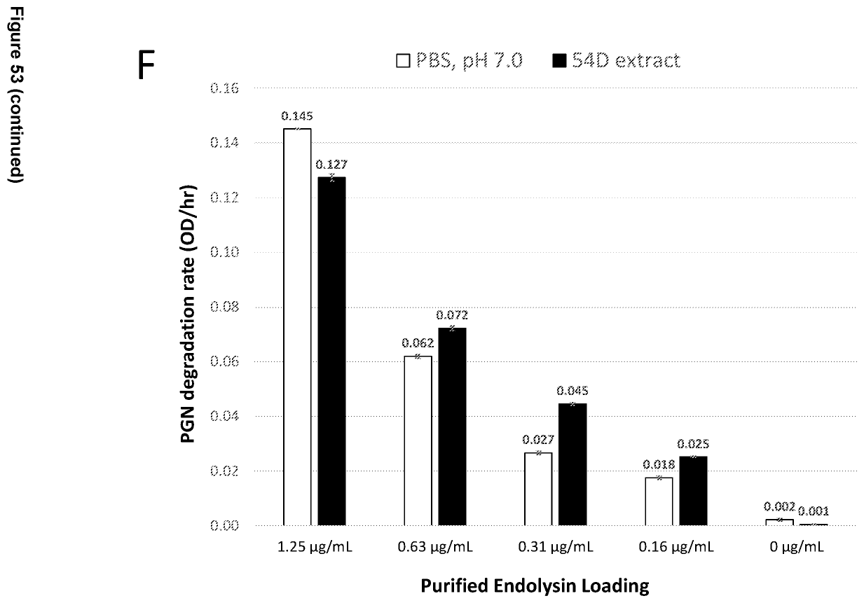

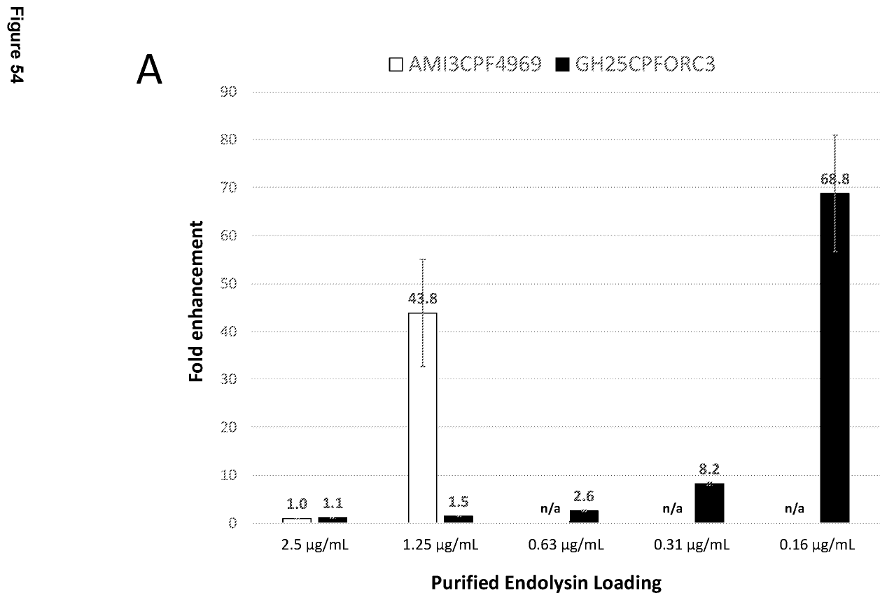

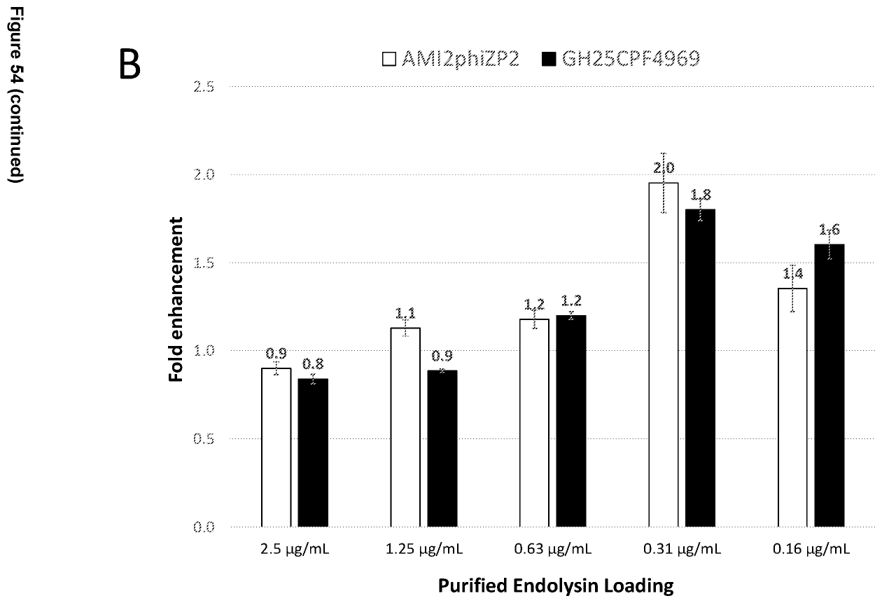

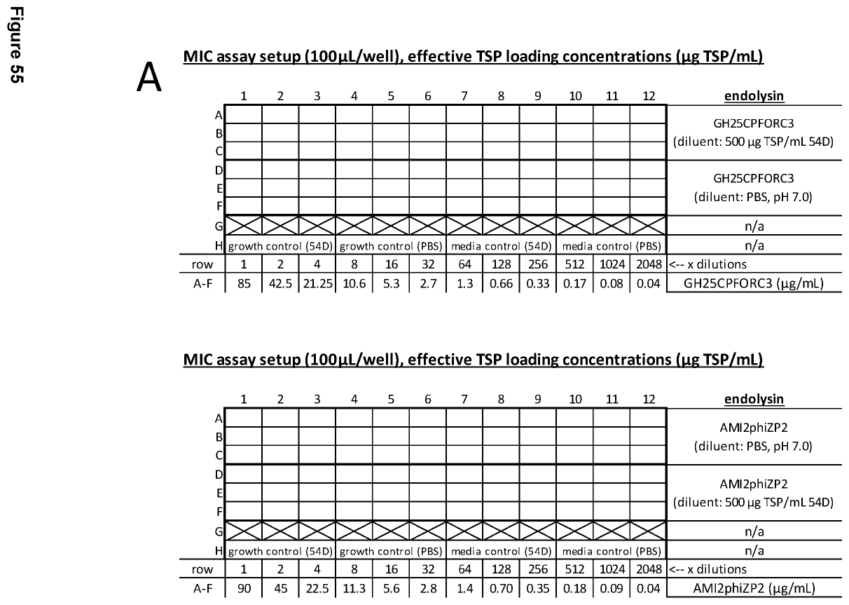

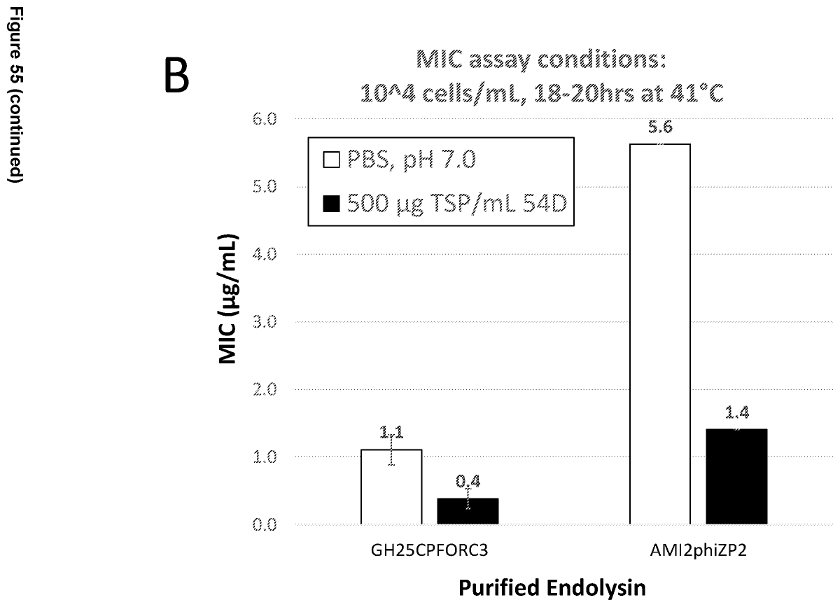

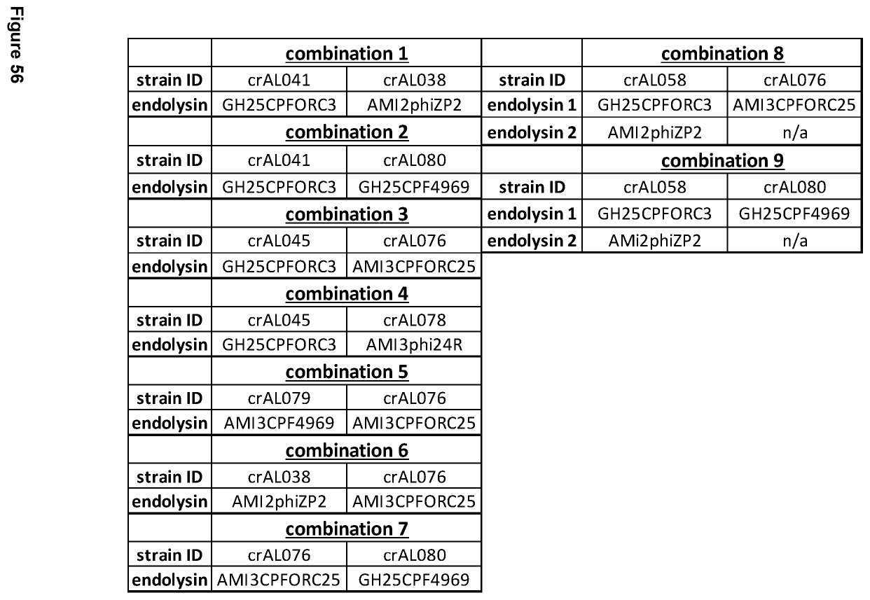

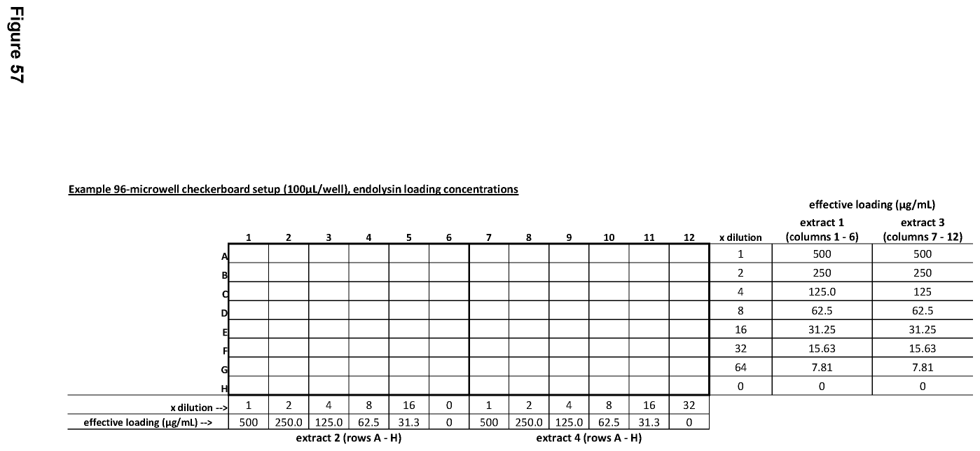

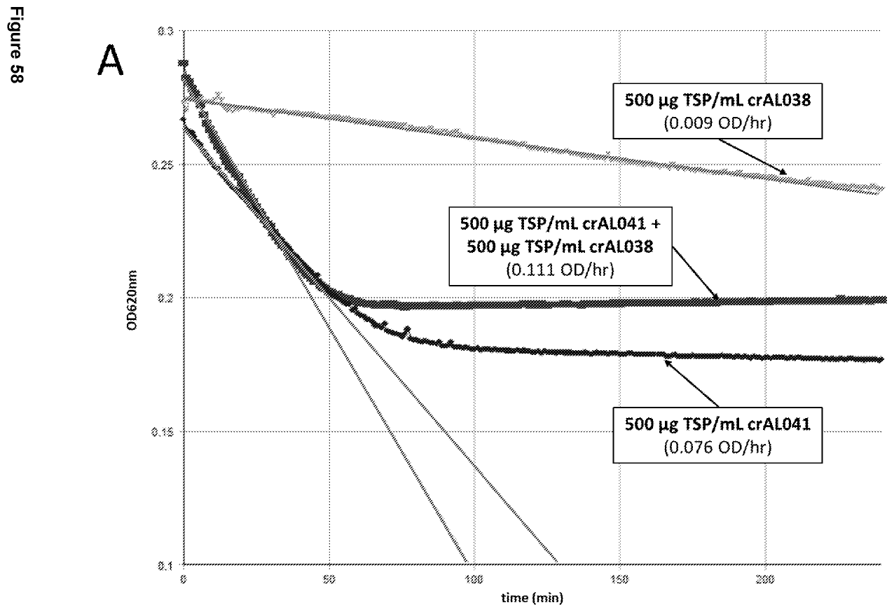

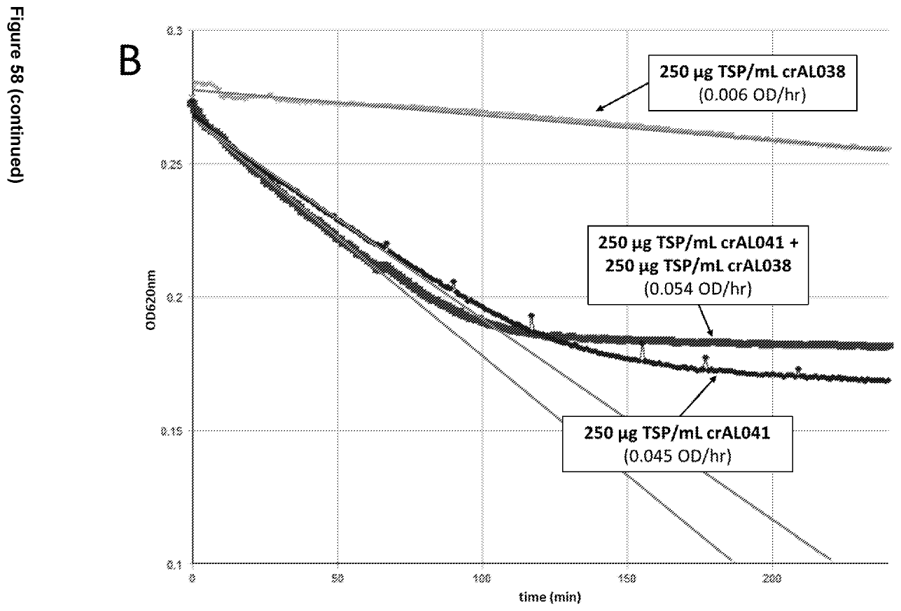

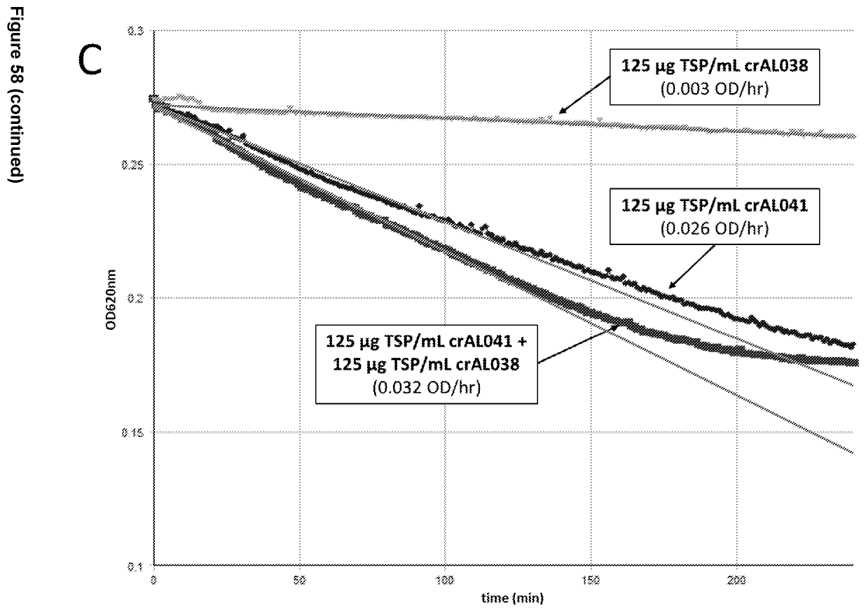

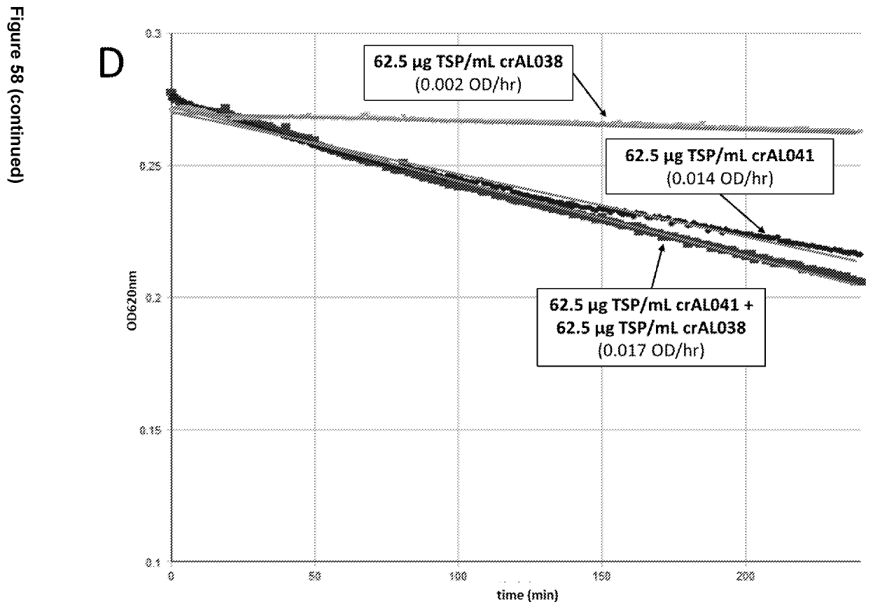

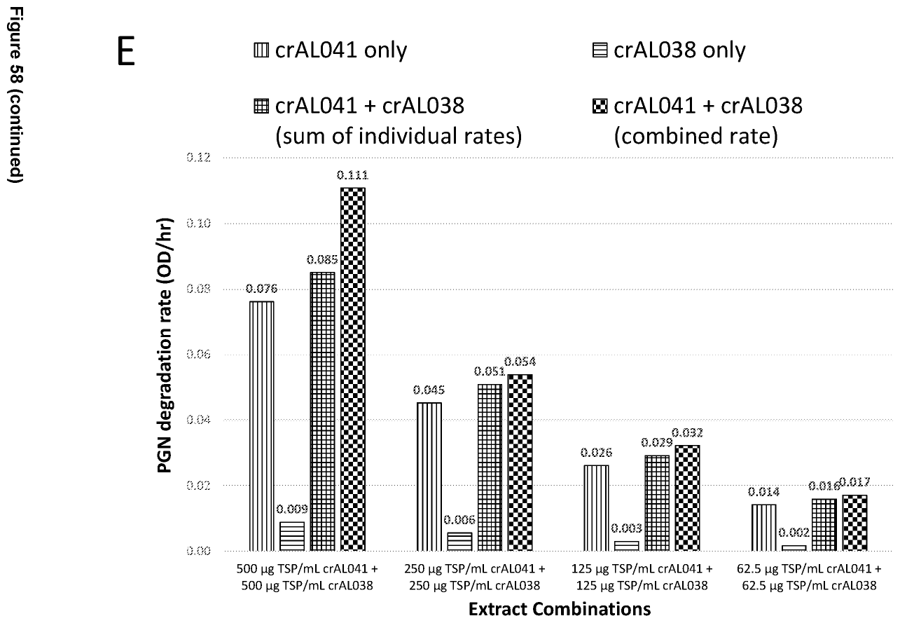

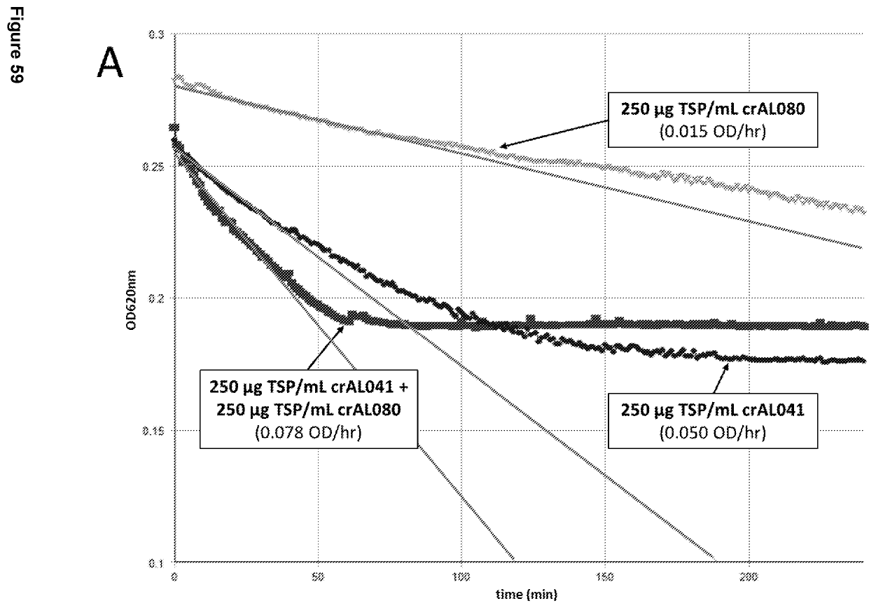

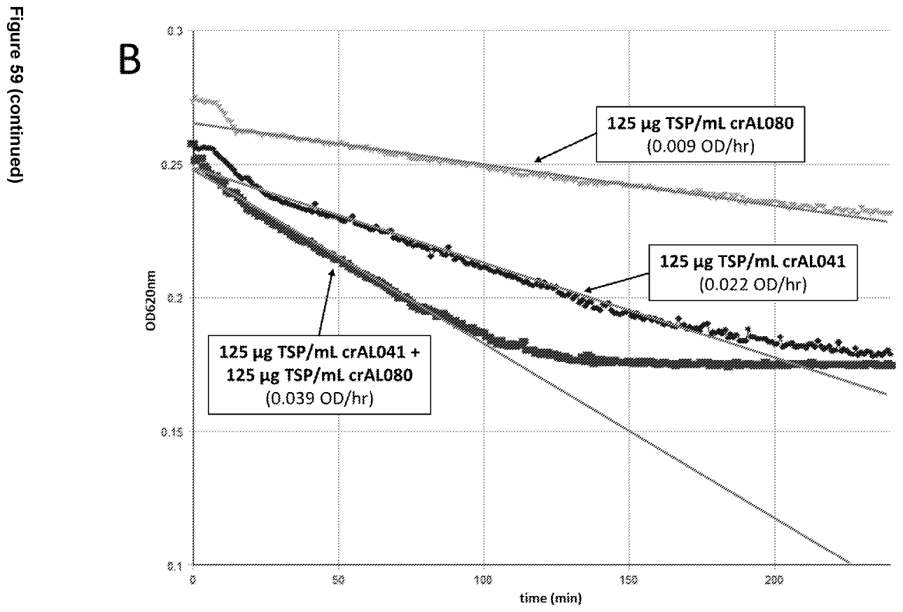

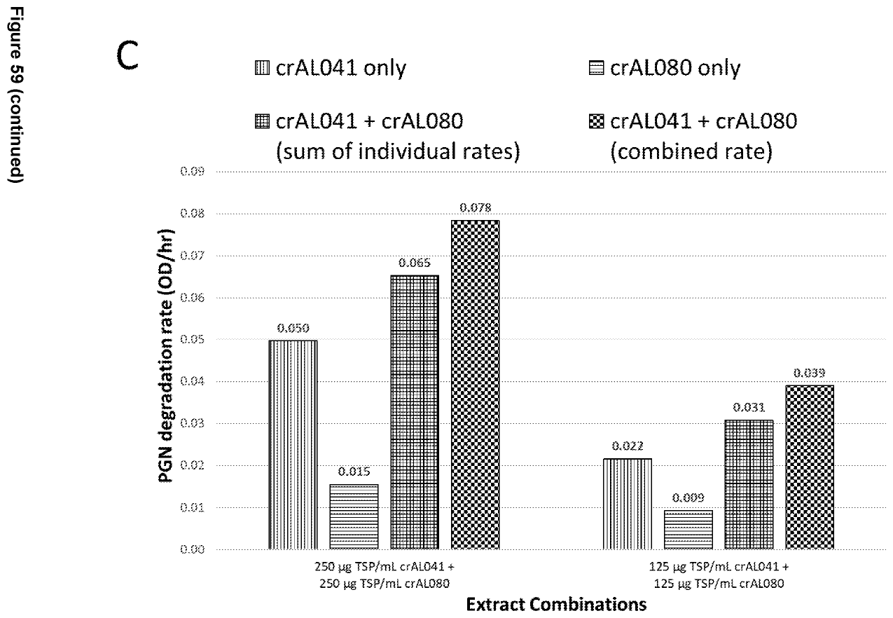

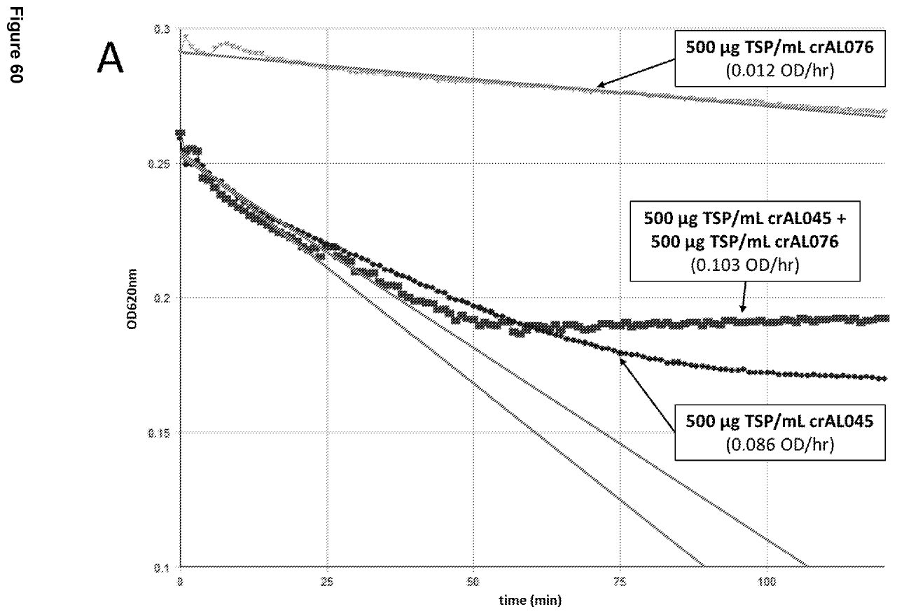

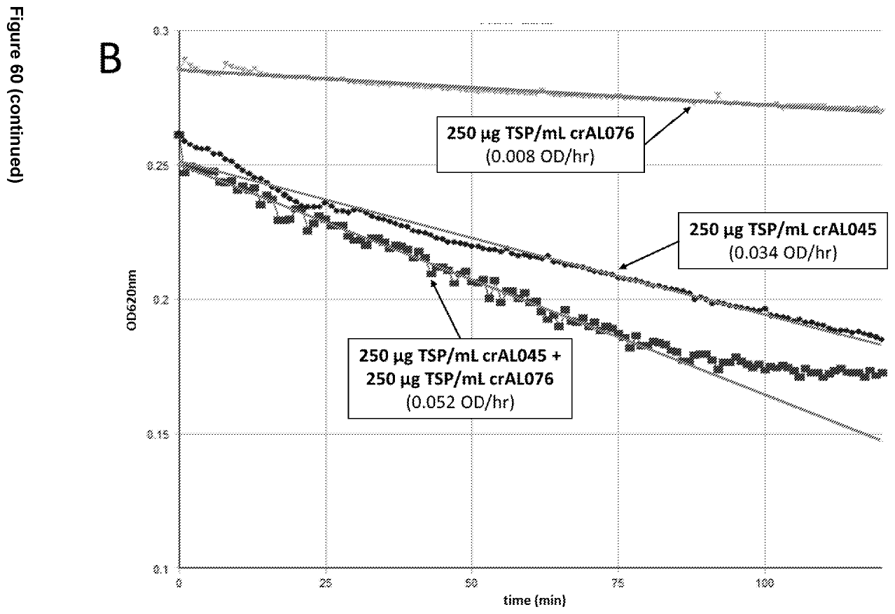

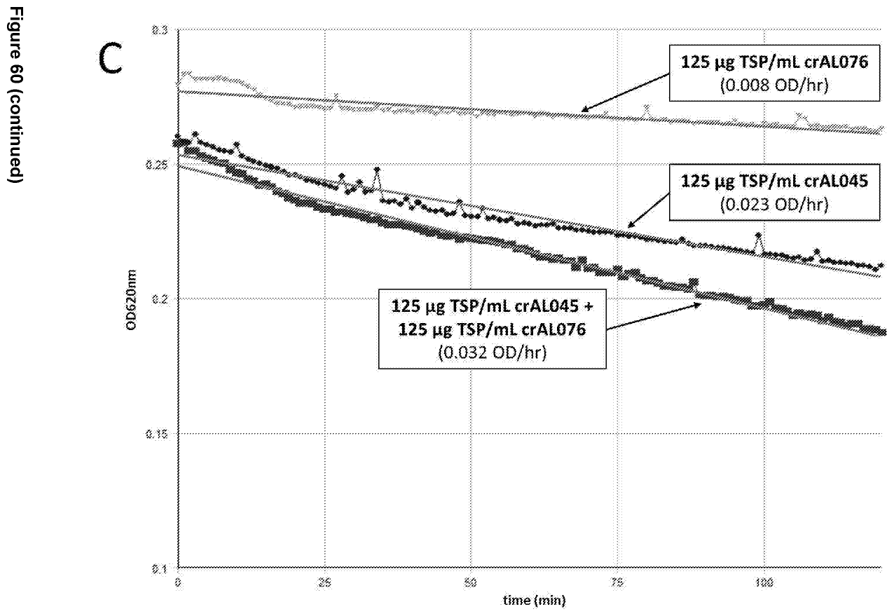

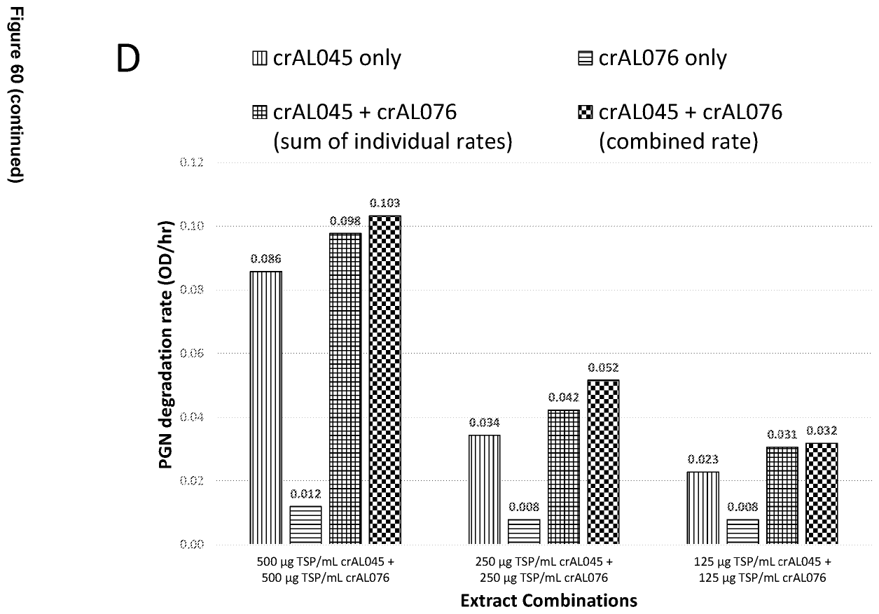

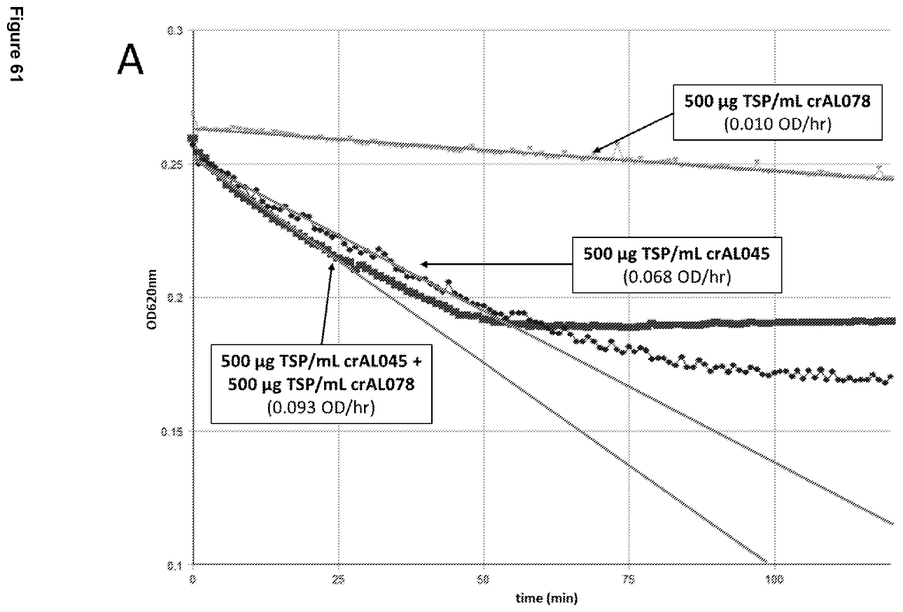

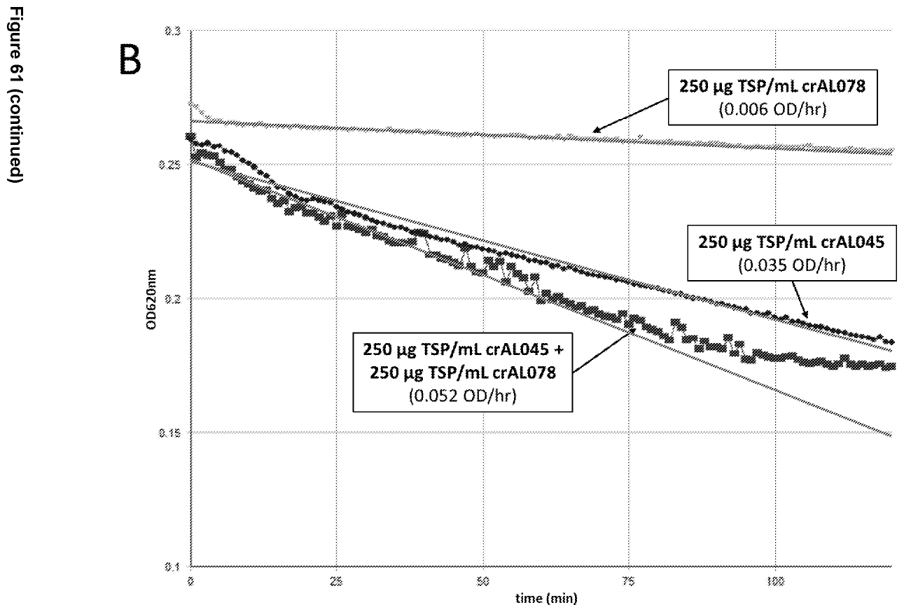

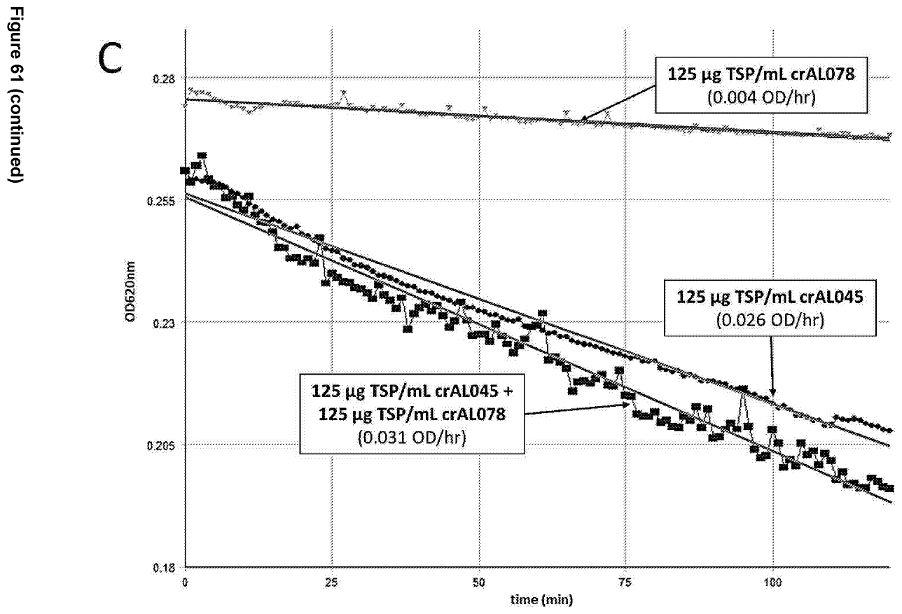

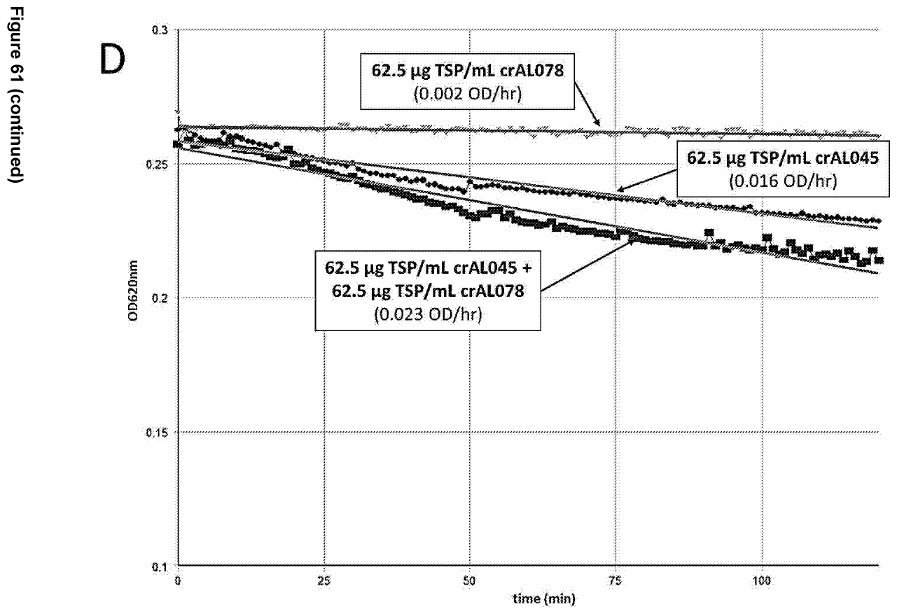

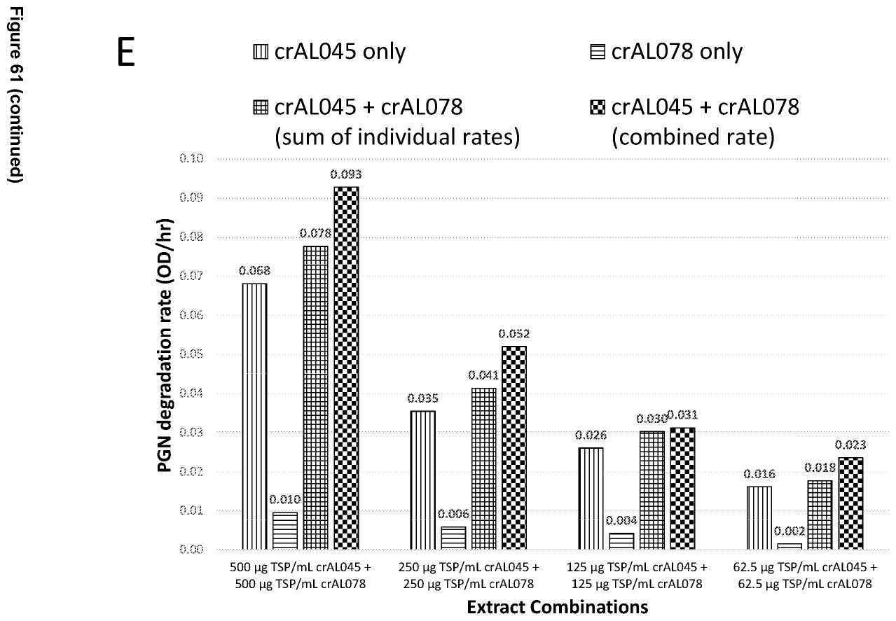

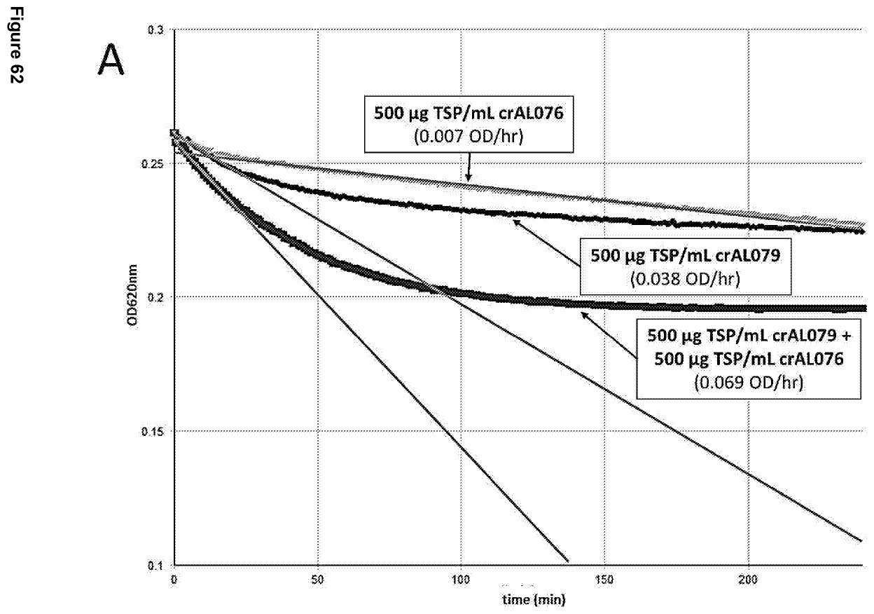

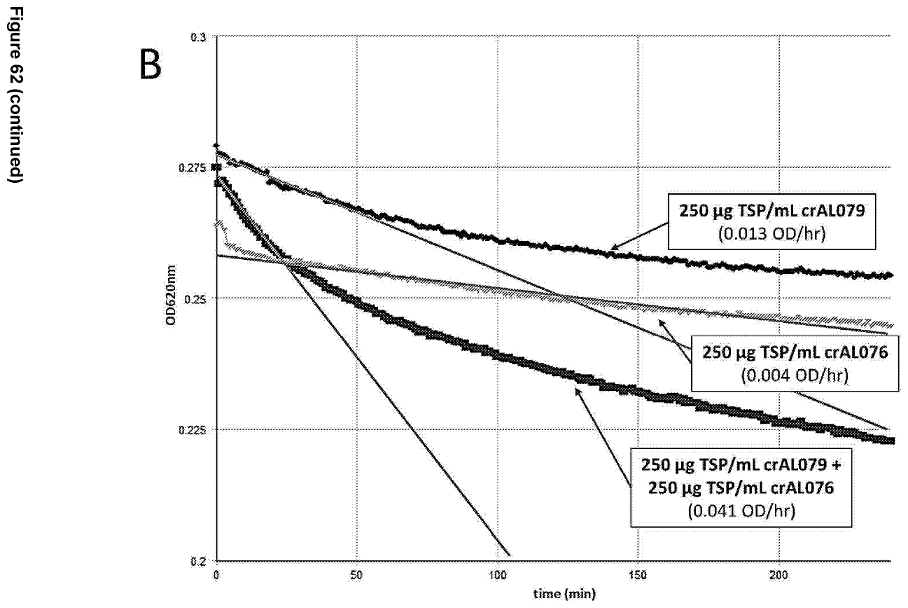

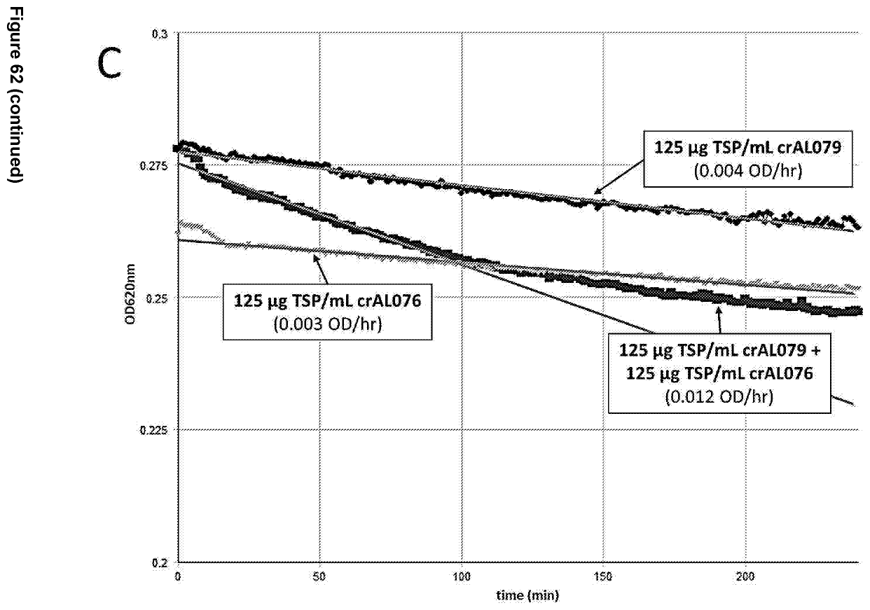

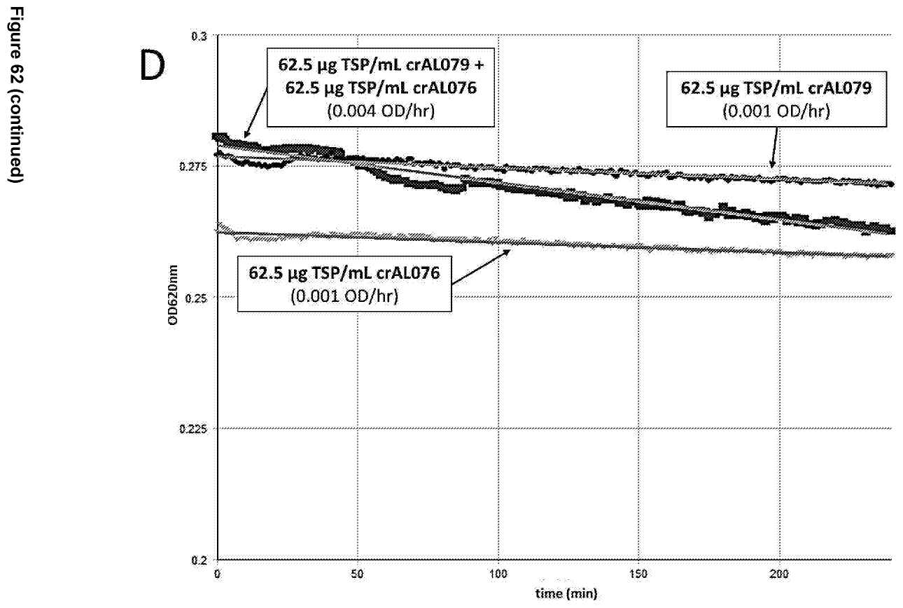

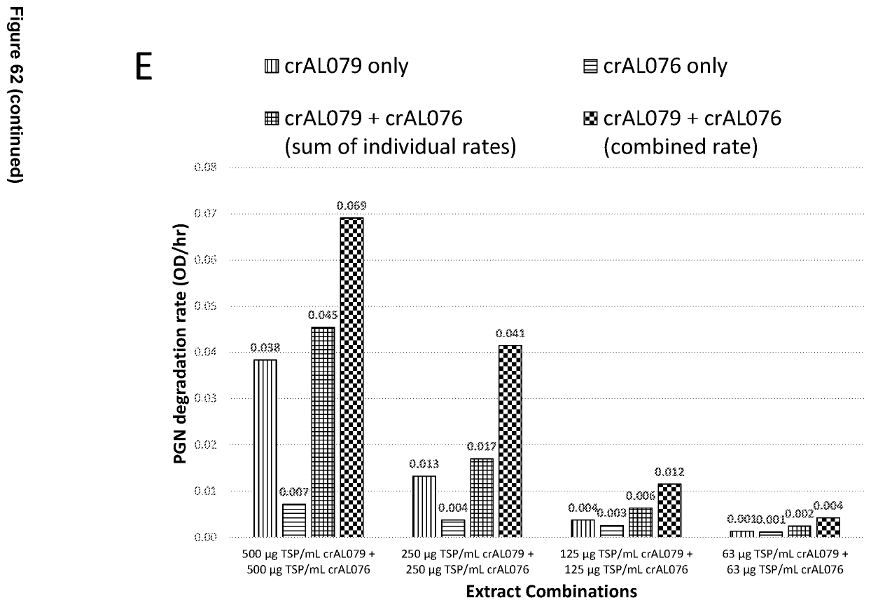

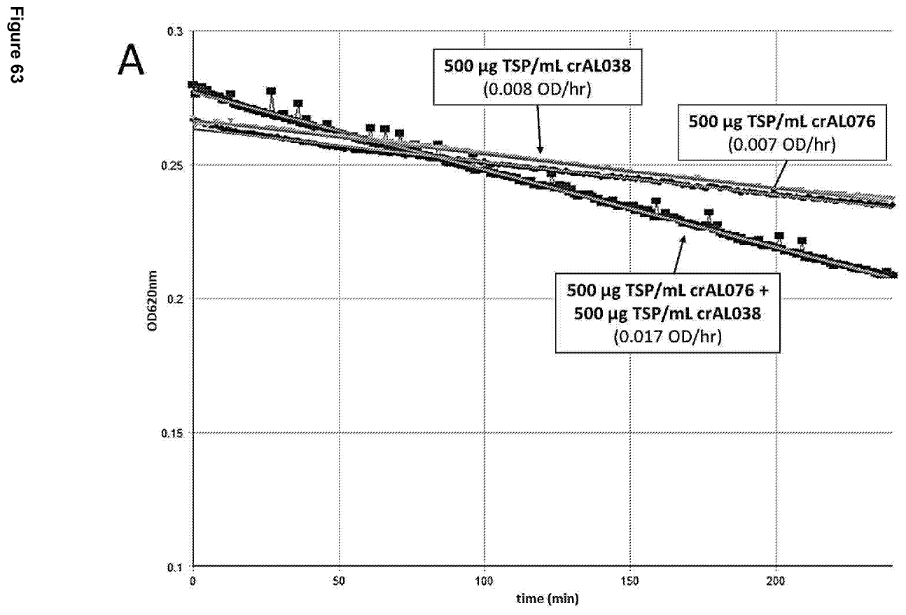

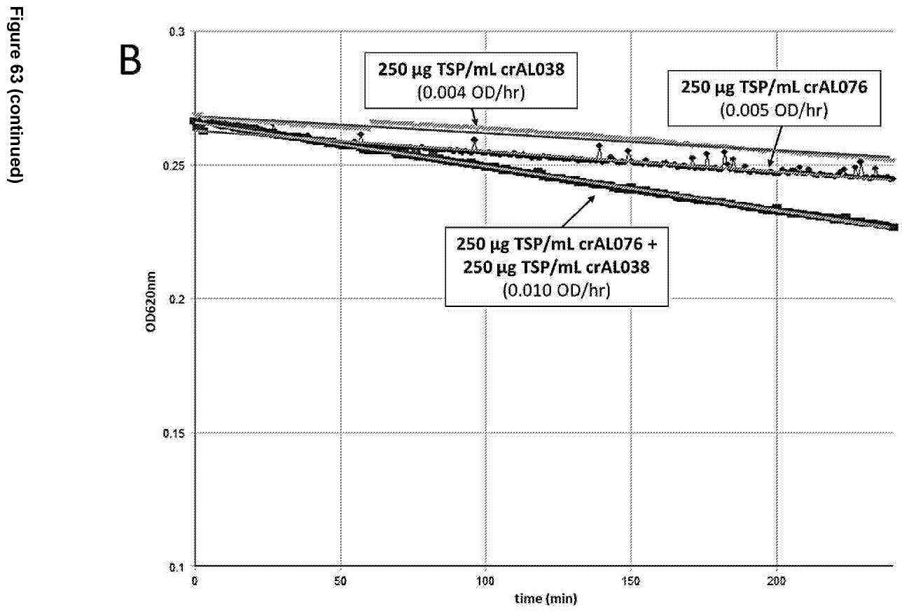

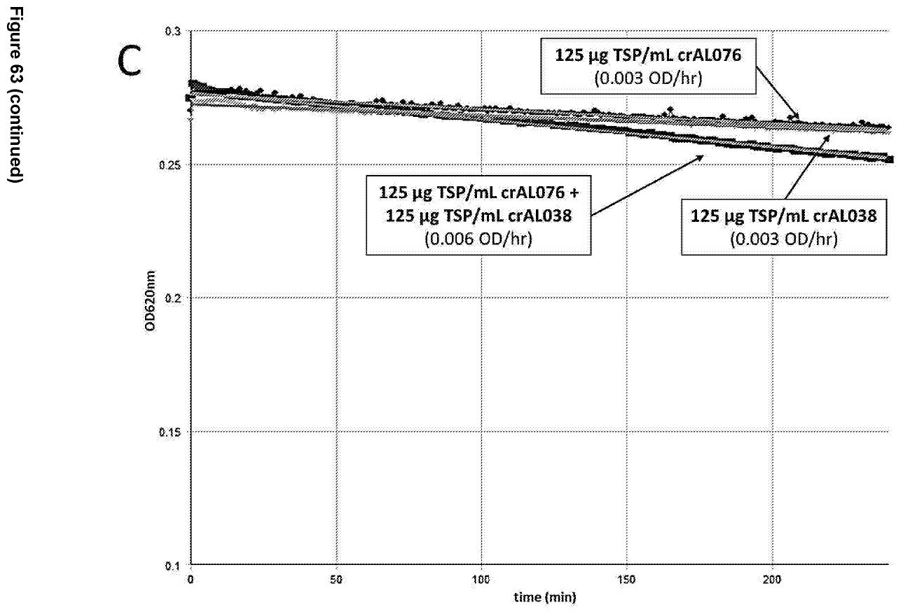

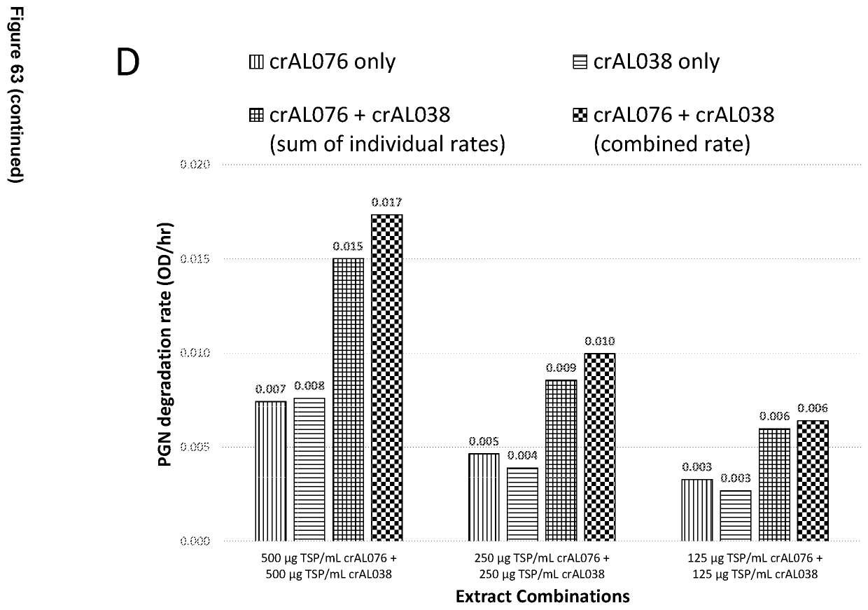

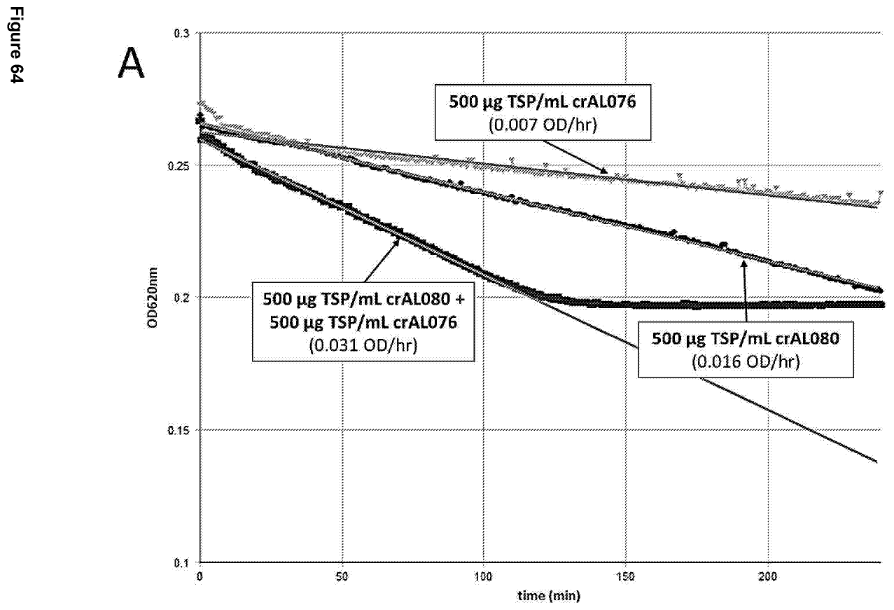

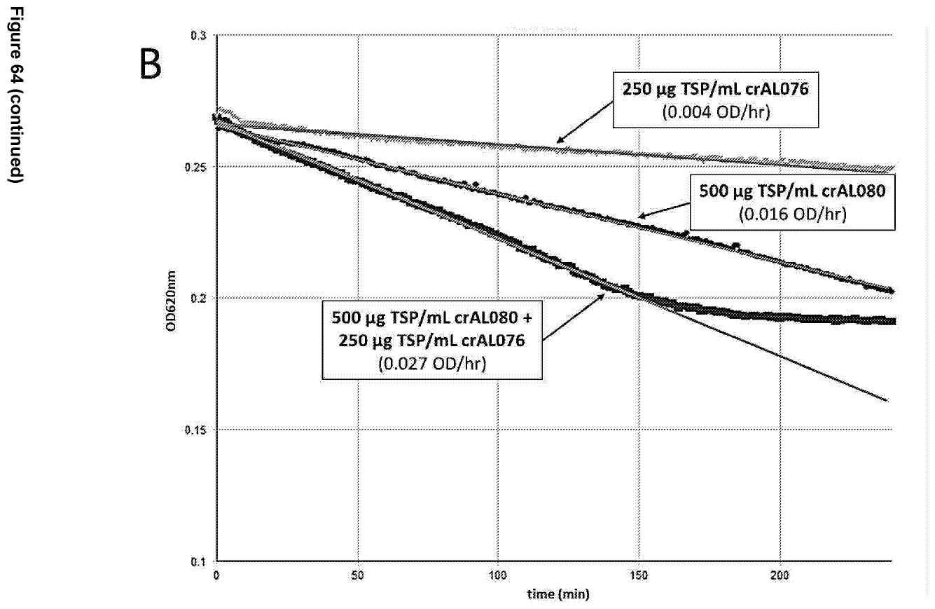

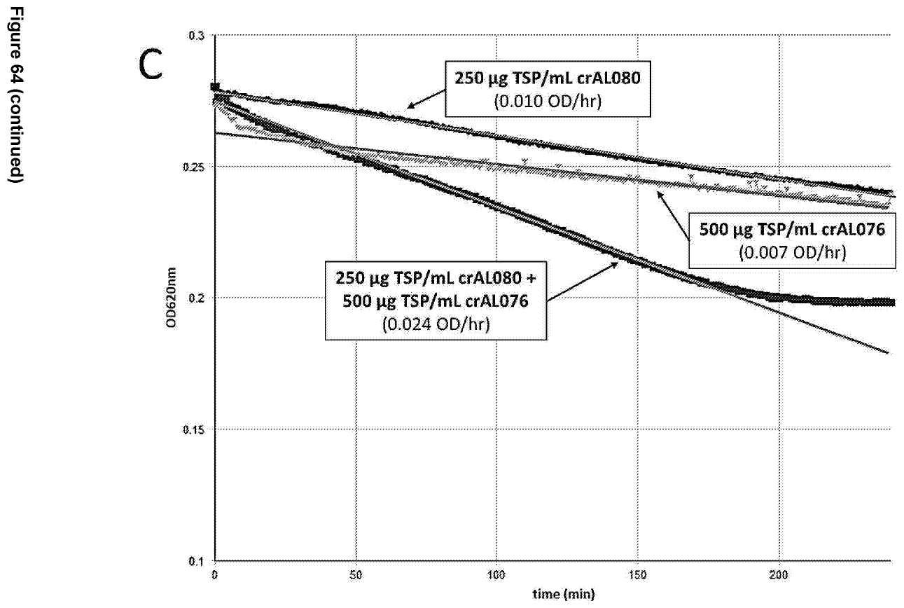

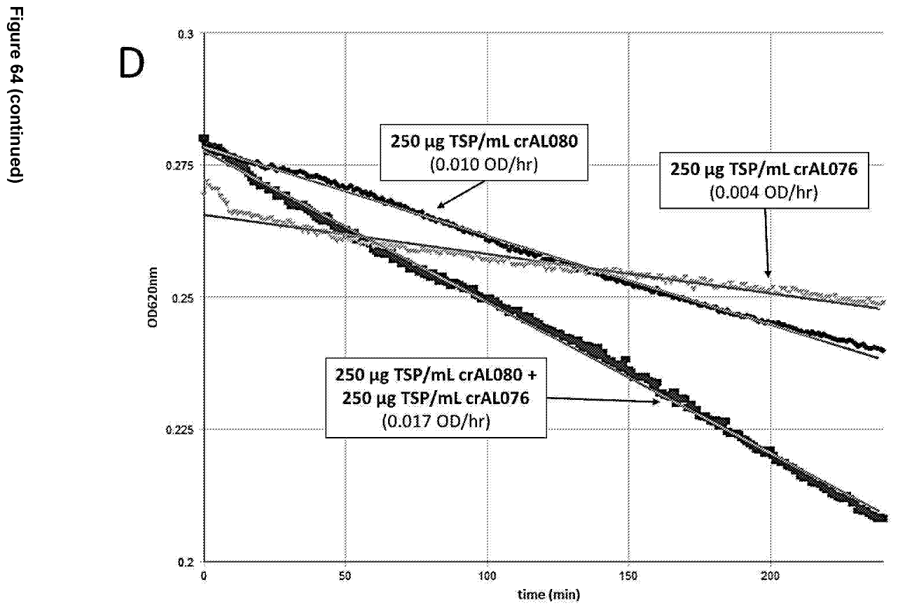

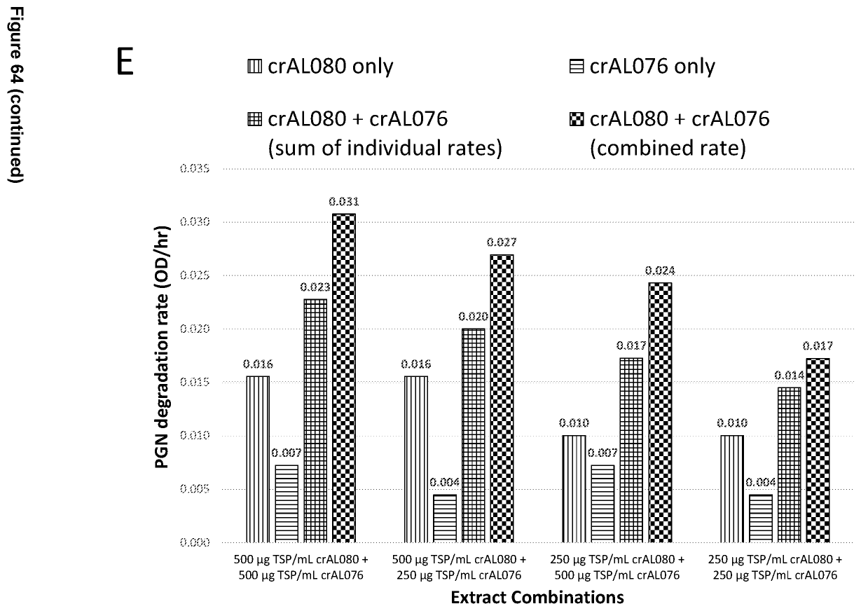

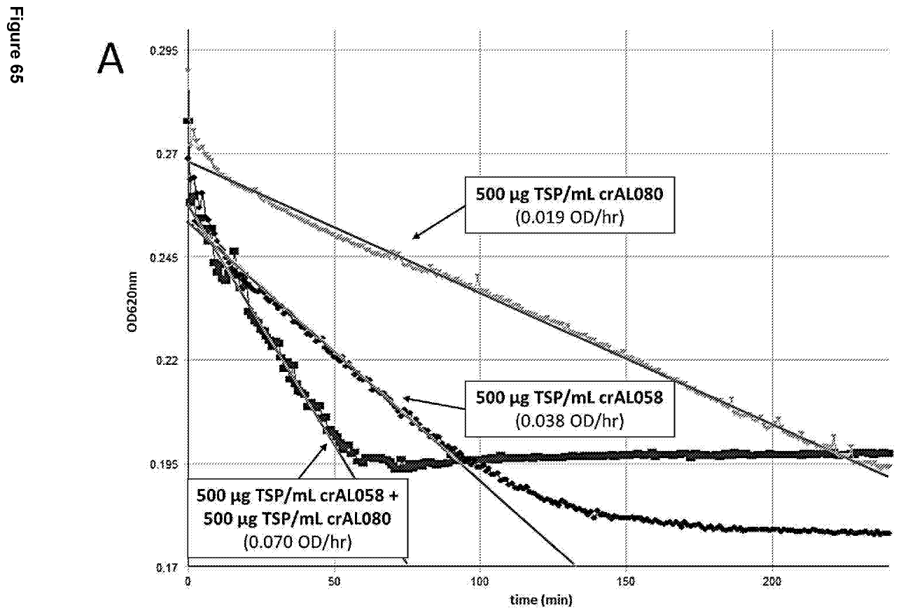

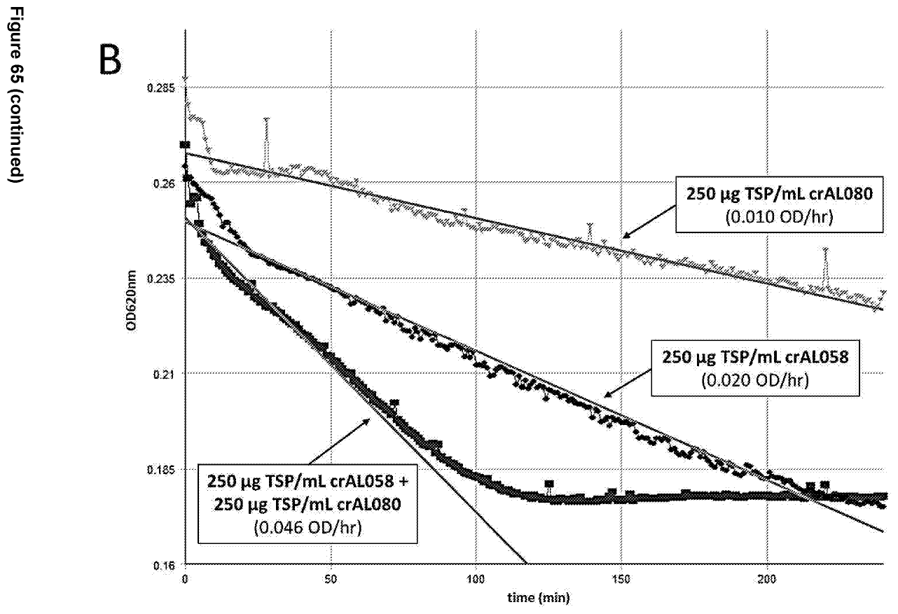

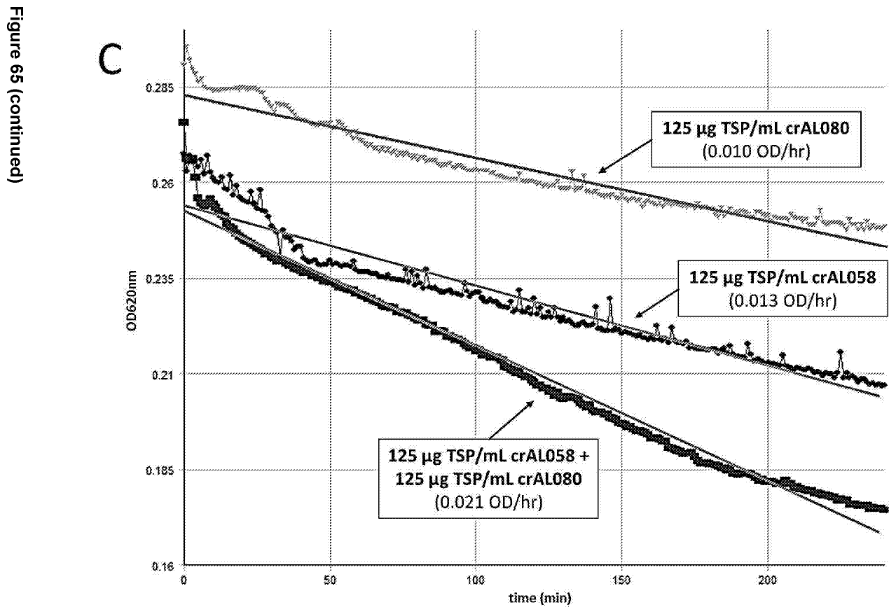

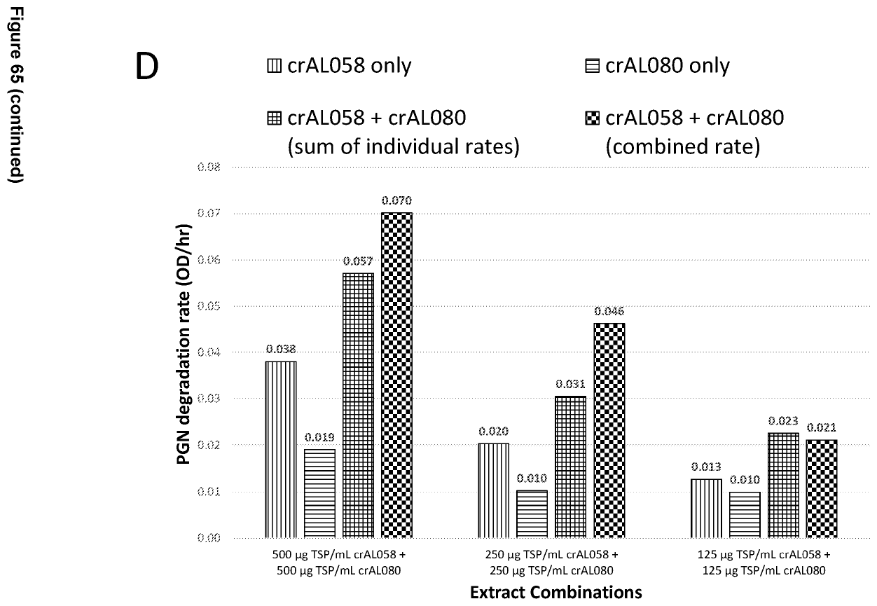

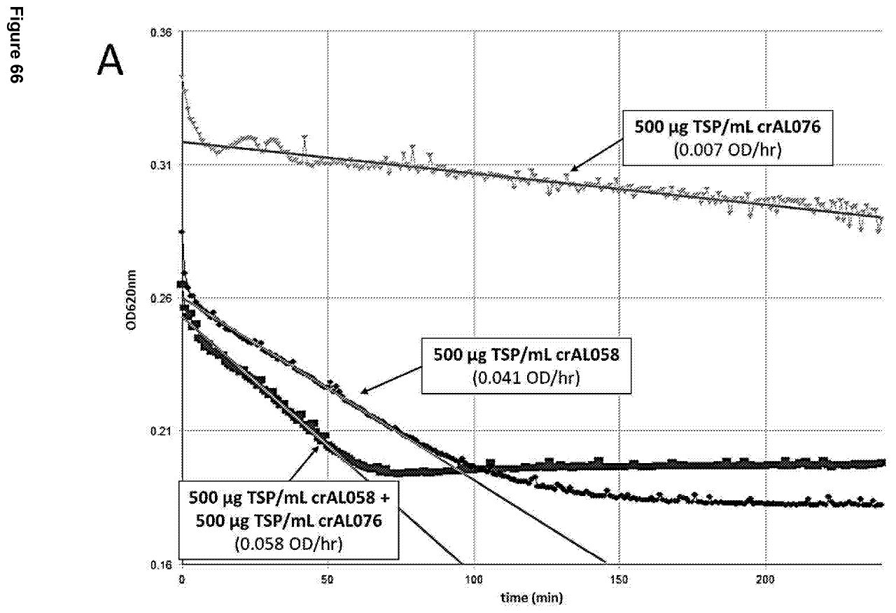

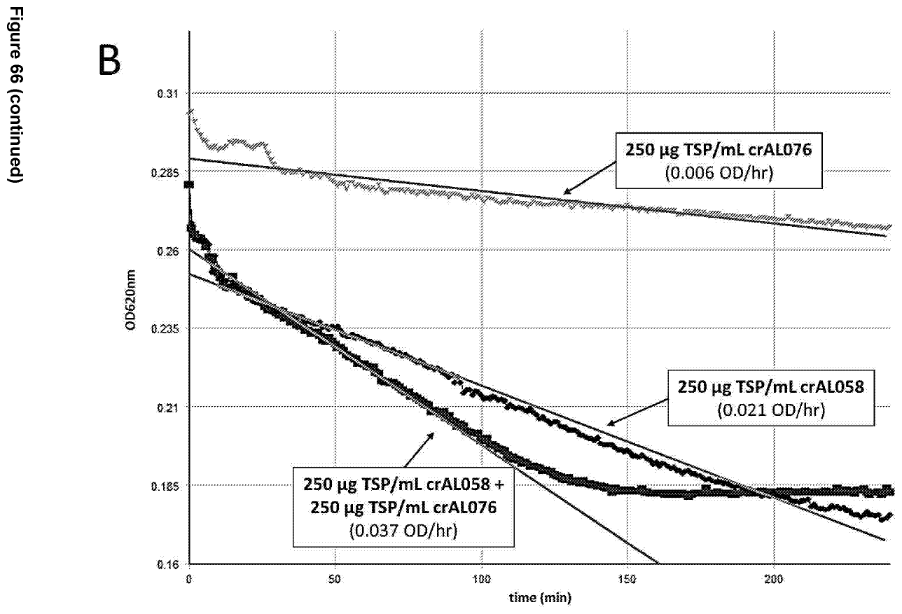

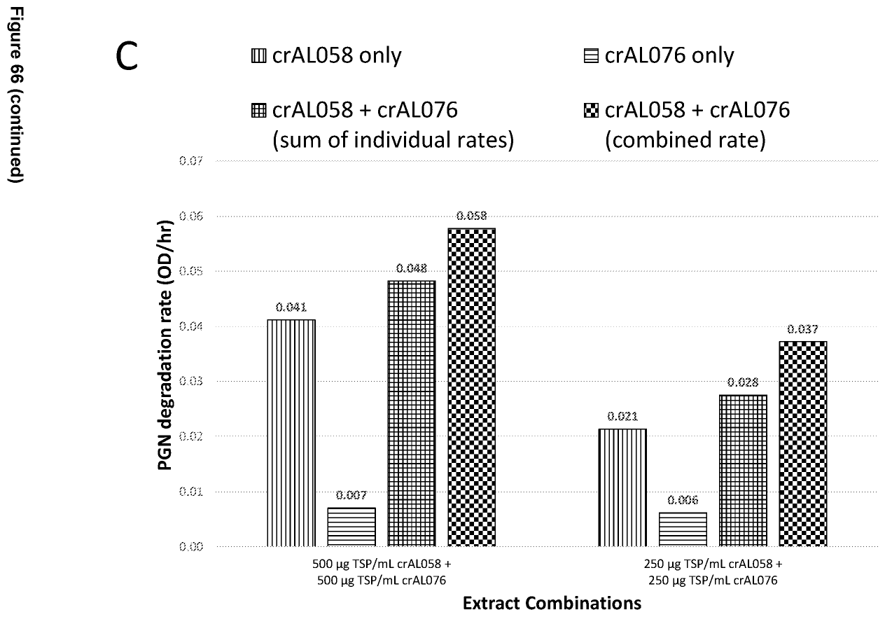

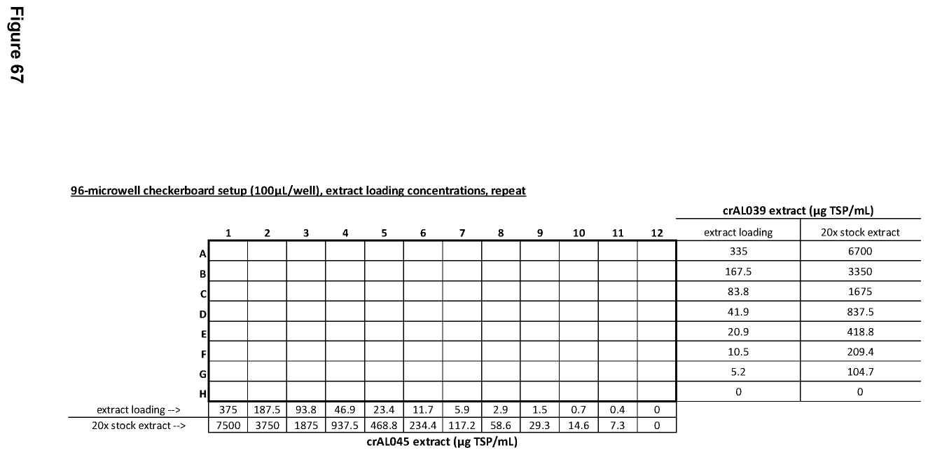

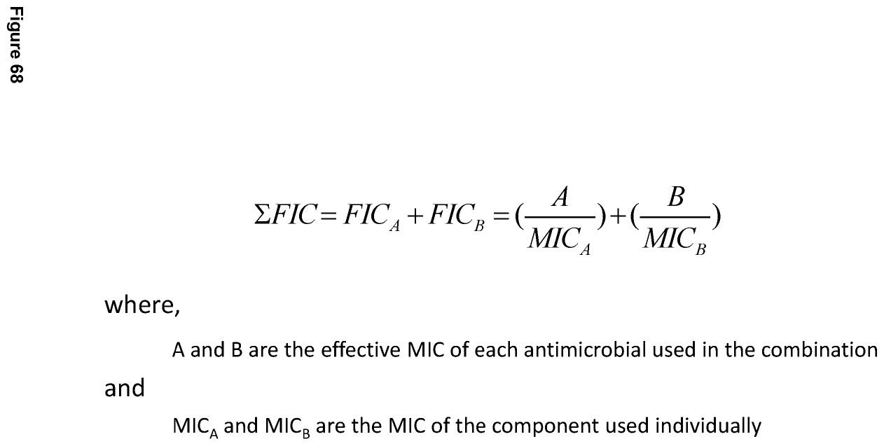

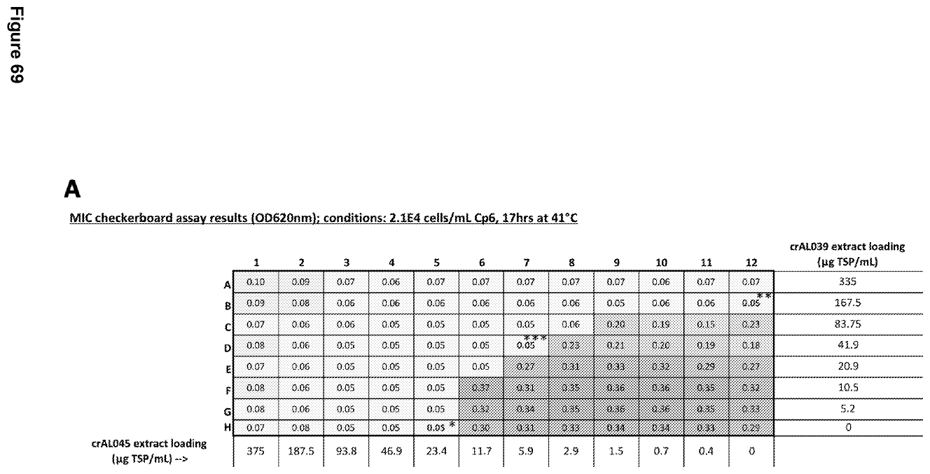

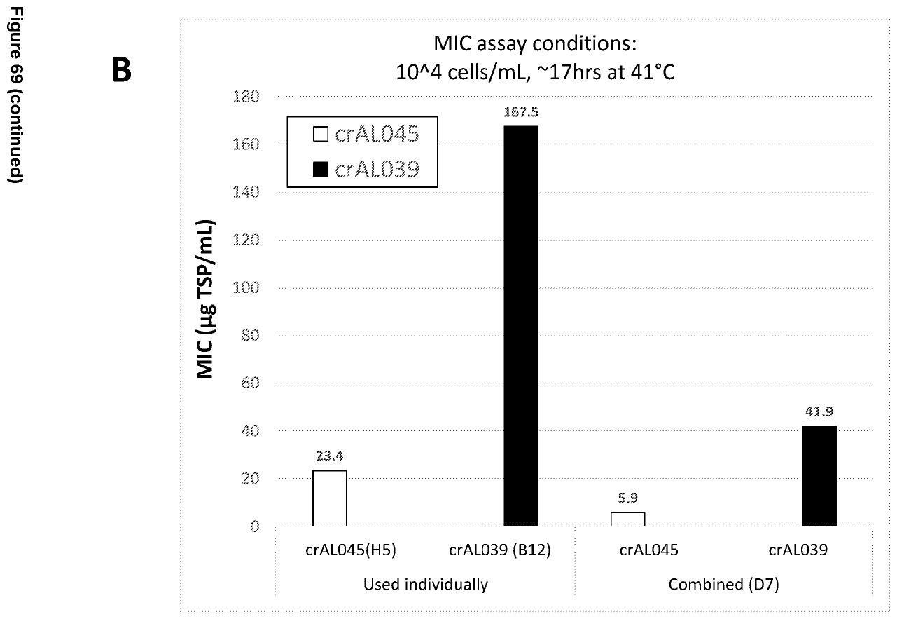

With respect to the growth of the three strains, all reached comparable OD600 nm after IPTG induction which suggested that the expression of tRNA-Wtca was not significantly toxic to the cell. RFP expression was confirmed visually when cells carrying the ecAL004 plasmid were visibly red after 6 h post-induction (FIG. 1A), and clearly fluorescent when illuminated under UV light (FIG. 1B). In contrast, cells carrying either the ecAL002 or the ecAL003 plasmids exhibited no color nor fluorescence. SDS-PAGE analysis of clarified lysates provided additional confirmation. Staining of the gels with InstantBlue (Expedeon) revealed a protein band over the 25 kDa size protein marker, which was present only in cell extracts carrying the ecAL004 plasmid and that harboured both tRNA-Wtca and RFP coding sequences (FIG. 1C). As the expected molecular weight of RFP was 27 kDa, it confirmed that RFP was being expressed as the samples also coincided with the indicated visual observations. In contrast, this band at 27 kDa was absent in the protein extracts obtained from the other two strains. The results therefore strongly indicated that the tRNA-Wtca sequence cloned into the pETDuet construct was correctly being transcribed and processed, and that without it, expression of synthetic genes with WTGG-TGA codons could not be observed.

### Example 3: Construction of Endolysin Expression Vectors and E. coli Transformation

The ecAL002 vector described in Example 2 was used as the expression vector for phage-derived endolysins. Endolysin coding sequences described in Example 1 were modified to replace one or two tryptophan codons “TGG” with the stop codon “TGA” (i.e. WTGG-TGA). In this way, the recombinant endolysin is translated only when a modified tRNA-Wtca molecule is co-expressed alongside, as described in Example 2. Additionally, N-terminal FLAG (DYKDDDDK), C-terminal HA (YPYDVPDYA), C-terminal Strep (WSHPQFEK) or C-terminal 6×HIS (HHHHHH) tags were added to the endolysins coding sequences (Table 5). FLAG-tags were added to the N-terminus of endolysins AMI3CPF4969 (SEQ ID NO: 11), GH25CPFORC3 (SEQ ID NO: 1) and GH25phiS63 (SEQ ID NO: 5), HA-tags were added to the C-terminus of endolysins AMI2CPJP838 (SEQ ID NO: 9), AMI2phiZP2 (SEQ ID NO: 3), AMI2phiCPV4 (SEQ ID NO: 2) and GH25CPF4969 (SEQ ID NO: 8), and Strep-tags were added to the C-terminus of endolysins AMI3CPFORC25 (SEQ ID NO: 6), AMI3CPJP55 (SEQ ID NO: 10), AMI3CPSM101 (SEQ ID NO: 4) and AMI3phi24R (SEQ ID NO: 7). Additional expression vectors were constructed to express AMI2phiCPV4 (SEQ ID NO: 2) and GH25CPF4969 (SEQ ID NO: 8) endolysins with C-terminal 6×HIS-tag. For C-terminal HA-, Strep- and 6×HIS-tags, a ‘GSAGSG’ flexible linker was also included in between the tag and the endolysin protein. The endolysin coding sequences with W(TGG->TGA) codon modifications fused to N-terminal FLAG tag or C-terminal HA and Strep tags were externally synthesized (Eurofins Genomics). The synthesized genes were then PCR-amplified using specific primers with 20-nt tails (Table 18) which matched to sequences of the MCS2 on ecAL002. This allowed directional cloning into the vector by the Gibson Assembly method. The PCR conditions consisted of an initial denaturation step at 94° C. for 5 minutes, followed by 30 cycles at 98° C. for 10 s, 63-68° C. for 30 s and 72° C. for 50 s, and a final extension step at 72° C. for 10 min. PCR was performed using the High-Fidelity DNA polymerase Phusion (New England Biolabs), following manufacturer instructions. Amplified DNA fragments were purified using the QIAquick Gel extraction kit (QIAgen), and cloned into the ecAL002 vector using the NEBuilder HiFi DNA assembly kit (New England Biolabs) following manufacturer's instructions. Cloning reactions were transformed into NEB® 10-beta competent E. coli cells, and the transformants were selected on LB media containing 100 μg/mL ampicillin. DNA plasmids were extracted from transformant cultures using the QIAprep Miniprep kit (QIAgen) and sequence-verified by Sanger sequencing (Eurofins Genomics).

Plasmids ecAL006 and ecAL007 described in Table 5 were modified in order to replace the C-terminal HA tags fused to AMI2phiZP2 and AMI2phiCPV4 with C-terminal 6×HIS tags. Plasmid modification was performed by site-directed mutagenesis using the Q5 site-directed mutagenesis kit (New England Biolabs). Primer pairs O_341/O_351 and O_342/O_351, described in Table 18, were used to amplify by PCR the full length of ecAL006 and ecAL007, respectively. Primer 0351 carried the sequence required for the synthesis of the 6×HIS tag. Amplification and enzymatic modification of the amplified DNA fragment were performed as recommended by the site-directed mutagenesis kit manufacturer. Reactions were transformed into NEB®10-beta competent E. coli cells, and the transformants were selected on LB media containing 100 μg/mL ampicillin. The resultant plasmids, plAL073 (encoding 6×HIS-tagged AMI2phiZP2) and plAL071 (encoding 6×HIS-tagged AMI2phiCPV4), were extracted from transformant cultures using the QIAprep Miniprep kit (QIAgen) and subsequently sequence-verified (Eurofins Genomics).

Table 5 tabulates the vectors constructed for expression of tagged endolysins in E. coli and Table 18 the primer sequences used for the construction of expression vectors. The plasmids were subsequently transformed into the Rosetta™ (DE3) pLysS E. coli strain (Merck Millipore) following manufacturer instructions. The corresponding bacteria strain ID is also tabulated in Table 5.

### Example 4: Endolysin Expressions from E. coli

E. coli strains bAL007 to bAL017, in addition to the bAL002 control strain (Table 5), prepared as indicated in Example 3, were cultured to confirm endolysin expressions prior to performing any biochemical assays.

Culture of the expression strain of interest was initiated by inoculating 5 mL LB medium containing 100 μg/mL ampicillin and 25 μg/mL chloramphenicol with a small scrape sample of frozen 50% glycerol stock. The 5 mL inoculum was cultured overnight (14-18 hrs) in an incubator at 37° C. with 275 RPM shaking. After overnight growth when the E. coli culture has reached stationary phase, 1 mL of the overnight inoculum was inoculated into 100 mL LB medium containing 100 μg/mL ampicillin and 25 μg/mL chloramphenicol (i.e. effectively 100× dilution). The 100 mL LB medium in 500 mL shake flask was cultured at 37° C. with 275 RPM shaking. After 2.5 hrs of pre-induction growth, endolysin expression was induced by adding 1 mM IPTG to the 100 mL culture. Thereafter, the incubator temperature was lowered to 30° C. and incubation of the culture was continued for an additional 5 hrs. Harvest of the cells was done by transferring 25 mL of each culture into a 50 mL Falcon tube and centrifuging at 3800×g at 4° C. for 15 min in a centrifuge. The medium was removed and the cell pellet was stored at −20° C. if not immediately used. Protein extraction from a cell pellet derived from 25 mL of culture was done via sonication-lysis. The cell pellet was first resuspended in 1 mL PBS, pH 7.4 before being lysed using sonication (10 s on/off at 10 μm amplitude for 10 cycles). Afterwards, the crude lysate was centrifuged at 16600×g for 30 min at 15° C. The clarified lysate was subsequently diluted in PBS, pH 7.4, for SDS-PAGE analysis.

Confirmation of endolysin expression was done using SDS-PAGE. Clarified lysates were prepared for SDS-PAGE by mixing 1:1 with 2× Laemmli buffer and adding 100 mM DTT. Thereafter, the samples were boiled at 99° C. for 15 min. A 500 μg/ml BSA standard was also prepared similarly to serve as a reference. 10 μL of the prepared samples and BSA standard were loaded onto a 4-15%, 15-well TGX gel (Bio-rad) setup in a Bio-rad Mini Protean gel tank (Bio-rad) with 1×TGS running buffer (Bio-rad). SDS-PAGE was run for 60 min with constant 150V using a PowerPac Basic power supply (Bio-rad). After the SDS-PAGE run, the protein gel was stained with InstantBlue (Expedeon) and de-stained with MQ H2O thereafter. The de-stained gel was imaged on a Vilber Bio-Print imager (Vilber) with Bio-Vision software (Vilber).

SDS-PAGE results showed that endolysins were expressed in all strains except for bAL014, when compared to the background protein profile of the control strain bAL002 (FIG. 2).

### Example 5: Culturing and Banking of C. perfringens Reference Strains

As C. perfringens is an anaerobe, in order to maintain viability of the bacteria, all plastic consumables, buffers, growth medium, and agar plates were pre-reduced in a Don Whitley MACS MG 500 anaerobic workstation (Don Whitley Scientific) to remove oxygen exposure to the organism. All manipulations of the bacteria were also performed in the anaerobic workstation unless otherwise stated.

Eight C. perfringens Type A reference strains (NCTC: 2837, 8235, 8237, 8238, 8239, 8359, 8678, 10578) were procured from the Public Health England culture collection and banked for future use in enzybiotic assays. Briefly, the freeze-dried culture powder was reconstituted and resuspended in 500 μL RCM (Oxoid). After which, 150 μL of the reconstituted mixture was used to inoculate double cooked meat medium (Southern Group Laboratory) in universal glass bottles. The cultures were grown overnight at 37° C. in the anaerobic workstation. Stocks of each strain were stored at 4° C.

### Example 6: Culturing and Banking of DSMZ Strains

As the non-C. perfringens strains procured are anaerobes and to maintain viability of the bacteria, all plastic consumables, buffers, growth medium, and agar plates were pre-reduced in a Don Whitley MACS MG 500 anaerobic workstation to remove oxygen exposure to the organism. All manipulations of the bacteria were also performed in the anaerobic workstation unless otherwise stated.

Clostridium colinum (DSMZ 6011), Clostridium leptum (DSMZ 753), Clostridium cellobioparum (DSMZ 1351), and Bifidobacterium adolescentis (DSMZ 20083) were procured from the Leibniz Institute DSMZ-German Collection of Microorganisms and Cell Cultures and banked for future use in enzybiotic assays. Briefly, the freeze-dried culture powder was reconstituted and resuspended in 500μL RCM. After which, 150 μL of the reconstituted mixture was then used to inoculate double cooked meat medium in universal glass bottles. The cultures were grown overnight at 37° C. in the anaerobic chamber. Stocks of each strain were stored at 4° C.

### Example 7: Culturing and Banking of C. perfringens Field Isolates

As C. perfringens is an anaerobe and to maintain viability of the bacteria, all plastic consumables, buffers, growth medium, and agar plates were pre-reduced in a Don Whitley MG500 anaerobic workstation to remove oxygen exposure to the organism. All manipulations of the bacteria were also performed in the anaerobic chamber unless otherwise stated.

Eight C. perfringens Type A field isolates (APHA: B00907, B00917, B00954, B00964, B00976, G00033, W000101, W00102) were procured from the United Kingdom Animal and Plant Health Agency and banked for future use in enzybiotic assays. Briefly, the culture swab transported in Amies agar was used to inoculate 15 mL double cooked meat medium universal glass bottles. The cultures were grown overnight at 37° C. in the anaerobic chamber. Stocks of each strain were stored at 4° C.

### Example 8: Screening of C. perfringens Reference Strains Using Clarified Lysates

The endolysin bacteriolytic activity of clarified lysates obtained from shake flask cultures expressing AMI2CPF4969, AMI2CPJP838, AMI2phiCPV4, AMI2phiZP2, AMI3CPFORC25, AMI3CPJP55, AMI3CPSM101, AMI3phi24R, GH25CPF4969 and GH25CPFORC3, as well as from a culture transformed with the empty plasmid ecAL002, were tested against the eight C. perfringens reference strains described in Example 5 using spot assays. The clarified lysate from the strain expressing AMI3phi24R was also screened since it was possible that the endolysin may still be expressed but not abundant enough to be observed on a protein gel.

Working stock culture of each reference strain was inoculated at 100-fold dilution in BHI+C medium and incubated overnight at 37° C. in a Don Whitley MACS MG 500 anaerobic workstation. For each strain, overnight stationary phase Cp cultures were diluted 10-fold using RCM and 900 μL of the of the diluted culture was plated on RCM+10% agar plates. These plates were allowed to grow into cell lawns after incubating overnight at 37° C. in the anaerobic workstation. These plates were designated as “cell-lawn” plates. Additionally, another set of RCM+1% agar plates were plated with 900 μL 10-fold diluted culture. These plates were designated “freshly-plated cells” plates and were used immediately for spot assays. bAL002 and bAL007—bAL017 E. coli cell pellets derived from 20 mL cultures were resuspended in 500 μL in vitro GIT buffer (2.4 g liter—1 beef extract, 5.0 g liter—1 yeast extract, 2.5 g liter—1 glucose, 10.0 g liter—1 tryptose, 0.6 g liter—1 L-cysteine hydrochloride, and 5.0 g liter—1 NaCl; pH 6.4). The resuspended cells were lysed using sonication (10 s on/off at 10 um amplitude for 10 cycles). Thereafter, the crude lysate was centrifuged at 16600×g for 30 min at 15° C. 10 μL clarified lysates were dispensed onto Whatman filter papers placed on either cell-lawns or a freshly-plated cells. 10 μL clarified lysate from a bAL002 culture was spotted on the plates as a negative control. Additional negative controls spotted include PBS 7.4 and in vitro GIT buffers with and without overnight pre-reduction in the anaerobic chamber. Positive controls prepared and also spotted consisted of 20 μg/ml ampicillin, 10 μg/ml chloramphenicol, 20 μg/ml spectinomycin, 1 mg/mL lysozyme, 1 mg/mL or 10 mM disodium EDTA pH 7.8, and 1 mg/mL lysozyme+1 mg/mL or 10 mM disodium EDTA. After spotting 10 μL sample onto either cell-lawns and freshly-plated cells prepared from each strain, the agar plates were incubated overnight at 37° C. in the anaerobic workstation. Spot assay results were imaged on a Vilber Bio-Print with Bio-vision software. The C. perfringens strains were screened in 2 sets: 1) NCTC 2837, 8235, 8238, 8678, and 10578; 2) NCTC 8237, 8239, and 8359.

The relative antimicrobial activities of the endolysins on the 8 reference strains were qualitatively assessed based on the presence and size of the clearance zones generated from freshly-plated cells and cell lawns. Spot assays on freshly-plated cells (FP) were used to evaluate the endolysin's inhibitory effects on bacterial growth. Alternatively, spot assays on cell lawns (CL) were used to assess the bacteriolytic activity of the endolysin. In both tests, if the endolysin exhibits either inhibitory effects or bacteriolytic activity, a clearance zone would appear around the filter paper to which the endolysins was added. Example spot assay results from each screening set are respectively shown in FIG. 3 and FIG. 4. A summary of the antimicrobial responses of the various C. perfringens strains to the E. coli-expressed endolysin lysates is shown in Table 6. Screening set 1 revealed that generally the expressed endolysins are active against the strains tested and their relative activities depended on the strain it was screened against. The bacteriolytic activity of an endolysin varied depending on whether the C. perfringens strains screened on freshly-plated cells or cell lawns. These differences could be due to the strain's growth rate, cell wall re-modelling during stationary phase, and/or the bacterial loading on the surface of the agar plate. Additionally, set 1 results showed that ampicillin was effective on freshly-plated cells but not on cell lawns which have already grown. This result agrees with the bacteriostatic activity of ampicillin. In contrast, some endolysins exhibited antimicrobial activities on both type of plated cells. Chloramphenicol and spectinomycin antibiotics generally had no effect on inhibiting C. perfringens growth, nor 1 mg/mL lysozyme and 1 mg/mL disodium EDTA. Similar results were observed in screening set 2. In this set, lysates bAL007 and bAL012 were not screened as they exhibited no activity at all from the prior screen. A higher concentration of EDTA (10 mM) was shown to have an inhibitory effect on freshly-plated cells. 1 mg/mL Lysozyme had a low effect on the strains tested in set 2 but only when freshly-plated. From this semi-quantitative assay, five endolysins (AMI2phiCPV4 (bAL009), AMI2phiZP2 (bAL010), AMI3phi24R (bAL014), GH25CPFORC3 (bAL016), and GH25phiS63 (bAL017)) were selected to take forward for additional characterization. They were selected based on their broad antimicrobial activity on different C. perfringens strains, activity against both freshly-plated cells and cell lawns, including their expression levels estimated from SDS-PAGE analyses performed in Example 4.

### Example 9: Screening of E. coli-Expressed Endolysins on 4 Non-CP Bacteria Species

In addition to screening against C. perfringens strains, E. coli-expressed endolysins were also screened against 4 non-Cp bacteria species commonly found in poultry gut to determine whether the endolysins identified were specific to only Cp. Screening of the 4 non-CP species Clostridium colinum (DSMZ 6011), Clostridium leptum (DSMZ 753), Clostridium cellobioparum (DSMZ 1351), and Bifidobacterium adolescentis (DSMZ 20083) were done in parallel to screening set 2 in Example 8. Thus, the methodology and spot assay sample layout are also as described previously.

Screening results and summary of E. coli-expressed endolysins on the 4 non-target bacterial species are shown in FIG. 5 and Table 7, respectively. Depending on the species, both 20 μg/ml ampicillin and 10 mM disodium EDTA exhibited inhibitory effects on freshly-plated cells but did not have any effects on cell-lawns. This was similarly observed for chloramphenicol. Neither endolysin clarified lysate, nor lysozyme, had antimicrobial activity on any of the non-target bacteria species screened. This result suggests that the endolysins identified might be specific only to Cp strains.

### Example 10: Affinity Purification of AMI2phiCPV4, AMI2phiZP2, and GH25CPFORC3 Endolysins

Affinity purifications of the top 3 endolysins from bAL009 (AMI2phiCPV4), bAL010 (AMI2phiZP2), and bAL016 (GH25CPFORC3) E. coli strains was performed. This was to produce purified and concentrated endolysin stocks without contaminant native E. coli lysate proteins that were found to be interfering with preliminary antimicrobial assays (unpublished results).

Shake flask expression of the respective endolysins from bAL009, bAL010, and bAL016 E. coli strains were done as previously described in Example 4. 200 mL LB growth medium instead of 100 mL in a 1 L shake flask was used for each endolysin expression. The inoculation and antibiotic volumes were also scaled accordingly. After harvesting, for each strain, 4 cell pellets each derived from approximately 50 mL of culture were obtained. Protein extraction from each cell pellet was done via sonication-lysis. The cell pellet was first resuspended in 1 mL PBS, pH 7.0, before being lysed using sonication (5 s on/off at 10 μm amplitude for 10 cycles). Afterwards, the crude lysate was centrifuged at 16600×g for 30 min at 15° C. The clarified lysate from each pellet was mixed with 24 mL PBS, pH 7.0+10% glycerol in a 50 mL Falcon tube. One tablet of cOmplete, mini, EDTA-free protease inhibitor was added to each tube. Monoclonal anti-HA agarose, clone HA-7 affinity matrix was used to bind HA-tagged AMI2phiCPV4 and AMI2phiZP2 endolysins whereas Anti-FLAG M2 affinity gel was used to bind FLAG-tagged GH25CPFORC3 endolysin. After washing the affinity matrix in accordance to vendor instructions, 400 μL suspension of the corresponding matrix in PBS, pH 7.4 was mixed with 25 mL of the lysate/PBS/glycerol solution. The tagged endolysins were bound to the affinity matrix overnight (˜14-16 hrs) with gentle mixing at 4° C. After overnight binding, each protein binding suspension was centrifuged at 3800×g at 20° C. for 5 min to pellet the affinity matrix. The supernatant was carefully removed from each tube and the affinity matrix was carefully resuspended in 500 μL PBS, pH 7.0 buffer and transferred to a Pierce spin column (Thermofisher). Each spin column with affinity matrix was centrifuged at 8000 RPM, 30 sec to remove the PBS. 500 μL PBS, pH 7.0 buffer was added to the spin column and centrifuged again to wash the affinity matrix. This was repeated 6 times. FLAG-tagged and HA-tagged endolysins were eluted by incubating the affinity matrix respectively in 500 μL 100 μg/mL FLAG peptide in PBS, pH 7.0 or 100 μg/mL HA peptide in PBS, pH 7.0. Each elution was incubated for greater than 15 min at room temperature with gentle mixing. The elution fractions were collected in a new 2 mL microfuge tube by centrifuging the spin columns at 8000RPM, 30 sec. This was repeated 6 times for each spin column with affinity matrix. The elution fractions derived from the same endolysin expression were pooled and concentrated using a Vivaspin 20, 10 MWCO PES membrane concentrator (Sartorius AG). The concentrators were centrifuged at 3000×g for 30 min at 20° C. or until the concentrate was less than 200 μL. 10 mL PBS pH 7.0 buffer was added to the concentrate and centrifuged again at the same conditions to remove residual elution peptide. The concentrate was concentrated until it was less than 200 μL and then transferred into a 1.5 mL microfuge tube. After aliquoting the purified and concentrated endolysin stocks for SDS-PAGE analysis, 5 μL aliquots were dispensed into 8-well strip tube wells and stored by freezing at −80° C.

SDS-PAGE and densitometry analysis of the purified endolysin stocks was done to quantify the concentration of the purified endolysins. A BSA standard curve consisting of the following final concentrations in 2× Laemmli buffer was prepared: 12.5 μg/mL, 25 μg/mL, 50 μg/mL, 100 μg/mL, 200 μg/mL. 10×, 20×, and 40× dilutions of the purified endolysin stocks were done using 2× Laemmli buffer. 100 mM DTT was added to the BSA and diluted endolysin Laemmli samples and boiled at 99° C. for 15 min. 10 uL of the prepared samples and BSA standards were loaded onto a 4-15%, 15-well TGX gel setup in a Bio-rad Mini Protean gel tank with 1×TGS running buffer. SDS-PAGE was run for 80 min with constant 150V. After the SDS-PAGE run, the protein gel was stained with InstantBlue and de-stained with MQ H2O. The de-stained gel was imaged on a Vilber Bio-Print with Bio-vision software. Densitometry analyses for quantification of the purified endolysin stock concentrations were done also using the onboard software.

SDS-PAGE analyses of buffer-exchanged and concentrated elution fractions showed that the enzymes were purified. SDS-PAGE migration of the endolysin protein bands do not coincide exactly with the theoretical molecular weights of GH25CPFORC3 (˜40 kDa, lanes 7-9), AMI2phiCPV4 (˜26 kDa, lanes 10-12) and AMI2phiZP2 (˜26 kDa, lanes 13-15). The reasoning for this was not investigated further but it's clear that the protein of interest was purified abundantly with minimal contaminant proteins. The BSA standards curve was used for densitometry analysis and quantification of the purified endolysin stocks. While the BSA standards showed some degraded protein fragments, it was considered negligible compared to its major band at ˜66.5 kDa. Densitometry analysis and quantification showed that the GH25CPFORC3, AMI2phiCPV4, and AMI2phiZP2 stock concentrations were approximately 2400 μg/mL, 750 μg/mL, and 1000 μg/mL, respectively.

### Example 11: Spot Assay on C. perfringens Strains Using Purified Endolysins

In Example 8, spot assays using clarified lysates were performed to screen for antimicrobial activities of E. coli-expressed endolysins. In the present Example, endolysin antimicrobial activities using purified proteins were examined and tested against additional C. perfringens strains.

NCTC reference strains and APHA field isolates were cultured as described in Example 5 and 7 respectively. Purified endolysins were immuno-purified as described in Example 10. The antimicrobial activity of each endolysin was evaluated using freshly plated cells or cell lawns and the preparations were similarly to that described in Example 8, except that 750 μL of stationary phase culture was used to inoculate 140 mm diameter RCM, 1% agar, plates. Endolysins at concentrations 100 μg/mL, 50 μg/mL and 10 μg/mL were also spotted similarly onto freshly-plated cells and cell lawns. Briefly, 10 mm paper disks were placed on top of freshly-plated cells or cell lawns and 10 μl of purified endolysins (100 μg/mL, 50 μg/mL, or 10 μg/mL) were spotted onto the disks. Two replicates of buffer controls (PBS, pH 7.4) and 50 μg/mL lysozyme controls were also spotted. Thereafter, the agar plates were incubated at 37° C. for 44-48 h for the development of clearance zones.

A summary of the spot assay results is shown in Table 8 and Table 9. The GH25CPFORC3 endolysin was able to inhibit bacterial growth of most strains and isolates, while AMI2phiCPV4 and AMI2phiZP2 were not able to inhibit bacterial growth at the concentrations tested (Table 8). The level of inhibition by GH25CPFORC3 varied from strain to strain, although the field isolates tended to be more sensitive to endolysin action than the reference strains. At the concentrations tested, GH25CPFORC3 exhibited no inhibitory effects against reference strains NCTC 8237 and NCTC 8238. All three endolysins were bacteriolytic and created clearance zones in most strains, with GH25CPFORC3 clearance zones being the most prominent (Table 9). In general, it appeared that GH25CPFORC3 exhibited the most antimicrobial activity of the three endolysins tested and also had a broader spectrum of antimicrobial activities against the C. perfringens strains screened. The current spot assay results also suggested that the antimicrobial activities of AMI2phiCPV4 and AMI2phiZP2 are similar. In contrast to purified endolysins, 50 μg/mL lysozyme did not show any inhibitory effect nor any bacteriolytic activity against any of the strains tested.

In conclusion, GH25CPFORC3 was ranked as the highest performing endolysin whereas AMI2phiV4 and AMI2phiZP2 performed similarly in the spot assays. Additionally, GH25FORC3 exhibited a broad spectrum of antimicrobial activities against different C. perfringens strains.

### Example 12: Turbidity Reduction Assays

The relative bacteriolytic activities of the purified GH25CPFORC3 (i.e. 'FORC3), AMI2phiCPV4 (i.e. 'V4), and AMI2phiZP2 (i.e. 'ZP2) endolysins were assessed using a turbidity reduction assay at room temperature (i.e. 23° C.) and in PBS, pH 7.0 buffer where it is assumed that cell growth is minimal. The assay was performed to rank the lytic performances between the 3 endolysins as well as to characterize each endolysin's dose responses on different C. perfringens strains. Chicken hen egg white lysozyme (sigma, known to be bacteriolytic against C. perfringens, was also included in the assay as a reference enzybiotic.

4 reference strains (NCTC 2837, 8237, 8238, 8239) were inoculated and grown overnight to stationary phase as described in Example 8. Stationary phase cells from each culture were used to inoculate into fresh BHI+C medium and incubated in the anaerobic chamber at 37° C. for 1.5-2 hrs in order for the cultures to reach exponential phase. The cultures were then pelleted at 3800×g for 30 min at 10° C. when the OD620 nm was ˜1.0 and the spent BHI+C supernatant removed. The cell pellets were kept at room temperature until they were required for initiating the turbidity reduction assay. The cells were resuspended in half the volume in PBS, pH 7.0 buffer to concentrate the cells. The resuspended C. perfringens cells were subsequently used to initiate the turbidity reduction assay by mixing 90 μL per well of the resuspended cells with 10 μL of 10× concentrated lysozyme (Sigma) or endolysin (prepared as described herein) in the following 96-microwell plate format (FIG. 8). Except for lysozyme, each endolysin was assayed in 3× replicates at each loading concentration. The 2× serially-diluted lysozyme and endolysin stocks were prepared using PBS, pH 7.0 buffer. Column 1 and 2 correspond to the PBS control (100 μL/well PBS, pH 7.0 only) and blank control (10 μL/well PBS, pH 7.0+90 μL/well resuspended cells). Effective loading concentrations of each 96-microwell turbidity reduction assay plate are also shown in FIG. 8.

The turbidity reduction of the plate was monitored by performing an OD620 nm kinetic read with intervals on a plate reader at 23° C. (i.e. room temperature). The assay plate was monitored for 30-60 min, or until the OD620 nm reduced significantly such that it began to plateau with the higher endolysin loadings.

Turbidity reduction assay results showed that all 3 endolysin had bacteriolytic activity against the 4 C. perfringens strains (NCTC 2837, 8237, 8238, 8239) tested. This was compared against the blank wells (i.e. cell suspension control) as some background cell lysis was observed. Two sets of plots were prepared for each Cp strain: 1) endolysin ranking at each loading concentration (FIG. 10, FIG. 12, FIG. 14, and FIG. 16) and 2) dose responses for each endolysin (FIG. 9, FIG. 11, FIG. 13, and FIG. 15). At high endolysin loadings (i.e. 5 μg/mL), the bacteriolytic activities between the 3 endolysins ranked in the following order: GH25CPFORC3, AMI2phiCPV4, AMI2phiZP2. However, at loading concentrations lower than 1.25 μg/mL, AMI2phiCPV4 generally exhibited higher lytic activities compared to GH25CPFORC3. The dose responses of the endolysins on each strain were appropriate with higher turbidity reduction drops with increasing loading concentrations. The dose response varied depending on the endolysin as well as the C. perfringens strain. With 0.31 μg/mL loading concentrations, the bacteriolytic activities were effectively similar to background lysis for all 3 endolysins against the four C. perfringens strains. Except for strains NCTC 2837 and NCTC 8238, lysozyme generally exhibited minimal bacteriolytic activities. Additionally, at loading concentrations higher than 31 μg/mL, lysozyme stocks precipitated upon mixing with the resuspended cells in PBS, pH 7.0 buffer. Lysozyme's bacteriolytic activities against NCTC 8238 was the most significant but required higher loading and incubation time to reach similar levels of turbidity reduction compared to the 3 endolysins.

Comparison of turbidity reduction assay results with that of the spot assays performed in Example 10 suggested that the growth and recovery of freshly-plated cells likely had an effect on the lack of clearance zones observed. All 3 endolysins exhibited bacteriolytic activities against the NCTC strains harvested at their exponential phase of growth. These included AMI2phiCPV4 and AMI2phiZP2 endolysins that exhibited no clearance zones for freshly-plated cells. Also, NCTC 8237 and NCTC 8238 strains that appeared to be resistant to GH25CPFORC3 in spot assays on freshly-plated cells, here exhibited sensitivity to the endolysin. Interestingly, NCTC 8237 and NCTC 8238 were observed to be the most susceptible and the most resistant C. perfringens strains tested in the turbidity reduction assay, respectively. Similarly, 50 μg/mL lysozyme which was shown to have no antimicrobial activity at all in spot assays was revealed to have minor activities against the reference strains tested here. Consequently, turbidity reduction assays on resuspended cells allowed direct assessment of an endolysin's bacteriolytic activities without convoluting factors such as C. perfringens growth and recovery rate.

A summary on the bacteriolytic activities of each endolysin on the 4 strains is tabulated in Table 10 and plotted in FIG. 17. The table and scatter plots summarize the t50% lysis parameter, which is defined as the time required for an endolysin, at a particular loading concentration, to lyse 50% of a cell suspension. Calculation was done by comparing an endolysin's lysis profile against the blank control OD at the beginning of the kinetic read (i.e. t=0 h) and with the PBS control OD (i.e. 0.03) where it's assumed to correspond to complete cell lysis. Equation 1 below describes the calculation performed to extract the t50% lysis parameter from a turbidity reduction profile. The parameter was able to convey more concisely the rankings between the endolysins as well as each endolysin's dose responses in FIG. 17. The scatter plots revealed clearly that NCTC 8237 appeared to be the most susceptible to the endolysins while NCTC 8238 was the most resistant. t50% lysis values could not be obtained for lysozyme turbidity reduction assays except for NCTC 8238, as it was the most sensitive to lysozyme. Interestingly, NCTC 8238 was found to be the most resistant strain against the 3 endolysins.

In conclusion and similarly to that in spot assay results in Example 11, turbidity reduction assay results confirmed that GH25CPFORC3 had the highest antimicrobial performance. The turbidity reduction assay was also able to elucidate that AMI2phiCPV4 was more bacteriolytic than AMI2phiZP2, which the spot assay could not inform.

### Equation 1

Formula used to determine the time at which 50% lysis of has occurred. The equation was used to solve for OD_{E_t50% lysis}, corresponding to 50% reduction of the initial cell suspension loading at time 0 h (i.e. OD_{b_t=0}). The time at which 50% lysis occurred (i.e. t_{50% lysis}) was then determined from the turbidity reduction profile when the OD reached OD_{E_t50% lysis}.

$$50 \% = \frac{{OE}_{{E \_ t}_{50 \% lysis}} - 0.03}{{OD}_{b \_ t = 0} - 0.03} \times 100 \%$$

### Example 13: Log₁₀ Reduction Assays

To better characterize the antimicrobial activities of the purified endolysins, log 10 reduction assays were carried out for each endolysin using four C. perfringens reference strains (NCTC2837, NCTC8237, NCTC8238, NCTC8239). The assay provided a more accurate determination on the endolysins' antimicrobial activities as it tests a reference strain's level of viability with treatment.

The assay consisted of determining the reduction in viability of actively growing cells after endolysin treatment. C. perfringens strains were grown in BHI+C under anaerobic conditions at 37° C. to approximately 0.6 OD600 nm. The 1.8 mL culture was centrifuged at 16600×g for 5 min at room temperature. The media was removed and the cell pellet was resuspended in 0.9 mL of resuspension buffer (NaCl 127 mM, Na2HPO4 70 mM, NaH2PO4 30 mM pH 7.0). Cell suspension was then diluted with buffer to 0.6 OD600 nm (˜10-20 million cells/ml). 20 μL of purified endolysin at a concentration of 10 μg/mL or 50 μg/mL, or PBS pH7.4 (for the “untreated” cells control), was added to the 180 μL of bacterial suspension, resulting in a final concentration of 1 or 5 μg/mL of endolysin. Reactions were incubated at 25° C. for 1 h and then viable cell count was determined by serial dilutions and plating on RCM media.

A summary of the log 10 reduction assay results for the 3 endolysins on each reference strain is shown in Table 11. From diluted cultures of ˜0.6OD600 nm, it was observed that the average initial bacterial loading across all reference strains used for the log 10 reduction assays was approximately 15.3 million cells/mL. However, spontaneous cell death was observed in resuspension buffer for 1 h and ranged from 10-95% of the initial loading. No clear correlation could be determined for the observations though the NCTC 8238 and NCTC 8239 cells' viabilities appeared to be consistently impacted negatively after resuspension in PBS, pH 7.0 buffer. To correct for endolysin-independent cell death during the assay, log 10 reduction values were calculated as the log 10 of the ratio of untreated cells control over endolysin-treated cells. Additionally, the log 10 reduction values were used to calculate the percentage reduction of viable bacteria for each endolysin. Assay results showed that the three endolysins were active against all four tested strains including at the lowest concentration (1 μg/ml), although with different efficacies. The log 10 reduction values ranged from 0.03 to 3.76, with GH25CPFORC3 generally being the endolysin with the highest activity against a C. perfringens strain (FIG. 19). At the highest concentration (5 μg/mL), the median percentage reduction across all strains was 85.80% for GH25CPFORC3, while the median percentage reduction for AMI2phiCPV4 and AMI2phiZP2 was 75.79% and 51.37%, respectively. These results supported the endolysin performance rankings established from Example 11, from highest performance to the lowest: 1) GH25CPFORC3, 2) AMI2phiCPV4, 3) AMI2phiZP2.

### Example 14: Algal Expression System for Endolysin Expression

The algal expression system used to express endolysin genes in microalgae consisted of the following steps:
(1) A modified strain of Chlamydomonas reinhardtii;
(2) DNA vectors designed to integrate the endolysin genes into the chloroplast genome of the algae;
(3) Alga transformation using the glass bead method [1]; and
(4) selection based on photosynthesis restoration applied to substitute all original chloroplast genome copies by the endolysin-containing genomes (homoplasmy).

### Strain

The recipient Chlamydomonas reinhardtii strain for endolysin expression is TN72 [1]. This strain is an mt+ cell-wall deficient derivative of the wild type isolate 137c, equivalent to CC-125 [2]. In the TN72 strain the psbH gene of the chloroplast genome was deleted by gene replacement with the spectinomycin resistance gene aadA [1]. The psbH gene encodes for a component of Photosystem II, which is involved in light assimilation for photosynthesis. Therefore, the absence of the psbH protein renders cells that cannot grow photo-autotrophically and that require acetate in the media in order to grow.

### Vectors

Recombinant endolysin genes were introduced into the Chlamydomonas reinhardtii chloroplast by partial integration of plasmids derived from pAxi2.0 or pL2_psbH plasmids. These plasmids carry a 0.8 Kb sequence homologous to a sequence downstream of the psbH gene of the algal chloroplast genome, and a 2.4 Kb sequence homologous to a region that contains the psbH gene and some of the upstream sequence. Endolysins coding sequences, flanked by 5′ and 3′ regulatory sequences, were cloned in between the two homologous sequences. The 5′ and 3′ regulatory sequences that drive the expression of the endolysin genes were derived from the psbA, psaA, 16S rDNA, atpA and rbcL chloroplast genes. In addition, both pAxi2.0 and pL2_psbH plasmids also carry the tRNA-W_{tca} gene, which transcribes for a modified tryptophan tRNA molecule that allows translation of codons “TGA” as Tryptophan instead of STOP. Endolysin genes were synthesized in vitro, and cloned into pAxi2.0 and pL2_psbH plasmids using BsaI sites.

### Transformation Method (Glass Bead Method)

For transformation, the TN72 strain was grown in 400 mL TAP media under constant light, 25° C. and 165 rpm orbital shaking, to an optical density of 0.3-0.6 OD750 nm. The culture was then centrifuged at 2000×g for 10 min, and the cell pellet was resuspended in 1 mL of TAP media. 0.5 mL of the algal suspension was mixed with 40-100 μg of a pAxi2.0 or pL2_psbH-derivative plasmid containing one or two endolysin genes. The mix was then transferred to a 15 mL FALCON tube containing 600 mg of acid-washed 425-600 μm diameter glass beads, and the tube was shaken for 15 sec on a vortex at maximum speed. Immediately after, 6 mL of 0.5% (w/v) agar TAP, which had been maintained at 42° C., was added to the glass bead/cell suspension, and the mix was poured and spread on 1% (w/v) agar HSM plates. After solidification of the top layer containing the transformed cells, plates were incubated in dark for 24 h, and then transferred to a growth cabinet with continuous light, 25° C.

### Selection and Homoplasmy Drive

Selection was based on photosynthesis restoration. The TN72 strain is unable to grow on HSM media, which lacks a carbon source. Upon partial integration of the pAxi2.0 or pL2_psbH derivative plasmids, the psbH gene function is restored, allowing transformants that have integrated the plasmid to grow on HSM media. The endolysin genes are integrated downstream of the 3′UTR of the psbH gene. Maintaining cells on HSM for several generations eventually displaces the original chloroplast genome copies, repopulating the chloroplast with the modified genome containing the restored psbH and the endolysin genes. To test whether clones have reached homoplasmy, which refers to the state in which cells present only one class of chloroplast genome, a multiplex PCR reaction is carried out with the following primers:

O_53 (GTAGGTATGATTAGCTTTACTAAGCTAGTCATTG),
O_172 (AAATAGTAACATACTAAAGCGGATGTAACTCAATC) and
O_328 (CACTTTTACAACAAAGTACATTAGGAAAAACACG).

PCR conditions are the following: 95° C. for 1 min, 30 cycles of 95° C. 15 sec, 61.9° C. for 30 sec and 72° C. for 1 min 30 sec, and a final extension at 72° C. for 10 min. In addition, loss of resistance to spectinomycin, is also used as indicative of homoplasmy

### Strain Storage

Microalgae strains expressing endolysins were stored at room temperature and under dim light on TAP media. Long-term storage was performed using the GeneArt Cryopreservation kit for Algae (Thermo Fisher Scientific).

### Media Composition

TAP and HSM media were prepared following methods described in [3] and in [4]

### REFERENCES

1. Wannathong, T., Waterhouse, J. C., Young, R. E. B., Economou, C. K. & Purton, S. New tools for chloroplast genetic engineering allow the synthesis of human growth hormone in the green alga Chlamydomonas reinhardtii. Appl. Microbiol. Biotechnol. 100, 5467-5477 (2016).
2. Davies, D. R. & Plaskitt, A. Genetical and structural analyses of cell-wall formation in Chlamydomonas reinhardtii. Genet. Res. 17, 33-43 (1971).
3. Kropat, J. et al. A revised mineral nutrient supplement increases biomass and
4. The Chlamydomonas source book. (Elsevier, 2008).

### Example 15: Endolysin Minimum Inhibitory Concentration (MIC) and Minimum Bactericidal Concentration (MBC) Assays

In previous examples described above it was shown that GH25CPFORC3, AMI2phiCPV4, and AMI2phiZP2 endolysins exhibited bacteriolytic activities against the Cp strains screened (NCTC 2837, 8237, 8238, 8239) at room temperature (i.e. ˜23° C.) and in PBS, pH 7.0, buffer when Cp cell growths were minimal. It was also desirable to determine whether the endolysins could exhibit antimicrobial activities against actively growing Cp cells in liquid cultures. Co-incubations of endolysin serial dilutions with known initial bacterial loading allowed determination of the endolysin minimum inhibitory concentration (MIC) required. Additionally, an endolysin's minimum bactericidal concentration (MBC) could also be determined. In particular, endolysin MIC and MBC values were investigated against the Cp strain, Cp6, known to induce necrotic enteritis in poultry.

Strain Cp6 was inoculated and grown overnight to stationary phase as described previously. 100 uL of 10\hat{ }4, 10\hat{ }5, and 10\hat{ }6 of overnight culture dilutions were plated on RCM+1% agar in 3× replicates and grown overnight in the anaerobic chamber at 41° C. Subsequently, colony counting on the overnight agar plates was done in order to determine the initial bacterial loading (i.e. CFU/mL) used in the following MIC assay plates. 2× serially-diluted lysozyme positive control and endolysin stocks were prepared using PBS, pH 7.0 buffer. 1000-fold dilutions of the overnight stationary phase Cp6 culture were prepared using LB medium. The MIC assay was initiated by mixing 45 μL/well of the 1000-fold culture dilution with 5 uL 10-fold concentrated lysozyme (Sigma, ˜89,000 U/mg) or endolysin stocks (purified as described above) in the following assay plate format (Table 12). Each sample concentration was assayed in 2× replicates except for AMI2phiZP2. Wells A1-6 (50 μL/well LB medium only) corresponded to media controls whereas wells A7-12 (5 μL/well PBS, pH 7.0+45 μL/well 1000-fold culture dilutions) corresponded to growth controls. Effective loading concentrations of the MIC assay plate are also shown in Table 12.

After initiation of the MIC assay plates, the plates were sealed with Xtra-Clear Advanced Polyolefin StarSeal (Starlabs) and incubated for 13 hrs at 41° C. in the anaerobic chamber. Relative growth inhibitions on the overnight MIC assay plate was measured using a plate reader. To determine which sample concentrations were able to fully kill the initial loading of Cp6 cells, 5 μL/well of the overnight MIC assay plate was re-inoculated into 95 μL/well BHI+C medium, sealed with Xtra-Clear Advanced Polyolefin StarSeal (Starlabs), and incubated overnight at 41° C. in the anaerobic chamber. The overnight re-inoculated MBC assay plate was subsequently measured on a plate reader to assess whether residual viable Cp cells were present in the overnight MIC assay plates.

Overnight co-incubation of live Cp cells with the samples showed that the 3 endolysins were able to inhibit overnight growth whereas up to 2.5 mg/mL lysozyme loading was not given an initial bacterial loading of ˜2.1E4 cells/mL (Table 13). On a weight basis, MIC values were determined to be approximately 1.6, 16, and 16 μg/mL loadings for GH25CPFORC3, AMI2phiCPV4, and AMI2phiZP2 endolysins, respectively. Calculated MIC values on a molar basis are also indicated in Table 13. Comparison to lysozyme shows that the endolysins were greater than 1563, 156, and 156-fold improved lysozyme for GH25CPFORC3, AMI2phiCPV4, and AMI2phiZP2, respectively. Re-inoculated overnight MBC results showed that similar inhibitory concentrations of endolysins were also bactericidal (Table 13).

### Example 16: Synergistic Effects of Endolysins Used in Combination (Spot Assays)

Previous examples described above demonstrated that the discovered endolysins exhibited inhibitory and bacteriolytic activities against the various C. perfringens strains tested when used individually. It is commonly known that reagents with orthogonal mechanisms of action can often exhibit surprising and improved synergistic effects. The same can be true for endolysins used in combination to synergistically enhance their individual antimicrobial effects. This is particularly true if the endolysins are structurally different and with distinctly different mechanisms of action. Enhancing synergistic interactions are more likely when endolysins used in combination have different N-terminal catalytic and/or C-terminal cell wall binding domains, including when there are multiple different catalytic and/or cell wall binding domains. An investigation was initiated to determine whether the GH25CPFORC3 endolysin, an N-acetyl-β-D-muramidase, can act synergistically when used in combination with AMI2phiCPV4 or with AMI2phiZP2 which are both N-acetylmuramoyl-L-alanine amidases. The reasoning for testing these endolysin combinations are that bioinformatics sequence and structural analyses suggest the catalytic domains between N-acetyl-β-D-muramidase and N-acetylmuramoyl-L-alanine amidases are structurally different and cut at different bonds on the peptidoglycan cell wall. Additionally, the GH25CPFORC3 endolysin possesses two SH3 cell wall binding domains whereas both the AMI2phiCPV4 and AMI2phiZP2 endolysins each possess only one SH3 cell wall binding domain. These structurally-different attributes suggest that synergistic interactions between the endolysins are possible. Investigation into the presence of synergistic effects between GH25FORC3 and AMI2phiCPV4 were performed utilizing spot assays similarly done in previous Examples described above.

NCTC reference strains 8237 and 8238 were cultured as described in previous Examples described above. Purified endolysins, GH25CPFORC3 and AMI2phiCPV4, were immuno-purified. The antimicrobial activities of each endolysin applied individually or in combination was evaluated using freshly plated cells or cell lawns and the preparations were similarly to as described previously, except that 750 μL of stationary phase culture was used to inoculate 140 mm diameter RCM, 10% agar, plates. Individual endolysins were prepared at 200 μg/mL, 100 μg/mL, and 50 μg/mL concentrations, which were then spotted similarly onto freshly-plated cells and cell lawns. To investigate synergistic interactions, pre-mixed endolysin stocks of GH25CPFORC3 and AMI2phiCPV4 at 100 μg/mL or 50 μg/mL each were also spotted similarly onto freshly-plated cells and cell lawns. Briefly, 10 mm paper disks were placed on top of freshly-plated cells or cell lawns and 10 μl of purified endolysins (200 μg/mL, 100 μg/mL, 50 μg/mL) were spotted individually onto the disks. Endolysins applied in combination were similarly spotted but with 10 μl of pre-mixed endolysins with concentrations of 100 μg/mL or 50 μg/mL each. Each sample was spotted in 2× replicates. Thereafter, the agar plates were incubated at 37° C. overnight for 16-24 h for the development of clearance zones.

Spot assays results showed synergistic interactions between the GH25CPFORC3 and AMI2phiCPV4 endolysins. The presence of synergy was assessed based on whether the clearance zones produced by a combined endolysin spot (e.g. 100 μg/mL each of GH25CPFORC3 and AMI2phiCPV4) was greater than that produced by the same total loading of an individual endolysin spot (e.g. 200 μg/mL GH25FORC3 or AMI2phiCPV4). An example of the visual evaluations and the endolysin sample layout for investigating the endolysins' synergistic interactions on NCTC 8238 cell lawns is shown in FIG. 21. Spot assay results on NCTC 8238 freshly-plated cells and cell lawns showed that the clearance zones of combined endolysins were greater than the same total loading of an individual spot. However, synergistic interactions were not observed when the endolysins were spotted on both NCTC 8237 freshly-plated cells or cell lawns. These results are summarized in Table 14. Combining GH25CPFORC3 and AMI2phiCPV4 endolysins showed that enhancing synergistic antimicrobial activities are possible and can be extended to additional Cp strains.

### Example 17: Synergistic Effects of Endolysins Used in Combination (MIC/MBC Assays in 96-Microwell Liquid Cultures)

An alternative method was used to determine more quantitatively the synergistic effects between different endolysins. The method allowed determination for the amount of endolysin loading each and the individual endolysin loading ratios required to inhibit Cp growth after overnight cultures given an initial bacterial loading.

The Cp strain was inoculated and grown overnight to stationary phase as described previously. 100 uL of 10\hat{ }4, 10\hat{ }5, and 10\hat{ }6 of overnight culture dilutions were plated on RCM+1% agar in 3× replicates and grown overnight in the anaerobic chamber at 41° C. Subsequently, colony counting on the overnight agar plates was done for determining the initial bacterial loading (i.e. CFU/mL) used in the following MIC assay plates. 2× serially-diluted endolysin stocks were prepared using LB medium. 1000-fold dilutions of the overnight stationary phase culture were prepared also using LB medium. The MIC assay was initiated by first dispensing 5 μL/well endolysin 1 and 5 μL/well endolysin 2 serially-diluted stocks according to the 96-microwell plate format in Table 15. 10 μL/well and 100 μL/well LB medium was dispensed into the growth control and media control wells, respectively. To initiate the MIC assay, 90 μL/well 1000-fold overnight culture dilution were dispensed into sample wells. After initiation of the MIC assay plates, the plates were sealed with Xtra-Clear Advanced Polyolefin StarSeal (Starlabs) and incubated overnight at 41° C. in the anaerobic chamber. The effective endolysin loading concentrations, and of the uM loading ratios of endolysin 2 and endolysin 1 are shown in Table 16.

Relative growth inhibitions on the overnight MIC assay plate was measured using a plate reader. To determine which endolysin concentrations and ratios were bactericidal, 5 μL/well of the overnight MIC assay plate was re-inoculated into 95 μL/well BHI+C medium, sealed with Xtra-Clear Advanced Polyolefin StarSeal (Starlabs), and incubated overnight at 41° C. in the anaerobic chamber. The overnight re-inoculated MBC assay plate was subsequently measured on plate reader to assess whether residual viable Cp cells were present in the overnight MIC assay plates.

### Example 18: Synergistic Effects of Endolysins Used in Combination (Spot Assays on 96-Microwell Solid Medium)

An alternative method was used to determine more quantitatively the synergistic effects between different endolysins, more specifically between GH25FORC3 and AMI2phiCPV4. The method allowed determination for the amount of endolysin loading each and the individual endolysin loading ratios required to inhibit Cp growth after overnight cultures. The method was also an improvement on the spot assay method used in previous examples for obtaining higher accuracy and precision results that relied on OD620 nm plate reader readings instead of by visual inspection.

The Cp strain was inoculated and grown overnight to stationary phase as described previously. 100 uL of 10\hat{ }4, 10\hat{ }5, and 10\hat{ }6 of overnight culture dilutions were plated on RCM+1% agar in 3× replicates and grown overnight in the anaerobic chamber at 41° C. Subsequently, colony counting on the overnight agar plates was done for determining the initial bacterial loading (i.e. CFU/mL) used in the following MIC assay plates. 2× serially-diluted endolysin stocks were prepared using LB medium. 1000-fold dilutions of the overnight stationary phase culture were prepared also using LB medium. Except for the agar control wells, 50 μL/well overnight culture dilutions was dispensed onto 100 μL/well RCM+1% agar presiding in 96-microwell plates. Two 96-microwell plates were prepared. One in which the cells were freshly-plated (FP) and one in which the cells were allowed to grow overnight into cell lawns (CL). For each prepared plate, the assay was initiated by dispensing 10 μL/well pre-mixed endolysin 1 and endolysin 2 serially-diluted stocks to give effective loading concentrations in the 96-microwell plate format in Table 15. After initiation of the MIC assay plates, the plates were sealed with Xtra-Clear Advanced Polyolefin StarSeal (Starlabs) and incubated overnight at 41° C. in the anaerobic chamber. The effective endolysin loading concentrations, and of the uM loading ratios of endolysin 2 and endolysin 1 are shown in Table 15. The inhibitory and the bacteriolytic effects of the endolysin combinations were assessed by visually inspecting the freshly-plated cell plates and the cell lawn plates, respectively.

### Example 19: Affinity Purification of 6×His-Tagged AMI2phiZP2, AMI2phiCPV4, and GH25CPF4969 Endolysins

Affinity purifications of 6×His-tagged AMI2phiZP2 (SEQ ID NO: 3), AMI2phiCPV4 (SEQ ID NO: 2), and GH25CPF4969 (SEQ ID NO: 8) endolysins expressed in E. coli strains bAL187, bAL188, and bAL198 were performed for use in later examples.

Pre-cultures of 6×His-tagged AMI2phiZP2, AMI2phiCPV4, and GH25CPF4969 endolysins expressed in E. coli were initiated in 10 mL LB media (+100 μg/mL ampicillin, 25 μg/mL chloramphenicol) and incubated overnight at 37° C. with shaking (150 rpm). The next day, 200 mL of LB media (+100 μg/mL Ampicillin, 25 μg/mL chloramphenicol) was inoculated with 2 mL of pre-culture and incubated at 37° C. for about 3 hours with shaking (˜150 rpm). Expression of the protein was induced by adding IPTG (isopropyl-2-D-thiogalactopyranoside) (Merck) to a final concentration of 1 mM and the culture was incubated at 30° C. for an additional 5 hr. Bacterial cells were harvested by centrifugation at 3800×g for 10 min using a SIGMA 3-16 KL centrifuge and the bacterial pellets were stored at −20° C. until the next day. The pellets were resuspended in 5 mL binding/wash buffer (20 mM Sodium Phosphate, 0.5M NaCl, 20 mM Imidazole, pH 7.4) with cOmplete, Mini, EDTA-free Protease Inhibitor cocktail (Merck). Cells were lysed by sonication (10 μm amplitude, 5 sec on/off, 10 cycles) using a MSE Soniprep 150 ultrasonic disintegrator. The clarified lysate was clarified by centrifugation at 31,030×g on an Eppendorf 5430R centrifuge and the supernatant collected. Affinity chromatography was performed using Ni-Sepharose™ 6 Fast flow resin (GE Healthcare). The clarified lysate was mixed with 1 ml of the Ni-sepharose resin in a 50 mL Falcon tube and the mixture incubated for 1 hour on ice on a rocking platform. The lysate/resin suspension was then poured into an empty PD-10 column (GE Healthcare) and the flow-through was collected for later analysis. Ni-sepharose resin with bound protein was washed with buffer (20 mM Sodium Phosphate, 0.5 M NaCl, 20 mM Imidazole, pH 7.4) and the protein was then eluted with elution buffer, consisting of 20 mM Sodium Phosphate, 0.5M NaCl, 0.5M Imidazole, pH 7.4. Absorbance at 280 nm was measured to confirm the presence of eluted protein in the elution fractions. Buffer-exchange and concentration of the pooled elution fractions were performed similarly to in Example 10. Verification on the purification of the proteins were determined by SDS-PAGE using 15-20% Mini-Protean TGX Precast Protein gels (Bio-Rad) in a Bio-Rad Mini-PROTEAN electrophoresis system. SDS-PAGE samples and a BSA standard curve were run also similarly as described in Example 10. Resultant protein gels were stained with InstantBlue™ staining solution (Expedeon) and de-stained with MQ H2O. The de-stained gel was imaged with the Vilber Bio-print gel documentation system. Densitometry analyses for quantification of the purified endolysins were performed using the onboard software.

SDS-PAGE and densitometry analyses (FIG. 22) show that endolysins were highly enriched with low amount of contaminant proteins. These contaminant proteins did not interfere in subsequent experiments. Quantification of the protein band of interested showed that the purified stock concentrations of AMI2phiZP2, AMI2phiCPV4, and GH25CPF4969 were 1700 μg/mL, 1000 μg/mL, and 3600 μg/mL, respectively.

### Example 20: Purification of C. perfringens Strain Cp6 Peptidoglycan

Peptidoglycan (PGN) from C. perfringens Cp6 strain was purified for use as an alternative substrate for assessing endolysin activity. A pre-culture consisting of 10 mL of BHI+C media was inoculated with 0.4 mL of a culture of C. perfringens Cp6 strain in meat stock and incubated in a MACS-MG-500 anaerobic chamber (Don Whitely) at 37° C. overnight. The next day, IL of BHI+C medium was inoculated with 0.5 ml of the overnight pre-culture and incubated in the anaerobic chamber at 37° C. for approximately 17 hours. Cells were then centrifuged at 3,800×g using a SIGMA 3-16 KL centrifuge (SciQuip) for 10 mins and pelleted cells were washed twice with PBS, pH 7.0 and resuspended in the same buffer to achieve a final of OD at 600 nm of approximately 20. A 20% (w/v) SDS solution in PBS, pH 7.0 buffer was prepared in a shake flask and heated in a boiling water-bath. The concentrated C. perfringens Cp6 cell suspension was added slowly dropwise into the boiling 20% (w/v) SDS solution with constant stirring for a final equal-part dilution (1:1) of the cell suspension and SDS solution. The cell/SDS suspension was boiled for an additional 2 hours before allowing the suspension to cool with continued stirring overnight. The following day, the cells/SDS suspension was centrifuged at 30,000×g on an Eppendorf 5430R centrifuge for 30 minutes to harvest a white peptidoglycan pellet. The supernatant containing SDS was discarded and the white pellet was washed with PBS, pH 7.0 buffer several times to remove residual SDS or until no more bubbles could be seen in the supernatant. The harvested peptidoglycan pellet was resuspended in buffer to give an approximate 16-20 OD at 600 nm. 0.02% (w/v) of sodium azide was added to the purified PGN suspension stock and stored at room temperature.

For use in PGN degradation assays, the purified PGN suspension stock was diluted 8 to 10-fold in PBS, pH 7.0 buffer to make a “PGN assay stock”.

### Example 21: Synergistic Effects of Purified Endolysins Used in Combination (PGN Assay)

The synergistic effects of six purified endolysins used in various combinations were investigated using the PGN degradation assay (i.e. PGN assay). The endolysins tested include AMI3CPF4969 (SEQ ID NO: 11), GH25CPFORC3 (SEQ ID NO: 1), GH25phiS63 (SEQ ID NO: 5), AMI2phiZP2 (SEQ ID NO: 3), AMI2phiCPV4 (SEQ ID NO: 2) and GH25CPF4969 (SEQ ID NO: 8). Sequence alignments of the endolysins using Pfam (https://pfam.xfam.org/) suggested their corresponding enzyme class (based on their N-terminal catalytic domain) as well as their C-terminal binding domains. However, these putative results cannot be verified without protein structure alignments and/or X-ray crystallography experiments of the purified endolysins. As such, the endolysins were tested in various combinations to determine if they might display synergistic effects when used together. Firstly, if the endolysins are from different enzyme classes, their combination might show synergistic effects on PGN degradation since the enzymes would have hydrolytic activities on different PGN bonds. Secondly, even if the endolysins are from the same enzyme class and have hydrolytic activities toward the same PGN bond, the irreversible binding of their binding domains onto C. perfringens cell wall ligands might differ and affect the efficiency of PGN degradation. Therefore, the combinations were tested to investigate whether endolysins used in combination would reveal surprising synergistic effects that increase their hydrolytic activities on C. perfringens cell wall PGN.

Checkerboard PGN assays were done as follows to investigate the synergistic effects of between different purified endolysin pairs. The combinations of purified endolysins tested include 1) GH25CPFORC3 and AMI2phiZP2, 2) GH25CPFORC3 and AMI2phiCPV4, 3) AMI3CPF4969 and GH25CPF4969, 4) GH25phiS63 and GH25CPF4969, 5) GH25CPFORC3 and AMI3CPF4969, and 6) AMI2phiZP2 and GH25CPF4969. E. coli-expressed GH25CPFORC3, AMI3CPF4969, GH25phiS63 were purified and quantified similarly to as described in Example 10. E. coli-expressed AMI2phiZP2, AMI2phiCPV4, and GH25CPF4969 were purified and quantified similarly to as described in Example 19.

All purified endolysin stocks were normalized and diluted to either 25 μg/mL or 100 μg/mL concentrations (10× stock) in PBS, pH 7.0 buffer. Two checkerboard PGN assays were done in one 96-microwell plate with an example sample layout for an 1× endolysin starting concentration of 10 μg/mL (FIG. 23). 10× stocks of column endolysins (i.e. endolysin 1 or 3) were 2-fold serially-diluted 6 times to 1.6 μg/mL, whereas 10× stocks of row endolysins (i.e. endolysins 2 or 4) were 2-fold serially-diluted 4 times to 6 μg/mL. 10 μL/well of the corresponding endolysin 1 or 3 10× stocks were dispensed into the appropriate columns and similarly 10 μL/well of the corresponding endolysin 2 or 4 10× stocks were dispensed into the appropriate rows. 10 μL/well of PBS, pH 7.0 was added to endolysin 1 or 3 only control columns 6 and 12, respectively. Similarly, 10 μL/well of PBS, pH 7.0 was added to endolysin 2 or 4 only control row H. An additional 10 μL/well of PBS, pH 7.0 was added to well H6 and H12 for the 0 μg/mL loading well. The PGN assay was initiated by dispensing 80 μL/well of PGN assay stock, thereby effectively diluting each endolysin 10-fold. Wells H6 and H12 are “PGN only” control wells with 0 μg/mL endolysin. Endolysin loadings in each well correspond to the row and column loading concentrations indicated. The assay plate was mixed briefly on a plate shaker and the reduction in turbidity was monitored continuously using the kinetic read function on a Labtech LT-4500 plate reader at 620 nm for 3-4 hours at room temperature (˜23° C.).

Assessment of the 6 different combinations of purified endolysins show that the combinations tested were synergistic. In all cases studied herein a synergistic effect was determined based on whether the initial PGN degradation rate, when two endolysins were used in combination, was greater than the sum of each endoysin's initial PGN degradation rate. An effect was also determined to be synergistic on the basis of the overall PGN degradation at the end-point of the assay as determined by optical density (OD), irrespective of the initial PGN degradation rates. In this situation an effect is considered to be synergistic if the final amount of degraded PGN as measured by OD reduction is greater for the combination of endolysins compared with the amount of degraded PGN observed for the individual endolysins when tested alone. An example of synergistic effects of extracts expressing endolysins from different enzyme classes can be seen in FIG. 24 where purified endolysins GH25CPFORC3 and AMI2phiZP2 were used. When 2.5 μg/mL GH25CPFORC3 and 2.5 μg/mL AMI2phiZP2 endolysins were used, their combined degradation rate (0.33 OD/hr) is greater than the sum of their individual rates (0.28 OD/hr) (see FIG. 24D). Similar synergistic effects were observed at also the following loading concentration ratios of GH25CPFORC3 and AMI2phiZP2, 2.5/0.63 and 1.25/2.5 μg/mL. In addition, the pair of endolysins was also able to effect higher overall maximum PGN degradations at the end-point of the assay (250 minutes) than either of the two endolysins could achieve when used individually on their own. Plots A, B, and C of FIG. 24 show that at those loading concentration ratios, the GH25CPFORC3/AMI2phiZP2 combination was able to degrade the PGN to lower ODs than either the GH25CPFORC3 or AMI2phiZP2 endolysin could achieve on its own. At the highest loading concentration ratio shown in FIG. 24A, the PGN degradation of GH25CPFORC3 and AMI2phiZP2, when used individually, both stabilized at ˜0.15 OD and could not degrade further. However, when 2.5 μg/mL GH25CPFORC3 and 2.5 μg/mL AMI2phiZP2 were used in combination, higher PGN degradation to lower ODs was observed. Similar higher PGN degradations of the endolysins when used in combination were also observed in plots B and C. In conclusion, result shows that the GH25CPFORC3/AMI2phiZP2 endolysin combination is synergistic.

The synergistic combination, GH25CPFORC3 and AMI2phiZP2, presented example PGN degradation results showing that synergistic effects are present when an endolysin combination exhibits 1) higher PGN degradation rate compared to the sum of each endolysin's individual rate and/or 2) higher maximum end-point PGN degradation (as determined by OD measurements in the above-described PGN degradation assay) than when the endolysins are used individually on their own. Qualifications for the presence of synergistic effects for different endolysin combinations were assessed by the same criteria. Therefore, it was found that the following endolysin combinations were additionally synergistic: 1) GH25CPFORC3/AMI2phiCPV4 as shown in FIG. 25; 2) AMI3CPF4969/GH25CPF4969 as shown in FIG. 26; 3) GH25phiS63/GH25CPF4969 as shown in FIG. 27; 4) GH25CPFORC3/AMI3CPF4969 as shown in FIG. 28; and 5) GH25CPF4969/AMI2phiZP2 as shown in FIG. 29.

### Example 22: General Description on the Construction of Endolysin Expression Vectors and Chlamydomonas reinhardtii Transformation

The algal expression system used to produce endolysin genes in microalgae has the following components:
(1) C. reinhardtii microalga host strains.
(2) DNA vectors designed to integrate the endolysin genes into the chloroplast genome of the microalga.
(3) A method for algal transformation.
(4) A method of selection and homoplasmy drive of microalga clones harbouring the endolysin-containing DNA vector. The method was based on photosynthesis restoration or antibiotic resistance.

### (1) Microalga Host Strains

The host C. reinhardtii strains used for endolysin expression were TN72 and crAL035. Both strains are mt+ cell-wall deficient mutants derived from the wild type strain CC-125, equivalent to 137c. In the TN72 strain, the psbH gene of the chloroplast genome has been deleted by gene replacement with the spectinomycin resistance gene aadA [1]. psbH encodes for a component of Photosystem II, which is required for light assimilation during photosynthesis. Absence of psbH renders cells that cannot grow photo-autotrophically and that require acetate in the media in order to grow. The crAL035 strain is derived from TN72 after transformation with plasmid plAL010. Plasmid plAL010 carries a 0.8 kb fragment of the downstream sequence of the psbH gene, a 2.4 kb fragment that contains the psbH gene and some of its upstream sequence, the lacZ gene and the tRNA-Wtca gene. Integration of plAL010 into the chloroplast genome restores psbH function and photosynthesis. Transformants were selected on minimal media without acetate, where only photo-autotrophic clones can grow. Transformants were passaged twice on minimal media and homoplasmy of the construct was tested by PCR as described below (point 4).

### (2) DNA Vectors

Recombinant endolysin genes were introduced into the C. reinhardtii chloroplast by integration of plasmids derived from pAxi2.0, plAL010 or plAL009 plasmids. pAxi2.0 and plAL010 carry a 0.8 kb fragment of the downstream sequence of the psbH gene, and a 2.4 kb fragment that contains the psbH gene and some of its upstream sequence. These fragments were gene synthesized (Eurofins Genomics) and cloned into a plasmid carrying a kanamycin resistance gene. A multi-cloning site (MCS) with inverted BsaI restriction enzyme sites for the cloning of endolysin genes by Golden Gate cloning was designed in between the two psbH homologous regions. Both pAxi2.0 and plAL010 have a tRNA-Wtca gene inserted into the MCS, which transcribes a modified C. reinhardtii tRNA molecule that translates TGA codons as Tryptophan. In addition, plAL010 also has the lacZ gene cloned into the MCS. plAL009 carries two 1 Kb sequences homologous to a region downstream of the petB gene in the chloroplast genome. Similarly to plAL010, plAL009 has a MCS in between the petB homologous sequences flanked by inverted BsaI restriction enzyme sites for the cloning of endolysin genes by Golden Gate. The MCS of plAL009 carries the same modified tRNA-Wtca gene and lacZ gene as described for plAL010.

Endolysins coding sequences, fused to FLAG, HA, Strep, or 6×His tag sequences and flanked by 5′ and 3′ gene expression regulatory sequences, were cloned at the BsaI restriction enzyme sites of the MCS of pAxi2.0, plAL010 and plAL009. The 5′ and 3′ regulatory sequences that drive the expression of the endolysin genes were derived from the psbA, psaA, 16S rDNA, atpA and rbcL chloroplast genes. Endolysin coding sequences and regulatory sequences were gene-synthesized (Eurofins Genomics) and assembled by Golden Gate cloning

### (3) Algal Transformation by the Glass Bead Method

For transformation, the host microalga strain was grown in 400 mL TAP media [2], [3] under constant light (50-500 μmol s⁻¹ m⁻²), 25° C. and 140 rpm orbital shaking, to an optical density of 0.3-0.6 OD750 nm. The culture was then centrifuged at 765×g for 10 min in a centrifuge Sigma 3-16 KL (SciQuip), and the cell pellet was resuspended in 1 mL of TAP media. 0.5 mL of the algal suspension was mixed with 40-100 μg of a DNA plasmid, and the mix was then transferred to a 15 mL conical tube (Starlab) containing 600 mg of acid-washed 425-600 μm diameter glass beads (Merck). The tube was shaken for 15 sec on a vortex (SciQuip) at maximum speed. For selection based on photosynthesis restoration, 6 mL of 0.5% (w/v) agar TAP media, pre-warmed at 42° C., was added to the glass bead/cell suspension, and the mix was poured and spread on 1% (w/v) agar HSM plates [2], [3]. After solidification of the top layer containing the transformed cells, plates were incubated in dark for 24 hr, and then transferred to a growth cabinet (LEEC) with continuous light (50-500 μmol s⁻¹ m⁻²), 25° C. For selection based on antibiotic resistance, after shaking with glass beads and DNA plasmid, cells were transferred to 5 mL TAP and incubated in dim light overnight with orbital shaking. The next day, cultures were centrifuged at 765×g for 10 min in a centrifuge Sigma 3-16 KL (SciQuip) and the pellet was spread on plates containing TAP media supplemented with 100 μg/mL spectinomycin (Merck). Plates were incubated in dark for 48 hr and then transferred to constant light (50-500 μmol s⁻¹ m⁻²) until the colonies grew.

### (4) Method of Selection and Homoplasmy Drive

The method of selection of microalga clones harbouring endolysin genes was based on photosynthesis restoration or antibiotic resistance. The host strain TN72 was used in the photosynthesis restoration method. This strain is unable to grow on HSM media by itself, but upon integration of the pAxi2.0 or plAL010 derivative plasmids the psbH gene function is restored and transformants can grow on this media. The endolysin genes are integrated downstream of the 3′UTR of the psbH gene. Maintaining cells on HSM for several generations eventually displaces the original chloroplast genome copies, re-populating the chloroplast with the modified genome containing the restored psbH and the endolysin genes. To test whether clones have reached homoplasmy, which refers to the state in which the chloroplast is populated only by the recombinant genome, two diagnostic PCRs were carried out with primer pairs O_53/O_328 and O_53/O_172 (Table 19). PCR conditions were the following: 95° C. for 1 min, 30 cycles of 95° C. 15 sec, 61.9° C. for 30 sec and 72° C. for 1 min 30 sec, and a final extension at 72° C. for 10 min. PCR was performed in a Prime Thermal Cycler (Techne) using PCRBIO Taq (PCR Biosystems), following manufacturer's recommendations. In addition, loss of spectinomycin resistance was also used to confirm homoplasmic clones.

For the antibiotic resistance selection method, the spectinomycin resistance gene aadA gene [4] was cloned together with the endolysin gene in the MCS of plasmid plAL009. After transformation and overnight recovery in TAP media, cells were spread on plates with TAP media supplemented with 100 μg/mL spectinomycin (Merck). Antibiotic resistant colonies appeared after two weeks of incubation at 25° C. in constant light (50-500 μmol s⁻¹ m²). Transformants were driven to homoplasmy after passage on TAP 100 μg/mL spectinomycin plates several times. Two diagnostic PCRs were carried out to screen for homoplasmic clones with primer pairs O_323/O_328 and O_323/O_336 (Table 19). PCR conditions were the following: 95° C. for 1 min, 30 cycles of 95° C. 15 sec, 61.9° C. for 30 sec and 72° C. for 1 min 30 sec, and a final extension at 72° C. for 10 min. PCR was performed in a Prime Thermal Cycler (Techne) using PCRBIO Taq (PCR Biosystems), following manufacturer's recommendations.

Homoplasmic strains were stored at room temperature and under dim light on TAP media supplemented with 10% (w/v) yeast extract (Merck). Long-term storage was prepared using the GeneArt Cryopreservation kit for Algae (Thermo Fisher Scientific), following manufacturer's instructions.

### REFERENCES

[1] T. Wannathong, J. C. Waterhouse, R. E. B. Young, C. K. Economou, and S. Purton, “New tools for chloroplast genetic engineering allow the synthesis of human growth hormone in the green alga Chlamydomonas reinhardtii,” Appl. Microbiol. Biotechnol., vol. 100, no. 12, pp. 5467-5477, 2016.
[2] E. H. Harris, The Chlamydomonas Sourcebook Volume 1: Introduction to Chlamydomonas and Its Laboratory Use, Second edi. Academic Press, 2009.
[3] J. Kropat et al., “A revised mineral nutrient supplement increases biomass and growth rate in Chlamydomonas reinhardtii,” Plant J, vol. 66, no. 5, pp. 770-780, 2011.
[4] M. Goldschmidt-clermont, “Transgenic expression of aminoglycoside adenine transferase in the chloroplast: A selectable marker for site-directed transformation of chlamydomonas,” Nucleic Acids Res., vol. 19, no. 15, pp. 4083-4089, 1991.
   Primers used to screen for homoplasmic C. reinhardtii transformants are listed in Table 19 below.

### Example 23: Construction of Engineered Chlamydomonas reinhardtii Strains Expressing Endolysins

Different strains of C. reinhardtii were constructed that express one or two endolysin genes.

Strains crAL036, crAL038, crAL039 and crAL045 were constructed using the selection method based on photosynthesis restoration described in Example 22. The host strain TN72 was transformed by the glass bead method with plasmids listed in Table 20. These plasmids were derived from pAxi2.0 or plAL010, which carry homology regions of the psbH gene. Plasmids were assembled from individual parts (promoters, 5′UTR, coding sequences and 3′UTR) following a hierarchical strategy and using Golden Gate cloning. Individual parts, which were gene-synthesized (Eurofins Genomics), were cloned into separated plasmids. Gene units were assembled from individual parts in a reaction containing SapI (New England Biolabs) and T4 DNA ligase (New England Biolabs). Gene units were then assembled into multigene constructs using BsaI (New England Biolabs) and T4 DNA ligase (New England Biolabs), into pAxi2.0 or plAL010 plasmids. Final plasmids were then sequenced verified and purified using the HiSpeed Plasmid Maxi kit (Qiagen). Table 21 summarises strains features. Transformation, selection and homoplasmy drive were carried out as described in Example 22.

Microalga strains crAL041, crAL061 crAL075, crAL076, crAL077, crAL078, crAL079 and crAL080 were generated as described in Example 14 using a selection method based on antibiotic resistance. The host strain crAL035 was transformed with plasmids plAL163, plAL166, plAL168, plAL169, plAL170, plAL171, plAL172 and plAL173, respectively (Table 21). These plasmids were derived from plAL009 and carry an endolysin gene and the aadA gene, which confers resistance to the antibiotic spectinomycin in the alga. These plasmids were assembled from individual parts (promoters, 5′UTR, coding sequences and 3′UTR) following a hierarchical strategy using Golden Gate cloning [1]. Individual parts, which were gene-synthesized, were cloned into separated plasmids. Gene units were assembled from individual parts in a reaction containing SapI (New England Biolabs) and T4 DNA ligase (New England Biolabs). Gene units were then assembled into multigene constructs using BsaI (New England Biolabs) and T4 DNA ligase (New England Biolabs), into plAL009. Final plasmids were then sequenced verified and purified using the HiSpeed Plasmid Maxi kit (Qiagen). Transformation, selection and homoplasmy drive were carried out as described in Example 22. Potential homoplasmic clones were further verified by gDNA extraction and amplification of the petB chromosomal locus, where integration took place, using primers O_323 and O_336 (Table 19). The conditions for this PCR were: 98° C. for 30 s, 35 cycles of 98° C. 10 s, 65° C. for 20 s and 72° C. for 5 min, and a final extension at 72° C. for 10 min. PCR was carried out in a Prime Thermal Cycler (Techne), using High-Fidelity DNA polymerase Phusion (New England Biolabs), following manufacturer's instructions.

Algal strains co-expressing two endolysin genes were generated by two consecutive transformations. Strain crAL057, which co-expresses AMI2phiZP2 and GH25CPFORC3 endolysins, was created by transforming an AMI2phiZP2-expressing strain (crAL038) with plasmid plAL166, which carries the GH25CPFORC3 gene (Table 21). In addition the plasmid plAL166 harbours the crCD-aadA gene, which confers resistance to the antibiotic spectinomycin. The plasmid plAL166 was constructed by Golden Gate using plAL009 (Table 20). The strain crAL038 was transformed with 20-50 μg of plasmid, and clones were selected based on antibiotic resistance, as described in Example 22. Homoplasmic clones were isolated after several passages on selective media. In order to verify the integration of plasmid plAL166 and homoplasmy, gDNA was extracted from transformants and the petB chromosomal locus, where integration took place, was amplified by PCR using primers O_323 and O_336 (Table 19). The conditions for this PCR were: 98° C. for 30 s, 35 cycles of 98° C. 10 s, 65° C. for 20 s and 72° C. for 5 min, and a final extension at 72° C. for 10 min. PCR was carried out in a Prime Thermal Cycler (Techne), using High-Fidelity DNA polymerase Phusion (New England Biolabs), following manufacturer's instructions. In addition, a PCR using specific primers to the AMI2phiZP2 gene (O_197/O_106) (Table 19) was carried out to test that the integration of GH25CPFORC3 at the petB site did not disrupt the AMI2phiZP2 gene integrated at the psbH site.

crAL058 strain was constructed by screening clones of crAL057 strain where spontaneous recombination between two identical sequences flanking the crCD-aadA gene has occurred. The screen was carried out by plating on TAP plates a serial dilution of a culture of the crAL057 strain grown to saturation in TAP in light (50-500 μmol s⁻¹ m⁻²). Plates were incubated in constant light (50-500 μmol s⁻¹ m⁻²) for a week at 25° C. Then, isolated colonies were streaked out on TAP and TAP supplemented with 100 μg/ml spectinomycin (Merck). Clones which were not able to grow on the TAP spectinomycin plates were picked from the replica plate on TAP. gDNA was extracted from these clones and the loss of the antibiotic resistance gene was confirmed by PCR using primers O_323/O_336 (Table 19) and the following conditions: 98° C. for 30 s, 35 cycles of 98° C. 10 s, 65° C. for 20 s and 72° C. for 5 min, and a final extension at 72° C. for 10 min. PCR was carried out in a Prime Thermal Cycler (Techne), using High-Fidelity DNA polymerase Phusion (New England Biolabs), following manufacturer's instructions.

Plasmids used to transform C. reinhardtii strains are listed in Table 20 below. Features of endolysin-carrying microalga strains are listed in Table 21 below.

### REFERENCE

[1] E. Weber, C. Engler, R. Gruetzner, S. Werner, and S. Marillonnet, “A modular cloning system for standardized assembly of multigene constructs,” PLoS One, vol. 6, no. 2, 2011.

### Example 24: Characterization of Extracts from Chlamydomonas reinhardtii Strains crAL035, crAL038, crAL039, crAL045, crAL057 and crAL061

In this example, the bacteriolytic and antimicrobial activities of extracts derived from C. reinhardtii strains expressing endolysin genes were evaluated. The experimental procedure is summarized as follows: (1) strain construction, described in Example 22 and 23; (2) lab scale culture of endolysin-expressing microalga strains; (3) extract preparation from the microalga biomass harvested; (4) bacteriolytic and antimicrobial activity assays of extracts. The bacteriolytic activity was assessed by PGN degradation and clearing zone formation, described below, while the antimicrobial activity was evaluated by minimal inhibitory concentration (MIC) assays.

The strains used in this example were crAL038, crAL039, crAL045, crAL057 and crAL061, which express either AMI2phiZP2 (SEQ ID NO: 3) (crAL038 and crAL039) or GH25CPFORC3 (SEQ ID NO: 1) endolysin (crAL045 and crAL061). Strain crAL035, which expresses no endolysin, was used as a control strain. Strain crAL057 expresses both AMI2phiZP2 and GH25CPFORC3 endolysins, and was used to evaluate the effect of co-expression on the total bacteriolytic and antimicrobial activity. Construction of these Chlamydomonas strains is summarized in Example 22 and 23. Recombinant strains were generated by integration of a vector downstream of the psbH and/or petB genes of the chloroplast genome. The sequence integrated carried an endolysin gene flanked with gene expression regulatory sequences (Table 20 and Table 21). The AMI2phiZP2 endolysin gene was expressed under psaA promoter and 5′UTR in crAL039, while in crAL038 it was cloned under the 16S promoter and the psaA 5′UTR. In both strains the constructs were integrated downstream of the psbH gene in the chloroplast genome. Strain crAL061 expresses the GH25CPFORC3 endolysin gene under the 16S promoter and atpA 5′UTR, and the construct is integrated downstream of the petB gene. Strain crAL045 expresses the GH25CPFORC3 gene using the 16S promoter and psaA 5′UTR and the expression cassette is integrated downstream of the psbH gene. The co-expressing strain crAL057 has the AMI2phiZP2 gene cloned under the 16S promoter and psaA 5′UTR and integrated downstream of the psbH gene, and the GH25CPFORC3 gene cloned under the 16S promoter and atpA 5′UTR and integrated under the petB gene.

Lab scale cultures of C. reinhardtii strains were grown in 200 mL TAP media, in 500 mL shake flasks. Strains were refreshed from storage cultures (solid TAP media) by streaking out cells in ˜1 cm2 patches onto TAP plates. Plates were incubated in the light cabinet (LEEC) under constant light (50-500 μmol s⁻¹ m⁻²) at 25° C. for 3-4 days, or until sufficient cells had grown. These cells were then used to inoculate 20 mL liquid TAP media, in 50 mL shake flasks. Flasks were incubated at 25° C., on an orbital shaker set at 140 rpm under dark conditions (i.e. ambient light only) for 3-5 days. These pre-cultures, which typically were at early-stationary growth phase, were used to inoculate 200 mL liquid TAP media at an initial OD750 nm of 0.05-0.1. The shake flask cultures were incubated at 25° C. under the same conditions. Cells were harvested at late-exponential to early-stationary growth phase where the OD750 nm varied from 0.7 to 1.5. Cultures were harvested by transferring ˜50 mL of culture into 50 mL conical tubes and pelleting the cells by centrifugation for 10 min at 765×g using the SIGMA 3-16KL centrifuge (SciQuip). The supernatant was discarded and pellets with residual TAP media were transferred to 1.5 mL tubes and centrifuged again. The residual supernatant TAP medium was discarded and pellets were stored at −20° C. until further processing.

To prepare soluble extracts, pellets stored at −20° C. were thawed and resuspended in 150-500 μL PBS, pH 7.0 depending on the pellet size. The cell suspension was lysed by repeated freeze-thaw cycles (×3) and then sonication (10 μm amplitude, 10 sec on/off, 10 cycles) in a Soniprep 150 ultrasonic disintegrator (MSE) Soluble extracts were obtained by centrifuging the whole-cell extract at 31,030×g for 30-45 min in a 5430R centrifuge (Eppendorf). The total soluble protein (TSP) content of the extracts was determined using the Bio-rad DC protein assay according to manufacturer instructions.

A whole-cell Cp6 spot assay consisted of spotting an endolysin-expressing algal extract onto solid agar media prepared with TAP agar and a Clostridium perfringens Cp6 cell suspension. The presence of endolysin activity in the algal extract would be manifested by a clearance zone on the solid media, signifying cell lysis. TAP agar plates with whole-cell Cp6 were prepared as follows. Firstly, a 400 mL culture of C. perfringens strain Cp6 grown in BHI+C mediaat 37° C. in a MACS-MG500 anaerobic chamber (Don Whitely) overnight. C. perfringens cells were pelleted at 3,800×g for 10 min using the SIGMA 3-16KL centrifuge (SciQuip) and washed with PBS, pH 7.0 buffer. Cp6 cell pellets were resuspended in 40-50 mL liquid TAP media and mixed in a 1:1 ratio with molten TAP agar, 10% (w/v). The mix was then poured onto Petri dishes and allowed to solidify. Once solidified, algal extracts prepared as described before were diluted to 10 mg TSP/mL using PBS, pH 7.0 buffer and 10 μL of the normalized extracts were spotted onto the whole-cell Cp6 TAP agar plate in duplicates. The spotted whole-cell Cp6 TAP agar plates were incubated at 37° C. overnight and the presence of endolysin activities was determined based on the appearance of clearance zones.

In the PGN degradation assay, purified peptidoglycan (PGN) from C. perfringens Cp6 cells was mixed with algal extract, and the degradation of the PGN over time was followed by reading the optical density of the solution at 620 nm. Algal extracts were 2-fold serially diluted up to 7 times in PBS pH 7.0 buffer, and 10 μL/well was dispensed into a microtiter plate according to the layout shown in FIG. 30A. To initiate the PGN assay, 90 μL/well of PGN stock was dispensed to give the effective loading concentrations. The reduction in turbidity of the well was immediately followed using the kinetic read function on a Labtech LT-4500 plate reader at 620 nm for 2 hours at room temperature (i.e. ˜23° C.). Initial rates of the PGN degradation were calculated using the Labtech LT-4500 plate reader's analysis software. MIC assay method and conditions were similar to Example 15 except for the following modifications: (1) an overnight culture of C. perfringens Cp6 strain was diluted in BHI+C medium instead of in LB medium; (2) assay was carried out with ˜10\hat{ }5 C. perfringens cells/mL; and (3) algal extracts instead of purified endolysins from E. coli were used. To initiate the MIC assay, 10 μL/well of 2-fold serially diluted algal extracts were dispensed according to the plate format shown in FIG. 30B, followed by the addition of 90 μL/well of a ˜10\hat{ }5 cells/mL of C. perfringens Cp6 strain, giving the effective loading concentrations. The MIC assay plates were sealed using adhesive aluminium seal (Starlab) and incubated at 41° C. overnight with moderate mixing on a plate shaker in the anaerobic chamber. Confirmation of bacterial loading in the MIC assay was similarly carried out as described in Example 15. Growth inhibition after overnight incubation was determined using both visual inspection as well as an OD620 nm read on the plate reader. The MIC value corresponds to the lowest extract concentration, expressed as mg TSP/mL, which fully inhibits bacteria growth. FIG. 31 shows results from the whole-cell Cp6 spot assay. Lytic activities were qualitatively assessed based on the size and clarity of the clearance zones around each spot. crAL035, a strain that was created by transformation with a vector without an endolysin gene, was used as a control. As expected, the extract from this strain did not create a clearance zone. In contrast, spot assay results show that the extracts produced from endolysin-expressing Chlamydomonas strains are active. Strains expressing the GH25CPFORC3 endolysin (crAL045, crAL057, and crAL061), all produced large clearance zones. The size and extent of clearance by crAL045 and crAL061 lysates appeared similar despite GH25CPFORC3 gene being expressed under different promoter/5′UTR elements. Clearance zones formed by strains expressing the AMI2phiZP2 endolysin (crAL038 and crAL039) were reduced in comparison with strains expressing GH25CPFORC3. The crAL039 spots showed fainter clearance zones in comparison to crAL038 spots. This difference is likely due to the different strength of promoter/5′UTR used for the expression of the AMI2phiZP2 endolysin. Interestingly, soluble extracts from crAL057, which co-expresses both GH25CPFORC3 and AMI2phiZP2, exhibited larger clearance zones than the crAL038 or crAL061 spots alone, which carry AMI2phiZP2 and GH25CPFORC3, respectively, under the same expression elements as in crAL057. This result suggests that co-expression of two different endolysins can increase overall bacteriolytic activity.

Results from the PGN degradation assay (FIG. 32) correlated well with whole-cell CP6 spot assay results. Extracts prepared from endolysin-expressing strains showed higher degradation rates than the background PGN degradation measured using extract from control strain crAL035, which does not express an endolysin gene. Strains which carried the GH25CPFORC3 gene produced the extracts with highest rates of PGN degradation, and these were more than 8-fold higher than those obtained from extracts containing the AMI2phiZP2 endolysin. The GH25CPFORC3 and AMI2phiZP2 co-expressing strain crAL057 exhibited the highest PGN degradation activity, higher than strains expressing GH25CPFORC3 (crAL061) or AMI2phiZP2 (crAL038) individually, further corroborating evidence that co-expression of two endolysins can achieve higher lytic activities.

In order to characterize the antimicrobial activity of the algal extracts, a MIC determination assay was carried out for the algal extracts (FIG. 33). MIC assay results generally correlate with spot and PGN assay results. MIC values could be determined from all extracts except from the control strain crAL035 and strain crAL039 (expressing AMI2phiZP2) where their MIC values could not be determined at higher than the highest amount of extract loaded (3 mg TSP/mL). GH25CPFORC3 and AMI2phiZP2 co-expressing strain crAL057 exhibited the highest antimicrobial activity with a MIC value of 188 g TSP/ml, and corroborates spot and PGN assay results that multiple expressions of two different endolysins is advantageous. The MIC values of extracts from crAL038, crAL045 and crAL61 were determined to be in the range of 750-375 μg TSP/ml. Differently to what was observed in the PGN assay and spot assay, antimicrobial activity determination showed that AMI2phiZP2-expressing strain crAL038 was more effective at inhibiting C. perfringens Cp6 growth than the GH25CPFORC3-expressing strains crAL045 and crAL061. PGN assay results had suggested that their lytic activities were 8-10-fold higher than that of crAL038, but found to be less antimicrobial in the MIC assay. Similarly, PGN assay results suggested that the lytic activity of crAL038 is only 2-3-fold higher than crAL039. However, their respective MIC values suggest that crAL038 is at least an order magnitude more inhibitory on C. perfringens CP6 than crAL039. Reasoning for these disparities could be that that there are various undefined factors present in the MIC assay that are not in a PGN assay. For example, endolysin activity and stability at 41° C., growth competition and PGN repair by the live C. perfringens cells, relative amounts of endogenous inhibitory components produced by the different Chlamydomonas strains, and/or that different cutting of bonds by the endolysins have different disruptive impacts to the cell wall integrity of a live cell. Nevertheless, results show that endolysins expressed in Chlamydomonas strains and used directly are antimicrobial against C. perfringens strain CP6.

### Example 25: Characterization of Extracts from Endolysin-Expressing Chlamydomonas reinhardtii Strains crAL075, crAL076, crAL077, crAL078, crAL079, and crAL080

This example compares the lytic activities of extracts prepared from strains of C. reinhardtii expressing additional endolysins discovered from the bioinformatics search. The strains constructed and characterized are summarized in Table 21. Putative GH25 endolysins based on Pfam sequence alignments characterized were GH25phiS63 (crAL075), AMI3CPF4969 (crAL079) and GH25CPFORC3 (crAL041 and crAL045). Putative AMI2 endolysins based on Pfam sequence alignments characterized were AMI2phiZP2 (crAL038 and crAL039) and GH25CPF4969 (crAL080). Putative AMI3 endolysins based on Pfam sequence alignments characterized were AMI3CPFORC25 (crAL076), AMI3CPJP55 (crAL077) and AMI3phi24R (crAL078). All strains except for crAL045, crAL038 and crAL039 were constructed by integration downstream of the petB gene in the chloroplast genome of a cassette containing a tagged version of the endolysin gene under the regulation of the 16S promoter and psaA 5′UTR. crAL038 strain carries the AMI2phiZP2 gene also under the regulation of the 16S promoter and psaA 5′UTR, but this cassette was integrated downstream of the psbH gene. Previously characterized strains in Example 24, crAL035, crAL038, crAL039 and crAL045, were used as controls. The co-expressing strain crAL058, derived from the crAL057 strain described in Example 24 and that co-expresses AMI2phiZP2 and GH25CPFORC3, was also included in this set as a control strain.

Lab-scale algal culturing, soluble extract preparation, whole-cell Cp6 spot assay, and PGN degradation assay were done similarly as described in Example 24. For the spot assay, algal extracts were prepared at 5 mg TSP/mL (1× stock), and then 2-fold serially diluted up to 2 times using PBS, pH 7.0 buffer. 5 μL of 1-fold, 2-fold and 4-fold dilutions of each extract was spotted onto solid agar media embedded with C. perfringens Cp6 cells, and incubated overnight at 37° C. for clearance zone development. For the PGN assay, each algal extract was prepared at 10 mg TSP/mL (10× stock) and then 2-fold serially-diluted up to 7 times to 78 μg TSP/mL. Extract from strain crAL035 was 2-fold serially diluted only 3 times to 1250 μg TSP/mL. 10 μL/well of extract dilutions were dispensed into a 96-microwell plate according to the plate layout shown in FIG. 34. For a “PGN only” control with no extract loaded, 10 μL/well PBS, pH 7.0, was dispensed. The PGN assay was initiated by dispensing 90 μL/well PGN assay stock. The PGN assay plate was mixed quickly on a plate shaker and the reduction in turbidity due to PGN degradation was monitored on a plate reader at OD620 nm for up to 4 hours at room temperature (i.e. ˜23° C.). Initial rates of the PGN degradation profiles were determined using the Labtech LT-4500 plate reader's analysis software. Activities were calculating by plotting the initial PGN degradation rate with the extract loading concentration, and extracting the slope of the linear regression curve.

FIG. 35B shows the results of the whole-cell CP6 spot assay. Endolysin activities were qualitatively assessed based on the size and clarity of the clearance zones. Control extracts behaved similarly to what was observed in Example 24. Chlamydomonas extracts from crAL078—crAL080 all exhibited activities similar to or greater than crAL038, but significantly less than that of crAL041. Of the three putative GH25 endolysin-expressing strains tested, the GH25CPFORC3-expressing strain (crAL041) produced distinctively larger clearance zones than the AMI3CPF4969-expressing strain (crAL079) or the GH25phiS63-expressing strain (crAL075), which showed no activity. This last result was surprising because in Example 4, GH25phiS63 was determined to be the highest overexpressed endolysin in E. coli and that also showed high activity. Results suggest that the expression host, whether E. coli or C. reinhardtii, has an effect on the endolysin expression and/or activity. crAL078 (expressing AMI3phi24R) seemed to be the more active AMI3-expressing extract than crAL076 (expressing AMI3CPFORC25) in the spot assay. No activity was observed for AMI3CPJP55 endolysin expressed in crAL077, similar to results seen in Example 8 when the endolysin was expressed in E. coli.

The PGN degradation assay allowed quantification and ranking of the lytic activities of the different algal extracts (FIG. 36 and FIG. 37). Results confirmed observations made from the whole-cell CP6 spot assays. PGN assay results confirmed the lack of activity from crAL075 and crAL077 extracts. The PGN degradation profiles of these extracts were similar to the degradation profile of the crAL035 control extract (FIG. 36F), which in turn was similar to the background degradation of the PGN suspended only in PBS, pH 7.0 (FIG. 36A and FIG. 36B). Additionally, PGN assay helped to identify the most active extracts within each endolysin enzyme class. crAL041 was the most active putative GH25-expressing extract, crAL080 was the most active AMI2-expressing extract, and crAL076 was the most active putative AMI3-expressing extract. Muramidase activity of crAL041 extract was approximately 2-fold higher than crAL079 extract activity. Similarly, L-alanine amidase activity of crAL076, crAL078 and crAL080 were approximately 2.4, 1.2, and 6.2-fold higher than that of crAL038. Overall, endolysin-expressing extracts can be ordered from highest to lowest PGN degradation activity as follows: crAL041, crAL079, crAL080, crAL076, crAL078, and crAL038. crAL075 and crAL077 extracts did not exhibit any PGN degradation activity, probably because the expressed endolysins were not active. This last result is particularly surprising because GH25phiS63 endolysin was overexpressed in E. coli and showed high activity. This result highlights the large influence of the host on the expression of an active endolysin.

### Example 26: Bioreactor Culture and Spray-Drying Production of Engineered Chlamydomonas reinhardtii Strains crAL039, and crAL045 Biomass

Bioreactor culture and spray-drying production of engineered Chlamydomonas strains crAL039 and crAL045 was performed to understand its practicality for large-scale production as well as to assess the level of endolysin antimicrobial activities that could be recovered from small-scale production.

A 5 L impeller-driven bioreactor (Applikon Biotechnolgy) was used for the cultivation of C. reinhardtii capable of operating under fed-batch conditions. The bioreactor was filled with 4.6 L of a modified TAP media. This modified TAP media was prepared by dissolving per litre: 576.4 mg NH₄CH₃CO₂ (Merck), 53.8 mg (CH₃COO)₂Ca.H₂O (Merck), 100 mg MgSO₄.7H₂O (Merck), 100 mg K₂HPO₄ (Merck), 60 mg KH₂PO₄(Merck), 35 mL of 1× concentrate Kropat's trace metals [1]. The bioreactor, all connected tubing, filters, glass bottles and media were subsequently autoclaved at 121° C. for 20 min at 31.18 psi before use in an AMA270 autoclave (Astell). A concentrated feed mimicking the basal media was prepared by mixing 1171 mL Milli-Q filtered water with 620 mL glacial acetic acid (Merck) and 209 mL 10× concentrate Kropat's trace metals solution into which 88.67 g NH₄CH₃CO₂, 22.06 g K₂HPO₄, 11.17 g KH₂PO₄, 14.30 g MgSO₄.7H₂O, 7.69 g (CH₃COO)₂Ca.H₂O was added to make 2 L. The feed was filter-sterilized with a 0.22 μm vacuum cup filter and stored at room temperature. Prior to each fed-batch bioreactor culture, 1.5 L of feed was transferred into a 2 L sterile glass bottle that connected to the bioreactor. An Applikon Biotechnolgy control unit was used to maintain culture temperature (25° C.), pH (7.00), agitation (400RPM), and air flow rate (3 L/min) throughout. Small amounts of antifoam (Polypropylene Glycol 2000, ThermoFisher Scientific) were injected manually with a syringe as needed.

To prepare the Chlamydomonas strain inoculum (crAL045 or crAL039), a shake flask containing 50 mL of TAP media [2] was inoculated with the desired strain from a TAP storage culture and grown to stationary phase on an orbital shaker at 140 rpm in the light cabinet (LEEC) under constant light conditions (50-500 μmol m⁻² s⁻¹) for 3-4 days. The culture was then diluted 1:10 by inoculating 400 mL fresh TAP medium in a 2 L shake flask with 40 mL of the stationary phase Chlamydomonas culture, and allowed to grow for an additional 3 days under the same conditions. The 400 mL inoculum was then sterilely-added to the bioreactor pre-filled with 4.6 L of modified TAP media, bringing the final volume to 5 L. 30 mL culture time points were drawn daily from sampling ports and the OD at 750 nm was measured to monitor culture growth and to estimate cell density. The fed-batch culture was grown for 5-7 days, depending on the requirement, without illumination.

The bioreactor culture was harvested in 1 L increments where the cells were pelleted by centrifuging at 15,000×g for 15 min in a Sorvall Lynx 6000 centrifuge (ThermoFisher Scientific) and the supernatant removed. The biomass from each 1 L centrifuge bottle was pooled and normalised to an OD750 nm of approximately 300-400. The concentrated biomass was subsequently spray-dried using a Lab scale B6chi-290 spray dryer (Büchi Labortechnik AG). Spray-drying conditions were as follows: 180° C. inlet, 6 mL/min pump rate, 38 m³/h aspirator airflow rate, and 667 L/h spray gas flow rate. Biomass was collected via a collection pot attached to extension placed underneath the cyclone to facilitate heat loss. Upon collection biomass was placed in a container, weighed to determine the yield, and stored at 4° C.

FIG. 38 shows the growth profiles of Chlamydomonas reinhardtii strains crAL045 and crAL039 grown in a 5 L bioreactor. Strain crAL045 (FIG. 38A) was harvested at approximately 41 OD750 nm after 6 days of growth when the culture was at late exponential/early stationary phase. Cells were spray-dried, giving a total yield of 25.8 g of dried biomass. Strain crAL039 (FIG. 38B) was harvested after 5 days of growth at approximately 116 OD750 nm, when the culture was at late exponential/early stationary phase. The culture yield was 42 g of spray dried biomass.

### REFERENCES

[1] J. Kropat et al., “A revised mineral nutrient supplement increases biomass and growth rate in Chlamydomonas reinhardtii,” Plant J., vol. 66, no. 5, pp. 770-780, 2011.
[2] E. H. Harris, The Chlamydomonas Sourcebook Volume 1: Introduction to Chlamydomonas and Its Laboratory Use, Second edi. Academic Press, 2009.

### Example 27: MIC/MBC Assay of C. perfringens Strain Cp6 Using crAL039 and crAL045 Biomass

The antimicrobial activities of Chlamydomonas reinhardtii strains crAL045 and crAL039 and their spray-dried biomass produced from Example 26 was assessed using a MIC/MBC assay.

MIC/MBC assay to assess the antimicrobial activities of soluble extracts produced from crAL045 and crAL039 biomass was performed similarly to Example 15 but with the following changes: 1) Cp6 overnight culture dilutions were in BHI+C medium instead of in LB medium; 2) ˜10\hat{ }5 cells/mL, one order of magnitude higher in bacterial suspension was loaded compared to that loaded in Example 15; 3) overnight incubations in the anaerobic chamber included moderate shaking; and 4) endolysin-expressing Chlamydomonas soluble extracts produced from spray-dried biomass were used instead of purified endolysins expressed from E. coli.

For preparation of soluble extracts from biomass, 300 mg/mL spray-dried crAL045 and crAL039 biomass (from Example 26) was prepared and resuspended in PBS, pH 7.0 in a 2 mL tube. The suspension was lysed by freeze-thawing 3 times and via sonication (10 μm amplitude, 10 sec on/off, 10 cycles) using a Soniprep 150 sonicator (MSE). The whole-cell extracts were centrifuged at 31,030×g for 45 min in a 5430R centrifuge (Eppendorf), and the soluble extract supernatant transferred to a fresh tube. TSP content of the soluble extracts were determined using the Bio-Rad DC protein assay. Soluble extracts of crAL045 and crAL039 were both diluted to 30 mg TSP/mL using PBS, pH 7.0 buffer to make a 10× stock. The soluble extracts were 2-fold serially-diluted using PBS, pH 7.0 buffer 11 times, or to effectively 2048-fold dilution. To initiate the MIC assay, 10 μL/well of 2-fold serially-diluted soluble extracts were dispensed into a 96-well microtiter plate (Starlab) according to the layout in FIG. 39, followed by 90 μL/well of ˜10\hat{ }5 cells/mL Cp6, giving the effective loading concentrations. Serial-dilutions of crAL045 extracts were assayed in 3× replicates whereas crAL039 extracts were assayed in duplicates. The MIC assay plate was sealed using adhesive aluminium seal (Starlab) and incubated at 41° C. overnight with moderate mixing on a plate shaker in a MACS-MG500 anaerobic chamber (Don Whitely). Confirmation of CP6 bacterial loading in the MIC assay was similarly carried out as described in Example 15. Growth inhibition after overnight incubation (˜16 hrs) was determined using both visual inspection as well as an OD620 nm read on a Labtech LT-4500 plate reader. MIC values were assessed based on the lowest extract concentration found that there was no visible growth of bacteria. To assess the bactericidal effects of the extracts, 5 μL/well of the overnight MIC assay plate was inoculated into 100 μL/well BHI+C medium to prepare an MBC assay plate. The MBC assay plate was sealed using adhesive aluminium seal (Starlab) and incubated at 37° C. overnight with moderate mixing on a plate shaker in the anaerobic chamber. Bactericidal effects of the extracts were assessed after overnight incubation (˜20 hrs) by visually inspecting the wells. MBC values were assessed based on the lowest extract concentration found that there was no visible growth.

Antimicrobial activity determination of strains crAL045 and crAL039 produced from fed-batch bioreactor cultures and spray-dried using the MIC/MBC assay showed that they are highly active against C. perfringens strain Cp6. MIC values for crAL045 and crAL039 extracts were determined to be 47 μg TSP/mL and 375 μg TSP/mL, respectively (FIG. 40). Each extracts' MBC values increased 2-fold compared to their corresponding MIC values to 94 μg TSP/mL and 750 μg TSP/mL for crAL045 and crAL039, respectively. Results also show that ranking of their antimicrobial activities matched those using soluble extracts obtained from wet shake flask pellets (Example 24) with crAL045 being much more active than crAL039. MIC and MBC values show that crAL045 extract is approximately 10-fold more active than crAL039 extract. Interestingly, bioreactor-produced extracts seemed to be much more active than shake flask extracts, as the bioreactor extracts was able to inhibit at much lower MIC values. Therefore, results show that fed-batch bioreactor culture and spray-drying is a viable method for producing engineered Chlamydomonas strains with high antimicrobial activity.

### Example 28: Chlamydomonas reinhardtii Extracts Enhance Activities of Purified Endolysins (PGN Assay)

Results from Examples 24, 25, and 26 show that extracts from C. reinhardtii strains expressing recombinant endolysins have high activity against C. perfringens cells as well as against peptidoglycan isolated from these cells. Surprisingly, the activities of these extracts were higher than expected based on the level of recombinant protein expression in the alga and on the activities of the purified endolysins purified from E. coli. A plausible explanation for this high activity is that the algal cell extracts act to enhance the activity of the endolysin, an effect which has not been previously reported. In order to confirm the enhancement effect of C. reinhardtii extract on endolysin activity, and to assess whether this also applies to endolysins from different enzyme classes, extracts made from different C. reinhardtii strains were combined with different enzyme classes of endolysins recombinantly expressed and purified from E. coli. Algal extract enhancement of the endolysins was investigated and was determined to be present using the PGN degradation assay.

Four different C. reinhardtii extracts were tested to investigate their enhancement effect on endolysin activities. These included host strain crAL035 (described in Example 22), cultured in both dark and light conditions, and wild-type strains crAL053 and crAL054, cultured in the dark. Strains crAL053 and crAL054 correspond to commonly used C. reinhardtii lab strains CC-124 and CC-1690, respectively. CC-124 (137c) is an mt-wild type strain isolated in 1945 near Amherst Mass. (US). It is a cell walled strain, mutant for the nit genes. This strain was purchased from the Chlamydomonas Resource Center (www.chlamycollection.org). CC-1690, also known as Sager21gr, is an mt+ wild type cell walled strain with functional nit genes. This strain was a kind gift from Saul Purton (UCL, London, UK). None of the three C. reinhardtii strains contain endolysin genes.

C. reinhardtii strains crAL035, crAL053, and crAL054 were cultured under dark conditions similarly as described in Example 24 (hereafter referred as “35D”, “53D”, and “54D” respectively). For crAL035 cultured under light conditions (hereafter referred as “35 L”), shake flask cultures were incubated at 25° C. on an orbital shaker set at 140 RPM in the light cabinet (50-500 μmol m⁻² s⁻¹). Shake flask cultures were grown, harvested, and the soluble extracts prepared similarly to in Example 24. Total soluble protein (TSP) concentration was determined using the Bio-Rad DC protein assay kit. Algal extracts were diluted to 5 mg TSP/mL using PBS, pH 7.0 buffer. The potential enhancement effects of C. reinhardtii extracts were investigated using purified endolysins GH25CPFORC3 (SEQ ID NO: 1), AMI3CPF4969 (SEQ ID NO: 11), AMI2phiZP2 (SEQ ID NO: 3), and GH25CPF4969 (SEQ ID NO: 8). Briefly, E. coli-expressed GH25CPFORC3 and AMI3CPF4969 endolysins were fused to a FLAG tag whereas the AMI2phiZP2 and GH25CPF4969 endolysins were fused to a 6×HIS tag to facilitate purification. Endolysin purification was carried out as described previously in Example 10 and Example 19.

A PGN degradation assay was performed using purified endolysins mixed with either PBS, pH 7.0 buffer or C. reinhardtii extracts. A 10× stock solution of the purified endolysin (50-100 μg/mL) was prepared in either PBS pH 7.0 buffer or C. reinhardtii extract, and then was serially diluted 2-fold up to 6 or 7 times using the corresponding diluent (buffer or 5 mg TSP/mL algal extract). To initiate the PGN assay, 10 μL/well of 2× serially-diluted endolysins were dispensed according to the plate layout in FIG. 41A or B followed by 90 μL/well of PGN assay stock, giving the effective loading concentrations. The number of replicates depended on the PGN assay layout. For “PGN only” control wells, 10 μL/well of each diluent was mixed with 90 μL/well of PGN assay stock in 4× replicates. PGN only controls with 0 μg/mL endolysin loaded were included for the diluents to determine their background degradation rates, if any. For “PBS blank” control wells, 100 μL/well of PBS, pH 7.0 buffer was dispensed in 4× replicates. The assay plate was mixed briefly on a plate shaker and the reduction in PGN turbidity was monitored continuously using the kinetic read function on a Labtech LT-4500 plate reader at 620 nm for 3-6 hours at room temperature (i.e. ˜23° C.), and the initial rate of the PGN degradation profiles was extracted using Labtech analysis software. Calculations to determine the fold enhancement of the algal extracts on the PGN degradation activities of the endolysins in comparison to in PBS, pH 7.0 buffer were done using the equation described in FIG. 42.

First, the effects of the four different algal extracts (35D, 35 L, 53D and 54D) on the activity of purified GH25CPFORC3 and AMI2phiZP2 endolysins were investigated. These endolysins belong to different classes of enzymes, respectively, and catalytically cut at different bonds of the bacterial peptidoglycan layer. It was desirable to determine whether algal extracts enhance both endolysin enzyme classes. The endolysins were mixed with either buffer or algal extracts at different concentrations and relative degradation rates of the PGN were monitored.

PGN degradation profiles at 2.5, 1.25, and 0.63 μg/mL loading concentrations of GH25CPFORC3 are shown in FIGS. 43, 44, and 45, respectively. Plots A to D in each figure compare the PGN degradations of GH25CPFORC3 diluted in PBS or in the different algal extracts. Degradation profiles from 2.5, 1.25, and 0.63 μg/mL concentrations were analysed for assessing the fold enhancement of the endolysin in algal extracts compared to in buffer (FIG. 42). For the three endolysin concentrations (2.5, 1.25, 0.63 μg/mL), the initial rates of the PGN degradation curves were higher when the endolysin was diluted in algal extract than when they were in buffer. The enhancement effects of the algal extracts on the endolysin's activity were more apparent and higher at reduced endolysin loading concentrations at 1.25 or 0.63 μg/mL, and reached ˜2.5-fold enhancement over its activity in buffer depending on the algal extract (FIG. 46). With 2.5 μg/mL endolysin loading, algal extract enhancement of the already-fast PGN degradation rate could not be seen. However, additional dilutions of the endolysins improved the sensitivity of the assay to detect the enhancement effects provided by the algal extracts. At these reduced loading concentrations, the purified GH25CPFORC3 endolysin in buffer exhibited low degradation activity but was improved significantly in the presence of algal extract at 1.25 or 0.63 μg/mL concentrations. Algal extract enhancement of the endolysin at 0.63 μg/mL and below could also be observed but determining its fold enhancement was difficult. PGN degradation profiles at 0.31, 0.16, and 0.08 μg/mL loading concentrations of GH25CPFORC3 are presented in FIG. 47. Here, PGN degradation profiles comparing the endolysin diluted in buffer or in the different algal extracts are shown in a single plot for each concentration, where plots A, B, and C in the figure correspond to loading concentrations of 0.31, 0.16, and 0.08 μg/mL, respectively. It's clear that PGN degradation of the endolysin diluted in the different algal extracts was enhanced than when the endolysin was only in buffer. However, estimating fold enhancement at these concentrations were not performed due to the precipitous, “cliff-like” degradation behaviour of the endolysin diluted in buffer, making initial rate fittings problematic at these concentrations and point to the complicated degradation of an undefined, 3D mesh-like PGN substrate. The observation of a precipitous drop in PGN degradation can be seen from 1.25 to 0.08 μg/mL concentrations of the endolysin in buffer, where onset of the precipitous drop in PGN degradation is delayed with decreasing concentration. Interestingly, this precipitous drop in PGN degradation was not observed with 2.5 μg/mL loading. It was thought that the onset of the precipitous drop in the profiles were due to the more gradual accumulation of PGN degradation at reduced loadings before the PGN's structural integrity fails abruptly. Following, the PGN degradations continue at a gradual rate commensurate with the endolysin loading concentration. In contrast, this precipitous drop in PGN degradation was not observed at all for the endolysin in the presence of algal extract, suggesting that algal extract enzymes were aiding PGN degradations in parallel with the endolysin. Consequently, the four algal extracts tested all were able to enhance the PGN degradation activities of GH25CPFORC3 similarly, suggesting that enhancement by C. reinhardtii extracts are not limited to a single species.

Enhancement was also observed for the AMI2phiZP2 endolysin when diluted in the four algal extracts compared to when diluted in buffer. FIG. 48 shows that, similarly to what was observed for the GH25-type endolysin, GH25CPFORC3, the four algal extracts also were able to enhance the AMI2phiZP2 endolysin's activity. Plots A to C in FIG. 48 present the PGN degradation profiles of the endolysin diluted in buffer or diluted in 54D algal extract. The PGN degradation profiles show that enhancement effects were more apparent at a lower endolysin concentration of 1.25 μg/mL, where approximately 2.2-fold enhancement was seen depending on the extract (FIG. 49). Estimating fold enhancement at and below 0.63 μg/mL concentration could not be done as the AMI2phiZP2 endolysin's activities when diluted in buffer were effectively the same as the background degradation rate (0 μg/mL, FIG. 48C). In contrast, the activities of AMI2phiZP2 diluted to the same concentration but in algal extracts were still measurable, which may even be considered to be “infinitely” enhanced over the endolysin diluted in buffer. Therefore, results show that C. reinhardtii extracts are able to enhance the activities of endolysins from two different enzyme classes, independently of strain and algal growth conditions.

Second, it was desirable to investigate whether additional endolysins from each enzyme class would also be enhanced by an algal extract. For this purpose, AMI3CPF4969 and GH25CPF4969 endolysins were purified and tested. Previously tested endolysins, GH25CPFORC3 and AMI2phiZP2 endolysins were also included as controls. The PGN assay was modified slightly to ensure reproducibility and higher sensitivity of the assay at reduced loading concentrations for estimating fold extract enhancement. Here, only PBS, pH 7.0 buffer and 5 mg TSP/mL 54D extract were used as the diluents. Each serially-diluted endolysins were assayed in 3× replicates and a freshly-purified CP6 PGN stock was used to ensure high reproducibility and smooth degradation profiles.

Results show that algal extract enhancement of GH25CPFORC3 (FIG. 50) and AMI2phiZP2 (FIG. 51) is reproducible and consistent using 3× replicates. Similar to what was observed previously, the extent of extract enhancement is revealed more clearly at reduced loading concentrations. Interestingly, the freshly-purified PGN stock produced smoother degradation profiles and seemed less recalcitrant than the previous PGN stock. This was difficult to rationalize as CP6 PGN isolation was performed as described in Example 20. However, it can be understood that the PGN purification protocol is crude and many characteristics of the PGN is undefined, including on how its storage conditions and length may affect its degradation characteristics. Use of the fresh PGN assay stock showed that algal extract enhancement of GH25CPFORC3 is significant. Plots A-E of FIG. 50 show degradation profiles of the endolysin at different loading concentrations and either diluted in buffer or in algal extract. At 0.31 μg/mL concentration, a significant reduction in PGN degradation activity can be observed for the endolysin diluted in buffer compared to its activity at 0.63 μg/mL. This was thought to be similar to what was observed previously where at the reduced loading concentration, degradations are occurring at a more gradual rate but not sufficiently to disrupt the PGN's structural integrity. And, if given more time, a “cliff-like,” precipitous drop in PGN degradation would be observed. In contrast, the endolysin diluted in algal extract showed fast PGN degradations at 0.31 μg/mL loading concentration and even at 0.16 μg/mL. Surprisingly, the GH25CPFORC3 endolysin at 0.16 and 0.31 μg/mL concentrations diluted in buffer exhibited degradation rates almost as low as background degradation rates, suggesting significant enhancement by the algal extract. Algal extract enhancement of the AMI2phiZP2 endolysin was also found to be reproducible and consistent using 3× replicates. However, enhancement effects were not as significant. This could be due to the specific site, abundance, and/or accessibility of the PGN bonds cut by the AMI2-type endolysin. Nevertheless, algal extract enhancement effects on GH25CPFORC3 and AMI2phiZP2 endolysins were reproduced and were significant depending on the endolysin.

Additional endolysins tested, AMI3CPF4969 and GH25CPF4969, also showed enhancement by the algal extract in FIG. 52 and FIG. 53, respectively. Similar analysis can be done to assess the enhancement effect of the algal extract on the endolysins. The AMI3CPF4969 endolysin showed a behaviour reminiscent to the GH25CPFORC3 endolysin regarding the enhancement effect of the algal extract. At the 1.25 μg/mL concentration, the AMI3CPF4969 endolysin diluted in buffer exhibited a significant reduction in activity compared to at 2.5 μg/mL. In contrast, the endolysin diluted in algal extract continued to show fast degradation rates from 2.5 to 1.25 μg/mL concentrations, and also significantly higher degradation rates compared to the background at below 0.63 μg/mL concentrations. This was not true for the endolysin diluted in buffer as 0.63, 0.31, and 0.16 μg/mL concentrations all showed degradation rates similar to background. Therefore, results show that the algal extract enhances the PGN degradation activity of AMI3CPF4969 significantly. PGN assays carried out with another endolysin, GH25CPF4969, also showed enhancement by the algal extract (FIG. 53). Enhancement was observed at reduced endolysin concentrations below 1.25 μg/mL. It was concluded that the algal extracts enhance the PGN degradation activities specifically of GH25CPFORC3, AMI3CPF4969, AMI2phiZP2, and GH25CPF4969 endolysins, and also generally to both endolysin classes.

In summary, algal extracts enhance the PGN degradation activities of GH25CPFORC3, AMI3CPF4969, AMI2phiZP2, and GH25CPF4969 endolysins. The fold enhancement of endolysins from different classes are shown in FIG. 54. Extract enhancement was up to approximately 69-fold and 44-fold for GH25CPFORC3 and AMI3CPF4969 endolysins, respectively in this specific system tested. Extract enhancement for AMI3CPF4969 could also be said to be “infinitely” enhanced as the endolysin diluted in buffer exhibited degradation rates similar to background at concentrations below 0.63 μg/mL. Both AMI2phiZP2 and GH25CPF4969 endolysins were enhanced by algal extracts up to approximately 2-fold in this specific system tested.

### Example 29: Chlamydomonas reinhardtii Extract Enhances Activity of Purified Endolysins (MIC Assay)

Example 28 showed that algal extracts derived from different C. reinhardtii strains were able to enhance the PGN degradation activity of endolysins. It was desirable to determine whether this enhanced activity would also translate into higher antimicrobial activity against actively growing C. perfringens strain Cp6 cells. Furthermore, it is known that C. reinhardtii extracts contain endogenous non-enzymatic inhibitory compounds such as fatty acids and chlorophyll derivatives have antimicrobial activities that could additionally enhance effect the endolysin's activities. To investigate this, endolysins GH25CPFORC3 (SEQ ID NO: 1) and AMI2phiZP2 (SEQ ID NO: 3) were chosen as representative endolysins from different classes, and their antimicrobial activities was assessed in the presence of crAL054 algal extract in a MIC assay.

crAL054 extracts were prepared as descried in Example 28, from cells grown in the dark, and diluted to 5 mg TSP/mL using PBS, pH 7.0. Purified endolysins, GH25CPFORC3 and AMI2phiZP2, expressed in E. coli, were prepared as described previously (Example 10 and Example 19, respectively). The purified endolysin stocks were diluted to 850 μg/mL GH25CPFORC3 and 900 μg/mL AMI2phiZP2 (10× endolysin stock solutions) using either PBS pH 7.0 or 5 mg TSP/mL 54D algal extract diluent. Subsequently, the endolysins were 2× serially-diluted up to 11 times using the corresponding diluent.

MIC assay method and conditions were similar to Example 15 except for the following modifications: (1) an overnight culture of C. perfringens Cp6 strain was diluted in BHI+C medium instead of in LB medium; and (2) purified endolysins were diluted in either PBS, pH 7.0 buffer or 5 mg TSP/mL of 54D algal extract. To initiate the MIC assay, 10 μL/well of 2× serially-diluted soluble extracts were dispensed according to the plate format in FIG. 55A followed by 90 μL/well of ˜10\hat{ }4 cells/mL culture of the C. perfringens Cp6 strain, giving the effective loading concentrations. “Growth control” and “media control” wells were also included in 3× replicates for each diluent. For growth controls, 10 μL/well diluent was mixed with 90 μL/well 10\hat{ }4-fold diluted bacterial suspension. For media controls, 10 μL/well diluent was mixed with 90 μL/well BHI+C medium. The MIC assay plates were sealed using adhesive aluminium seal and incubated at 41° C. overnight with moderate mixing on a plate shaker in the anaerobic chamber. CFU determination of C. perfringens Cp6 cultures used for the MIC assay was carried out similarly as described in Example 15. Growth inhibition after overnight incubation was determined using both visual inspection as well as an OD620 nm read on the plate reader. The MIC value corresponds to the lowest endolysin concentration which fully inhibits bacteria growth. MIC assay results show that both purified endolysins GH25CPFORC3 and AMI2phiZP2 are enhanced when in the presence of 500 μg TSP/mL than with PBS pH 7.0.

FIG. 55B shows that MIC values for GH25CPFORC3 and AMI2phiZP2 endolysins were determined to be 1.1 and 5.6 μg/mL, respectively, when they were diluted in PBS buffer. Interestingly, when the endolysins were diluted in algal extract, their MIC values were reduced to 0.4 and 1.4 μg/mL for GH25CPFORC3 and AMI2phiZP2 endolysins, respectively. Results show that the extract enhances purified GH25CPFORC3 and AMI2phiZP2 endolysin activity approximately 2.5 and 4-fold, respectively, than when in the buffer, in the MIC assay. Growth controls for PBS and the algal extract show that 500 μg TSP/mL extract, on its own, is not inhibitory against C. perfringens Cp6 strain. In conclusion, this result indicates that the algal extract enhances the antimicrobial activity of endolysins. Because this effect was seen for both GH25CPFORC3 and AMI2phiZP2 endolysins, which belong to two different endolysin families, it is safe to conclude that the effect of the algal extract is not limited to one particular class of endolysin.

### Example 30: Synergistic Effects of Chlamydomonas Extracts Used in Combination (PGN Assay)

Previously, it had been shown using PGN degradation assays that there existed synergies between endolysins when used in combination (Example 21) and that Chlamydomonas extracts also enhanced endolysin activities (Example 28 and 29). Therefore, it was desirable to assess whether these synergies would still be present when Chlamydomonas extracts expressing endolysin were used directly and in combination. Furthermore, two Chlamydomonas extracts expressing different endolysins as well as a GH25CPFORC3/AMI2phiZP2 co-expressing extract were included to increase the number of combinations to be tested for synergistic effects. Here, an extract combination was determined to be synergistic if its initial degradation rate, was greater than the sum of each extract's rates when used individually.

Synergistic effects between different Chlamydomonas extract combinations in PGN degradation assays were investigated. They include extracts expressing endolysins from the following combinations: 1) crAL041/crAL038, 2) crAL041/crAL080, 3) crAL045/crAL076, 4) crAL045/crAL078, 5) crAL079/crAL076, 6) crAL038/crAL076, and 7) crAL076/crAL080. Additionally, crAL058 extract that co-expresses both GH25CPFORC3 and AMI2phiZP2 endolysins were combined with either crAL076 extract (expressing AMI3CPFORC25 only) to make combination 8 or crAL080 extract (expressing GH25CPF4969 only) to make combination 9 to investigate the synergistic effects of 3 endolysins used in combination. FIG. 56 summarizes the extracts combined and the corresponding endolysin(s) expressed in each extract.

Chlamydomonas strains tabulated shown in FIG. 56 were cultured in TAP media in flasks shaken at ˜140 rpm under dark conditions (i.e. ambient light) at 25° C. Soluble extracts prepared similarly as described in Example 24. TSP content of the soluble extracts was determined using Bio-Rad DC protein assay, and normalized to 5 mg TSP/mL (10× stock) using PBS, pH 7.0. Checkerboard PGN assay was setup similarly as described in Example 21 except that Chlamydomonas extracts were used instead of purified endolysins. Briefly, two checkerboard PGN assays were done in one 96-microwell plate with an example sample layout for a 1× extract starting concentration of 500 μg TSP/mL (FIG. 57). 10× stocks of column extracts (i.e. extract 1 or 3) were 2-fold serially-diluted 6 times to 78.1 μg/mL, whereas 10× stocks of row extracts (i.e. extracts 2 or 4) were 2-fold serially-diluted 4 times to 313 μg/mL. 10 μL/well of the corresponding extract 1 or 3 dilutions were dispensed into the appropriate columns and similarly 10 μL/well of the corresponding extract 2 or 4 dilutions were dispensed into the appropriate rows. 10 μL/well of PBS, pH 7.0 was added to columns 6 and 12 (extract 1 or 3 dilutions only, respectively). Similarly, 10 μL/well of PBS, pH 7.0 was added to row H wells (extract 2 or 4 dilutions only). Well H6 and H12 (“PGN only” control, with 0 μg/mL endolysin) was ensured to contain a total of 20 μL/well PBS, pH 7.0. The PGN assay was initiated by dispensing 80 μL/well PGN assay stock, thereby effectively diluting each extract 10-fold. Extract loadings in each well correspond to the row and column loading concentrations indicated. The assay plate was mixed briefly on a plate shaker and the reduction in PGN turbidity was monitored continuously using the kinetic read function a Labtech LT-4500 plate reader at 620 nm for 2-4 hours at room temperature (i.e. ˜23° C.).

Assessment of the 9 different combinations of Chlamydomonas extracts show that the combinations were synergistic. This was determined based on whether the combined rate, when the two extracts were used together, was greater than the sum of each extract's initial PGN degradation rate, or by the degradation amount at the end-point of the assay as noted previously. Synergistic effects were found to be present from various Chlamydomonas extract combinations. An example of synergistic effects of between two different extracts can be seen in FIG. 58 where crAL041 (expressing GH25CPFORC3) and crAL038 (expressing AMI2phiZP2) extracts were used. When 500 μg TSP/mL crAL041 and 500 μg/mL crAL038 extracts were used together, their combined rate (0.111 OD/hr) was determined to be greater than the sum of their individual rates (0.085 OD/hr). Similar synergistic effects with improved combined rates were observed at 250/250, 125/125, and 62.5/62.5 μg TSP/mL loading concentration ratios of crAL041 and crAL038 extracts. Assessment and qualification on the presence of synergistic effects between extracts were similarly applied for the following extract combinations: crAL041/crAL080 (FIG. 59), crAL045/crAL076 (FIG. 60), crAL045/crAL078 (FIG. 61), crAL079/crAL076 (FIG. 62), crAL038/crAL076 (FIG. 63), and crAL076/crAL080 (FIG. 64). Results show that the extract combinations were indeed synergistic.

These results corroborate evidence from Example 21 where different purified endolysin combinations were found to be synergistic. For example, combination of purified GH25CPFORC3 and AMI2phiZP2 endolysins were found to be synergistic (FIG. 21) and their corresponding Chlamydomonas extracts, crAL041 and crAL038 respectively, that express the same endolysins were also found to be synergistic. This suggest that synergistic effects of endolysin combinations continue to be present in addition to effects from Chlamydomonas extract enhancement. Furthermore, purified endolysin combinations not tested in Example 21 would also be synergistic. For example, combination of purified GH25CPFORC3 and AMI3CPFORC25 endolysins were not tested in Example 21 but their expression in corresponding Chlamydomonas extracts, crAL045 and crAL076, respectively were shown to be synergistic.

Testing of Chlamydomonas extracts also revealed that the combination of 3 endolysins can be synergistic. As discussed above, purified GH25CPFORC3 and AMI2phiZP2 endolysins (Example 21), as well as Chlamydomonas extracts crAL041 (expressing GH25CPFORC3) and crAL038 (expressing AMI2phiZP2), were all found to be synergistic when used in combination. Therefore, it was surprising that crAL058 extract (expressing both GH25CPFORC3 and AMI2phiZP2) was found to be also synergistic when combined with a third GH25CPF4969 endolysin expressed in crAL080 extract (FIG. 65). A synergistic effect was also observed for when the co-expressing crAL058 extract was used in combination with the crAL076 extract (expressing AMI3CPFORC25) as seen in FIG. 66. Therefore, results in the current example show that synergistic effects between endolysins are not negatively impacted by extract enhancement effects. In addition, results show two examples of synergistic effects when 3 endolysins were used in combination.

Overall, results in the current example and from Example 21 show that synergistic effects on the degradation of C. perfringens strain CP6 PGN between endolysins can be achieved by routine optimisation of the specific combination of enzymes used, as well as their loading concentrations, and endolysin ratios.

### Example 31: Synergistic Effects of Chlamydomonas-Expressed Endolysin Extracts Used in Combination (MIC Assay)

Following from the results of Example 30, a checkerboard MIC assay was also used to corroborate the synergistic effects of Chlamydomonas-expressed endolysin extracts from algal biomass when used in combination. Biomass crAL045 and crAL039 produced as in example 26, expressing GH25CPFORC3 and AMI2phiZP2 respectively, were used as representative Chlamydomonas-expressed endolysin extracts to show that PGN assay results correlate with MIC assays.

The checkerboard MIC assay was executed as follows. MIC assay method and conditions were similar to Example 15 except for the following changes: 1) Cp6 overnight culture dilutions were in BHI+C medium instead of in LB medium; 2) Chlamydomonas soluble extracts produced from biomass were used instead of purified endolysins; and 3) a checkerboard plate layout was used.

50 mg/mL spray-dried crAL045 and crAL039 biomass (from Example 26) was prepared and resuspended in PBS, pH 7.0. The suspension was lysed via repeated freeze-thaw (3×) and sonication (10 μm amplitude, 5 sec on/off, for 15 cycles)) using a Soniprep 150 ultrasonic disintegrator. The whole-cell extracts were centrifuged at 31,030×g for 30-45 min on an Eppendorf 5430R centrifuge, and the soluble extract supernatant transferred to a separate tube. Bio-Rad DC protein assay was done to determine the TSP of the soluble extracts. TSP content was determined to be 7.5 mg TSP/mL and 6.7 mg TSP/mL for neat crAL045 and crAL039 extracts respectively (10× stocks). A checkerboard MIC assay was setup according to the 96-microwell sample layout in FIG. 67. Neat crAL039 extract was 2× serially-diluted 6 times to 104.7 μg/mL, whereas neat crAL045 extract was 2× serially-diluted 10 times to 7.3 μg/mL. 5 μL/well of the corresponding crAL039 extract dilution was dispensed into the appropriate columns and similarly 5 μL/well of the corresponding crAL045 extract dilutions were dispensed into the appropriate rows. 5 μL/well of PBS, pH 7.0 was added to column 12 wells (crAL039 extract dilutions only) and also row H wells (crAL045 extract dilutions only). Well H12 (growth control, with 0 μg/mL endolysin) was ensured to contain a total of 10 μL/well PBS, pH 7.0. To initiate the MIC assay, 10 L/well of 2× serially-diluted soluble extracts were dispensed according to the plate format in FIG. 67 followed by 90 μL/well of ˜10⁴ cells/mL Cp6, giving the effective loading concentrations. Extract loadings in each well correspond to the row and column loading concentrations indicated. The MIC assay plate was sealed with adhesive aluminium seal and incubated overnight with moderate shaking 41° C. in a MACS-MG500 anaerobic chamber (Don Whitely). Growth inhibition after overnight incubation was determined using both visual inspection as well as an OD620 nm read in a Labtech LT-4500 plate reader. MIC values for crAL045 and crAL039 used individually were determined based on the lowest concentration found that there was no visible growth of bacteria. The presence of synergistic effects between crAL045 and crAL039 extracts was determined by calculating for the Fractional Inhibitory Concentration (FIC) index [1-4] using the equation described in FIG. 68. A synergistic combination is when the combination results in a FIC value of <0.5. An additive effect is defined as 0.5<FIC≤4, whereas an antagonistic effect is when FIC>4.

Checkerboard MIC assay results show that crAL045 and crAL039 endolysin extracts used in combination are synergistically improved over their individual MIC values against ˜2.1E4 cells/mL of C. perfringens strain Cp6 (FIG. 69A). crAL045 extract alone had a MIC value of 23.4 μg TSP/mL (well H5 in the plate layout), whereas crAL039 extract alone had a MIC values of 167.5 μg TSP/mL (well B12 in the plate layout). However, when the two extracts were combined at loading concentrations of 5.9 μg TSP/mL and 41.9 μg TSP/mL for crAL045 and crAL039 extracts, respectively, (well D7 in the plate layout), the combination was found to be synergistic after calculating for their fractional inhibition concentration index, giving a value of 0.5 (example calculation: 5.9/23.4+41.9/167.5=0.5). FIG. 69B summarizes clearly the significant drop of each extract, crAL045 and crAL039, required to inhibit Cp6 cell growth compared to when each extract is used on its own. This synergy is corroborated by checkerboard PGN assay results using crAL045 and crAL039 extracts as well as by checkerboard PGN assay results using purified GH25CPFORC3 and AMI2phiZP2 endolysins. This suggests that endolysins revealed to be synergistic when used in combinations in Examples 21 and 30 would also be synergistic in a checkerboard MIC assay.

### REFERENCES

[1] E G Moellering Jr. Antimicrobial combinations. In: Antibiotics in Laboratory Medicine. Lorian V (Ed.), The Williams & Wilkins Co. MDUSA, 330-396 (1996).
[2] Anonymous. Instructions to authors. Antimicrobial Agents and Chemotherapy, 2019, pp. 1-22
[3] Letrado P, Corsini B, Díez-Martinez R, Bustamante N, Yuste J E and Garcia P. Bactericidal synergism between antibiotics and phage endolysin Cp1-711 to kill multidrug-resistant pneumococcus. Future Microbiol. (2018) 13(11): 1215-1223. DOI: 10.2217/fmb-2018-0077
[4] Vizquez R, and Garcia P. Synergy Between Two Chimeric Lysins to Kill Streptococcus pneumoniae. Frontiers in Microbiology (2019)10: 1251. DOI: 10.3389/fmicb.2019.01251.

TABLE 1

| Name | Endolysin Sequence |
|---|---|
| SEQ ID NO: 1 | TKLRGIDVSEHQGRINWEQVKDQVDFVMLRAGYGRNNIDKQFVRN |
| (GH25CPFORC3) | IQECNRLNIPVGIYWFSYAWNEEMAKNEAKYVLEAIKPYKVDYPI |
| | SYDLEYDTLNYAKKNGVTIGKRLATDMINVFCSTIENAGYKAMNY |
| | ANPDFINNKFYSNEVNYPLWLAWYGVSEDKAKAYNSAIWQFSESG |
| | SMDGIGINSVDMNYCYEDFLKKDFTLDNATTKNVSTKLNIRAKGT |
| | TNSRVVGSIPANEKFKIKWVDEDYLGWYYIEYNGIVGYVNADYVE |
| | KLQMATTHNVSTFLNVREEGSLNSRIVDKINVGDIFRIDWVDSDF |
| | IGWYRVTTKNGKVGFVNAEFVKKL |
| SEQ ID NO: 2 | KIIQSNIHFNGNKAGGNNPKEIIVHHSEHSTANVYDIDRWHKDKG |
| (AMI2PHICPV4) | WCGIGYHYFIDKQGNIYTGRPEDWTGAHCIDEINTKSIGICLQGRL |
| | QTEKVTDPQYKALLWLIQDIKNRRGNMPVYGHKELNSTDCPGNLD |
| | LDKLRRDLNNEVVDTNDDEYRENATVVNVSSYLNVRSKPSDEIIG |
| | KLFPNERLQVNWVDSDYLGWYYVTYRVNGTNKLKNGYVSAKYIKK |
| | D |
| SEQ ID NO: 3 | KIIQSNIYFNGNKAGGNNPKEIIVETHSEHSTANVYDIDKWHKDKG |
| (AMI2PHIZP2) | WCGIGYHYFIDKQGNIYTGRPEDWTGAHCINHNTKSIGICLQGRL |
| | QIEKVTDAQYKALLWLIQDIRNRRGNMPVYGHKELNSTDCPGNLD |
| | LNKLRTDVNNKVVDSNGGYTENATVVNVNSYLNVRSKPSDEIIGK |
| | LFPNERIQVNWVDSNYLGWYYITYRVNETNKLKSGYVSAKYIKKD |
| SEQ ID NO: 4 | KIAVRGGHNFQAKGSSALIDETIENRKVYKALIKYLSIAGHNVID |
| (AMI3CPSM101) | VTPGECDVNTDLYLGVQKAEDNNVELFISIHFDKAYDKYEGALGT |
| | GTWIYGRGGKAEIYAKRIVDNLSKGTGLKNRGVKENSKLYELRKT |
| | SMPAVLVEVCFCEATEDVRIYKEKGTDLIGKLIAEAINGDEIVGG |
| | DTRPENPEGSLKEKFLKSTNAKAIANLDPRDNPSSIYKDLGEIYK |
| | GERIRVLPEVCDKKDYLPIIYWKDTPNTESQKVWVNANQNYLKID |
| | TNATVINVVTELDARYTKSKTSSKMGYVKNGERLYIHKIENGYAL |
| | GTYFASDGYKTAWFTAKYISLD |
| SEQ ID NO: 5 | QSRNNNNLKGIDVSNWKGNINFESVKNDGVEVVYIKATEGNYFKD |
| (GH25PHIS63) | KYAKQNYEGAKEQGLKVGFYHFFRANKGAKDQANFFIDYLNEIGA |
| | VNYDCKLALDIETTEGVGVRDLTSMCIEFLEEVKRLTGKEVVVYT |
| | YTSFANNNLDSRLGNYPVWIAHYGVNTPGANNIWGSWVGFQYSEN |
| | RSVAGVNGGCDMNEFTEEIFIDSSNFNLDNATTKNVSTKLNIRAK |
| | GTTNSKVIGSIPADETFKIKWVDEDYLGWYYVEYNGVVGYVNADY |
| | VEKLQMATTYNVSTFLNVREEGSLNSRIVDKINSGDIFRIDWVDS |
| | DFIGWYRITTKNGKVGFVNAEFVKKL |
| SEQ ID NO: 6 | SKIFGLDAGHCTSGADTGAQGNGYKEQDLTRQVVTYLSEYLEKEG |
| (AMI3CPFORC25) | HTTKYCHCNSASTVNESLRYRVNKANSIGVDYFVSIHLNAGGGVG |
| | TETYICARGGEAERVAKRVNSKLVQYGYRDRGVKVGNLYVIKNTN |
| | APAILVEICFIDSSSDIAKFNAKAIAKAIAEGLLDKTIDEVEKKP |
| | ESVPDNTENSNTYFRVVVGSYKDRENAVKKQEELKAKGEDSFLLA |
| | YKE |
| SEQ ID NO: 7 | KIGIRDGHSPNCKGAIGLRDEQSCMRVLCKEVIEILEKHGHEVVY |
| (AMI3PHI24R) | CGSDASTQNSELSEGVRKANNSNVDIFISLHMNSFNGQAQGTESL |
| | IAVGARNSIKEIASRLCKNFASLGLVNRGVKEVNLYEMKNVKAPN |
| | IIFETMFCDNEHDINEVWSPTPYEKMALLIANAIDPTIKENELYR |
| | VVVQYFNSKKDAENCQQEIAKRWYCFVEECN |
| SEQ ID NO: 8 | KINKRLSTINVILNANNPKHIIIHEIDNISKGAGAETHCKAQANG |
| (GH25CPF4969) | NIGKASVHYYVDDTGVYQAVEHKHATWNCGDGNNRYGINNKNTIS |
| | IEICVNSDSDYNKAVDNTVELVRYLKNGYYSNCQVVRHYDASRKN |
| | CPRRILANGYWNTFLERVNSKDSSSQTPANTSYKGFYESSETRIN |
| | ATLVGEGSIKVLDEECNHVSGRWIDSLDRLFVIGIYPSRKFIEVV |
| | YPAGDKKYHAYIGIEYYNRILFDYHKEYINDDGVTYVWWNASDVN |
| | VKDHNEELQPNQKASPMYRTGEWLRITFYRENGIPSDGYVRYEGS |
| | QNKKFYENIQYGIVKVNSSLNVRENPNGEVIGSVYKDEKVQVLKE |
| | ENGWCYIEYSTSKGEKRGYVSSKYIELV |
| SEQ ID NO: 9 | YINQSNIKFNGLKYGNNPNKIIIHNADHPNCSVYDIDKWHKGNGW |
| (AMI2CPJP838) | SGIGYHYFIRKDGSIWTGRPENAIGAHTIGQNSSSIGICLEGALM |
| | REKPTRAQLNSIYELIADVRKRRGNLPVYGHKDFNNTDCPGKNFP |
| | LSEFKNNSYRPTGVSSETVVSENGFYTSNEERTNATIVGVGDIEV |
| | LDEKGKVIQGRHISSLDRVFVLGIYPSRNHIELIYPGKDEKYHAY |
| | ISIENYSRLSFDYHMQYKNDDGVTYVWWDSKNVNVKEHDEELQPH |
| | QKASPMYRTGGWLRITFYREDGTPSDGYVRYEGEQKERFYRKGKV |
| | VNVRTSLTVRSGAGTNYSAIGSLEPNENVDILGKAEGWYYIEYNT |
| | SNGKKKGYVSEKYIETIQ |
| SEQ ID NO: 10 | KISERGGHNFQATGAVGLINETVEDRKVLAAAYKYTKAAGHDVLN |
| (AMI3CPJP55) | VTPGNCDANTDLILGVNKAESFGAELFLSFHFDKCYDEYNGALGV |
| | ACWICEEGGKAEKYARSIVNTIVAGTGLINRGVKVNPRLYELRKT |
| | SMPAVIVEVCFCEATEDVRIYKEKGPDLIGKLIAEGVCKIAGGQV |
| | PGAVPETDDNKPVEPKPVPVYDRNKFKTNARALVALDPRDNASEV |
| | YEDLGEIYENERFYVLPEVCDKGNYLPVLYWKDGANRASNKVWVK |
| | SKQNYMMIDTYHKIVNVRTELDARYEPSPDSSRMGYVRNGERLYV |
| | HRTEGNYSLCTYFAGDGYKTAWFTAKYLERI |
| SEQ ID NO: 11 | QSRSDSNFKGIDISNWQKGINLNQLKERGYDVCYIKITEGKGYVD |
| (AMI3CPF4969) | PCFEENYNKAIAAGMKVGVYHYWRGTSSAIEQANNIVRTLGNKHI |
| | DCKIAIDVEQTDGLSYGELNNSVLQLAEELERLIGAEVCIYCNITNI |
| | YARNVLDSRLGKYSLWVAHYGVNKPGDNPIWDKWAGFQYSENGTS |
| | NVNGSLDLDEFTEEIFINKESSKVTENKLFSTNARALVALDPRDN |
| | PSDNYNDLGEIYEGERIQVLAEVCDKEDYLPVKYWKDSEGRESGK |
| | VWIRSKQDYMMIDTYHRVFNVITQLDARYEPSSDSATMGYVKNGE |
| | RLYVHRTEGNYSLCTYFAGNGYKTAWFTAKYLER |

Eleven full-length endolysin sequences were uncovered during the bioinformatics search. Each full length sequence is given a SEQ ID number with the Inventors' designations provided in brackets.

TABLE 2

| Name | N-Terminal catalytic domain sequences |
|---|---|
| SEQ ID NO: 12 | IDVSEHQGRINWEQVKDQVDFVMLRAGYGRNNIDKQFVRNIQEC |
| (GH25CPFORC3) | NRLNIPVGIYWFSYAWNEEMAKNEAKYVLEAIKPYKVDYPISYD |
| Amino acids 6-185 | LEYDTLNYAKKNGVTIGKRLATDMINVFCSTIENAGYKAMNYAN |
| of SEQ ID NO: 1 | PDFINNKFYSNEVNYPLWLAWYGVSEDKAKAYNSAIWQFSESGS |
| | MDGI |
| SEQ ID NO: 13 | NNPKEIIVHHSEHSTANVYDIDRWHKDKGWCGIGYHYFIDKQGN |
| (AMI2PHICPV4) | IYTGRPEDWTGAHCIDHNTKSIGICLQGRLQTEKVTDPQYKALL |
| Amino acids 17-132 | WLIQDIKNRRGNMPVYGHKELNSTDCPG |
| of SEQ ID NO: 2 | |
| SEQ ID NO: 14 | NNPKEIIVHHSEHSTANVYDIDKWHKDKGWCGIGYHYFIDKQGN |
| (AMI2PHIZP2) | IYTGRPEDWTGAHCINHNTKSIGICLQGRLQIEKVTDAQYKALL |
| Amino acids 17-132 | WLIQDIRNRRGNMPVYGHKELNSTDCPG |
| of SEQ ID NO: 3 | |
| SEQ ID NO: 15 | IAVRGGHNFQAKGSSALIDETIENRKVYKALIKYLSIAGHNVID |
| (AMI3CPSM101) | VTPGECDVNTDLYLGVQKAEDNNVELFISIHFDKAYDKYEGALG |
| Amino acids 2-173 | TGTWIYGRGGKAEIYAKRIVDNLSKGTGLKNRGVKENSKLYELR |
| of SEQ ID NO: 4 | KTSMPAVLVEVCFCEATEDVRIYKEKGTDLIGKLIAEAIN |
| SEQ ID NO: 16 | IDVSNWKGNINFESVKNDGVEVVYIKATEGNYFKDKYAKQNYEG |
| (GH25PHI563) | AKEQGLKVGFYHFFRANKGAKDQANFFIDYLNEIGAVNYDCKLA |
| Amino acids 11-186 | LDIETTEGVGVRDLTSMCIEFLEEVKRLTGKEVVVYTYTSFANN |
| of SEQ ID NO: 5 | NLDSRLGNYPVWIAHYGVNTPGANNIWGSWVGFQYSENRSVAGV |
| SEQ ID NO: 17 | LDAGHCTSGADTGAQGNGYKEQDLTRQVVTYLSEYLEKEGHTTK |
| (AMI3CPFORC25) | YCHCNSASTVNESLRYRVNKANSIGVDYFVSIHLNAGGGVGTET |
| Amino acids 6-168 | YICARGGEAERVAKRVNSKLVQYGYRDRGVKVGNLYVIKNTNAP |
| of SEQ ID NO: 6 | AILVEICFIDSSSDIAKFNAKAIAKAIAEGL |
| SEQ ID NO: 18 | LDAGHCTSGADTGAQGNGYKEQDLTRQVVTYLSEYLEKEGHTTK |
| (AMI3CPFORC25) | YCHCNSASTVNESLRYRVNKANSIGVDYFVSIHLNAGGGVGTET |
| Amino acids 6-154 | YICARGGEAERVAKRVNSKLVQYGYRDRGVKVGNLYVIKNTNAP |
| of SEQ ID NO: 6 | AILVEICFIDSSSDIAK |
| SEQ ID NO: 19 | GIRDGHSPNCKGAIGLRDEQSCMRVLCKEVIEILEKHGHEVVYC |
| (AMI3PHI24R) | GSDASTQNSELSEGVRKANNSNVDIFISLHMNSFNGQAQGTESL |
| Amino acids 3-170 | IAVGARNSIKEIASRLCKNFASLGLVNRGVKEVNLYEMKNVKAP |
| of SEQ ID NO: 7 | NIIFETMFCDNEHDINEVWSPTPYEKMALLIANAID |
| SEQ ID NO: 20 | NNPKHIIIHETDNTSKGAGAETHCKAQANGNIGKASVHYYVDDT |
| (GH25CPF4969) | GVYQAVEHKHATWNCGDGNNRYGINNKNTISIEICVNSDSDYNK |
| Amino acids 16-137 | AVDNTVELVRYLKNGYYSNCQVVRHYDASRKNCP |
| of SEQ ID NO: 8 | |
| SEQ ID NO: 21 | NNPNKIIIHNADHPNCSVYDIDKWHKGNGWSGIGYHYFIRKDGS |
| (AMI2CPJP838) | IWTGRPENAIGAHTIGQNSSSIGICLEGALMREKPTRAQLNSIY |
| Amino acids 16-131 | ELIADVRKRRGNLPVYGHKDFNNTDCPG |
| of SEQ ID NO: 9 | |
| SEQ ID NO: 22 | GHNFQATGAVGLINETVEDRKVLAAAYKYTKAAGHDVLNVTPGN |
| (AMI3CPJP55) | CDANTDLILGVNKAESFGAELFLSFHFDKCYDEYNGALGVACWI |
| Amino acids 7-172 | CEEGGKAEKYARSIVNTIVAGTGLINRGVKVNPRLYELRKTSMP |
| of SEQ ID NO: 10 | AVIVEVCFCEATEDVRIYKEKGPDLIGKLIAEGV |
| SEQ ID NO: 23 | IDISNWQKGINLNQLKERGYDVCYIKITEGKGYVDPCFEENYNK |
| (AMI2CPF4969) | AIAAGMKVGVYHYWRGTSSAIEQANNIVRTLGNKHIDCKIAIDV |
| Amino acids 11-179 | EQTDGLSYGELNNSVLQLAEELERLIGAEVCIYCNTNYARNVLD |
| of SEQ ID NO: 11 | SRLGKYSLWVAHYGVNKPGDNPIWDKWAGFQYSENGT |

All eleven full length sequences possess an N-terminal catalytic domain. Amino acid sequences of N-terminal catalytic domains are listed in the table below.

TABLE 3

| Name | Linker Sequence |
|---|---|
| SEQ ID NO: 24 | GTNSVDMNYCYEDFLKKDFTLDNATTKNVSTK |
| (GH25CPFORC3) | |
| Amino acids 186- | |
| 217 of SEQ ID NO: | |
| 1 | |
| SEQ ID NO: 25 | KLQMATTHNVSTF |
| (GH25CPFORC3) | |
| Amino acids 271- | |
| 283 of SEQ ID NO: | |
| 1 | |
| SEQ ID NO: 26 | NLDLDKLRRDLNNEVVDTNDDEYRENATVVNVS |
| (AMI2PHICPV4) | |
| Amino acids 133- | |
| 165 of SEQ ID NO: | |
| 2 | |
| SEQ ID NO: 27 | NLDLNKLRTDVNNKVVDSNGGYTENATVVNVN |
| (AMI2PHIZP2) | |
| Amino acids 133- | |
| 164 of SEQ ID NO: | |
| 3 | |
| SEQ ID NO: 28 | NGGCDMNEFTEEIFIDSSNFNLDNATTKNVSTK |
| (GH25PHIS63) | |
| Amino acids 187- | |
| 219 of SEQ ID NO: | |
| 5 | |
| SEQ ID NO: 29 | YNGVVGYVNADYVEKLQMATTYNVSTF |
| (GH25PHIS63) | |
| Amino acids 259- | |
| 285 of SEQ ID NO: | |
| 5 | |
| SEQ ID NO: 30 | FNAKAIAKAIAEGL |
| (AMI3CPFORC25) | |
| Amino acids 155- | |
| 168 of SEQ ID NO: | |
| 6 | |
| SEQ ID NO: 31 | RRILANGYWNTFLERVNSKDSSSQTPANTSYKGFYESSETRTNA |
| (GH25CPF4969) | TLVGEGSIKVLDEECNHVSGRWIDSLDRLFVIGIYPSRKFIEVV |
| Amino acids 138- | YPAGDKKYHAYIGIEYYNRILFDYHKEYINDDGVTYVWWNASDV |
| 332 of SEQ ID NO: | NVKDHNEELQPNQKASPMYRTGEWLRITFYRENGIPSDGYVRYE |
| 8 | GSQNKKFYENIQYGIVKVN |
| SEQ ID NO: 32 | KNFPLSEFKNNSYRPTGVSSETVVSENGFYTSNEERTNATIVGV |
| (AMI2CPJP838) | GDIEVLDEKGKVIQGRHISSLDRVFVLGIYPSRNHIELIYPGKD |
| Amino acids 132- | EKYHAYISIENYSRLSFDYHMQYKNDDGVTYVWWDSKNVNVKEH |
| 320 of SEQ ID NO: | DEELQPHQKASPMYRTGGWLRITFYREDGTPSDGYVRYEGEQKE |
| 9 | RFYRKGKVVNVRT |

The full length sequences designated by SEQ ID NOS 1, 2, 3, 5, 6, 8 and 9 each have a C-terminal cell binding domain, and therefore possess linker sequences connecting the N- and C-terminal domains, and connecting both C-terminal domains if two are present. Amino acid sequences of linker sequences are set out in the table below.

TABLE 4

| Name | C-terminal binding domain sequence |
|---|---|
| SEQ ID NO: 33 | LNIRAKGTTNSRVVGSIPANEKFKIKWVDEDYLGWYYIEYNGIV |
| GYVNADYVE | |
| (GH25CPFORC3) | |
| Amino acids 218- | |
| 270 of SEQ ID NO: | |
| 1 | |
| SEQ ID NO: 34 | LNVREEGSLNSRIVDKINVGDIFRIDWVDSDFIGWYRVTTKNGK |
| (GH25CPFORC3) | VGFVNAEFV |
| Amino acids 284- | |
| 336 of SEQ ID NO: | |
| 1 | |
| SEQ ID NO: 35 | LNIRAKGTTNSRVVGSIPANEKFKIKWVDEDYLGWYYIEYNGIV |
| (GH25CPFORC3) | GYVNADYVEKLQMATTHNVSTFLNVREEGSLNSRIVDKINVGDI |
| Amino acids 218- | FRIDWVDSDFIGWYRVTTKNGKVGFVNAEFV |
| 336 of SEQ ID NO: | |
| 1 | |
| SEQ ID NO: 36 | SYLNVRSKPSDEIIGKLFPNERLQVNWVDSDYLGWYYVTYRVNG |
| (AMI2PHICPV4) | TNKLKNGYVSAKYIK |
| Amino acids 166- | |
| 224 of SEQ ID NO: | |
| 2 | |
| SEQ ID NO: 37 | SYLNVRSKPSDEIIGKLFPNERIQVNWVDSNYLGWYYITYRVNE |
| (AMI2PHIZP2) | TNKLKSGYVSAKYIK |
| Amino acids 165- | |
| 223 of SEQ ID NO: | |
| 3 | |
| SEQ ID NO: 38 | LNIRAKGTTNSKVIGSIPADETFKIKWVDEDYLGWYYVE |
| (GH25PHIS63) | |
| Amino acids 220- | |
| 258 of SEQ ID NO: | |
| 5 | |
| SEQ ID NO: 39 | LNVREEGSLNSRIVDKINSGDIFRIDWVDSDFIGWYRITTKNGK |
| VGFVNAEFV | |
| (GH25PHIS63) | |
| Amino acids 286- | |
| 338 of SEQ ID NO: | |
| 5 | |
| SEQ ID NO: 40 | LNIRAKGTTNSKVIGSIPADETFKIKWVDEDYLGWYYVEYNGVV |
| (GH25PHIS63) | GYVNADYVEKLQMATTYNVSTFLNVREEGSLNSRIVDKINSGDI |
| Amino acids 220- | FRIDWVDSDFIGWYRITTKNGKVGFVNAEFV |
| 338 of SEQ ID NO: | |
| 5 | |
| SEQ ID NO: 41 | FNAKAIAKAIAEGLLDKTIDEVEKKPESVPDNTENSNTYFRVVV |
| (AMI3CPFORC25) | GSYKDRENAVKKQEELKAKGEDSFLL |
| Amino acids 155- | |
| 224 of SEQ ID NO: | |
| 6 | |
| SEQ ID NO: 42 | LDKTIDEVEKKPESVPDNTENSNTYFRVVVGSYKDRENAVKKQE |
| (AMI3CPFORC25) | ELKAKGEDSFLL |
| Amino acids 169- | |
| 224 of SEQ ID NO: | |
| 6 | |
| SEQ ID NO: 43 | SSLNVRENPNGEVIGSVYKDEKVQVLKEENGWCYIEYSTSKGEK |
| (GH25CPF4969) | RGYVSSKYIE |
| Amino acids 333- | |
| 386 of SEQ ID NO: | |
| 8 | |
| SEQ ID NO: 44 | SLTVRSGAGTNYSAIGSLEPNENVDILGKAEGWYYIEYNTSNGK |
| (AMI2CPJP838) | KKGYVSEKYIE |
| Amino acids 321- | |
| 375 of SEQ ID NO: | |
| 9 | |

The full length sequences designated by SEQ ID NOS: 1, 2, 3, 5, 6, 8 and 9 each have one or more C-terminal cell binding domains. Amino acid sequences of C-terminal binding domains are set out in the table below. The polypeptide designated by SEQ ID NOS: 1 and 5 possess two C-terminal cell binding domains. These are listed separately and together.

TABLE 5

| | | | Linker |
|---|---|---|---|
| Plasmid ID | Endolysin | Tag | sequence¹ |
| ecAL002 | — | — | — |
| ecAL005 | AMI2CPJP838 | C-terminal HA | GSAGSG |
| ecAL006 | AMI2phiCPV4 | C-terminal HA | GSAGSG |
| ecAL007 | AMI2phiZP2 | C-terminal HA | GSAGSG |
| ecAL008 | AMI3CPFORC25 | C-terminal Strep | GSAGSG |
| ecAL009 | AMI3CPJP55 | C-terminal Strep | GSAGSG |
| ecAL010 | AMI3CP5M101 | C-terminal Strep | GSAGSG |
| ecAL011 | AMI3phi24R | C-terminal Strep | GSAGSG |
| ecAL012 | AMI3CPF4969 | N-terminal FLAG | — |
| ecAL013 | GH25CPFORC3 | N-terminal FLAG | — |
| ecAL014 | GH25phiS63 | N-terminal FLAG | — |
| ecAL015 | GH25CPF4969 | C-terminal HA | GSAGSG |
| plAL073 | AMI2phiZP2 | C-terminal 6XHIS | GSAGSG |
| plAL071 | AMI2phiCPV4 | C-terminal 6XHIS | GSAGSG |
| plAL193 | GH25CPF4969 | C-terminal 6XHIS | GSAGSG |

¹Linker sequence between endolysin protein and tag.

List of plasmids constructed for endolysin recombinant expressions in the E. coli expression strain, Rosetta™ (DE3) pLysS, and their corresponding bacteria strain ID. Endolysin expression vectors were constructed using the parent ecAL002 plasmid, where endolysin gene sequences were cloned into the MCS2 of the plasmid. Subsequently, the plasmids were transformed into the Rosetta™ (DE3) pLysS expression strain.

TABLE 6

| Reference strain | 2837 | | 8235 | | 8237 | | 8238 | | 8239 | | 8359 | | 8678 | | 10578 | |
|---|---|---|---|---|---|---|---|---|---|---|---|---|---|---|---|---|
| (NCTC) | CL | FP | CL | FP | CL | FP | CL | FP | CL | FP | CL | FP | CL | FP | CL | FP |
| 20 μg/ml | − | + | − | + | − | + | − | + | − | + | − | + | − | + | − | + |
| Ampicillin | | | | | | | | | | | | | | | | |
| 1 mg/mL Lysozyme | − | − | − | − | − | − | − | − | − | − | − | + | − | − | − | − |
| bAL002 | − | − | − | − | − | − | − | − | − | − | − | − | − | − | − | − |
| bAL007 | − | − | − | − | n/a | n/a | − | − | n/a | n/a | n/a | n/a | − | − | − | − |
| (AMI2CPF4969) | | | | | | | | | | | | | | | | |
| bAL008 | − | + | − | + | + | − | − | − | + | + | + | − | − | − | − | − |
| (AMI2CPJP838) | | | | | | | | | | | | | | | | |
| bAL009 | + | + | + | + | + | + | + | + | + | + | + | + | + | + | + | − |
| (AMI2phiCPV4) | | | | | | | | | | | | | | | | |
| bAL010 | + | + | + | + | + | + | + | + | + | + | + | + | + | + | + | − |
| (AMI2phiZP2) | | | | | | | | | | | | | | | | |
| bAL011 | + | + | + | − | + | + | + | − | + | + | + | + | + | + | + | − |
| (AMI3CPFORC25) | | | | | | | | | | | | | | | | |
| bAL012 | − | − | − | − | n/a | n/a | − | − | n/a | n/a | n/a | n/a | − | − | − | − |
| (AMI3CPJP55) | | | | | | | | | | | | | | | | |
| bAL013 | + | + | + | + | + | − | + | + | + | + | + | + | + | + | + | − |
| (AMI3CPSM101) | | | | | | | | | | | | | | | | |
| bAL014 | + | + | + | + | + | + | + | − | + | + | + | + | + | + | − | − |
| (AMI3phi24R) | | | | | | | | | | | | | | | | |
| bAL015 | + | − | + | − | + | − | + | − | + | − | + | − | + | − | − | − |
| (GH25CPF4969) | | | | | | | | | | | | | | | | |
| bAL016 | + | + | + | + | + | + | + | + | + | + | + | + | + | + | + | + |
| (GH25CPFORC3) | | | | | | | | | | | | | | | | |
| bAL017 | + | + | + | − | + | + | + | + | + | + | + | + | + | + | + | − |
| (GH25phiS63) | | | | | | | | | | | | | | | | |

Summary of E. coli-expressed endolysins and their antimicrobial activities on different C. perfringens strains (NCTC 2837, 8235, 8237, 8238, 8239, 8359, 8678, and 10578). The table summarizes the antimicrobial activities from clarified lysates in spot assays on different C. perfringens strains, and on either freshly-plated cells or cell lawns. The E. coli strain and the corresponding endolysin expressed is also indicated. The antimicrobial activities of 20 μg/ml ampicillin and 1 mg/mL lysozyme were also included for reference purposes. The presence of antimicrobial activity is denoted by a ‘+’ whereas the lack of antimicrobial activity is denoted by a ‘-’. If a spot assay was not done for a particular clarified lysate or strain, it was denoted as ‘n/a’. ‘CL’—cell lawn; ‘FP’—freshly-plated cells. Evaluation for antimicrobial activity was based solely on the presence of a clearance zone and did not take into account the size of the halo. This was because endolysin concentrations (as determined from Example 4) in the spotted clarified lysates were not normalized. Spot assay results generally showed that the 20 μg/ml ampicillin exhibited inhibitory effect on freshly-plated cells but had no effect on cell lawn across the strains tested. 1 mg/mL lysozyme generally had no effect on both freshly-plated cells and cell lawns across the strains tested. In contrast, depending on the Cp strain and the particular endolysin, some endolysins exhibited bacteriolytic activities on both freshly-plated cells and cell lawns. For example, the clarified lysate of bAL016 (GH25CPFORC3) was able to produce clearance zones on both freshly-plated cells and cell lawns, and on all the Cp strains screened.

TABLE 7

| Non-Cp strain | 753 | | 1351 | | 6011 | | 20083 | |
|---|---|---|---|---|---|---|---|---|
| (DSMZ) | CL | FP | CL | FP | CL | FP | CL | FP |
| 20 μg/ml | − | n/a | − | + | − | − | − | + |
| Ampicillin | | | | | | | | |
| 1 mg/mL Lysozyme | − | n/a | − | − | − | − | − | − |
| bAL002 | − | − | − | − | − | − | − | − |
| bAL007 | n/a | n/a | n/a | n/a | n/a | n/a | n/a | n/a |
| (AMI2CPF4969) | | | | | | | | |
| bAL008 | − | n/a | − | − | − | − | − | − |
| (AMI2CPJP838) | | | | | | | | |
| bAL009 | − | n/a | − | − | − | − | − | − |
| (AMI2phiCPV4) | | | | | | | | |
| bAL010 | − | n/a | − | − | − | − | − | − |
| (AMI2phiZP2) | | | | | | | | |
| bAL011 | − | n/a | − | − | − | − | − | − |
| (AMI3CPFORC25) | | | | | | | | |
| bAL012 | n/a | n/a | n/a | n/a | n/a | n/a | n/a | n/a |
| (AMI3CPJP55) | | | | | | | | |
| bAL013 | − | n/a | − | − | − | − | − | − |
| (AMI3CPSM101) | | | | | | | | |
| bAL014 | − | n/a | − | − | − | − | − | − |
| (AMI3phi24R) | | | | | | | | |
| bAL015 | − | n/a | − | − | − | − | − | − |
| (GH25CPF4969) | | | | | | | | |
| bAL016 | − | n/a | − | − | − | − | − | − |
| (GH25CPFORC3) | | | | | | | | |
| bAL017 | − | n/a | − | − | − | − | − | − |
| (GH25phiS63) | | | | | | | | |

Summary of E. coli-expressed endolysins and their antimicrobial activities on different non-C. perfringens strains (DSMZ 753, 1351, 6011, 20083). The table summarizes the antimicrobial activities of the clarified lysates in spot assays on different non-C. perfringens species, tested on exponentially growing cells or stationary phase cells. The E. coli strain and the corresponding endolysin expressed is also indicated. The antimicrobial activities of 20 μg/ml ampicillin and 1 mg/mL lysozyme were also included as antimicrobial controls. The presence of antimicrobial activity is denoted by a ‘+’ whereas the lack of antimicrobial activity is denoted by a ‘−’. If a spot assay was not done for a particular clarified lysate or strain, or if the spot assay results was unclear, it was denoted as ‘n/a’. ‘CL’—cell lawn; ‘FP’—freshly-plated cells. Evaluation for antimicrobial activity was similar to what's indicated in Table 6. Spot assay results showed that the 20 μg/ml ampicillin exhibited inhibitory effect on freshly-plated cells depending on the bacteria species and had no effect on cell lawns. 1 mg/mL lysozyme generally had no effect on both freshly-plated cells and cell lawns across the bacteria species tested. The spot assay results also showed that the E. coli-expressed endolysins had no bacteriolytic activities on the non-C. perfringens species screened.

TABLE 8

| | GH25CPFORC3 | | | AMI2phiCPV4 | | | AMI2phiZP2 | | | Lysozyme | |
|---|---|---|---|---|---|---|---|---|---|---|---|
| | 100 | 50 | 10 | 100 | 50 | 10 | 100 | 50 | 10 | 50 | |
| | μg/ml | μg/ml | μg/ml | μg/ml | μg/ml | μg/ml | μg/ml | μg/ml | μg/ml | μg/ml | PBS |
| APHA | ++ | ++ | − | − | − | − | − | − | − | − | − |
| B00907 | | | | | | | | | | | |
| APHA | ++ | ++ | − | − | − | − | − | − | − | − | − |
| B00917 | | | | | | | | | | | |
| APHA | +++ | ++ | − | − | − | − | − | − | − | − | − |
| B00954 | | | | | | | | | | | |
| APHA | ++ | + | − | − | − | − | − | − | − | − | − |
| B00964 | | | | | | | | | | | |
| APHA | +++ | ++ | − | − | − | − | − | − | − | − | − |
| B00976 | | | | | | | | | | | |
| APHA | +++ | ++ | − | − | − | − | − | − | − | − | − |
| G00033 | | | | | | | | | | | |
| APHA | + | +/− | − | − | − | − | − | − | − | − | − |
| W100102 | | | | | | | | | | | |
| NCTC | +/− | +/− | − | − | − | − | − | − | − | − | − |
| 2837 | | | | | | | | | | | |
| NCTC | − | − | − | − | − | − | − | − | − | − | − |
| 8237 | | | | | | | | | | | |
| NCTC | − | − | − | − | − | − | − | − | − | − | − |
| 8238 | | | | | | | | | | | |
| NCTC | + | + | − | − | − | − | − | − | − | − | − |
| 8239 | | | | | | | | | | | |

Summary on the purified endolysins' growth inhibition effects on C. perfringens isolates and reference strains (freshly plated cells). (−) means no detectable bacterial growth inhibition, (+/−) detectable, (+) low, (++) good, (+++) very good. An example of a spot assay plate can be seen in FIG. 7A.

TABLE 9

| | GH25CPFORC3 | | | AMI2phiCPV4 | | | AMI2phiZP2 | | | Lysozyme | |
|---|---|---|---|---|---|---|---|---|---|---|---|
| | 100 | 50 | 10 | 100 | 50 | 10 | 100 | 50 | 10 | 50 | |
| | μg/ml | μg/ml | μg/ml | μg/ml | μg/ml | μg/ml | μg/ml | μg/ml | μg/ml | μg/ml | PBS |
| APHA | ++ | ++ | − | − | − | − | − | − | − | − | − |
| B00907 | | | | | | | | | | | |
| APHA | − | − | − | − | − | − | − | − | − | − | − |
| B00917 | | | | | | | | | | | |
| APHA | ++ | + | +/− | + | +/− | − | + | +/− | − | − | − |
| B00954 | | | | | | | | | | | |
| APHA | ++ | + | − | +/− | − | − | +/− | − | − | − | − |
| B00964 | | | | | | | | | | | |
| APHA | +++ | ++ | +/− | + | +/− | − | +/− | − | − | − | − |
| B00976 | | | | | | | | | | | |
| APHA | +++ | ++ | +/− | +/− | − | − | +/− | − | − | − | − |
| G00033 | | | | | | | | | | | |
| APHA | ++ | + | − | − | − | − | − | − | − | − | − |
| W100102 | | | | | | | | | | | |
| NCTC | ++ | + | − | − | − | − | − | − | − | − | − |
| 2837 | | | | | | | | | | | |
| NCTC | +++ | ++ | − | + | − | − | +/− | − | − | − | − |
| 8237 | | | | | | | | | | | |
| NCTC | + | + | +/− | +/− | − | − | +/− | − | − | − | − |
| 8238 | | | | | | | | | | | |
| NCTC | ++ | ++ | − | +/− | − | − | +/− | − | − | − | − |
| 8239 | | | | | | | | | | | |

Test 2 summary on the purified endolysins' bacteriolytic activities against C. perfringens isolates and reference strains. (−) indicates no visible lysis, and +/−, +, ++, +++ correspond to increasingly clear lysis halos around the paper disks. An example of a spot assay plate can be seen in FIG. 7B.

TABLE 10

| | Cp 2837 | | | Cp 8237 | | | Cp 8238 | | | Cp 8239 | | |
|---|---|---|---|---|---|---|---|---|---|---|---|---|
| lysin loading | ′FORC3 | ′V4 | ′ZP2 | ′FORC3 | ′V4 | ′ZP2 | ′FORC3 | ′V4 | ′ZP2 | ′FORC3 | ′V4 | ′ZP2 |

t_50% lysis (min), AVG

| | | | | | | | | | | | | |
|---|---|---|---|---|---|---|---|---|---|---|---|---|
| 5 μg/ml | 6.5 | 15.4 | 15.6 | 2.9 | 6.0 | 6.5 | 10.1 | 11.4 | 11.7 | 5.4 | 9.6 | 9.6 |
| 2.5 μg/ml | 9.2 | 13.7 | 14.1 | 6.0 | 6.5 | 8.7 | 15.2 | 14.9 | 19.4 | 7.6 | 10.4 | 12.6 |
| 1.25 μg/ml | 19.0 | 14.7 | 25.4 | 21.2 | 9.0 | 19.7 | 49.5 | 19.4 | 36.4 | 19.5 | 12.0 | 25.5 |
| 0.63 μg/ml | >27 | 34.5 | >27 | >50 | 20.6 | >50 | >55 | 35.2 | >55 | >43 | 17.4 | >43 |

t_50% lysis (min), STDEV

| | | | | | | | | | | | | |
|---|---|---|---|---|---|---|---|---|---|---|---|---|
| 5 μg/ml | 0.19 | 0.19 | 0.42 | 0.10 | 0.10 | 0.10 | 0.35 | 1.30 | 1.34 | 0.12 | 0.63 | 0.29 |
| 2.5 μg/ml | 0.25 | 0.25 | 0.19 | 0.25 | 0.03 | 0.35 | 0.59 | 0.42 | 0.35 | 0.76 | 0.19 | 0.25 |
| 1.25 μg/ml | 1.01 | 0.59 | 1.07 | 0.82 | 0.25 | 0.86 | 6.00 | 0.38 | 1.23 | 1.89 | 0.69 | 1.55 |
| 0.63 μg/ml | n/a | 1.96 | n/a | n/a | 1.55 | n/a | n/a | 2.38 | n/a | n/a | n/a | n/a |

Summary of an endolysin's bacteriolytic activity at different loading concentrations (5, 2.5, 1.25, and 0.63 μg/mL) and on different C. perfringens reference strains (NCTC 2837, 8237, 8238, 8239). The 't_50% lysis' parameter is defined as the time required for an endolysin to lyse 50% of its initial cell suspension loading. Averages and standard deviations were based on 3× replicates. Except for AMI2phiCPV4, no 't_50% lysis' values could be extracted from GH25CPFORC3 and AMI2phiZP2 lysis profiles at the lower 0.63 μg/mL loading concentration. This was because turbidity reduction was very slow and the OD620 nm kinetic read was terminated before lysis was >50%.

TABLE 11

| | | | | | Median |
|---|---|---|---|---|---|
| | | | | | across |
| | NCTC2837 | NCTC8237 | NCTC8238 | NCTC8239 | strains |
| GH25CPFORC3 | | | | | |
| (1 μg/ml) | | | | | |
| ΔLog10 | 0.31 ± 0.03 | 3.52 ± 0.91 | 0.51 ± 0.20 | 0.45 ± 0.07 | |
| % reduction | 50.37 ± 3.66 | 99.93 ± 0.07 | 67.07 ± 2.83 | 64.18 ± 5.80 | 65.63 |
| GH25CPFORC3 | | | | | |
| (5 μg/ml) | | | | | |
| ΔLog10 | 0.45 ± 0.11 | 3.76 ± 0.08 | 0.83 ± 0.19 | 0.91 ± 0.12 | |
| % reduction | 64.08 ± 8.66 | 99.98 ± 0.00 | 84.14 ± 6.53 | 87.45 ± 3.35 | 85.80 |
| AMI2phiCPV4 | | | | | |
| (1 μg/ml) | | | | | |
| ΔLog10 | 0.52 ± 0.12 | 0.40 ± 0.05 | 0.11 ± 0.06 | 0.61 ± 0.12 | |
| % reduction | 68.78 ± 8.76 | 60.36 ± 4.26 | 22.51 ± 10.26 | 74.93 ± 6.39 | 68.78 |
| AMI2phiCPV4 | | | | | |
| (5 μg/ml) | | | | | |
| ΔLog10 | 1.01 ± 0.18 | 0.44 ± 0.16 | 0.16 ± 0.18 | 1.30 ± 0.12 | |
| % reduction | 89.74 ± 4.45 | 61.85 ± 12.86 | 27.29 ± 29.06 | 94.88 ± 1.46 | 75.79 |
| AMI2phiZP2 | | | | | |
| (1 μg/ml) | | | | | |
| ΔLog10 | 0.19 ± 0.18 | 1.18 ± 0.27 | 0.09 ± 0.02 | 0.03 ± 0.05 | |
| % reduction | 31.92 ± 24.47 | 92.43 ± 4.95 | 12.31 ± 11.01 | 7.30 ± 10.68 | 22.11 |
| AMI2phiZP2 | | | | | |
| (5 μg/ml) | | | | | |
| ΔLog10 | 0.51 ± 0.16 | 2.07 ± 0.30 | 0.19 ± 0.08 | 0.09 ± 0.08 | |
| % reduction | 67.42 ± 12.65 | 98.99 ± 0.75 | 35.33 ± 12.88 | 18.04 ± 15.10 | 51.37 |

Summary of Δ log 10, % reduction, and median % reduction values of endolysin antimicrobial effects on the C. perfringens reference strains. Log 10 reduction values were calculated as the log 10 of the ratio of untreated cell control reactions over endolysin-treated cell reactions. The log 10 reduction values were then used to calculate the percentage reduction of viable bacteria for each endolysin. The median percentage reduction values were determined from percentage reduction values across all strains, including the 3 biological replicates, at each endolysin loading concentration.

TABLE 12
MIC assay plate (loading concentrations)

| | 1 | 2 | 3 | 4 | 5 | 6 | 7 | 8 | 9 | 10 | 11 | 12 | antimicrobial | |
|---|---|---|---|---|---|---|---|---|---|---|---|---|---|---|
| A | media control | | | | | | growth control | | | | | | n/a | |
| B | | | | | | | | | | | | | lysozyme | |
| C | | | | | | | | | | | | | | |
| D | | | | | | | | | | | | | GH25FORC3 | |
| E | | | | | | | | | | | | | | |
| F | | | | | | | | | | | | | AMI2phiCPV4 | |
| G | | | | | | | | | | | | | | |
| H | | | | | | | | | | | | | AMI2phiZP2 | |
| row | 1 | 2 | 4 | 8 | 16 | 32 | 64 | 128 | 256 | 512 | 1024 | 2048 | <-- x dilutions | |
| B, C | 2500 | 1250 | 625 | 312.5 | 156.3 | 78.13 | 39.06 | 19.5 | 9.8 | 4.9 | 2.4 | 1.2 | [lysozyme], | ug/mL |
| D, E | 100 | 50 | 25 | 12.5 | 6.25 | 3.125 | 1.563 | 0.781 | 0.391 | 0.195 | 0.098 | 0.049 | [GH25FORC3], | ug/mL |
| F, G | 65 | 32.5 | 16.25 | 8.125 | 4.063 | 2.031 | 1.016 | 0.508 | 0.254 | 0.127 | 0.063 | 0.032 | [AMI2phiCPV4], | ug/mL |
| H | 65 | 32.5 | 16.25 | 8.125 | 4.063 | 2.031 | 1.016 | 0.508 | 0.254 | 0.127 | 0.063 | 0.032 | [AMI2phiZP2], | ug/mL |
| row | 1 | 2 | 4 | 8 | 16 | 32 | 64 | 128 | 256 | 512 | 1024 | 2048 | <-- x dilutions | |
| B, C | 173.6 | 86.81 | 43.4 | 21.7 | 10.85 | 5.425 | 2.713 | 1.36 | 0.68 | 0.34 | 0.17 | 0.08 | [lysozyme], | uM |
| D, E | 2.5 | 1.25 | 0.625 | 0.313 | 0.156 | 0.078 | 0.039 | 0.02 | 0.010 | 0.005 | 0.002 | 0.001 | [GH25FORC3], | uM |
| F, G | 2.5 | 1.25 | 0.625 | 0.313 | 0.156 | 0.078 | 0.039 | 0.02 | 0.010 | 0.005 | 0.002 | 0.001 | [AMI2phiCPV4], | uM |
| H | 2.5 | 1.25 | 0.625 | 0.313 | 0.156 | 0.078 | 0.039 | 0.02 | 0.010 | 0.005 | 0.002 | 0.001 | [AMI2phiZP2], | uM |

Overnight MIC assay sample layout. All samples were assayed in 2× replicates except for AMI2phiZP2 at each loading concentration. The assay was initiated by mixing 5 μL/well 10-fold concentrated sample with 45 μL/well 1000-fold diluted stationary phase culture. 6× replicates of both media control (5 μL/well PBS, pH 7.0 buffer+45 uL 50 μL/well LB medium) and growth control (5 μL/well PBS, pH 7.0 buffer+45 uL 1000-fold diluted stationary phase culture) wells were also included.

TABLE 13

| | | | |
|---|---|---|---|
| lysozyme | FORC3 | V4 | ZP2 |

MIC/MBC (ug/mL)

| | | | | |
|---|---|---|---|---|
| MIC | >2500 | 1.6 | 16 | 16 |
| MBC | >2500 | 1.6 | 16 | 16 |

MIC/MBC (uM)

| | | | | |
|---|---|---|---|---|
| MIC | >174 | 0.04 | 0.63 | 0.63 |
| MBC | >174 | 0.04 | 0.63 | 0.63 |

fold improvement compared to lysozyme

| | | | | |
|---|---|---|---|---|
| MIC | n/a | >1563 | >156 | >156 |
| MBC | n/a | >1563 | >156 | >156 |

Minimum Inhibitory and Minimum Bactericidal Concentrations of GH25FORC3, AMI2phiCPV4, and AMI2phiZP2 endolysins on Cp6.

Summary of the purified endolysins' growth inhibition and bacteriolytic effects on C. perfringens reference strains. FP correspond to freshly-plated cells and CL correspond to cell lawns. (−) means no detectable bacterial growth inhibition, (+/−) detectable, (+) low, (++) good, (+++) very good. An example of a spot assay plate can be seen in FIG. 21. Results showed that the presence of synergistic interactions between GH25FORC3 and AMI2phiCPV4 depended on the Cp strain.

TABLE 14

| | NCTC8237 (FP) | NCTC8237 (CL) | NCTC8238 (FP) | NCTC8238 (CL) |
|---|---|---|---|---|
| 200 μg/ml FORC3 | − | +++ | +/− | + |
| 100 μg/ml FORC3 | − | +++ | + | + |
| 50 μg/ml FORC3 | − | ++ | + | + |
| 200 μg/ml V4 | − | ++ | +/− | + |
| 100 μg/ml V4 | − | + | +/− | + |
| 50 μg/ml V4 | − | +/− | +/− | + |
| 100 μg/ml FORC3 + 100 μg/ml V4 | − | ++ | +++ | +++ |
| 50 μg/ml FORC3 + 50 μg/ml V4 | − | ++ | ++ | ++ |

Table 15

96-microwell plate format of the serially-diluted endolysin stocks dispensed. 5 μL/well endolysin 1 and 5 μL/well endolysin 2 serially-diluted stocks according to the

96-microwell plate format 10 μL/well and 100 μL/well LB medium was dispensed into the growth control and media control wells, respectively.

### Table 16

Effective individual endolysin loadings, and of the ratios of endolysin 2 to endolysin 1 loaded, in the 96-microwell MIC assay plate. The uM ratios of endolysin 2 to endolysin 1 loaded are indicated in their respective wells. The dashed line illustrates the shape of the inhibitory line in the case of additive effects between the endolysins. Inhibited wells with no growth below and to the right of the dashed line would suggest that enhanced synergistic antimicrobial effects are present.

### Table 17

Effective individual endolysin loadings, and of the ratios of endolysin 2 to endolysin 1 loaded, in the 96-microwell MIC assay plate. The uM ratios of endolysin 2 to endolysin 1 loaded are indicated in their respective wells. The dashed line illustrates the shape of the inhibitory line in the case of additive effects between the endolysins. Inhibited wells with no growth below and to the right of the dashed line would suggest that enhanced synergistic antimicrobial effects are present.

TABLE 18

| Primer | | |
|---|---|---|
| name | Sequence (5′-3′) | Purpose |
| O_86 | TATAAGAAGGAGATATACATATGTAT | Cloning of AMI2CPJP838-HA |
| | ATCAATCAGTCCAACATTAAGTTCAA | into ecAL002 to construct |
| | TGGTC | ecAL005 |
| O_211 | GTTTCTTTACCAGACTCGAGTTATTA | Cloning of AMI2CPJP838-HA |
| | TGCGTAATCTGGAACATCATAGGGAT | into ecAL002 to construct |
| | AACCAC | ecAL005 |
| O_112 | TATAAGAAGGAGATATACATATGAA | Cloning of AMI2phiCPV4-HA |
| | AATTATCCAATCCAATATTCATTTTA | into ecAL002 to construct |
| | ATGGGAAC | ecAL006 |
| O_231 | GTTTCTTTACCAGACTCGAGTTATTA | Cloning of AMI2phiCPV4-HA |
| | AGCGTAATCTGGTACGTCATAAGGAT | into ecAL002 to construct |
| | AACC | ecAL006 |
| O_115 | TATAAGAAGGAGATATACATATGAA | Cloning of AMI2phiZP2-HA |
| | AATTATCCAGTCAAATATTTACTTTA | into ecAL002 to construct |
| | ATGGGAATAAAGC | ecAL007 |
| O_234 | GTTTCTTTACCAGACTCGAGTTATTA | Cloning of AMI2phiZP2-HA |
| | AGCGTAATCTGGAACGTCATAAGGA | into ecAL002 to construct |
| | TATCC | ecAL007 |
| O_105 | TTTATAGGTCTCAACATATGTCGAAA | Cloning of AMI3CPFORC25- |
| | ATTTTCGGTTTAGACGCAGGTC | Strep into ecAL002 to |
| | | construct ecAL008 |
| O_215 | GTTTCTTTACCAGACTCGAGTTATTA | Cloning of AMI3CPFORC25- |
| | TTTTTCAAATTGTGGATGTGATCATC | Strep into ecAL002 to |
| | CTGAACCTG | construct ecAL008 |
| O_92 | TATAAGAAGGAGATATACATATGAA | Cloning of AMI3CPJP55-Strep |
| | AATTTCAGAACGTGGTGG | into ecAL002 to construct |
| | | ecAL009 |
| O_212 | GTTTCTTTACCAGACTCGAGTTATTA | Cloning of AMI3CPJP55-Strep |
| | CTTTTCAAATTGAGGATGTGACCAAC | into ecAL002 to construct |
| | CTGAACC | ecAL009 |
| O_93 | TATAAGAAGGAGATATACATATGAA | Cloning of AMI3CPSM101- |
| | AATTGCTGTTCGAGGTGGCCATAATT | Strep into ecAL002 to |
| | TCC | construct ecAL0010 |
| O_213 | GTTTCTTTACCAGACTCGAGTTATTA | Cloning of AMI3CPSM101- |
| | TTTTTCAAATTGTGGATGACTCCAAC | Strep into ecAL002 to |
| | CTGAACCTG | construct ecAL0010 |
| O_75 | TATAAGAAGGAGATATACATATGAA | Cloning of AMI3phi24R-Strep |
| | AATCGGTATTAGAGATGGTCATAGTC | into ecAL002 to construct |
| | C | ecAL011 |
| O_195 | GTTTCTTTACCAGACTCGAGTTATTA | Cloning of AMI3phi24R-Strep |
| | TTTTTCGAATTGTGGATGACTCCAAC | into ecAL002 to construct |
| | CTG | ecAL011 |
| O_82 | TATAAGAAGGAGATATACATATGGA | Cloning of FLAG- |
| | CTATAAAGATGATGATGACAAAATG | AMI3CPF4969 into ecAL002 to |
| | CAATCACG | construct ecAL012 |
| O_205 | GTTTCTTTACCAGACTCGAGTTATTA | Cloning of FLAG- |
| | GCGCTCTAAGTATTTAGCAGTAAACC | AM13CPF4969 into ecAL002 |
| | ATGC | to construct ecAL012 |
| O_95 | TATAAGAAGGAGATATACATATGGA | Cloning of FLAG- |
| | CTATAAAGACGATGATGATAAAATG | GH25CPFORC3 into ecAL002 |
| | ACAAAATTACG | to construct ecAL013 |
| O_216 | GTTTCTTTACCAGACTCGAGTTATTA | Cloning of FLAG- |
| | TAATTTCTTAACGAATTCAGCATTTA | GH25CPFORC3 into ecAL002 |
| | CAAAGCCTACTTTGCC | to construct ecAL013 |
| O_74 | TATAAGAAGGAGATATACATATGGA | Cloning of FLAG-GH25phiS63 |
| | TTACAAAGACGATGACGACAAAATG | into ecAL002 to construct |
| | C | ecAL014 |
| O_232 | GTTTCTTTACCAGACTCGAGTTATTA | Cloning of FLAG-GH25phiS63 |
| | TAGCTTTTTCACGAATTCTGCGTTAA | into ecAL002 to construct |
| | CAAACC | ecAL014 |
| O_341 | ACCTGAACCAGCAGAACCGTCTT | Modification of ecAL006 |
| O_351 | CACCATCATCATCATCATTAATAACT | Modification of ecAL006 and |
| | CGAGTCTGGTAAAGAAACCGCTG | ecAL007 |
| O_342 | TCCGCTACCAGCAGAACCATCTTTTT | Modification of ecAL007 |
| | TAATG | |
| O_81 | TATAAGAAGGAGATATACATATGAA | Cloning of GH25CPF4969 - |
| | AATAAACAAGCGTTTATCAACTACGA | 6XHIS into ecAL002 to |
| | ATGTAACC | construct plAL193 |
| O_434 | GTTTCTTTACCAGACTCGAGTTATTA | Cloning of GH25CPF4969 - |
| | ATGATGATGATGATGGTGACCTGAAC | 6XHIS into ecAL002 to |
| | CAGCAGAACCAACTAATTC | construct plAL193 |

Primer sequences used for the construction of E. coli expression vectors

TABLE 19

| Primer | |
|---|---|
| name | Sequence (5′-3′) |
| O_53 | GTAGGTATGATTAGCTTTACTAAGCTAGTCATTG |
| O_328 | CACTTTTACAACAAAGTACATTAGGAAAAACACG |
| O_172 | AAATAGTAACATACTAAAGCGGATGTAACTCAATC |
| O_323 | AAATTTAAATTAGCAGAATCTTTGTCTTGATTAGGTG |
| O_336 | CACCCTAGTAAAGTAAATAAAATATACAACTCAATGAATC |
| O_197 | AATATAGGTCTCAATTAATCTTTTTTAATGTACTTGGCAGAAACGTAACCTG |
| O_106 | TTTATAGGTCTCAATGAAAATTATCCAGTCAAATATTTACTTTAATGG |

Primers used to screen for homoplasmic C. reinhardtii transformants

TABLE 20

| | | Targeted | | | | Antibiotic |
|---|---|---|---|---|---|---|
| | Backbone | integration | Endolysin gene | | | resistance |
| Plasmid | plasmid | site | Promoter/5’UTR | CDS | 3’UTR | gene |
| plAL151 | plAL010 | psbH | 16S/atpA | AMI2phiCPV4-HA | rbcL | — |
| plAL135 | plAL010 | psbH | 16S/psaA | AMI2phiZP2 | rbcL | — |
| plAL051 | pAxi2.0 | psbH | psaA/psaA | AMI2phiZP2-HA | rbcL | — |
| plAL163 | plAL009 | petB | 16S/psaA | FLAG-GH25CPFORC3 | rbcL | aadA |
| plAL136 | plAL010 | psbH | 16S/psaA | GH25CPFORC3 | atpA | — |
| plAL166 | plAL009 | petB | 16S/atpA | GH25CPFORC3 | atpA/psbA | crCD-aadA |
| plAL168 | plAL009 | petB | 16S/psaA | FLAG-GH25phiS63 | psbA | aadA |
| plAL169 | plAL009 | petB | 16S/psaA | AMI3CPFORC25-FLAG | psbA | aadA |
| plAL170 | plAL009 | petB | 16S/psaA | AMI3CPJP55-FLAG | psbA | aadA |
| plAL171 | plAL009 | petB | 16S/psaA | AMI3phi24R-FLAG | psbA | aadA |
| plAL172 | plAL009 | petB | 16S/psaA | FLAG-AMI3CPF4969 | psbA | aadA |
| plAL173 | plAL009 | petB | 16S/psaA | GH25CPF4969-FLAG | psbA | aadA |

Plasmids used to transform C. reinhardtii strains.

TABLE 21

| Strain | Host | | | | Selection | |
|---|---|---|---|---|---|---|
| ID | strain | Plasmid | Endolysin | Tag | method | Genotype |
| crAL035 | TN72 | plAL010 | — | None | Photosynthesis | psbH::[plAL010] |
| | | | | | restoration | cw15 mt+ |
| crAL036 | TN72 | plAL151 | AMI2phiCPV4 | C- | Photosynthesis | psbH::[16SatpA- |
| | | | | terminal | restoration | AMI2phiCPV4- |
| | | | | HA | | HA-rbcL] cw15 |
| | | | | | | mt+ |
| crAL038 | TN72 | plAL135 | AMI2phiZP2 | None | Photosynthesis | psbH::[16SpsaA- |
| | | | | | restoration | AMI2phiZP2- |
| | | | | | | rbcL] cw15 mt+ |
| crAL039 | TN72 | plAL051 | AMI2phiZP2 | C- | Photosynthesis | psbH::[psaApsa |
| | | | | terminal | restoration | A-AMI2phiZP2- |
| | | | | HA | | HA-rbcL] cw15 |
| | | | | | | mt+ |
| crAL041 | crAL035 | plAL163 | GH25CPFORC3 | N- | Antibiotic | psbH::[plAL010] |
| | | | | terminal | selection | petB::[16SpsaA- |
| | | | | FLAG | | FLAG- |
| | | | | | | GH25CPFORC3- |
| | | | | | | rbcL; atpAatpA- |
| | | | | | | aadA-rbcL] |
| | | | | | | cw15 mt+ |
| crAL045 | TN72 | plAL136 | GH25CPFORC3 | None | Photosynthesis | psbH::[16SpsaA- |
| | | | | | restoration | GH25CPFORC3- |
| | | | | | | atpA] cw15 mt+ |
| crAL057 | crAL038 | plAL166 | AMI2phiZP2 | None | Antibiotic | psbH::[16SpsaA- |
| | | | & | | selection | AMI2phiZP2- |
| | | | GH25CPFORC3 | | | rbcL] |
| | | | | | | petB::[16SatpA- |
| | | | | | | GH25CPFORC3- |
| | | | | | | atpA/psbA;psaAp |
| | | | | | | saA-crCD-aadA- |
| | | | | | | atpA/psbA] cw15 |
| | | | | | | mt+ |
| crAL058 | crAL057 | — | AMI2phiZP2 | None | Spontaneous | psbH::[16SpsaA- |
| | | | & | | loss of | AMI2phiZP2- |
| | | | GH25CPFORC3 | | antibiotic | rbcL] |
| | | | | | marker | petB::[16SatpA- |
| | | | | | | GH25CPFORC3- |
| | | | | | | atpA/psbA] |
| | | | | | | cw15 mt+ |
| crAL061 | crAL035 | plAL166 | GH25CPFORC3 | None | Antibiotic | psbH:: |
| | | | | | selection | [plAL010] |
| | | | | | | petB::[16SatpA- |
| | | | | | | GH25CPFORC3- |
| | | | | | | atpA/psbA; |
| | | | | | | psaApsaA- |
| | | | | | | crCD/aadA- |
| | | | | | | atpA/psbA] cw15 |
| | | | | | | mt+ |
| crAL075 | crAL035 | plAL168 | GH25phiS63 | N- | Antibiotic | psbH::[plAL010] |
| | | | | terminal | selection | petB::[16SpsaA - |
| | | | | FLAG | | FLAG- |
| | | | | | | GH25phiS63- |
| | | | | | | psbA; atpAatpA- |
| | | | | | | aadA-rbcL] |
| | | | | | | cw15 mt+ |
| crAL076 | crAL035 | plAL169 | AMI3CPFORC25 | C- | Antibiotic | psbH::[plAL010] |
| | | | | terminal | selection | petB::[16SpsaA- |
| | | | | FLAG | | AMI3CPFORC2 |
| | | | | | | 5-FLAG-psbA; |
| | | | | | | atpAatpA-aadA- |
| | | | | | | rbcL] cw15 mt+ |
| crAL077 | crAL035 | plAL170 | AMI3CPJP55 | C- | Antibiotic | psbH::[plAL010] |
| | | | | terminal | selection | petB::[16SpsaA- |
| | | | | FLAG | | AMI3CPJP55- |
| | | | | | | FLAG-psbA; |
| | | | | | | atpAatpA-aadA- |
| | | | | | | rbcL] cw15 mt+ |
| crAL078 | crAL035 | plAL171 | AMI3phi24R | C- | Antibiotic | psbH::[plAL010] |
| | | | | terminal | selection | petB::[16SpsaA- |
| | | | | FLAG | | AMI3phi24R- |
| | | | | | | FLAG-psbA; |
| | | | | | | atpAatpA-aadA- |
| | | | | | | rbcL] cw15 mt+ |
| crAL079 | crAL035 | plAL172 | AMI3CPF4969 | N- | Antibiotic | psbH::[plAL010] |
| | | | | terminal | selection | petB::[16SpsaA- |
| | | | | FLAG | | FLAG- |
| | | | | | | AMI3CPF4969- |
| | | | | | | psbA; atpAatpA- |
| | | | | | | aadA-rbcL] |
| | | | | | | cw15 mt+ |
| crAL080 | crAL035 | plAL173 | GH25CPF4969 | C- | Antibiotic | psbH::[plAL010] |
| | | | | terminal | selection | petB::[16SpsaA- |
| | | | | FLAG | | GH25CPF4969- |
| | | | | | | FLAG-psbA; |
| | | | | | | atpAatpA-aadA- |
| | | | | | | rbcL] cw15 mt+ |

Features of endolysin-carrying microalga strains

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, use of the singular forms “a”, “an”, and “the” include use of the plural unless the content clearly dictates otherwise. Thus, for example, reference to “a polypeptide” includes “polypeptides”, and the like.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “polypeptide” is used interchangeably with the term “protein”. As used herein, the term “amino acid” refers to either natural and/or unnatural or synthetic amino acids, including both D or L optical isomers, and amino acid analogs and peptidomimetics.

All publications, patents and patent applications cited herein, whether supra or infra, are hereby incorporated by reference in their entirety

1-20. (canceled)

21. An algal host cell comprising a population of nucleic acid molecules encoding an endolysin polypeptide, wherein each one of the molecules of the population encodes the same endolysin polypeptide or encode two or more different endolysin polypeptides, optionally wherein the host cell additionally comprises an endolysin polypeptide which has been expressed from said molecules; wherein the polypeptide has antimicrobial activity and comprises an amino acid sequence:
which is an N-terminal Clostridium perfringens cell wall peptidoglycan catalytic domain polypeptide,
or which is a fragment of said N-terminal catalytic domain polypeptide; or
which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11,
or which is a fragment of said N-terminal catalytic domain polypeptide, and which is at least 80% identical to the amino acid sequence of the sequence set forth in SEQ ID NO: 1, 2, 3, 4, 5, 6, 7, 8 or 11 which corresponds to the amino acid sequence of the fragment.

22. (canceled)

23. A cell lysate of a population of algal cells of claim 21, wherein the algal cells of the population comprise said optional endolysin polypeptide.

24. A composition comprising a population of endolysin polypeptides, wherein the population consists of the same endolysin polypeptide or consists of two or more different endolysin polypeptides; wherein the polypeptide has antimicrobial activity and comprises an amino acid sequence:
which is an N-terminal Clostridium perfringens cell wall peptidoglycan catalytic domain polypeptide,
or which is a fragment of said N-terminal catalytic domain polypeptide; or
which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11,
or which is a fragment of said N-terminal catalytic domain polypeptide, and which is at least 80% identical to the amino acid sequence of the sequence set forth in SEQ ID NO: 1, 2, 3, 4, 5, 6, 7, 8 or 11 which corresponds to the amino acid sequence of the fragment; and
wherein the population of endolysin polypeptides or fragments which have antimicrobial activity are: (a) added to the composition as components of one or more cell extracts; or (b) added to the composition as purified polypeptides or fragments and as components of one or more cell extracts; wherein in (a) or (b) the cell extract is an extract of an algal cell.

25. (canceled)

26. A composition comprising a population of algal host cells of claim 21, wherein the algal host cells of the population comprise said optional endolysin polypeptide.

27. The composition of claim 26, wherein the algal host cells are whole algal host cells.

28. (canceled)

29. (canceled)

30. A composition according to claim 26, wherein host cells, which optionally are whole cells, of the population are unicellular algal cells, more preferably Chlamydomonas sp., yet more preferably Chlamydomonas reinhardtii cells,
optionally wherein the composition comprises a dried biomass comprising the endolysin polypeptide or fragment which has antimicrobial activity expressed within algal cells, preferably wherein the composition or whole-cell composition is spray-dried.

31. (canceled)

32. A dried biomass composition comprising a population of algal host cells of claim 21, a cell lysate thereof, or a whole cell composition thereof, wherein the dried biomass composition comprises a cell lysate comprising said endolysin polypeptide(s) or fragment(s) which has antimicrobial activity and/or host cells comprising said endolysin polypeptide(s) or fragment(s) which has antimicrobial activity expressed within the host cells,
optionally wherein host cells of the population are unicellular algal cells, more preferably Chlamydomonas sp., yet more preferably Chlamydomonas reinhardtii cells; preferably wherein the dried biomass composition is spray-dried.

33. (canceled)

34. An antimicrobial formulation comprising an endolysin polypeptide and a pharmaceutically acceptable carrier/excipient; wherein the polypeptide has antimicrobial activity and comprises an amino acid sequence:
which is an N-terminal Clostridium perfringens cell wall peptidoglycan catalytic domain polypeptide, or which is a fragment of said N-terminal catalytic domain polypeptide; or
which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11,
or which is a fragment of said N-terminal catalytic domain polypeptide, and which is at least 80% identical to the amino acid sequence of the sequence set forth in SEQ ID NO: 1, 2, 3, 4, 5, 6, 7, 8 or 11 which corresponds to the amino acid sequence of the fragment; and
wherein the formulation comprises a population of host cells, a cell lysate thereof, a composition thereof, a whole cell composition thereof, or a dried biomass thereof, and the host cells of the population are defined according to claim 21.

35. (canceled)

36. An animal foodstuff comprising: (a) one or more foodstuffs; and (b) an isolated endolysin polypeptide; wherein the endolysin polypeptide consists of the same endolysin polypeptide or consists of two or more different endolysin polypeptides, and wherein the endolysin polypeptide has antimicrobial activity and comprises an amino acid sequence:
which is an N-terminal Clostridium perfringens cell wall peptidoglycan catalytic domain polypeptide,
or which is a fragment of said N-terminal catalytic domain polypeptide; or
which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11,
or which is a fragment of said N-terminal catalytic domain polypeptide, and which is at least 80% identical to the amino acid sequence of the sequence set forth in SEQ ID NO: 1, 2, 3, 4, 5, 6, 7, 8 or 11 which corresponds to the amino acid sequence of the fragment;
optionally wherein the foodstuff is suitable for consumption by poultry, optionally a broiler chicken, preferably Gallus gallus domesticus; or wherein the foodstuff is suitable for consumption by a pig, preferably Sus scrofa domesticus; or wherein the foodstuff is suitable for consumption by a rodent, optionally a mouse or rat.

37. An animal foodstuff comprising:
(a) one or more foodstuffs; and
(b) further comprising:
(i) a host cell according to claim 21;
(ii) a cell lysate thereof;
(iii) a composition comprising said host cell;
(iv) a whole cell composition of said host cell;
(v) a dried biomass composition comprising said host cell, cell lysate, or composition; and/or
(vi) an antimicrobial formulation comprising said host cell, cell lysate, composition, or dried biomass;
optionally wherein the foodstuff is suitable for consumption by poultry, optionally a broiler chicken, preferably Gallus gallus domesticus; or wherein the foodstuff is suitable for consumption by a pig, preferably Sus scrofa domesticus; or wherein the foodstuff is suitable for consumption by a rodent, optionally a mouse or rat.

38. (canceled)

39. The host cell according to claim 21, cell lysate thereof, composition comprising said host cell, whole cell composition comprising said host cell, dried biomass composition comprising said host cell, cell lysate, or composition, antimicrobial formulation comprising an endolysin polypeptide of said host cell, or animal foodstuff comprising said host cell, cell lysate, composition, dried biomass, or antimicrobial composition, comprising two or more different endolysin polypeptides or fragments which have antimicrobial activity;
optionally wherein the exhibited antimicrobial activity provided by the two or more different endolysin polypeptides or fragments is a synergistic activity.

40. (canceled)

41. The host cell, cell lysate, composition, whole-cell composition, dried biomass composition, antimicrobial formulation, or animal foodstuff according to claim 39,
A. further comprising one or more additional agents having antimicrobial activity and wherein the additional agent(s) is not an endolysin polypeptide or fragment; optionally wherein the one or more additional agents are selected form the group consisting of antibiotic agents, biofilm-degrading agents, biofilm-suppressing agents, sequestering agents such as chitosan, EDTA and citric acid, bacteriostatic agents such as glycerol; and/or
B. further comprising one or more additional agents selected form the group consisting of a stabilising agent, an anti-clumping agent, a prebiotic agent, a probiotic agent and an edible gel such as congealed water nutrient matrix; and/or
C. wherein the lysate, composition, whole-cell composition, antimicrobial formulation, or animal foodstuff is spray-dried, lyophilized or in powdered form.

42-58. (canceled)

59. The host cell according to claim 21, cell lysate thereof, composition comprising said host cell, composition comprising a population of endolysin polypeptides thereof, whole cell composition thereof, dried biomass composition comprising said host cell, lysate thereof, or composition, antimicrobial formulation comprising said endolysin polypeptide of said host cell, or animal foodstuff comprising said host cell, cell lysate, composition, dried biomass, or antimicrobial composition:
A. wherein the isolated polypeptide comprises an N-terminal Clostridium perfringens cell wall peptidoglycan catalytic domain polypeptide or fragment thereof, a C-terminal Clostridium perfringens cell wall binding domain polypeptide or fragment thereof and optionally a linker polypeptide or fragment thereof connecting the N- and C-terminal domains; wherein the isolated polypeptide comprises:
a) an amino acid sequence which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11, or an amino acid sequence which is a fragment of said N-terminal catalytic domain polypeptide and which is at least 80% identical to the amino acid sequence of the sequence set forth in SEQ ID NO: 1, 2, 3, 4, 5, 6, 7, 8 or 11 which corresponds to the amino acid sequence of the fragment; and
b) an amino acid sequence which is at least 80% identical to an endolysin C-terminal Clostridium perfringens cell wall binding domain polypeptide, or a fragment of said polypeptide; or which is an amino acid sequence which is at least 80% identical to the amino acid sequence of the C-terminal cell wall binding domain of the Clostridium perfringens bacteriophage endolysin polypeptide set forth in SEQ ID NO: 1, 2, 3, 5, 6, or 8, or an amino acid sequence which is a fragment of said C-terminal binding domain polypeptide and which is at least 80% identical to the amino acid sequence of the sequence set forth in SEQ ID NO: 1, 2, 3, 5, 6, or 8 which corresponds to the amino acid sequence of the fragment; and
c) an optional linker amino acid sequence which is at least 80% identical to the linker of an endolysin polypeptide, or a fragment of said polypeptide; or an optional amino acid sequence which is at least 80% identical to the amino acid sequence of a linker of the Clostridium perfringens bacteriophage endolysin polypeptide set forth in SEQ ID NO: 1, 2, 3, 5, 6, or 8, or an amino acid sequence which is a fragment of said linker and which is at least 80% identical to the amino acid sequence of the sequence set forth in SEQ ID NO: 1, 2, 3, 5, 6, or 8 which corresponds to the amino acid sequence of the fragment; wherein the linker amino acid sequence connects the amino acid sequence of the N-terminal cell wall peptidoglycan catalytic domain of the Clostridium perfringens bacteriophage endolysin polypeptide or fragment thereof and the C-terminal cell wall binding domain of the Clostridium perfringens bacteriophage endolysin polypeptide or fragment thereof; and/or
B. wherein the isolated polypeptide comprises an amino acid sequence comprising or consisting of an N-terminal Clostridium perfringens cell wall peptidoglycan catalytic domain polypeptide or fragment thereof, an amino acid sequence comprising or consisting of a C-terminal Clostridium perfringens cell wall binding domain polypeptide or fragment thereof and an amino acid sequence comprising or consisting of a linker polypeptide or fragment thereof connecting the N- and C-terminal domains; wherein the isolated polypeptide comprises:
a) an amino acid sequence which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11, or an amino acid sequence which is a fragment of said N-terminal catalytic domain polypeptide and which is at least 80% identical to the amino acid sequence of the sequence set forth in SEQ ID NO: 1, 2, 3, 4, 5, 6, 7, 8 or 11 which corresponds to the amino acid sequence of the fragment; and
b) an amino acid sequence which is at least 80% identical to the amino acid sequence of a C-terminal Clostridium perfringens cell wall binding domain of the Clostridium perfringens bacteriophage endolysin polypeptide set forth in SEQ ID NO: 1, 2, 3, 5, 6, or 8, or an amino acid sequence which is a fragment of said C-terminal binding domain polypeptide and which is at least 80% identical to the amino acid sequence of the sequence set forth in SEQ ID NO: 1, 2, 3, 5, 6, or 8 which corresponds to the amino acid sequence of the fragment; and
c) an amino acid sequence which is at least 80% identical to the amino acid sequence of a linker of the Clostridium perfringens bacteriophage endolysin polypeptide set forth in SEQ ID NO: 1, 2, 3, 5, 6, or 8, or an amino acid sequence which is a fragment of said linker and which is at least 80% identical to the amino acid sequence of the sequence set forth in SEQ ID NO: 1, 2, 3, 5, 6, or 8 which corresponds to the amino acid sequence of the fragment;
optionally wherein the isolated polypeptide consists of or comprises:
d) an amino acid sequence consisting of or comprising a fragment of said N-terminal catalytic domain and which is at least 80% identical to the amino acid sequence of the sequence set forth in SEQ ID NO: 1, 2, 3, 4, 5, 6, 7, 8 or 11 which corresponds to the amino acid sequence of the fragment; and/or
e) an amino acid sequence consisting of or comprising a fragment of said C-terminal binding domain and which is at least 80% identical to the amino acid sequence of the sequence set forth in SEQ ID NO: 1, 2, 3, 5, 6, or 8 which corresponds to the amino acid sequence of the fragment; and/or
f) an amino acid sequence consisting of or comprising a fragment of said linker and which is at least 80% identical to the amino acid sequence of the sequence set forth in SEQ ID NO: 1, 2, 3, 5, 6, or 8 which corresponds to the amino acid sequence of the fragment.

60. The host cell, cell lysate, composition, dried biomass composition, antimicrobial formulation or animal foodstuff according to part B of claim 59, wherein the isolated polypeptide comprises: an amino acid sequence wherein the N-terminal catalytic domain, the C-terminal cell wall binding domain and the linker are all at least 80%, 85%, 90%, 91%, 92%, 93%, 94%, 95%, 96%, 97%, 98%, 99% or 100% identical to corresponding amino acid sequences respectively of the N-terminal domain, a C-terminal domain and a linker of the Clostridium perfringens bacteriophage endolysin polypeptide set forth in SEQ ID NO: 1, 2, 3, 4, 5, 6, 7, 8 or 11;
preferably wherein the polypeptide comprises or consists of an amino acid sequence which is 80%, 85%, 90%, 91%, 92%, 93%, 94%, 95%, 96%, 97%, 98%, 99% or 100% identical to the amino acid sequence of the Clostridium perfringens bacteriophage endolysin polypeptide set forth in SEQ ID NO: 1, 2, 3, 4, 5, 6, 7, 8 or 11;
more preferably wherein the polypeptide comprises or consists of an amino acid sequence which is 100% identical to the amino acid sequence of the Clostridium perfringens bacteriophage endolysin polypeptide set forth in SEQ ID NO: 1, 2, 3, 4, 5, 6, 7, 8 or 11.

61. The host cell according to claim 21, cell lysate thereof, composition thereof, dried biomass composition thereof, antimicrobial formulation thereof, or animal foodstuff thereof, wherein the endolysin polypeptide or fragment has bacteriolytic activity and/or bacterial growth inhibitory activity, preferably against Clostridium perfringens, and more preferably against a Type A strain of Clostridium perfringens.

62. The algal host cell of claim 21, wherein the algal host cell comprises said endolysin polypeptide.

63. The algal host cell of claim 62, wherein the polypeptide has antimicrobial activity and comprises an amino acid sequence which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11.

64. The algal host cell of claim 63, wherein the polypeptide has antimicrobial activity and comprises an amino acid sequence which is at least 90% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11.

65. The algal host cell of claim 64, wherein the polypeptide has antimicrobial activity and comprises an amino acid sequence set forth in SEQ ID NO: 1, 2, 3, 4, 5, 6, 7, 8 or 11.

66. A method of prevention or treatment of a disease or disorder in an animal, the method comprising administering to the animal a prophylactically or therapeutically effective amount of an isolated endolysin polypeptide or fragment thereof, or a host cell of claim 21, a lysate thereof, a composition comprising the host cell (which optionally is a whole cell), a dried biomass comprising the host cell, an antimicrobial formulation comprising the host cell or polypeptide, or an animal foodstuff comprising the host cell or polypeptide, wherein optionally the polypeptide has antimicrobial activity and comprises an amino acid sequence:
which is an N-terminal Clostridium perfringens cell wall peptidoglycan catalytic domain polypeptide,
or which is a fragment of said N-terminal catalytic domain polypeptide; or
which is at least 80% identical to the amino acid sequence of the N-terminal cell wall peptidoglycan catalytic domain of the Clostridium perfringens bacteriophage endolysin polypeptide set forth in SEQ ID NO: 1, 2, 3, 4, 5, 6, 7, 8 or 11,
or which is a fragment of said N-terminal catalytic domain polypeptide, and which is at least 80% identical to the amino acid sequence of the sequence set forth in SEQ ID NO: 1, 2, 3, 4, 5, 6, 7, 8 or 11 which corresponds to the amino acid sequence of the fragment.


SEQUENCE LISTING (81 sequences)

SEQ ID NO: 1 - protein, 339 aa, Artificial Sequence
  GH25CPFORC3
TKLRGIDVSEHQGRINWEQVKDQVDFVMLRAGYGRNNIDKQFVRNIQECNRLNIPVGIYWFSYAWNEEMAKNEAKYVLEAIKPYKVDYPISYDLEYDTLNYAKKNGVTIGKRLATDMINVFCSTIENAGYKAMNYANPDFINNKFYSNEVNYPLWLAWYGVSEDKAKAYNSAIWQFSESGSMDGIGTNSVDMNYCYEDFLKKDFTLDNATTKNVSTKLNIRAKGTTNSRVVGSIPANEKFKIKWVDEDYLGWYYIEYNGIVGYVNADYVEKLQMATTHNVSTFLNVREEGSLNSRIVDKINVGDIFRIDWVDSDFIGWYRVTTKNGKVGFVNAEFVKKL

SEQ ID NO: 2 - protein, 226 aa, Artificial Sequence
  AMI2PHICPV4
KIIQSNIHFNGNKAGGNNPKEIIVHHSEHSTANVYDIDRWHKDKGWCGIGYHYFIDKQGNIYTGRPEDWTGAHCIDHNTKSIGICLQGRLQTEKVTDPQYKALLWLIQDIKNRRGNMPVYGHKELNSTDCPGNLDLDKLRRDLNNEVVDTNDDEYRENATVVNVSSYLNVRSKPSDEIIGKLFPNERLQVNWVDSDYLGWYYVTYRVNGTNKLKNGYVSAKYIKKD

SEQ ID NO: 3 - protein, 225 aa, Artificial Sequence
  AMI2PHIZP2
KIIQSNIYFNGNKAGGNNPKEIIVHHSEHSTANVYDIDKWHKDKGWCGIGYHYFIDKQGNIYTGRPEDWTGAHCINHNTKSIGICLQGRLQIEKVTDAQYKALLWLIQDIRNRRGNMPVYGHKELNSTDCPGNLDLNKLRTDVNNKVVDSNGGYTENATVVNVNSYLNVRSKPSDEIIGKLFPNERIQVNWVDSNYLGWYYITYRVNETNKLKSGYVSAKYIKKD

SEQ ID NO: 4 - protein, 337 aa, Artificial Sequence
  AMI3CPSM101
KIAVRGGHNFQAKGSSALIDETIENRKVYKALIKYLSIAGHNVIDVTPGECDVNTDLYLGVQKAEDNNVELFISIHFDKAYDKYEGALGTGTWIYGRGGKAEIYAKRIVDNLSKGTGLKNRGVKENSKLYELRKTSMPAVLVEVCFCEATEDVRIYKEKGTDLIGKLIAEAINGDEIVGGDTRPENPEGSLKEKFLKSTNAKAIANLDPRDNPSSIYKDLGEIYKGERIRVLPEVCDKKDYLPIIYWKDTPNTESQKVWVNANQNYLKIDTNATVINVVTELDARYTKSKTSSKMGYVKNGERLYIHKIENGYALGTYFASDGYKTAWFTAKYISLD

SEQ ID NO: 5 - protein, 341 aa, Artificial Sequence
  GH25PHIS63
QSRNNNNLKGIDVSNWKGNINFESVKNDGVEVVYIKATEGNYFKDKYAKQNYEGAKEQGLKVGFYHFFRANKGAKDQANFFIDYLNEIGAVNYDCKLALDIETTEGVGVRDLTSMCIEFLEEVKRLTGKEVVVYTYTSFANNNLDSRLGNYPVWIAHYGVNTPGANNIWGSWVGFQYSENRSVAGVNGGCDMNEFTEEIFIDSSNFNLDNATTKNVSTKLNIRAKGTTNSKVIGSIPADETFKIKWVDEDYLGWYYVEYNGVVGYVNADYVEKLQMATTYNVSTFLNVREEGSLNSRIVDKINSGDIFRIDWVDSDFIGWYRITTKNGKVGFVNAEFVKKL

SEQ ID NO: 6 - protein, 228 aa, Artificial Sequence
  AMI3CPFORC25
SKIFGLDAGHCTSGADTGAQGNGYKEQDLTRQVVTYLSEYLEKEGHTTKYCHCNSASTVNESLRYRVNKANSIGVDYFVSIHLNAGGGVGTETYICARGGEAERVAKRVNSKLVQYGYRDRGVKVGNLYVIKNTNAPAILVEICFIDSSSDIAKFNAKAIAKAIAEGLLDKTIDEVEKKPESVPDNTENSNTYFRVVVGSYKDRENAVKKQEELKAKGEDSFLLAYKE

SEQ ID NO: 7 - protein, 211 aa, Artificial Sequence
  AMI3PHI24R
KIGIRDGHSPNCKGAIGLRDEQSCMRVLCKEVIEILEKHGHEVVYCGSDASTQNSELSEGVRKANNSNVDIFISLHMNSFNGQAQGTESLIAVGARNSIKEIASRLCKNFASLGLVNRGVKEVNLYEMKNVKAPNIIFETMFCDNEHDINEVWSPTPYEKMALLIANAIDPTIKENELYRVVVQYFNSKKDAENCQQEIAKRWYCFVEECN

SEQ ID NO: 8 - protein, 388 aa, Artificial Sequence
  GH25CPF4969
KINKRLSTTNVTLNANNPKHIIIHETDNTSKGAGAETHCKAQANGNIGKASVHYYVDDTGVYQAVEHKHATWNCGDGNNRYGINNKNTISIEICVNSDSDYNKAVDNTVELVRYLKNGYYSNCQVVRHYDASRKNCPRRILANGYWNTFLERVNSKDSSSQTPANTSYKGFYESSETRTNATLVGEGSIKVLDEECNHVSGRWIDSLDRLFVIGIYPSRKFIEVVYPAGDKKYHAYIGIEYYNRILFDYHKEYINDDGVTYVWWNASDVNVKDHNEELQPNQKASPMYRTGEWLRITFYRENGIPSDGYVRYEGSQNKKFYENIQYGIVKVNSSLNVRENPNGEVIGSVYKDEKVQVLKEENGWCYIEYSTSKGEKRGYVSSKYIELV

SEQ ID NO: 9 - protein, 378 aa, Artificial Sequence
  AMI2CPJP838
YINQSNIKFNGLKYGNNPNKIIIHNADHPNCSVYDIDKWHKGNGWSGIGYHYFIRKDGSIWTGRPENAIGAHTIGQNSSSIGICLEGALMREKPTRAQLNSIYELIADVRKRRGNLPVYGHKDFNNTDCPGKNFPLSEFKNNSYRPTGVSSETVVSENGFYTSNEERTNATIVGVGDIEVLDEKGKVIQGRHISSLDRVFVLGIYPSRNHIELIYPGKDEKYHAYISIENYSRLSFDYHMQYKNDDGVTYVWWDSKNVNVKEHDEELQPHQKASPMYRTGGWLRITFYREDGTPSDGYVRYEGEQKERFYRKGKVVNVRTSLTVRSGAGTNYSAIGSLEPNENVDILGKAEGWYYIEYNTSNGKKKGYVSEKYIETIQ

SEQ ID NO: 10 - protein, 346 aa, Artificial Sequence
  AMI3CPJP55
KISERGGHNFQATGAVGLINETVEDRKVLAAAYKYTKAAGHDVLNVTPGNCDANTDLILGVNKAESFGAELFLSFHFDKCYDEYNGALGVACWICEEGGKAEKYARSIVNTIVAGTGLINRGVKVNPRLYELRKTSMPAVIVEVCFCEATEDVRIYKEKGPDLIGKLIAEGVCKIAGGQVPGAVPETDDNKPVEPKPVPVYDRNKFKTNARALVALDPRDNASEVYEDLGEIYENERFYVLPEVCDKGNYLPVLYWKDGANRASNKVWVKSKQNYMMIDTYHKIVNVRTELDARYEPSPDSSRMGYVRNGERLYVHRTEGNYSLCTYFAGDGYKTAWFTAKYLERI

SEQ ID NO: 11 - protein, 349 aa, Artificial Sequence
  AMI3CPF4969
QSRSDSNFKGIDISNWQKGINLNQLKERGYDVCYIKITEGKGYVDPCFEENYNKAIAAGMKVGVYHYWRGTSSAIEQANNIVRTLGNKHIDCKIAIDVEQTDGLSYGELNNSVLQLAEELERLIGAEVCIYCNTNYARNVLDSRLGKYSLWVAHYGVNKPGDNPIWDKWAGFQYSENGTSNVNGSLDLDEFTEEIFINKESSKVTENKLFSTNARALVALDPRDNPSDNYNDLGEIYEGERIQVLAEVCDKEDYLPVKYWKDSEGRESGKVWIRSKQDYMMIDTYHRVFNVITQLDARYEPSSDSATMGYVKNGERLYVHRTEGNYSLCTYFAGNGYKTAWFTAKYLER

SEQ ID NO: 12 - protein, 180 aa, Artificial Sequence
  N-terminal catalytic domain; Amino acids 6-185 of SEQ ID NO 1
IDVSEHQGRINWEQVKDQVDFVMLRAGYGRNNIDKQFVRNIQECNRLNIPVGIYWFSYAWNEEMAKNEAKYVLEAIKPYKVDYPISYDLEYDTLNYAKKNGVTIGKRLATDMINVFCSTIENAGYKAMNYANPDFINNKFYSNEVNYPLWLAWYGVSEDKAKAYNSAIWQFSESGSMDGI

SEQ ID NO: 13 - protein, 116 aa, Artificial Sequence
  N-terminal catalytic domain; Amino acids 17-132 of SEQ ID NO 2
NNPKEIIVHHSEHSTANVYDIDRWHKDKGWCGIGYHYFIDKQGNIYTGRPEDWTGAHCIDHNTKSIGICLQGRLQTEKVTDPQYKALLWLIQDIKNRRGNMPVYGHKELNSTDCPG

SEQ ID NO: 14 - protein, 116 aa, Artificial Sequence
  N-terminal catalytic domain; Amino acids 17-132 of SEQ ID NO 3
NNPKEIIVHHSEHSTANVYDIDKWHKDKGWCGIGYHYFIDKQGNIYTGRPEDWTGAHCINHNTKSIGICLQGRLQIEKVTDAQYKALLWLIQDIRNRRGNMPVYGHKELNSTDCPG

SEQ ID NO: 15 - protein, 172 aa, Artificial Sequence
  N-terminal catalytic domain; Amino acids 2-173 of SEQ ID NO 4
IAVRGGHNFQAKGSSALIDETIENRKVYKALIKYLSIAGHNVIDVTPGECDVNTDLYLGVQKAEDNNVELFISIHFDKAYDKYEGALGTGTWIYGRGGKAEIYAKRIVDNLSKGTGLKNRGVKENSKLYELRKTSMPAVLVEVCFCEATEDVRIYKEKGTDLIGKLIAEAIN

SEQ ID NO: 16 - protein, 176 aa, Artificial Sequence
  N-terminal catalytic domain; Amino acids 11-186 of SEQ ID NO 5
IDVSNWKGNINFESVKNDGVEVVYIKATEGNYFKDKYAKQNYEGAKEQGLKVGFYHFFRANKGAKDQANFFIDYLNEIGAVNYDCKLALDIETTEGVGVRDLTSMCIEFLEEVKRLTGKEVVVYTYTSFANNNLDSRLGNYPVWIAHYGVNTPGANNIWGSWVGFQYSENRSVAGV

SEQ ID NO: 17 - protein, 163 aa, Artificial Sequence
  N-terminal catalytic domain; Amino acids 6-168 of SEQ ID NO 6
LDAGHCTSGADTGAQGNGYKEQDLTRQVVTYLSEYLEKEGHTTKYCHCNSASTVNESLRYRVNKANSIGVDYFVSIHLNAGGGVGTETYICARGGEAERVAKRVNSKLVQYGYRDRGVKVGNLYVIKNTNAPAILVEICFIDSSSDIAKFNAKAIAKAIAEGL

SEQ ID NO: 18 - protein, 149 aa, Artificial Sequence
  N-terminal catalytic domain; Amino acids 6-154 of SEQ ID NO 6
LDAGHCTSGADTGAQGNGYKEQDLTRQVVTYLSEYLEKEGHTTKYCHCNSASTVNESLRYRVNKANSIGVDYFVSIHLNAGGGVGTETYICARGGEAERVAKRVNSKLVQYGYRDRGVKVGNLYVIKNTNAPAILVEICFIDSSSDIAK

SEQ ID NO: 19 - protein, 168 aa, Artificial Sequence
  N-terminal catalytic domain; Amino acids 3-170 of SEQ ID NO 7
GIRDGHSPNCKGAIGLRDEQSCMRVLCKEVIEILEKHGHEVVYCGSDASTQNSELSEGVRKANNSNVDIFISLHMNSFNGQAQGTESLIAVGARNSIKEIASRLCKNFASLGLVNRGVKEVNLYEMKNVKAPNIIFETMFCDNEHDINEVWSPTPYEKMALLIANAID

SEQ ID NO: 20 - protein, 122 aa, Artificial Sequence
  N-terminal catalytic domain; Amino acids 16-137 of SEQ ID NO 8
NNPKHIIIHETDNTSKGAGAETHCKAQANGNIGKASVHYYVDDTGVYQAVEHKHATWNCGDGNNRYGINNKNTISIEICVNSDSDYNKAVDNTVELVRYLKNGYYSNCQVVRHYDASRKNCP

SEQ ID NO: 21 - protein, 116 aa, Artificial Sequence
  N-terminal catalytic domain; Amino acids 16-131 of SEQ ID NO 9
NNPNKIIIHNADHPNCSVYDIDKWHKGNGWSGIGYHYFIRKDGSIWTGRPENAIGAHTIGQNSSSIGICLEGALMREKPTRAQLNSIYELIADVRKRRGNLPVYGHKDFNNTDCPG

SEQ ID NO: 22 - protein, 166 aa, Artificial Sequence
  N-terminal catalytic domain; Amino acids 7-172 of SEQ ID NO 10
GHNFQATGAVGLINETVEDRKVLAAAYKYTKAAGHDVLNVTPGNCDANTDLILGVNKAESFGAELFLSFHFDKCYDEYNGALGVACWICEEGGKAEKYARSIVNTIVAGTGLINRGVKVNPRLYELRKTSMPAVIVEVCFCEATEDVRIYKEKGPDLIGKLIAEGV

SEQ ID NO: 23 - protein, 169 aa, Artificial Sequence
  N-terminal catalytic domain; Amino acids 11-179 of SEQ ID NO 11
IDISNWQKGINLNQLKERGYDVCYIKITEGKGYVDPCFEENYNKAIAAGMKVGVYHYWRGTSSAIEQANNIVRTLGNKHIDCKIAIDVEQTDGLSYGELNNSVLQLAEELERLIGAEVCIYCNTNYARNVLDSRLGKYSLWVAHYGVNKPGDNPIWDKWAGFQYSENGT

SEQ ID NO: 24 - protein, 32 aa, Artificial Sequence
  Linker sequence; Amino acids 186-217 of SEQ ID NO 1
GTNSVDMNYCYEDFLKKDFTLDNATTKNVSTK

SEQ ID NO: 25 - protein, 13 aa, Artificial Sequence
  Linker sequence; Amino acids 271-283 of SEQ ID NO 1
KLQMATTHNVSTF

SEQ ID NO: 26 - protein, 33 aa, Artificial Sequence
  Linker sequence; Amino acids 133-165 of SEQ ID NO 2
NLDLDKLRRDLNNEVVDTNDDEYRENATVVNVS

SEQ ID NO: 27 - protein, 32 aa, Artificial Sequence
  Linker sequence; Amino acids 133-164 of SEQ ID NO 3
NLDLNKLRTDVNNKVVDSNGGYTENATVVNVN

SEQ ID NO: 28 - protein, 33 aa, Artificial Sequence
  Linker sequence; Amino acids 187-219 of SEQ ID NO 5
NGGCDMNEFTEEIFIDSSNFNLDNATTKNVSTK

SEQ ID NO: 29 - protein, 27 aa, Artificial Sequence
  Linker sequence; Amino acids 259-285 of SEQ ID NO 5
YNGVVGYVNADYVEKLQMATTYNVSTF

SEQ ID NO: 30 - protein, 14 aa, Artificial Sequence
  Linker sequence; Amino acids 155-168 of SEQ ID NO 6
FNAKAIAKAIAEGL

SEQ ID NO: 31 - protein, 195 aa, Artificial Sequence
  Linker sequence; Amino acids 138-332 of SEQ ID NO 8
RRILANGYWNTFLERVNSKDSSSQTPANTSYKGFYESSETRTNATLVGEGSIKVLDEECNHVSGRWIDSLDRLFVIGIYPSRKFIEVVYPAGDKKYHAYIGIEYYNRILFDYHKEYINDDGVTYVWWNASDVNVKDHNEELQPNQKASPMYRTGEWLRITFYRENGIPSDGYVRYEGSQNKKFYENIQYGIVKVN

SEQ ID NO: 32 - protein, 189 aa, Artificial Sequence
  Linker sequence; Amino acids 132-320 of SEQ ID NO 9
KNFPLSEFKNNSYRPTGVSSETVVSENGFYTSNEERTNATIVGVGDIEVLDEKGKVIQGRHISSLDRVFVLGIYPSRNHIELIYPGKDEKYHAYISIENYSRLSFDYHMQYKNDDGVTYVWWDSKNVNVKEHDEELQPHQKASPMYRTGGWLRITFYREDGTPSDGYVRYEGEQKERFYRKGKVVNVRT

SEQ ID NO: 33 - protein, 53 aa, Artificial Sequence
  C-terminal binding domain; Amino acids 218-270 of SEQ ID NO 1
LNIRAKGTTNSRVVGSIPANEKFKIKWVDEDYLGWYYIEYNGIVGYVNADYVE

SEQ ID NO: 34 - protein, 53 aa, Artificial Sequence
  C-terminal binding domain; Amino acids 284-336 of SEQ ID NO 1
LNVREEGSLNSRIVDKINVGDIFRIDWVDSDFIGWYRVTTKNGKVGFVNAEFV

SEQ ID NO: 35 - protein, 119 aa, Artificial Sequence
  C-terminal binding domain; Amino acids 218-336 of SEQ ID NO 1
LNIRAKGTTNSRVVGSIPANEKFKIKWVDEDYLGWYYIEYNGIVGYVNADYVEKLQMATTHNVSTFLNVREEGSLNSRIVDKINVGDIFRIDWVDSDFIGWYRVTTKNGKVGFVNAEFV

SEQ ID NO: 36 - protein, 59 aa, Artificial Sequence
  C-terminal binding domain; Amino acids 166-224 of SEQ ID NO 2
SYLNVRSKPSDEIIGKLFPNERLQVNWVDSDYLGWYYVTYRVNGTNKLKNGYVSAKYIK

SEQ ID NO: 37 - protein, 59 aa, Artificial Sequence
  C-terminal binding domain; Amino acids 165-223 of SEQ ID NO 3
SYLNVRSKPSDEIIGKLFPNERIQVNWVDSNYLGWYYITYRVNETNKLKSGYVSAKYIK

SEQ ID NO: 38 - protein, 39 aa, Artificial Sequence
  C-terminal binding domain; Amino acids 220-258 of SEQ ID NO 5
LNIRAKGTTNSKVIGSIPADETFKIKWVDEDYLGWYYVE

SEQ ID NO: 39 - protein, 53 aa, Artificial Sequence
  C-terminal binding domain; Amino acids 286-338 of SEQ ID NO 5
LNVREEGSLNSRIVDKINSGDIFRIDWVDSDFIGWYRITTKNGKVGFVNAEFV

SEQ ID NO: 40 - protein, 119 aa, Artificial Sequence
  C-terminal binding domain; Amino acids 220-338 of SEQ ID NO 5
LNIRAKGTTNSKVIGSIPADETFKIKWVDEDYLGWYYVEYNGVVGYVNADYVEKLQMATTYNVSTFLNVREEGSLNSRIVDKINSGDIFRIDWVDSDFIGWYRITTKNGKVGFVNAEFV

SEQ ID NO: 41 - protein, 70 aa, Artificial Sequence
  C-terminal binding domain; Amino acids 155-224 of SEQ ID NO 6
FNAKAIAKAIAEGLLDKTIDEVEKKPESVPDNTENSNTYFRVVVGSYKDRENAVKKQEELKAKGEDSFLL

SEQ ID NO: 42 - protein, 56 aa, Artificial Sequence
  C-terminal binding domain; Amino acids 169-224 of SEQ ID NO 6
LDKTIDEVEKKPESVPDNTENSNTYFRVVVGSYKDRENAVKKQEELKAKGEDSFLL

SEQ ID NO: 43 - protein, 54 aa, Artificial Sequence
  C-terminal binding domain; Amino acids 333-386 of SEQ ID NO 8
SSLNVRENPNGEVIGSVYKDEKVQVLKEENGWCYIEYSTSKGEKRGYVSSKYIE

SEQ ID NO: 44 - protein, 55 aa, Artificial Sequence
  C-terminal binding domain; Amino acids 321-375 of SEQ ID NO 9
SLTVRSGAGTNYSAIGSLEPNENVDILGKAEGWYYIEYNTSNGKKKGYVSEKYIE

SEQ ID NO: 45 - DNA, 34 bp, Artificial Sequence
  O_53 primer
gtaggtatgattagctttactaagctagtcattg

SEQ ID NO: 46 - DNA, 35 bp, Artificial Sequence
  O_172 primer
aaatagtaacatactaaagcggatgtaactcaatc

SEQ ID NO: 47 - DNA, 34 bp, Artificial Sequence
  O_328 primer
cacttttacaacaaagtacattaggaaaaacacg

SEQ ID NO: 48 - protein, 8 aa, Artificial Sequence
  N-terminal FLAG
DYKDDDDK

SEQ ID NO: 49 - protein, 9 aa, Artificial Sequence
  C-terminal HA
YPYDVPDYA

SEQ ID NO: 50 - protein, 8 aa, Artificial Sequence
  C-terminal Strep
WSHPQFEK

SEQ ID NO: 51 - protein, 6 aa, Artificial Sequence
  C-terminal 6xHIS
HHHHHH

SEQ ID NO: 52 - protein, 6 aa, Artificial Sequence
  Flexible linker
GSAGSG

SEQ ID NO: 53 - DNA, 57 bp, Artificial Sequence
  O_86 primer
tataagaaggagatatacatatgtatatcaatcagtccaacattaagttcaatggtc

SEQ ID NO: 54 - DNA, 58 bp, Artificial Sequence
  O_211 primer
gtttctttaccagactcgagttattatgcgtaatctggaacatcatagggataaccac

SEQ ID NO: 55 - DNA, 59 bp, Artificial Sequence
  O_112 primer
tataagaaggagatatacatatgaaaattatccaatccaatattcattttaatgggaac

SEQ ID NO: 56 - DNA, 56 bp, Artificial Sequence
  O_231 primer
gtttctttaccagactcgagttattaagcgtaatctggtacgtcataaggataacc

SEQ ID NO: 57 - DNA, 64 bp, Artificial Sequence
  O_115 primer
tataagaaggagatatacatatgaaaattatccagtcaaatatttactttaatgggaataaagc

SEQ ID NO: 58 - DNA, 56 bp, Artificial Sequence
  O_234 primer
gtttctttaccagactcgagttattaagcgtaatctggaacgtcataaggatatcc

SEQ ID NO: 59 - DNA, 48 bp, Artificial Sequence
  O_105
tttataggtctcaacatatgtcgaaaattttcggtttagacgcaggtc

SEQ ID NO: 60 - DNA, 61 bp, Artificial Sequence
  O_215 primer
gtttctttaccagactcgagttattatttttcaaattgtggatgtgatcatcctgaacctg

SEQ ID NO: 61 - DNA, 43 bp, Artificial Sequence
  O_92 primer
tataagaaggagatatacatatgaaaatttcagaacgtggtgg

SEQ ID NO: 62 - DNA, 59 bp, Artificial Sequence
  O_212 primer
gtttctttaccagactcgagttattacttttcaaattgaggatgtgaccaacctgaacc

SEQ ID NO: 63 - DNA, 54 bp, Artificial Sequence
  O_93 primer
tataagaaggagatatacatatgaaaattgctgttcgaggtggccataatttcc

SEQ ID NO: 64 - DNA, 61 bp, Artificial Sequence
  O_213 primer
gtttctttaccagactcgagttattatttttcaaattgtggatgactccaacctgaacctg

SEQ ID NO: 65 - DNA, 52 bp, Artificial Sequence
  O_75 primer
tataagaaggagatatacatatgaaaatcggtattagagatggtcatagtcc

SEQ ID NO: 66 - DNA, 55 bp, Artificial Sequence
  O_195 primer
gtttctttaccagactcgagttattatttttcgaattgtggatgactccaacctg

SEQ ID NO: 67 - DNA, 58 bp, Artificial Sequence
  O_82 primer
tataagaaggagatatacatatggactataaagatgatgatgacaaaatgcaatcacg

SEQ ID NO: 68 - DNA, 56 bp, Artificial Sequence
  O_205 primer
gtttctttaccagactcgagttattagcgctctaagtatttagcagtaaaccatgc

SEQ ID NO: 69 - DNA, 61 bp, Artificial Sequence
  O_95 primer
tataagaaggagatatacatatggactataaagacgatgatgataaaatgacaaaattacg

SEQ ID NO: 70 - DNA, 68 bp, Artificial Sequence
  O_216 primer
gtttctttaccagactcgagttattataatttcttaacgaattcagcatttacaaagcctactttgcc

SEQ ID NO: 71 - DNA, 51 bp, Artificial Sequence
  O_74 primer
tataagaaggagatatacatatggattacaaagacgatgacgacaaaatgc

SEQ ID NO: 72 - DNA, 58 bp, Artificial Sequence
  O_232 primer
gtttctttaccagactcgagttattatagctttttcacgaattctgcgttaacaaacc

SEQ ID NO: 73 - DNA, 23 bp, Artificial Sequence
  O_341 primer
acctgaaccagcagaaccgtctt

SEQ ID NO: 74 - DNA, 49 bp, Artificial Sequence
  O_351 primer
caccatcatcatcatcattaataactcgagtctggtaaagaaaccgctg

SEQ ID NO: 75 - DNA, 31 bp, Artificial Sequence
  O_342 primer
tccgctaccagcagaaccatcttttttaatg

SEQ ID NO: 76 - DNA, 59 bp, Artificial Sequence
  O_81 primer
tataagaaggagatatacatatgaaaataaacaagcgtttatcaactacgaatgtaacc

SEQ ID NO: 77 - DNA, 71 bp, Artificial Sequence
  O_434 primer
gtttctttaccagactcgagttattaatgatgatgatgatggtgacctgaaccagcagaaccaactaattc

SEQ ID NO: 78 - DNA, 37 bp, Artificial Sequence
  O_323 primer
aaatttaaattagcagaatctttgtcttgattaggtg

SEQ ID NO: 79 - DNA, 40 bp, Artificial Sequence
  O_336 primer
caccctagtaaagtaaataaaatatacaactcaatgaatc

SEQ ID NO: 80 - DNA, 52 bp, Artificial Sequence
  O_197 primer
aatataggtctcaattaatcttttttaatgtacttggcagaaacgtaacctg

SEQ ID NO: 81 - DNA, 48 bp, Artificial Sequence
  O_106 primer
tttataggtctcaatgaaaattatccagtcaaatatttactttaatgg